(19) United States
(12) Patent Application Publication
(10) Pub. No.: US 2024/0417470 A1
(43) Pub. Date: Dec. 19, 2024

(54) FUSIONS WITH CD8 ANTIGEN BINDING MOLECULES FOR MODULATING IMMUNE CELL FUNCTION

(71) Applicant: Asher Biotherapeutics, Inc., South San Francisco, CA (US)

(72) Inventors: Yik Andy YEUNG, South San Francisco, CA (US); Ivana DJURETIC, Pacifica, CA (US); Paul BESSETTE, San Francisco, CA (US); Wei CHEN, Cupertino, CA (US); Sherman Michael CHIN, Burlingame, CA (US); Kelly Dare MOYNIHAN, San Francisco, CA (US); Henry C. NGUYEN, San Francisco, CA (US); Irene NI, Foster City, CA (US); Danielle C. PAPPAS, San Mateo, CA (US); Terrence PARK, Hayward, CA (US)

(21) Appl. No.: 18/662,910

(22) Filed: May 13, 2024

Related U.S. Application Data
(62) Division of application No. 17/508,804, filed on Oct. 22, 2021, now Pat. No. 12,024,559.
(60) Provisional application No. 63/190,669, filed on May 19, 2021.
(60) Provisional application No. 63/121,663, filed on Dec. 4, 2020.
(60) Provisional application No. 63/105,162, filed on Oct. 23, 2020.

(51) Int. Cl. C07K 16/28; A61K 38/20; A61K 47/65; A61K 47/68; A61P 31/12; A61P 35/00

(52) U.S. Cl. CPC C07K 16/2815; A61K 38/2013; A61K 47/65; A61K 47/6849; A61P 31/12; A61P 35/00

(57) ABSTRACT

Provided herein are anti-CD8 antigen binding molecules and fusion polypeptides comprising the CD8 antigen binding molecules for selectively modulating the function of CD8+ T cells over other immune cells. In addition, the present disclosure also provides polynucleotides encoding the disclosed antigen binding molecules and fusion polypeptides, and vectors and host cells comprising such polynucleotides. The present disclosure further provides methods for producing the antigen binding molecules and fusion polypeptides, pharmaceutical compositions comprising the same, and uses thereof.

### CROSS-REFERENCE TO RELATED APPLICATIONS

This application claims the priority benefit of U.S. Provisional Application Nos. 63/105,162, filed Oct. 23, 2020; 63/121,663, filed Dec. 4, 2020; and 63/190,669, filed May 19, 2021; each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 182842000400SEQLIST.TXT, date recorded: Oct. 21, 2021, size: 557,283 bytes).

### FIELD

The present disclosure discloses CD8 antigen binding molecules and fusion polypeptides comprising the CD8 antigen binding molecules for selectively modulating the function of CD8+ T cells over other immune cells. In addition, the present disclosure also provides polynucleotides encoding the disclosed antigen binding molecules and fusion polypeptides, and vectors and host cells comprising such polynucleotides. The present disclosure further provides methods for producing the antigen binding molecules and fusion polypeptides, pharmaceutical compositions comprising the same, and uses thereof.

### BACKGROUND

CD8+ T cells expressing alpha beta T cell receptors are a large subset of major histocompatibility (MHC) class I-restricted T cells that mediate adaptive immunity to various pathogens and cancers. In addition, they can also be pathogenic and cause disease in certain autoimmune and inflammatory conditions. Modulating the function of CD8+ T cells, either by activating their function in the context of infection and cancer, or by inhibiting their function in the context of certain autoimmune diseases, can have therapeutic benefits.

CD8+ T cell activation and differentiation is in large part controlled by soluble immunomodulatory proteins such as cytokines. Biological activity of cytokines is mediated by binding to their respective cytokine receptors on the cell surface, typically with very high affinity, resulting in their ability to potently stimulate signal transduction downstream of their receptors triggering various cellular processes that regulate immune cell phenotype and function. Cytokines typically have pleiotropic effects, causing multiple downstream cellular events such as activation, proliferation, survival, apoptosis, and secretion of other immunomodulatory proteins. In addition, because their receptors are expressed on multiple immune cell subsets, cytokines act not only on CD8+ T cells but also on other immune and non-immune cells that express their receptors.

For example, interleukin-2 (IL-2) is a cytokine that regulates many lymphocyte subsets, including alpha beta CD4+ and CD8 T+ cells, and various innate and innate-like lymphocytes such as NK cells, NK T cells, gamma delta T cells (Tγδ) cells, and innate lymphoid cells (ILC1, ILC2, and ILC3 cells).

IL-2 can signal by binding with an intermediate affinity to a receptor complex consisting of IL-2Rβ and IL-2Rγ subunits (IL-2Rβγ, intermediate affinity receptor), both of which are required and sufficient to trigger downstream signaling in immune cells. In addition, IL-2 binds with high affinity to a receptor complex consisting of IL-2Rα, IL-2Rβ, and IL-2Rγ subunits (IL-2Rαβγ, high affinity receptor) (Stauber et al, Proc Natl Acad Sci USA. 2006 Feb. 21;103 (8): 2788-93). IL-2Rα expression is restricted to CD4+ Treg cells, activated T lymphocytes, and ILC2 and ILC3 cells, making these subsets the most sensitive to IL-2 signaling. IL-2Rβ and IL-2Rγ subunits are shared with another related cytokine, IL-15, and IL-2Rγ subunit is shared among other common gamma chain cytokines (IL-4, IL-7, IL-9, and IL-21). Most innate and innate-like lymphocytes including NK cells, NK T cells, Tγ8 cells, and ILC1, ILC2, and ILC3 cells express high levels of IL-2Rβ (ImmGen consortium; Heng TS et al, Immunological Genome Project Consortium. Nat Immunol. 2008 October;9 (10): 1091-4), which also makes them sensitive to both IL-2 and IL-15 cytokines.

Binding of IL-2 to its receptor induces the phosphorylation of receptor-associated Janus kinases, JAK3 and JAK1, which promote the phosphorylation of STAT5 transcription factor (pSTAT5) that regulates transcription of many genes in lymphocytes. IL-2 signaling in lymphocytes promotes cell survival, proliferation, and increased effector function, including pro-inflammatory cytokine secretion and cytotoxic function, and in some cases, activation-induced cell death (reviewed in Ross & Cantrell, Annu Rev Immunol. 2018 Apr. 26; 36:411-433).

CD8+ T cells express CD8, which is a type I transmembrane glycoprotein found on the cell surface as a CD8 alpha (CD8a, CD8a) homodimer and CD8 alpha-CD8 beta (CD8B, CD8b) heterodimer. CD8 dimers interact with the MHC class I molecules on target cells and this interaction keeps the TCR closely engaged with MHC during CD8+ T cell activation. The cytoplasmic tail of CD8a contains binding sites for a T cell kinase (Lck) that initiates signal transduction downstream of the TCR during T cell activation, while the role of CD8B is thought to be in increasing the avidity of CD8 binding to MHC class I and influencing specificity of the CD8/MHC/TCR interaction (Bosselut et al, Immunity. 2000 April; 12 (4): 409-18). TCR alpha beta-expressing CD8+ T cells typically express both CD8ab and CD8aa dimers, however high levels of CD8aa but not CD8ab dimers can be found on some innate lymphocytes such as NK cells, mucosal-associated invariant T (MAIT) cells, and gamma delta T cells.

Accordingly, there is a need for antibodies that selectively bind to CD8ab heterodimers over CD8aa homodimers. These antibodies can be used, e.g., for targeting certain immunomodulatory polypeptides, such as IL-2, to CD8ab+ T cells, and limiting their activity on CD8-immune cells and CD8aa+ immune cells such as NK cells.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

The present disclosure describes, inter alia, human or humanized antibodies, antigen binding fragments, and fusion proteins that bind human CD8b. In some embodiments, the human or humanized antibodies, antigen binding fragments, and fusion proteins bind human CD8b or a human CD8ab heterodimer preferentially over binding to a human CD8aa homodimer In some embodiments, the human or humanized antibodies, antigen binding fragments, and fusion proteins bind CD8b, CD8ab, or both.

Certain aspects of the present disclosure relate to human or humanized antibodies or antigen binding fragments thereof. In some embodiments, the antibody or fragment specifically binds human CD8b and/or human CD8ab with at least 10-fold higher affinity than its binding to human CD8a and/or human CD8aa. In some embodiments, the antibody or fragment specifically binds the extracellular domain(s) of human CD8b and/or human CD8ab with at least 10-fold higher affinity than its binding to the extracellular domain(s) of human CD8a and/or human CD8aa. In some embodiments, the antibody or fragment binds to a cell expressing a human CD8ab heterodimer on its surface with an EC50 that is less than 1000 nM. In some embodiments, the antibody or fragment binds human CD8+ T cells. In some embodiments, the antibody or fragment specifically binds human CD8b and/or human CD8ab with at least 10-fold higher affinity than its binding to human CD8a and/or human CD8aa expressed on the surface of an natural killer (NK) cell. In some embodiments, the antibody or fragment specifically binds a cell expressing human CD8b and/or human CD8ab on its surface (e.g., a T cell) with at least 10-fold higher affinity than its binding to a cell expressing human CD8a and/or human CD8aa on its surface (e.g., an NK cell).

In some embodiments, the antibody or fragment comprises a heavy chain variable (VH) domain and a light chain variable (VL) domain; wherein the VH domain comprises a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO:15; and wherein the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:18. In some embodiments, the antibody or fragment comprises a heavy chain variable (VH) domain and a light chain variable (VL) domain; wherein the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:15; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18. In some embodiments, the VH domain comprises an amino acid sequence that is at least 90%, at least 95%, at least 99%, or 100% identical to the sequence of SEQ ID NO:62, and wherein the VL domain comprises an amino acid sequence that is at least 90%, at least 95%, at least 99%, or 100% identical to the sequence of SEQ ID NO:63. In some embodiments, the antibody or fragment comprises a heavy chain variable (VH) domain and a light chain variable (VL) domain; wherein the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In some embodiments, the antibody or fragment comprises a heavy chain variable (VH) domain and a light chain variable (VL) domain; wherein the VH domain comprises a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO: 24. In some embodiments, the VH domain comprises an amino acid sequence that is at least 90%, at least 95%, at least 99%, or 100% identical to the sequence of SEQ ID NO:64, and wherein the VL domain comprises an amino acid sequence that is at least 90%, at least 95%, at least 99%, or 100% identical to the sequence of SEQ ID NO:65. In some embodiments, the antibody or fragment comprises a heavy chain variable (VH) domain and a light chain variable (VL) domain; wherein the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30. In some embodiments, the antibody or fragment comprises a heavy chain variable (VH) domain and a light chain variable (VL) domain; wherein the VH domain comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30. In some embodiments, the VH domain comprises an amino acid sequence that is at least 90%, at least 95%, at least 99%, or 100% identical to the sequence of SEQ ID NO:66, and wherein the VL domain comprises an amino acid sequence that is at least 90%, at least 95%, at least 99%, or 100% identical to the sequence of SEQ ID NO:67. In some embodiments, the antibody or fragment comprises a heavy chain variable (VH) domain and a light chain variable (VL) domain; wherein the VH domain comprises a CDR-H1 comprising the amino acid sequence of SEQ ID NO: 31, a CDR-H2 comprising the amino acid sequence of SEQ ID NO:32, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, the antibody or fragment comprises a heavy chain variable (VH) domain and a light chain variable (VL) domain; wherein the VH domain comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, the VH domain comprises an amino acid sequence that is at least 90%, at least 95%, at least 99%, or 100% identical to the sequence of SEQ ID NO:68, and wherein the VL domain comprises an amino acid sequence that is at least 90%, at least 95%, at least 99%, or 100% identical to the sequence of SEQ ID NO:69. In some embodiments, the antibody or fragment comprises a heavy chain variable (VH) domain and a light chain variable (VL) domain; wherein the VH domain comprises a CDR-H1 comprising the amino acid sequence of SEQ ID NO: 37, a CDR-H2 comprising the amino acid sequence of SEQ ID NO:38, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the antibody or fragment comprises a heavy chain variable (VH) domain and a light chain variable (VL) domain; wherein the VH domain comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:70, and wherein the VL domain comprises an amino acid sequence that is at least 90%, at least 95%, at least 99%, or 100% identical to the sequence of SEQ ID NO:71. In some embodiments, the antibody or fragment comprises a heavy chain variable (VH) domain and a light chain variable (VL) domain; wherein the VH domain comprises a CDR-H1 comprising the amino acid sequence of SEQ ID NO: 43, a CDR-H2 comprising the amino acid sequence of SEQ ID NO:44, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In some embodiments, the antibody or fragment comprises a heavy chain variable (VH) domain and a light chain variable (VL) domain; wherein the VH domain comprises a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In some embodiments, the VH domain comprises an amino acid sequence that is at least 90%, at least 95%, at least 99%, or 100% identical to the sequence of SEQ ID NO:72, and wherein the VL domain comprises an amino acid sequence that is at least 90%, at least 95%, at least 99%, or 100% identical to the sequence of SEQ ID NO:73. In some embodiments, the antibody or fragment comprises a heavy chain variable (VH) domain and a light chain variable (VL) domain; wherein the VH domain comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO:3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO: 6. In some embodiments, the antibody or fragment comprises a heavy chain variable (VH) domain and a light chain variable (VL) domain; wherein the VH domain comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO: 3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, the antibody or fragment comprises a heavy chain variable (VH) domain and a light chain variable (VL) domain; wherein the VH domain comprises a CDR-H1 comprising the amino acid sequence of SEQ ID NO: 177, a CDR-H2 comprising the amino acid sequence of SEQ ID NO:178, and a CDR-H3 comprising the amino acid sequence of SEQ ID NO:179; and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182. In some embodiments, the antibody or fragment comprises a heavy chain variable (VH) domain and a light chain variable (VL) domain; wherein the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182. In some embodiments, the VH domain comprises an amino acid sequence that is at least 90%, at least 95%, at least 99%, or 100% identical to the sequence of SEQ ID NO: 185, and wherein the VL domain comprises an amino acid sequence that is at least 90%, at least 95%, at least 99%, or 100% identical to the sequence of SEQ ID NO:186.

In some embodiments, the antibody or fragment comprises a heavy chain variable (VH) domain and a light chain variable (VL) domain; wherein the VH domain comprises a CDR-H1 comprising the amino acid sequence of X₁X₂AIS, wherein X₁ is S, K, G, N, R, D, T, or G, and wherein X₂ is Y, L, H, or F (SEQ ID NO:259), a CDR-H2 comprising the amino acid sequence of X₁X₂X₃PX₄X₅X₆X₇X₈X₉YX₁₀QKFX1G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is Nor K, X₁₀ is A or N, and Xn is Q or T (SEQ ID NO:260), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:261); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO: 262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO: 263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the antibody or fragment comprises a heavy chain variable (VH) domain and a light chain variable (VL) domain; wherein the VH domain comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO: 228. In some embodiments, the VH domain comprises an amino acid sequence that is at least 90%, at least 95%, at least 99%, or 100% identical to the sequence of SEQ ID NO:245, and wherein the VL domain comprises an amino acid sequence that is at least 90%, at least 95%, at least 99%, or 100% identical to the sequence of SEQ ID NO:246. In some embodiments, the antibody or fragment comprises a heavy chain variable (VH) domain and a light chain variable (VL) domain; wherein the VH domain comprises a CDR-H1 comprising the amino acid sequence of SEQ ID NO:225, a CDR-H2 comprising the amino acid sequence of SEQ ID NO: 232, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises an amino acid sequence that is at least 90%, at least 95%, at least 99%, or 100% identical to the sequence of SEQ ID NO:251, and wherein the VL domain comprises an amino acid sequence that is at least 90%, at least 95%, at least 99%, or 100% identical to the sequence of SEQ ID NO: 252. In some embodiments, the VH domain comprises the amino acid sequence of SEQ ID NO: 251; and the VL domain comprises the amino acid sequence of SEQ ID NO:252. In some embodiments, the antibody or fragment comprises a heavy chain variable (VH) domain and a light chain variable (VL) domain; wherein the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:253, and wherein the VL domain comprises an amino acid sequence that is at least 90%, at least 95%, at least 99%, or 100% identical to the sequence of SEQ ID NO:254. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, the antibody or fragment comprises a heavy chain variable (VH) domain and a light chain variable (VL) domain; wherein the VH domain comprises a CDR-H1 comprising the amino acid sequence of GX₁X₂FX3X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO:265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO:266), and a CDR-H3 comprising the amino acid sequence of X₁X₂X₃GX₄XsLFX6X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:267); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄XsPWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO: 264). In some embodiments, the VH domain comprises a CDR-H1 comprising the amino acid sequence of SEQ ID NO:238, a CDR-H2 comprising the amino acid sequence of SEQ ID NO: 239, and a CDR-H3 comprising the amino acid sequence of SEQ ID NO:233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:245; and wherein the VL domain comprises an amino acid sequence that is at least 90%, at least 95%, at least 99%, or 100% identical to the sequence of SEQ ID NO:246. In some embodiments, the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233; and the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises an amino acid sequence that is at least 90%, at least 95%, at least 99%, or 100% identical to the sequence of SEQ ID NO:251; and wherein the VL domain comprises an amino acid sequence that is at least 90%, at least 95%, at least 99%, or 100% identical to the sequence of SEQ ID NO:252. In some embodiments, the VH domain comprises the amino acid sequence of SEQ ID NO:251; and the VL domain comprises the amino acid sequence of SEQ ID NO:252. In some embodiments, the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:253; and wherein the VL domain comprises an amino acid sequence that is at least 90%, at least 95%, at least 99%, or 100% identical to the sequence of SEQ ID NO:254. In some embodiments, the VH domain further comprises a FW-1 comprising the sequence QVQLVQSGAEVKKPGSSVKVSCKAS

(SEQ ID NO:278), a FW-2 comprising the sequence AISWVRQAPGQGLEWMGGI (SEQ ID NO: 279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, the antibody or fragment comprises a heavy chain variable (VH) domain and a light chain variable (VL) domain; wherein the VH domain comprises a CDR-H1 comprising the amino acid sequence of X₁YX₂MS, wherein X₁ is S, D, E, A, or Q and X₂ is A, G, or T (SEQ ID NO:268), a CDR-H2 comprising the amino acid sequence of DIX1X₂X₃GX₄XsTX6 YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO:269), and a CDR-H3 comprising the amino acid sequence of X₁X₂X₃YX₄WX5X₆AX7DX8, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270) and a VL domain comprising a CDR-L1 comprising the amino acid sequence of RASQSVSSNLA (SEQ ID NO: 40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the antibody or fragment comprises a heavy chain variable (VH) domain and a light chain variable (VL) domain; wherein the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:247, and wherein the VL domain comprises an amino acid sequence that is at least 90%, at least 95%, at least 99%, or 100% identical to the sequence of SEQ ID NO:248. In some embodiments, the VH domain comprises an amino acid sequence that is at least 90%, at least 95%, at least 99%, or 100% identical to the sequence of SEQ ID NO: 249, and wherein the VL domain comprises an amino acid sequence that is at least 90%, at least 95%, at least 99%, or 100% identical to the sequence of SEQ ID NO:250. In some embodiments, the VH domain comprises the amino acid sequence of SEQ ID NO:249, and wherein the VL domain comprises the amino acid sequence of SEQ ID NO:250. In some embodiments, the antibody or fragment comprises a heavy chain variable (VH) domain and a light chain variable (VL) domain; wherein the VH domain comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:255, and wherein the VL domain comprises an amino acid sequence that is at least 90%, at least 95%, at least 99%, or 100% identical to the sequence of SEQ ID NO:256. In some embodiments, the VH domain comprises an amino acid sequence that is at least 90%, at least 95%, at least 99%, or 100% identical to the sequence of SEQ ID NO: 257, and wherein the VL domain comprises an amino acid sequence that is at least 90%, at least 95%, at least 99%, or 100% identical to the sequence of SEQ ID NO:258. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, the antibody or fragment comprises a heavy chain variable (VH) domain and a light chain variable (VL) domain; wherein the VH domain comprises a CDR-H1 comprising the amino acid sequence of GFTFX₁X₂Y, wherein X₁ is S, D, E, Q, S, or A and X₂ is S, D, E, A, or Q (SEQ ID NO:271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO:272), and a CDR-H3 comprising the amino acid sequence of X₁X₂X₃YX₄WX5X₆AX-DX8, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:273); and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42. In some embodiments, the VH domain comprises an amino acid sequence that is at least 90%, at least 95%, at least 99%, or 100% identical to the sequence of SEQ ID NO:247; and wherein the VL domain comprises an amino acid sequence that is at least 90%, at least 95%, at least 99%, or 100% identical to the sequence of SEQ ID NO:248. In some embodiments, the VH domain comprises an amino acid sequence that is at least 90%, at least 95%, at least 99%, or 100% identical to the sequence of SEQ ID NO:249; and wherein the VL domain comprises an amino acid sequence that is at least 90%, at least 95%, at least 99%, or 100% identical to the sequence of SEQ ID NO:250. In some embodiments, the VH domain comprises the amino acid sequence of SEQ ID NO:249, and wherein the VL domain comprises the amino acid sequence of SEQ ID NO:250. In some embodiments, the VH domain comprises a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:255; and wherein the VL domain comprises an amino acid sequence that is at least 90%, at least 95%, at least 99%, or 100% identical to the sequence of SEQ ID NO:256. In some embodiments, the VH domain comprises an amino acid sequence that is at least 90%, at least 95%, at least 99%, or 100% identical to the sequence of SEQ ID NO: 257; and wherein the VL domain comprises an amino acid sequence that is at least 90%, at least 95%, at least 99%, or 100% identical to the sequence of SEQ ID NO:258. In some embodiments, the VH domain further comprises a FW-1 comprising the sequence EVOLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and a FW-4 comprising the sequence FGQGTKVEIK (SEQ ID NO:296).

In some embodiments according to any of the embodiments described herein, the antibody is a multispecific antibody (e.g., a bispecific antibody).

Further provided herein are fusion proteins comprising a first moiety comprising the antibody or fragment of any one of the above embodiments and a second moiety comprising a cytokine, chemokine, or growth factor. In some embodiments, the first moiety is fused to the second moiety directly or via a linker. In some embodiments, the first moiety comprises a human or humanized antibody or antigen-binding fragment thereof that specifically binds CD8b and/or CD8ab, wherein the antibody or fragment comprises a heavy chain variable (VH) domain and a light chain variable (VL) domain, wherein: the VH domain comprises a CDR-H1 comprising the amino acid sequence of SEQ ID NO: 13, a CDR-H2 comprising the amino acid sequence of SEQ ID NO:14, and a CDR-H3 comprising the amino acid sequence of SEQ ID NO: 15; and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18; the VH domain comprises a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30; the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36; the VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; and wherein the VL domain comprises a CDR-L1 comprising the amino acid sequence of SEQ ID NO:4, a CDR-L2 comprising the amino acid sequence of SEQ ID NO: 5, and a CDR-L3 comprising the amino acid sequence of SEQ ID NO:6; the VH domain comprises a CDR-H1 comprising the amino acid sequence of SEQ ID NO:177, a CDR-H2 comprising the amino acid sequence of SEQ ID NO: 178, and a CDR-H3 comprising the amino acid sequence of SEQ ID NO: 179; and wherein the VL domain comprises a CDR-L1 comprising the amino acid sequence of SEQ ID NO: 180, a CDR-L2 comprising the amino acid sequence of SEQ ID NO:181, and a CDR-L3 comprising the amino acid sequence of SEQ ID NO: 182; the VH domain comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227; and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 228; the VH domain comprises a CDR-H1 comprising the amino acid sequence of SEQ ID NO: 229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42); the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO: 228; or the VH domain comprises a CDR-H1 comprising the amino acid sequence of SEQ ID NO: 229, a CDR-H2 comprising the amino acid sequence of SEQ ID NO:237, and a CDR-H3 comprising the amino acid sequence of SEQ ID NO:231, and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the first moiety comprises a human or humanized antibody or antigen-binding fragment thereof that specifically binds CD8b and/or CD8ab, wherein the antibody or fragment comprises a heavy chain variable (VH) domain and a light chain variable (VL) domain, wherein: the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15, and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18; the VH domain comprises a CDR-H1 comprising the amino acid sequence of SEQ ID NO: 53, a CDR-H2 comprising the amino acid sequence of SEQ ID NO:52, and a CDR-H3 comprising the amino acid sequence of SEQ ID NO:21, and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27, and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30; the VH domain comprises a CDR-H1 comprising the amino acid sequence of SEQ ID NO:54, a CDR-H2 comprising the amino acid sequence of SEQ ID NO: 52, and a CDR-H3 comprising the amino acid sequence of SEQ ID NO:33, and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36; the VH domain comprises a CDR-H1 comprising the amino acid sequence of SEQ ID NO:55, a CDR-H2 comprising the amino acid sequence of SEQ ID NO: 56, and a CDR-H3 comprising the amino acid sequence of SEQ ID NO:39, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:55, a CDR-H2 comprising the amino acid sequence of SEQ ID NO: 57, and a CDR-H3 comprising the amino acid sequence of SEQ ID NO:45, and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO:49, a CDR-H2 comprising the amino acid sequence of SEQ ID NO: 50, and a CDR-H3 comprising the amino acid sequence of SEQ ID NO:3, and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain comprises a CDR-H1 comprising the amino acid sequence of SEQ ID NO: 183, a CDR-H2 comprising the amino acid sequence of SEQ ID NO:184, and a CDR-H3 comprising the amino acid sequence of SEQ ID NO:179, and wherein the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182; the VH domain comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO: 233, and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228; the VH domain comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 228; or the VH domain comprises a CDR-H1 comprising the amino acid sequence of SEQ ID NO: 240, a CDR-H2 comprising the amino acid sequence of SEQ ID NO:244, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42.

In some embodiments according to any of the embodiments described herein (e.g., a fusion protein of the present disclosure), the second moiety induces activation of CD8+ T cells. In some embodiments, the fusion protein induces activation of cells expressing a human CD8ab heterodimer with at least 10-fold higher potency than activation of cells expressing a human CD8aa homodimer. In some embodiments, the fusion protein induces activation of CD8+ T cells with at least 10-fold higher potency than activation of NK cells. In some embodiments, potency of activation is measured by EC50, as assessed by cell proliferation. In some embodiments, the first moiety comprises two antibody heavy chain polypeptides comprising a structure according to formula [I], from N-terminus to C-terminus:

VH—CH1-hinge-CH2-CH3 [I]

and two antibody light chain polypeptides comprising a structure according to formula [II], from N-terminus to C-terminus:

VL-CL [II]

wherein VH is an antibody heavy chain variable (VH) domain, wherein CH1 is an antibody CH1 domain, wherein hinge is an antibody hinge domain, wherein CH2-CH3 is an antibody Fc domain, wherein VL is an antibody light chain variable (VL) domain, and wherein CL is an antibody constant light chain domain; and wherein the N-terminus of the second moiety is fused to the C-terminus of one of the two CH3 domains (e.g., via a linker of the present disclosure). In some embodiments, the first moiety comprises a first antibody heavy chain polypeptide comprising a structure according to formula [I], from N-terminus to C-terminus:

VH—CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], from N-terminus to C-terminus:

hinge-CH2-CH3 [III],

wherein VH is an antibody heavy chain variable (VH) domain, wherein CH1 is an antibody CH1 domain, wherein hinge is an antibody hinge domain, wherein CH2-CH3 is an antibody Fc domain, wherein VL is an antibody light chain variable (VL) domain, and wherein CL is an antibody constant light chain domain; and wherein the N-terminus of the second moiety is fused to the C-terminus of the CH3 domain of the second antibody heavy chain polypeptide (e.g., via a linker of the present disclosure). In some embodiments, the N-terminus of the second moiety is fused to the C-terminus of the CH3 domain of the first antibody heavy chain polypeptide (e.g., via a linker of the present disclosure). In some embodiments, the first moiety comprises a first antibody heavy chain polypeptide comprising a structure according to formula [I], from N-terminus to C-terminus:

VH—CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], from N-terminus to C-terminus:

hinge-CH2-CH3 [III],

wherein VH is an antibody heavy chain variable (VH) domain, wherein CH1 is an antibody CH1 domain, wherein hinge is an antibody hinge domain, wherein CH2-CH3 is an antibody Fc domain, wherein VL is an antibody light chain variable (VL) domain, and wherein CL is an antibody constant light chain domain; and wherein the C-terminus of the second moiety is fused to the N-terminus of the hinge domain of the second antibody heavy chain polypeptide (e.g., via a linker of the present disclosure). In some embodiments, the first moiety comprises one or two antibody heavy chain polypeptides and one or two antibody light chain polypeptides. In some embodiments, the first moiety comprises a single chain antibody or single chain variable fragment (scFv). In some embodiments, the first moiety comprises a VHH antibody. In some embodiments according to any of the embodiments described herein (e.g., the fusion proteins described above), VH and VL form an antigen binding site (e.g., that specifically binds CD8b and/or CD8ab). In some embodiments, the first moiety comprises a first antibody heavy chain polypeptide comprising a structure according to formula [I], from N-terminus to C-terminus:

VH—CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], from N-terminus to C-terminus:

hinge-CH2-CH3 [III],

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and wherein the N-terminus of the second moiety is fused to the C-terminus of the CH3 domain of the first antibody heavy chain polypeptide. In some embodiments, the VH domain of both antibody heavy chain polypeptides comprises a CDR-H1 comprising the amino acid sequence of SEQ ID NO: 13, a CDR-H2 comprising the amino acid sequence of SEQ ID NO:14, and a CDR-H3 comprising the amino acid sequence of SEQ ID NO:15, and wherein the VL domain of both antibody light chain polypeptides comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18; the VH domain of both antibody heavy chain polypeptides comprises a CDR-H1 comprising the amino acid sequence of SEQ ID NO: 19, a CDR-H2 comprising the amino acid sequence of SEQ ID NO:20, and a CDR-H3 comprising the amino acid sequence of SEQ ID NO:21, and wherein the VL domain of both antibody light chain polypeptides comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain of both antibody heavy chain polypeptides comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27, and wherein the VL domain of both antibody light chain polypeptides comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30; the VH domain of both antibody heavy chain polypeptides comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33, and wherein the VL domain of both antibody light chain polypeptides comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36; the VH domain of both antibody heavy chain polypeptides comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain of both antibody heavy chain polypeptides comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and wherein the VL domain of both antibody light chain polypeptides comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain of both antibody heavy chain polypeptides comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and wherein the VL domain of both antibody light chain polypeptides comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain of both antibody heavy chain polypeptides comprises a CDR-H1 comprising the amino acid sequence of SEQ ID NO:177, a CDR-H2 comprising the amino acid sequence of SEQ ID NO: 178, and a CDR-H3 comprising the amino acid sequence of SEQ ID NO:179, and wherein the VL domain of both antibody light chain polypeptides comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO:182; the VH domain of both antibody heavy chain polypeptides comprises a CDR-H1 comprising the amino acid sequence of SEQ ID NO: 225, a CDR-H2 comprising the amino acid sequence of SEQ ID NO:226, and a CDR-H3 comprising the amino acid sequence of SEQ ID NO:227, and wherein the VL domain of both antibody light chain polypeptides comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228; the VH domain of both antibody heavy chain polypeptides comprises a CDR-H1 comprising the amino acid sequence of SEQ ID NO: 229, a CDR-H2 comprising the amino acid sequence of SEQ ID NO:230, and a CDR-H3 comprising the amino acid sequence of SEQ ID NO:231, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain of both antibody heavy chain polypeptides comprises a CDR-H1 comprising the amino acid sequence of SEQ ID NO: 225, a CDR-H2 comprising the amino acid sequence of SEQ ID NO:232, and a CDR-H3 comprising the amino acid sequence of SEQ ID NO:233, and wherein the VL domain of both antibody light chain polypeptides comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain of both antibody heavy chain polypeptides comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233, and wherein the VL domain of both antibody light chain polypeptides comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228; or the VH domain of both antibody heavy chain polypeptides comprises a CDR-H1 comprising the amino acid sequence of SEQ ID NO:229, a CDR-H2 comprising the amino acid sequence of SEQ ID NO: 237, and a CDR-H3 comprising the amino acid sequence of SEQ ID NO:231, and wherein the VL domain of both antibody light chain polypeptides comprises a CDR-L1 comprising the amino acid sequence of SEQ ID NO:40, a CDR-L2 comprising the amino acid sequence of SEQ ID NO: 41, and a CDR-L3 comprising the amino acid sequence of SEQ ID NO:42. In some embodiments, the VH domain of both antibody heavy chain polypeptides comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15, and wherein the VL domain of both antibody light chain polypeptides comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18; the VH domain of both antibody heavy chain polypeptides comprises a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO: 21, and wherein the VL domain of both antibody light chain polypeptides comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO: 24; the VH domain of both antibody heavy chain polypeptides comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO: 27, and wherein the VL domain of both antibody light chain polypeptides comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO: 30; the VH domain of both antibody heavy chain polypeptides comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO: 33, and wherein the VL domain of both antibody light chain polypeptides comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO: 36; the VH domain of both antibody heavy chain polypeptides comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO: 39, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42; the VH domain of both antibody heavy chain polypeptides comprises a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO: 45, and wherein the VL domain of both antibody light chain polypeptides comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO: 48; the VH domain of both antibody heavy chain polypeptides comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO: 3, and wherein the VL domain of both antibody light chain polypeptides comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO: 6; the VH domain of both antibody heavy chain polypeptides comprises a CDR-H1 comprising the amino acid sequence of SEQ ID NO:183, a CDR-H2 comprising the amino acid sequence of SEQ ID NO: 184, and a CDR-H3 comprising the amino acid sequence of SEQ ID NO: 179, and wherein the VL domain of both antibody light chain polypeptides comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182; the VH domain of both antibody heavy chain polypeptides comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO: 233, and wherein the VL domain of both antibody light chain polypeptides comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228; the VH domain of both antibody heavy chain polypeptides comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO: 242, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain of both antibody heavy chain polypeptides comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO: 233, and wherein the VL domain of both antibody light chain polypeptides comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain of both antibody heavy chain polypeptides comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO: 233, and wherein the VL domain of both antibody light chain polypeptides comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228; or the VH domain of both antibody heavy chain polypeptides comprises a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises a CDR-H1 comprising the amino acid sequence of SEQ ID NO:13, a CDR-H2 comprising the amino acid sequence of SEQ ID NO: 14, and a CDR-H3 comprising the amino acid sequence of SEQ ID NO: 15, and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18; the VH domain comprises a CDR-H1 comprising the amino acid sequence of SEQ ID NO: 19, a CDR-H2 comprising the amino acid sequence of SEQ ID NO:20, and a CDR-H3 comprising the amino acid sequence of SEQ ID NO:21, and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27, and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30; the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33, and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36; the VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO:3, and wherein the VL domain comprises a CDR-L1 comprising the amino acid sequence of SEQ ID NO:4, a CDR-L2 comprising the amino acid sequence of SEQ ID NO: 5, and a CDR-L3 comprising the amino acid sequence of SEQ ID NO:6; the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179, and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182; the VH domain comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227, and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; the VH domain comprises a CDR-H1 comprising the amino acid sequence of SEQ ID NO: 229, a CDR-H2 comprising the amino acid sequence of SEQ ID NO:230, and a CDR-H3 comprising the amino acid sequence of SEQ ID NO:231, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233, and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO:225, a CDR-H2 comprising the amino acid sequence of SEQ ID NO: 232, and a CDR-H3 comprising the amino acid sequence of SEQ ID NO:233, and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228; or the VH domain comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231, and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42. In some embodiments, the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15, and wherein the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:18; the VH domain comprises a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21, and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27, and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30; the VH domain comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33, and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36; the VH domain comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45, and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3, and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain comprises a CDR-H1 comprising the amino acid sequence of SEQ ID NO:183, a CDR-H2 comprising the amino acid sequence of SEQ ID NO: 184, and a CDR-H3 comprising the amino acid sequence of SEQ ID NO: 179, and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO: 182; the VH domain comprises a CDR-H1 comprising the amino acid sequence of SEQ ID NO: 238, a CDR-H2 comprising the amino acid sequence of SEQ ID NO:239, and a CDR-H3 comprising the amino acid sequence of SEQ ID NO:233, and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228; the VH domain comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:238, a CDR-H2 comprising the amino acid sequence of SEQ ID NO: 243, and a CDR-H3 comprising the amino acid sequence of SEQ ID NO:233, and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228; or the VH domain comprises a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO: 242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain of both antibody heavy chain polypeptides comprises an amino acid sequence that is at least 90%, at least 95%, at least 99%, or 100% identical to the sequence of SEQ ID NO: 62, and wherein the VL domain of both antibody light chain polypeptides comprises an amino acid sequence that is at least 90%, at least 95%, at least 99%, or 100% identical to the sequence of SEQ ID NO:63; the VH domain of both antibody heavy chain polypeptides comprises an amino acid sequence that is at least 90%, at least 95%, at least 99%, or 100% identical to the sequence of SEQ ID NO:64, and wherein the VL domain of both antibody light chain polypeptides comprises an amino acid sequence that is at least 90%, at least 95%, at least 99%, or 100% identical to the sequence of SEQ ID NO:65; the VH domain of both antibody heavy chain polypeptides comprises an amino acid sequence that is at least 90%, at least 95%, at least 99%, or 100% identical to the sequence of SEQ ID NO:66, and wherein the VL domain of both antibody light chain polypeptides comprises an amino acid sequence that is at least 90%, at least 95%, at least 99%, or 100% identical to the sequence of SEQ ID NO:67; the VH domain of both antibody heavy chain polypeptides comprises an amino acid sequence that is at least 90%, at least 95%, at least 99%, or 100% identical to the sequence of SEQ ID NO:68, and wherein the VL domain of both antibody light chain polypeptides comprises an amino acid sequence that is at least 90%, at least 95%, at least 99%, or 100% identical to the sequence of SEQ ID NO:69; the VH domain of both antibody heavy chain polypeptides comprises an amino acid sequence that is at least 90%, at least 95%, at least 99%, or 100% identical to the sequence of SEQ ID NO:70, and wherein the VL domain of both antibody light chain polypeptides comprises an amino acid sequence that is at least 90%, at least 95%, at least 99%, or 100% identical to the sequence of SEQ ID NO:71; the VH domain of both antibody heavy chain polypeptides comprises an amino acid sequence that is at least 90%, at least 95%, at least 99%, or 100% identical to the sequence of SEQ ID NO:72, and wherein the VL domain of both antibody light chain polypeptides comprises an amino acid sequence that is at least 90%, at least 95%, at least 99%, or 100% identical to the sequence of SEQ ID NO:73; the VH domain of both antibody heavy chain polypeptides comprises an amino acid sequence that is at least 90%, at least 95%, at least 99%, or 100% identical to the sequence of SEQ ID NO: 185, and wherein the VL domain of both antibody light chain polypeptides comprises an amino acid sequence that is at least 90%, at least 95%, at least 99%, or 100% identical to the sequence of SEQ ID NO: 186; the VH domain of both antibody heavy chain polypeptides comprises an amino acid sequence that is at least 90%, at least 95%, at least 99%, or 100% identical to the sequence of SEQ ID NO:245, and wherein the VL domain of both antibody light chain polypeptides comprises an amino acid sequence that is at least 90%, at least 95%, at least 99%, or 100% identical to the sequence of SEQ ID NO:246; the VH domain of both antibody heavy chain polypeptides comprises an amino acid sequence that is at least 90%, at least 95%, at least 99%, or 100% identical to the sequence of SEQ ID NO: 251, and wherein the VL domain of both antibody light chain polypeptides comprises an amino acid sequence that is at least 90%, at least 95%, at least 99%, or 100% identical to the sequence of SEQ ID NO:252; the VH domain comprises the amino acid sequence of SEQ ID NO: 251, and wherein the VL domain comprises the amino acid sequence of SEQ ID NO:252; the VH domain of both antibody heavy chain polypeptides comprises an amino acid sequence that is at least 90%, at least 95%, at least 99%, or 100% identical to the sequence of SEQ ID NO: 253, and wherein the VL domain of both antibody light chain polypeptides comprises an amino acid sequence that is at least 90%, at least 95%, at least 99%, or 100% identical to the sequence of SEQ ID NO:254; the VH domain of both antibody heavy chain polypeptides comprises an amino acid sequence that is at least 90%, at least 95%, at least 99%, or 100% identical to the sequence of SEQ ID NO:247, and wherein the VL domain of both antibody light chain polypeptides comprises an amino acid sequence that is at least 90%, at least 95%, at least 99%, or 100% identical to the sequence of SEQ ID NO:248; the VH domain of both antibody heavy chain polypeptides comprises an amino acid sequence that is at least 90%, at least 95%, at least 99%, or 100% identical to the sequence of SEQ ID NO:249, and wherein the VL domain of both antibody light chain polypeptides comprises an amino acid sequence that is at least 90%, at least 95%, at least 99%, or 100% identical to the sequence of SEQ ID NO:250; the VH domain comprises the amino acid sequence of SEQ ID NO:249, and wherein the VL domain comprises the amino acid sequence of SEQ ID NO:250; the VH domain of both antibody heavy chain polypeptides comprises an amino acid sequence that is at least 90%, at least 95%, at least 99%, or 100% identical to the sequence of SEQ ID NO:255, and wherein the VL domain of both antibody light chain polypeptides comprises an amino acid sequence that is at least 90%, at least 95%, at least 99%, or 100% identical to the sequence of SEQ ID NO:256; or the VH domain of both antibody heavy chain polypeptides comprises an amino acid sequence that is at least 90%, at least 95%, at least 99%, or 100% identical to the sequence of SEQ ID NO:257, and wherein the VL domain of both antibody light chain polypeptides comprises an amino acid sequence that is at least 90%, at least 95%, at least 99%, or 100% identical to the sequence of SEQ ID NO:258. In some embodiments, the VH domain comprises an amino acid sequence that is at least 90%, at least 95%, at least 99%, or 100% identical to the sequence of SEQ ID NO:62, and wherein the VL domain comprises an amino acid sequence that is at least 90%, at least 95%, at least 99%, or 100% identical to the sequence of SEQ ID NO:63; the VH domain comprises an amino acid sequence that is at least 90%, at least 95%, at least 99%, or 100% identical to the sequence of SEQ ID NO:64, and wherein the VL domain comprises an amino acid sequence that is at least 90%, at least 95%, at least 99%, or 100% identical to the sequence of SEQ ID NO:65; the VH domain comprises an amino acid sequence that is at least 90%, at least 95%, at least 99%, or 100% identical to the sequence of SEQ ID NO:66, and wherein the VL domain comprises an amino acid sequence that is at least 90%, at least 95%, at least 99%, or 100% identical to the sequence of SEQ ID NO:67; the VH domain comprises an amino acid sequence that is at least 90%, at least 95%, at least 99%, or 100% identical to the sequence of SEQ ID NO:68, and wherein the VL domain comprises an amino acid sequence that is at least 90%, at least 95%, at least 99%, or 100% identical to the sequence of SEQ ID NO:69; the VH domain comprises an amino acid sequence that is at least 90%, at least 95%, at least 99%, or 100% identical to the sequence of SEQ ID NO:70, and wherein the VL domain comprises an amino acid sequence that is at least 90%, at least 95%, at least 99%, or 100% identical to the sequence of SEQ ID NO:71; the VH domain comprises an amino acid sequence that is at least 90%, at least 95%, at least 99%, or 100% identical to the sequence of SEQ ID NO:72, and wherein the VL domain comprises an amino acid sequence that is at least 90%, at least 95%, at least 99%, or 100% identical to the sequence of SEQ ID NO:73; the VH domain comprises an amino acid sequence that is at least 90%, at least 95%, at least 99%, or 100% identical to the sequence of SEQ ID NO: 185, and wherein the VL domain comprises an amino acid sequence that is at least 90%, at least 95%, at least 99%, or 100% identical to the sequence of SEQ ID NO:186; the VH domain comprises an amino acid sequence that is at least 90%, at least 95%, at least 99%, or 100% identical to the sequence of SEQ ID NO:245, and wherein the VL domain comprises an amino acid sequence that is at least 90%, at least 95%, at least 99%, or 100% identical to the sequence of SEQ ID NO:246; the VH domain comprises an amino acid sequence that is at least 90%, at least 95%, at least 99%, or 100% identical to the sequence of SEQ ID NO:251, and wherein the VL domain comprises an amino acid sequence that is at least 90%, at least 95%, at least 99%, or 100% identical to the sequence of SEQ ID NO:252; the VH domain comprises the amino acid sequence of SEQ ID NO:251, and wherein the VL domain comprises the amino acid sequence of SEQ ID NO:252; the VH domain comprises an amino acid sequence that is at least 90%, at least 95%, at least 99%, or 100% identical to the sequence of SEQ ID NO:253, and wherein the VL domain comprises an amino acid sequence that is at least 90%, at least 95%, at least 99%, or 100% identical to the sequence of SEQ ID NO:254; the VH domain comprises an amino acid sequence that is at least 90%, at least 95%, at least 99%, or 100% identical to the sequence of SEQ ID NO:247, and wherein the VL domain comprises an amino acid sequence that is at least 90%, at least 95%, at least 99%, or 100% identical to the sequence of SEQ ID NO:248; the VH domain comprises an amino acid sequence that is at least 90%, at least 95%, at least 99%, or 100% identical to the sequence of SEQ ID NO:249, and wherein the VL domain comprises an amino acid sequence that is at least 90%, at least 95%, at least 99%, or 100% identical to the sequence of SEQ ID NO:250; the VH domain comprises the amino acid sequence of SEQ ID NO: 249, and wherein the VL domain comprises the amino acid sequence of SEQ ID NO:250; the VH domain comprises an amino acid sequence that is at least 90%, at least 95%, at least 99%, or 100% identical to the sequence of SEQ ID NO:255, and wherein the VL domain comprises an amino acid sequence that is at least 90%, at least 95%, at least 99%, or 100% identical to the sequence of SEQ ID NO:256; or the VH domain comprises an amino acid sequence that is at least 90%, at least 95%, at least 99%, or 100% identical to the sequence of SEQ ID NO:257, and wherein the VL domain comprises an amino acid sequence that is at least 90%, at least 95%, at least 99%, or 100% identical to the sequence of SEQ ID NO:258. In some embodiments, one or both of the antibody heavy chain polypeptides comprise(s) the following amino acid substitutions: L234A, L235A, and G237A, numbering according to EU index. In some embodiments, a first of the antibody heavy chain polypeptides comprises amino acid substitutions Y349C and T366W, and a second of the antibody heavy chain polypeptides comprises amino acid substitutions S354C, T366S, L368A and Y407V, numbering according to EU index.

In some embodiments according to any of the embodiments described herein, the second moiety comprises an IL-2 polypeptide. In some embodiments, the IL-2 polypeptide is a mutant IL-2 polypeptide comprising one or more mutations relative to a human IL-2 polypeptide comprising the sequence of APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO:81). In some embodiments, the mutant IL-2 polypeptide has a binding affinity to IL-2Rx that is reduced by 50% or more, compared to binding affinity of a wild-type IL-2 polypeptide comprising the sequence of SEQ ID: 81 for IL-2Rα. In some embodiments, the mutant IL-2 polypeptide has a binding affinity to IL-2Rβ that is reduced by 50% or more, compared to binding affinity of a wild-type IL-2 polypeptide comprising the sequence of SEQ ID: 81 for IL-2Rβ; and/or the mutant IL-2 polypeptide has a binding affinity to IL-2Rγ that is reduced by 50% or more, compared to binding affinity of a wild-type IL-2 polypeptide comprising the sequence of SEQ ID: 81 for IL-2Ry. In some embodiments, the IL-2 polypeptide comprises the sequence of SEQ ID NO:81 with one, two, three, four, or five amino acid substitutions relative to SEQ ID NO:81, and wherein the one, two, three, four, or five substitution(s) comprise substitution(s) at positions of SEQ ID NO:81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130. In some embodiments, the IL-2 polypeptide comprises the sequence of SEQ ID NO:81 with one of the following sets of amino acid substitutions (relative to the sequence of SEQ ID NO:81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S. In some embodiments, the IL-2 polypeptide comprises the sequence of SEQ ID NO:81 with a further amino acid substitution relative to SEQ ID NO: 81 at position C125. In some embodiments, the IL-2 polypeptide comprises the sequence of SEQ ID NO:81 with one of the following sets of amino acid substitutions (relative to the sequence of SEQ ID NO:81): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88T, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the IL-2 polypeptide comprises the sequence APTSSSTKKTQLQLEHLLLDLQMILNGINNYKNPKLTEMLTAKFYMPKKATELKHLQCL EEELKPLEEVLNLAQSKNFHLRPRDLISAINVIVLELKGSETTFMCEYADETATIVEFLNR WITFAQSIISTLT (SEQ ID NO:80). In some embodiments, the IL-2 polypeptide comprises the sequence APTSSSTKKTQLQLEELLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:297). In some embodiments, the IL-2 polypeptide comprises a sequence selected from the group consisting of SEQ ID Nos: 85-155 and 190-216. In some embodiments, the IL-2 polypeptide comprises a sequence selected from the group consisting of SEQ ID Nos: 80, 85-155, 190-216, 297, and 354-383. In some embodiments, the second moiety comprises a polypeptide that induces signaling via IL2RBy. In some embodiments, the second moiety comprises an IL-21 polypeptide.

In some embodiments according to any of the embodiments described herein (e.g., a fusion protein of the present disclosure), one or both of the antibody Fc domains comprise(s) human IgG1 Fc domains with the following amino acid substitutions: L234A, L235A, G237A, and K322A, numbering according to EU index. In some embodiments, one or both of the antibody Fc domains do not have a C-terminal lysine. In some embodiments, one or both of the antibody Fc domains comprise(s) human IgG1 Fc domains with the following amino acid substitutions: L234A, L235A, and G237A, numbering according to EU index. In some embodiments, one or both of the antibody Fc domains do not have a C-terminal lysine. In some embodiments, a first of the two Fc domains comprises a human IgG1 Fc domain with amino acid substitutions Y349C and T366W, and a second of the two Fc domain comprises a human IgG1 Fc domain with amino acid substitutions S354C, T366S, L368A and Y407V, numbering according to EU index. In some embodiments, one or both of the antibody Fc domains do not have a C-terminal lysine. In some embodiments (e.g., a fusion protein of the present disclosure), the linker comprises the sequence (GGGS)xGn (SEQ ID NO:74), (GGGGS)xGn (SEQ ID NO:75), or (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3. In some embodiments, the linker comprises the sequence GGGGSGGGGSGGGGS (SEQ ID NO:79) or SGGGGSGGGGSGGGGS (SEQ ID NO:389). In some embodiments (e.g., in a fusion protein of the present disclosure), the linker connects a first moiety of the present disclosure (e.g., a human or humanized antibody or antigen-binding fragment thereof that specifically binds CD8b and/or CD8ab) and a second moiety of the present disclosure (e.g., an IL-2 polypeptide of the present disclosure, IL-21 polypeptide of the present disclosure, or a polypeptide that induces signaling via IL2Rβγ of the present disclosure).

In some embodiments, a fusion protein of the present disclosure comprises one or two light chains comprising the amino acid sequence of SEQ ID NO: 156, a heavy chain comprising the amino acid sequence of SEQ ID NO: 157, and a heavy chain comprising the amino acid sequence of SEQ ID NO:158. In some embodiments, a fusion protein of the present disclosure comprises one or two light chains comprising the amino acid sequence of SEQ ID NO:156, a heavy chain comprising the amino acid sequence of SEQ ID NO: 157, and a heavy chain comprising the amino acid sequence of SEQ ID NO:217. In some embodiments, a fusion protein of the present disclosure comprises one or two light chains comprising the amino acid sequence of SEQ ID NO: 159, a heavy chain comprising the amino acid sequence of SEQ ID NO: 160, and a heavy chain comprising the amino acid sequence of SEQ ID NO:161. In some embodiments, a fusion protein of the present disclosure comprises one or two light chains comprising the amino acid sequence of SEQ ID NO: 159, a heavy chain comprising the amino acid sequence of SEQ ID NO: 160, and a heavy chain comprising the amino acid sequence of SEQ ID NO:218. In some embodiments, a fusion protein of the present disclosure comprises one or two light chains comprising the amino acid sequence of SEQ ID NO: 162, a heavy chain comprising the amino acid sequence of SEQ ID NO:163, and a heavy chain comprising the amino acid sequence of SEQ ID NO:164. In some embodiments, a fusion protein of the present disclosure comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO:219. In some embodiments, a fusion protein of the present disclosure comprises one or two light chains comprising the amino acid sequence of SEQ ID NO: 165, a heavy chain comprising the amino acid sequence of SEQ ID NO:166, and a heavy chain comprising the amino acid sequence of SEQ ID NO:167. In some embodiments, a fusion protein of the present disclosure comprises one or two light chains comprising the amino acid sequence of SEQ ID NO: 165, a heavy chain comprising the amino acid sequence of SEQ ID NO: 166, and a heavy chain comprising the amino acid sequence of SEQ ID NO:220. In some embodiments, a fusion protein of the present disclosure comprises one or two light chains comprising the amino acid sequence of SEQ ID NO: 168, a heavy chain comprising the amino acid sequence of SEQ ID NO:169, and a heavy chain comprising the amino acid sequence of SEQ ID NO:170. In some embodiments, a fusion protein of the present disclosure comprises one or two light chains comprising the amino acid sequence of SEQ ID NO:168, a heavy chain comprising the amino acid sequence of SEQ ID NO:169, and a heavy chain comprising the amino acid sequence of SEQ ID NO:221. In some embodiments, a fusion protein of the present disclosure comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO:173. In some embodiments, a fusion protein of the present disclosure comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO:222. In some embodiments, a fusion protein of the present disclosure comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO:176. In some embodiments, a fusion protein of the present disclosure comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO:223. In some embodiments, a fusion protein of the present disclosure comprises one or two light chains comprising the amino acid sequence of SEQ ID NO: 187, a heavy chain comprising the amino acid sequence of SEQ ID NO: 188, and a heavy chain comprising the amino acid sequence of SEQ ID NO:189. In some embodiments, a fusion protein of the present disclosure comprises one or two light chains comprising the amino acid sequence of SEQ ID NO:187, a heavy chain comprising the amino acid sequence of SEQ ID NO: 188, and a heavy chain comprising the amino acid sequence of SEQ ID NO:224. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:300. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:301. In some embodiments, a fusion protein of the present disclosure comprises one or two light chains comprising the amino acid sequence of SEQ ID NO: 302, a heavy chain comprising the amino acid sequence of SEQ ID NO:303, and a heavy chain comprising the amino acid sequence of SEQ ID NO:304. In some embodiments, a fusion protein of the present disclosure comprises one or two light chains comprising the amino acid sequence of SEQ ID NO:302, a heavy chain comprising the amino acid sequence of SEQ ID NO: 303, and a heavy chain comprising the amino acid sequence of SEQ ID NO:305. In some embodiments, a fusion protein of the present disclosure comprises one or two light chains comprising the amino acid sequence of SEQ ID NO:306, a heavy chain comprising the amino acid sequence of SEQ ID NO:307, and a heavy chain comprising the amino acid sequence of SEQ ID NO:308. In some embodiments, a fusion protein of the present disclosure comprises one or two light chains comprising the amino acid sequence of SEQ ID NO:306, a heavy chain comprising the amino acid sequence of SEQ ID NO:307, and a heavy chain comprising the amino acid sequence of SEQ ID NO:309. In some embodiments, a fusion protein of the present disclosure comprises one or two light chains comprising the amino acid sequence of SEQ ID NO: 310, a heavy chain comprising the amino acid sequence of SEQ ID NO:311, and a heavy chain comprising the amino acid sequence of SEQ ID NO:312. In some embodiments, a fusion protein of the present disclosure comprises one or two light chains comprising the amino acid sequence of SEQ ID NO:310, a heavy chain comprising the amino acid sequence of SEQ ID NO: 311, and a heavy chain comprising the amino acid sequence of SEQ ID NO:313. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:316. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:317. In some embodiments, a fusion protein of the present disclosure comprises one or two light chains comprising the amino acid sequence of SEQ ID NO: 318, a heavy chain comprising the amino acid sequence of SEQ ID NO:319, and a heavy chain comprising the amino acid sequence of SEQ ID NO:320. In some embodiments, a fusion protein of the present disclosure comprises one or two light chains comprising the amino acid sequence of SEQ ID NO:318, a heavy chain comprising the amino acid sequence of SEQ ID NO: 319, and a heavy chain comprising the amino acid sequence of SEQ ID NO:321. In some embodiments, a fusion protein of the present disclosure comprises one or two light chains comprising the amino acid sequence of SEQ ID NO:322, a heavy chain comprising the amino acid sequence of SEQ ID NO:323, and a heavy chain comprising the amino acid sequence of SEQ ID NO:324. In some embodiments, a fusion protein of the present disclosure comprises one or two light chains comprising the amino acid sequence of SEQ ID NO:322, a heavy chain comprising the amino acid sequence of SEQ ID NO:323, and a heavy chain comprising the amino acid sequence of SEQ ID NO:325. In some embodiments, a fusion protein of the present disclosure comprises one or two light chains comprising the amino acid sequence of SEQ ID NO: 326, a heavy chain comprising the amino acid sequence of SEQ ID NO:327, and a heavy chain comprising the amino acid sequence of SEQ ID NO:328. In some embodiments, a fusion protein of the present disclosure comprises one or two light chains comprising the amino acid sequence of SEQ ID NO:326, a heavy chain comprising the amino acid sequence of SEQ ID NO: 327, and a heavy chain comprising the amino acid sequence of SEQ ID NO:329. In some embodiments, a fusion protein of the present disclosure comprises one or two light chains comprising the amino acid sequence of SEQ ID NO:330, a heavy chain comprising the amino acid sequence of SEQ ID NO:331, and a heavy chain comprising the amino acid sequence of SEQ ID NO:332. In some embodiments, a fusion protein of the present disclosure comprises one or two light chains comprising the amino acid sequence of SEQ ID NO:330, a heavy chain comprising the amino acid sequence of SEQ ID NO:331, and a heavy chain comprising the amino acid sequence of SEQ ID NO:333. In some embodiments, a fusion protein of the present disclosure comprises one or two light chains comprising the amino acid sequence of SEQ ID NO: 334, a heavy chain comprising the amino acid sequence of SEQ ID NO:335, and a heavy chain comprising the amino acid sequence of SEQ ID NO:336. In some embodiments, a fusion protein of the present disclosure comprises one or two light chains comprising the amino acid sequence of SEQ ID NO:334, a heavy chain comprising the amino acid sequence of SEQ ID NO: 335, and a heavy chain comprising the amino acid sequence of SEQ ID NO:337. In some embodiments, a fusion protein of the present disclosure comprises one or two light chains comprising the amino acid sequence of SEQ ID NO:338, a heavy chain comprising the amino acid sequence of SEQ ID NO:339, and a heavy chain comprising the amino acid sequence of SEQ ID NO:340. In some embodiments, a fusion protein of the present disclosure comprises one or two light chains comprising the amino acid sequence of SEQ ID NO:338, a heavy chain comprising the amino acid sequence of SEQ ID NO:339, and a heavy chain comprising the amino acid sequence of SEQ ID NO:341. In some embodiments, a fusion protein of the present disclosure comprises one or two light chains comprising the amino acid sequence of SEQ ID NO: 342, a heavy chain comprising the amino acid sequence of SEQ ID NO:343, and a heavy chain comprising the amino acid sequence of SEQ ID NO:344. In some embodiments, a fusion protein of the present disclosure comprises one or two light chains comprising the amino acid sequence of SEQ ID NO:342, a heavy chain comprising the amino acid sequence of SEQ ID NO: 343, and a heavy chain comprising the amino acid sequence of SEQ ID NO:345. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:348. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:349. In some embodiments, a fusion protein of the present disclosure comprises one or two light chains comprising the amino acid sequence of SEQ ID NO: 350, a heavy chain comprising the amino acid sequence of SEQ ID NO:351, and a heavy chain comprising the amino acid sequence of SEQ ID NO:352. In some embodiments, a fusion protein of the present disclosure comprises one or two light chains one or two light chains comprising the amino acid sequence of SEQ ID NO: 156, a heavy chain comprising the amino acid sequence of SEQ ID NO:157, and a heavy chain comprising the amino acid sequence of SEQ ID NO:217. In some embodiments, a fusion protein of the present disclosure comprises one or two light chains one or two light chains comprising the amino acid sequence of SEQ ID NO: 159, a heavy chain comprising the amino acid sequence of SEQ ID NO: 160, and a heavy chain comprising the amino acid sequence of SEQ ID NO:218. In some embodiments, a fusion protein of the present disclosure comprises one or two light chains one or two light chains comprising the amino acid sequence of SEQ ID NO: 162, a heavy chain comprising the amino acid sequence of SEQ ID NO: 163, and a heavy chain comprising the amino acid sequence of SEQ ID NO:219. In some embodiments, a fusion protein of the present disclosure comprises one or two light chains one or two light chains comprising the amino acid sequence of SEQ ID NO: 165, a heavy chain comprising the amino acid sequence of SEQ ID NO:166, and a heavy chain comprising the amino acid sequence of SEQ ID NO:220. In some embodiments, a fusion protein of the present disclosure comprises one or two light chains one or two light chains comprising the amino acid sequence of SEQ ID NO:168, a heavy chain comprising the amino acid sequence of SEQ ID NO:169, and a heavy chain comprising the amino acid sequence of SEQ ID NO:221. In some embodiments, a fusion protein of the present disclosure comprises one or two light chains one or two light chains comprising the amino acid sequence of SEQ ID NO: 171, a heavy chain comprising the amino acid sequence of SEQ ID NO: 172, and a heavy chain comprising the amino acid sequence of SEQ ID NO:222. In some embodiments, a fusion protein of the present disclosure comprises one or two light chains one or two light chains comprising the amino acid sequence of SEQ ID NO:174, a heavy chain comprising the amino acid sequence of SEQ ID NO: 175, and a heavy chain comprising the amino acid sequence of SEQ ID NO:223. In some embodiments, a fusion protein of the present disclosure comprises one or two light chains one or two light chains comprising the amino acid sequence of SEQ ID NO: 187, a heavy chain comprising the amino acid sequence of SEQ ID NO: 188, and a heavy chain comprising the amino acid sequence of SEQ ID NO:224. In some embodiments, a fusion protein of the present disclosure comprises one or two light chains one or two light chains comprising the amino acid sequence of SEQ ID NO:298, a heavy chain comprising the amino acid sequence of SEQ ID NO:299, and a heavy chain comprising the amino acid sequence of SEQ ID NO:301. In some embodiments, a fusion protein of the present disclosure comprises one or two light chains one or two light chains comprising the amino acid sequence of SEQ ID NO: 302, a heavy chain comprising the amino acid sequence of SEQ ID NO:303, and a heavy chain comprising the amino acid sequence of SEQ ID NO:305. In some embodiments, a fusion protein of the present disclosure comprises one or two light chains one or two light chains comprising the amino acid sequence of SEQ ID NO:306, a heavy chain comprising the amino acid sequence of SEQ ID NO:307, and a heavy chain comprising the amino acid sequence of SEQ ID NO:309. In some embodiments, a fusion protein of the present disclosure comprises one or two light chains one or two light chains comprising the amino acid sequence of SEQ ID NO: 310, a heavy chain comprising the amino acid sequence of SEQ ID NO:311, and a heavy chain comprising the amino acid sequence of SEQ ID NO:313. In some embodiments, a fusion protein of the present disclosure comprises one or two light chains one or two light chains comprising the amino acid sequence of SEQ ID NO:314, a heavy chain comprising the amino acid sequence of SEQ ID NO:315, and a heavy chain comprising the amino acid sequence of SEQ ID NO:317. In some embodiments, a fusion protein of the present disclosure comprises one or two light chains one or two light chains comprising the amino acid sequence of SEQ ID NO: 318, a heavy chain comprising the amino acid sequence of SEQ ID NO:319, and a heavy chain comprising the amino acid sequence of SEQ ID NO:321. In some embodiments, a fusion protein of the present disclosure comprises one or two light chains one or two light chains comprising the amino acid sequence of SEQ ID NO:322, a heavy chain comprising the amino acid sequence of SEQ ID NO:323, and a heavy chain comprising the amino acid sequence of SEQ ID NO:325. In some embodiments, a fusion protein of the present disclosure comprises one or two light chains one or two light chains comprising the amino acid sequence of SEQ ID NO: 326, a heavy chain comprising the amino acid sequence of SEQ ID NO:327, and a heavy chain comprising the amino acid sequence of SEQ ID NO:329. In some embodiments, a fusion protein of the present disclosure comprises one or two light chains one or two light chains comprising the amino acid sequence of SEQ ID NO:330, a heavy chain comprising the amino acid sequence of SEQ ID NO:331, and a heavy chain comprising the amino acid sequence of SEQ ID NO:333. In some embodiments, a fusion protein of the present disclosure comprises one or two light chains one or two light chains comprising the amino acid sequence of SEQ ID NO: 334, a heavy chain comprising the amino acid sequence of SEQ ID NO:335, and a heavy chain comprising the amino acid sequence of SEQ ID NO:337. In some embodiments, a fusion protein of the present disclosure comprises one or two light chains one or two light chains comprising the amino acid sequence of SEQ ID NO:338, a heavy chain comprising the amino acid sequence of SEQ ID NO:339, and a heavy chain comprising the amino acid sequence of SEQ ID NO:341. In some embodiments, a fusion protein of the present disclosure comprises one or two light chains one or two light chains comprising the amino acid sequence of SEQ ID NO: 342, a heavy chain comprising the amino acid sequence of SEQ ID NO:343, and a heavy chain comprising the amino acid sequence of SEQ ID NO:345. In some embodiments, a fusion protein of the present disclosure comprises one or two light chains one or two light chains comprising the amino acid sequence of SEQ ID NO:346, a heavy chain comprising the amino acid sequence of SEQ ID NO:347, and a heavy chain comprising the amino acid sequence of SEQ ID NO:349. In some embodiments, a fusion protein of the present disclosure comprises one or two light chains one or two light chains comprising the amino acid sequence of SEQ ID NO: 350, a heavy chain comprising the amino acid sequence of SEQ ID NO:351, and a heavy chain comprising the amino acid sequence of SEQ ID NO:353. In some embodiments, the fusion protein comprises one or two antigen binding sites, each antigen binding site comprising a VL domain from one of the light chain(s) and a VH domain from one of the heavy chains (e.g., the fusion protein comprises two antigen binding sites: one comprising a VL domain from one of the two light chains and a VH domain from one of the heavy chains, and another comprising a VL domain from the other light chain and a VH domain from the other heavy chain).

In some embodiments, a fusion protein of the present disclosure comprises one or two polypeptides comprising the amino acid sequence of SEQ ID NO: 156, a polypeptide comprising the amino acid sequence of SEQ ID NO: 157, and a polypeptide comprising the amino acid sequence of SEQ ID NO:158. In some embodiments, a fusion protein of the present disclosure comprises one or two polypeptides comprising the amino acid sequence of SEQ ID NO:156, a polypeptide comprising the amino acid sequence of SEQ ID NO: 157, and a polypeptide comprising the amino acid sequence of SEQ ID NO:217. In some embodiments, a fusion protein of the present disclosure comprises one or two polypeptides comprising the amino acid sequence of SEQ ID NO: 159, a polypeptide comprising the amino acid sequence of SEQ ID NO:160, and a polypeptide comprising the amino acid sequence of SEQ ID NO:161. In some embodiments, a fusion protein of the present disclosure comprises one or two polypeptides comprising the amino acid sequence of SEQ ID NO: 159, a polypeptide comprising the amino acid sequence of SEQ ID NO: 160, and a polypeptide comprising the amino acid sequence of SEQ ID NO:218. In some embodiments, a fusion protein of the present disclosure comprises one or two polypeptides comprising the amino acid sequence of SEQ ID NO:162, a polypeptide comprising the amino acid sequence of SEQ ID NO: 163, and a polypeptide comprising the amino acid sequence of SEQ ID NO:164. In some embodiments, a fusion protein of the present disclosure comprises one or two polypeptides comprising the amino acid sequence of SEQ ID NO:162, a polypeptide comprising the amino acid sequence of SEQ ID NO: 163, and a polypeptide comprising the amino acid sequence of SEQ ID NO:219. In some embodiments, a fusion protein of the present disclosure comprises one or two polypeptides comprising the amino acid sequence of SEQ ID NO: 165, a polypeptide comprising the amino acid sequence of SEQ ID NO: 166, and a polypeptide comprising the amino acid sequence of SEQ ID NO:167. In some embodiments, a fusion protein of the present disclosure comprises one or two polypeptides comprising the amino acid sequence of SEQ ID NO:165, a polypeptide comprising the amino acid sequence of SEQ ID NO: 166, and a polypeptide comprising the amino acid sequence of SEQ ID NO:220. In some embodiments, a fusion protein of the present disclosure comprises one or two polypeptides comprising the amino acid sequence of SEQ ID NO:168, a polypeptide comprising the amino acid sequence of SEQ ID NO: 169, and a polypeptide comprising the amino acid sequence of SEQ ID NO:170. In some embodiments, a fusion protein of the present disclosure comprises one or two polypeptides comprising the amino acid sequence of SEQ ID NO:168, a polypeptide comprising the amino acid sequence of SEQ ID NO: 169, and a polypeptide comprising the amino acid sequence of SEQ ID NO:221. In some embodiments, a fusion protein of the present disclosure comprises one or two polypeptides comprising the amino acid sequence of SEQ ID NO: 171, a polypeptide comprising the amino acid sequence of SEQ ID NO:172, and a polypeptide comprising the amino acid sequence of SEQ ID NO:173. In some embodiments, a fusion protein of the present disclosure comprises one or two polypeptides comprising the amino acid sequence of SEQ ID NO: 171, a polypeptide comprising the amino acid sequence of SEQ ID NO: 172, and a polypeptide comprising the amino acid sequence of SEQ ID NO:222. In some embodiments, a fusion protein of the present disclosure comprises one or two polypeptides comprising the amino acid sequence of SEQ ID NO: 174, a polypeptide comprising the amino acid sequence of SEQ ID NO:175, and a polypeptide comprising the amino acid sequence of SEQ ID NO:176. In some embodiments, a fusion protein of the present disclosure comprises one or two polypeptides comprising the amino acid sequence of SEQ ID NO: 174, a polypeptide comprising the amino acid sequence of SEQ ID NO: 175, and a polypeptide comprising the amino acid sequence of SEQ ID NO:223. In some embodiments, a fusion protein of the present disclosure comprises one or two polypeptides comprising the amino acid sequence of SEQ ID NO: 187, a polypeptide comprising the amino acid sequence of SEQ ID NO:188, and a polypeptide comprising the amino acid sequence of SEQ ID NO:189. In some embodiments, a fusion protein of the present disclosure comprises one or two polypeptides comprising the amino acid sequence of SEQ ID NO:187, a polypeptide comprising the amino acid sequence of SEQ ID NO: 188, and a polypeptide comprising the amino acid sequence of SEQ ID NO:224. In some embodiments, a fusion protein of the present disclosure comprises one or two polypeptides comprising the amino acid sequence of SEQ ID NO:298, a polypeptide comprising the amino acid sequence of SEQ ID NO:299, and a polypeptide comprising the amino acid sequence of SEQ ID NO:300. In some embodiments, a fusion protein of the present disclosure comprises one or two polypeptides comprising the amino acid sequence of SEQ ID NO:298, a polypeptide comprising the amino acid sequence of SEQ ID NO:299, and a polypeptide comprising the amino acid sequence of SEQ ID NO:301. In some embodiments, a fusion protein of the present disclosure comprises one or two polypeptides comprising the amino acid sequence of SEQ ID NO: 302, a polypeptide comprising the amino acid sequence of SEQ ID NO:303, and a polypeptide comprising the amino acid sequence of SEQ ID NO:304. In some embodiments, a fusion protein of the present disclosure comprises one or two polypeptides comprising the amino acid sequence of SEQ ID NO:302, a polypeptide comprising the amino acid sequence of SEQ ID NO: 303, and a polypeptide comprising the amino acid sequence of SEQ ID NO:305. In some embodiments, a fusion protein of the present disclosure comprises one or two polypeptides comprising the amino acid sequence of SEQ ID NO:306, a polypeptide comprising the amino acid sequence of SEQ ID NO:307, and a polypeptide comprising the amino acid sequence of SEQ ID NO:308. In some embodiments, a fusion protein of the present disclosure comprises one or two polypeptides comprising the amino acid sequence of SEQ ID NO:306, a polypeptide comprising the amino acid sequence of SEQ ID NO:307, and a polypeptide comprising the amino acid sequence of SEQ ID NO:309. In some embodiments, a fusion protein of the present disclosure comprises one or two polypeptides comprising the amino acid sequence of SEQ ID NO: 310, a polypeptide comprising the amino acid sequence of SEQ ID NO:311, and a polypeptide comprising the amino acid sequence of SEQ ID NO:312. In some embodiments, a fusion protein of the present disclosure comprises one or two polypeptides comprising the amino acid sequence of SEQ ID NO:310, a polypeptide comprising the amino acid sequence of SEQ ID NO: 311, and a polypeptide comprising the amino acid sequence of SEQ ID NO:313. In some embodiments, a fusion protein of the present disclosure comprises one or two polypeptides comprising the amino acid sequence of SEQ ID NO:314, a polypeptide comprising the amino acid sequence of SEQ ID NO:315, and a polypeptide comprising the amino acid sequence of SEQ ID NO:316. In some embodiments, a fusion protein of the present disclosure comprises one or two polypeptides comprising the amino acid sequence of SEQ ID NO:314, a polypeptide comprising the amino acid sequence of SEQ ID NO:315, and a polypeptide comprising the amino acid sequence of SEQ ID NO:317. In some embodiments, a fusion protein of the present disclosure comprises one or two polypeptides comprising the amino acid sequence of SEQ ID NO: 318, a polypeptide comprising the amino acid sequence of SEQ ID NO:319, and a polypeptide comprising the amino acid sequence of SEQ ID NO:320. In some embodiments, a fusion protein of the present disclosure comprises one or two polypeptides comprising the amino acid sequence of SEQ ID NO:318, a polypeptide comprising the amino acid sequence of SEQ ID NO: 319, and a polypeptide comprising the amino acid sequence of SEQ ID NO:321. In some embodiments, a fusion protein of the present disclosure comprises one or two polypeptides comprising the amino acid sequence of SEQ ID NO:322, a polypeptide comprising the amino acid sequence of SEQ ID NO:323, and a polypeptide comprising the amino acid sequence of SEQ ID NO:324. In some embodiments, a fusion protein of the present disclosure comprises one or two polypeptides comprising the amino acid sequence of SEQ ID NO:322, a polypeptide comprising the amino acid sequence of SEQ ID NO:323, and a polypeptide comprising the amino acid sequence of SEQ ID NO:325. In some embodiments, a fusion protein of the present disclosure comprises one or two polypeptides comprising the amino acid sequence of SEQ ID NO: 326, a polypeptide comprising the amino acid sequence of SEQ ID NO:327, and a polypeptide comprising the amino acid sequence of SEQ ID NO:328. In some embodiments, a fusion protein of the present disclosure comprises one or two polypeptides comprising the amino acid sequence of SEQ ID NO:326, a polypeptide comprising the amino acid sequence of SEQ ID NO: 327, and a polypeptide comprising the amino acid sequence of SEQ ID NO:329. In some embodiments, a fusion protein of the present disclosure comprises one or two polypeptides comprising the amino acid sequence of SEQ ID NO:330, a polypeptide comprising the amino acid sequence of SEQ ID NO:331, and a polypeptide comprising the amino acid sequence of SEQ ID NO:332. In some embodiments, a fusion protein of the present disclosure comprises one or two polypeptides comprising the amino acid sequence of SEQ ID NO:330, a polypeptide comprising the amino acid sequence of SEQ ID NO:331, and a polypeptide comprising the amino acid sequence of SEQ ID NO:333. In some embodiments, a fusion protein of the present disclosure comprises one or two polypeptides comprising the amino acid sequence of SEQ ID NO: 334, a polypeptide comprising the amino acid sequence of SEQ ID NO:335, and a polypeptide comprising the amino acid sequence of SEQ ID NO:336. In some embodiments, a fusion protein of the present disclosure comprises one or two polypeptides comprising the amino acid sequence of SEQ ID NO:334, a polypeptide comprising the amino acid sequence of SEQ ID NO: 335, and a polypeptide comprising the amino acid sequence of SEQ ID NO:337. In some embodiments, a fusion protein of the present disclosure comprises one or two polypeptides comprising the amino acid sequence of SEQ ID NO:338, a polypeptide comprising the amino acid sequence of SEQ ID NO:339, and a polypeptide comprising the amino acid sequence of SEQ ID NO:340. In some embodiments, a fusion protein of the present disclosure comprises one or two polypeptides comprising the amino acid sequence of SEQ ID NO:338, a polypeptide comprising the amino acid sequence of SEQ ID NO:339, and a polypeptide comprising the amino acid sequence of SEQ ID NO:341. In some embodiments, a fusion protein of the present disclosure comprises one or two polypeptides comprising the amino acid sequence of SEQ ID NO: 342, a polypeptide comprising the amino acid sequence of SEQ ID NO:343, and a polypeptide comprising the amino acid sequence of SEQ ID NO:344. In some embodiments, a fusion protein of the present disclosure comprises one or two polypeptides comprising the amino acid sequence of SEQ ID NO:342, a polypeptide comprising the amino acid sequence of SEQ ID NO: 343, and a polypeptide comprising the amino acid sequence of SEQ ID NO:345. In some embodiments, a fusion protein of the present disclosure comprises one or two polypeptides comprising the amino acid sequence of SEQ ID NO:346, a polypeptide comprising the amino acid sequence of SEQ ID NO:347, and a polypeptide comprising the amino acid sequence of SEQ ID NO:348. In some embodiments, a fusion protein of the present disclosure comprises one or two polypeptides comprising the amino acid sequence of SEQ ID NO:346, a polypeptide comprising the amino acid sequence of SEQ ID NO:347, and a polypeptide comprising the amino acid sequence of SEQ ID NO:349. In some embodiments, a fusion protein of the present disclosure comprises one or two polypeptides comprising the amino acid sequence of SEQ ID NO: 350, a polypeptide comprising the amino acid sequence of SEQ ID NO:351, and a polypeptide comprising the amino acid sequence of SEQ ID NO:352. In some embodiments, a fusion protein of the present disclosure comprises one or two polypeptides comprising the amino acid sequence of SEQ ID NO:350, a polypeptide comprising the amino acid sequence of SEQ ID NO: 351, and a polypeptide comprising the amino acid sequence of SEQ ID NO:353.

Further provided herein are fusion proteins, comprising a first moiety that binds to a human CD8b and a second moiety comprising an IL2 polypeptide, wherein the fusion protein comprises four polypeptide chains, wherein: (1) the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:336, and the fourth polypeptide chain comprises the amino acid sequence of SEQ ID NO: 334; (2) the first polypeptide chain comprises the amino acid sequence of SEQ ID NO: 334, the second polypeptide chain comprises the amino acid sequence of SEQ ID NO:335, the third polypeptide chain comprises the amino acid sequence of SEQ ID NO:337, and the fourth polypeptide chain comprises the amino acid sequence of SEQ ID NO:334; (3) the first polypeptide chain comprises the amino acid sequence of SEQ ID NO:338, the second polypeptide chain comprises the amino acid sequence of SEQ ID NO:339, the third polypeptide chain comprises the amino acid sequence of SEQ ID NO:340, and the fourth polypeptide chain comprises the amino acid sequence of SEQ ID NO:338; or (4) the first polypeptide chain comprises the amino acid sequence of SEQ ID NO:338, the second polypeptide chain comprises the amino acid sequence of SEQ ID NO:339, the third polypeptide chain comprises the amino acid sequence of SEQ ID NO:341, and the fourth polypeptide chain comprises the amino acid sequence of SEQ ID NO:338.

Further provided herein are polynucleotides (e.g., isolated polynucleotides) encoding the antibody or fusion protein according to any one of the above embodiments. Further provided herein are vectors (e.g., expression vectors) comprising the polynucleotide(s) according to any one of the above embodiments. Further provided herein are host cells (e.g., isolated host cells or cell lines) comprising the polynucleotide(s) or vector(s) according to any one of the above embodiments. Further provided herein are methods of producing an antibody or fusion protein, comprising culturing the host cell according to any one of the above embodiments under conditions suitable for production of the antibody or fusion protein. In some embodiments, the methods further comprise recovering the antibody or fusion protein from the host cell.

Further provided herein are pharmaceutical compositions comprising the antibody or fusion protein according to any one of the above embodiments and a pharmaceutically acceptable carrier.

Further provided herein is the use of the antibody, fusion protein, or composition according to any one of the above embodiments as a medicament. Further provided herein is the use of the antibody, fusion protein, or composition according to any one of the above embodiments in a method for treating cancer or infection (e.g., chronic infection). Further provided herein is the use of the antibody, fusion protein, or composition according to any one of the above embodiments for the manufacture of a medicament for treating cancer or infection (e.g., chronic infection). Further provided herein are methods of treating cancer comprising administering to an individual with cancer an effective amount of the antibody, fusion protein, or composition according to any one of the above embodiments. In some embodiments, the methods further comprise administering to the individual a T cell therapy, cancer vaccine, chemotherapeutic agent, or immune checkpoint inhibitor (ICI). Further provided herein are methods of treating infection (e.g., chronic infection) comprising administering to an individual with infection an effective amount of the antibody, fusion protein, or composition according to any one of the above embodiments. Further provided herein are methods of expanding T cells (e.g., ex vivo) comprising contacting one or more T cells (e.g., tumor infiltrating lymphocytes) ex vivo (e.g., ex vivo) with an effective amount of the antibody, fusion protein, or composition according to any one of the above embodiments.

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

### BRIEF DESCRIPTION OF THE DRAWINGS

FIGS. 1A-1C depict the three types of CD8ab antibodies that could be identified according to their binding preference for CD8a, CD8b and CD8ab heterodimer. Antibodies binding CD8a epitopes are shown in FIG. 1A, antibodies binding epitopes that span both CD8a and CD8b are shown in FIG. 1B, and antibodies binding to CD8b epitopes are shown in FIG. 1C. Binding preference for CD8a, CD8b and CD8ab heterodimer by each antibody type is also depicted.

FIGS. 2A-2C show the results of an ELISA assay used to distinguish the three types of CD8ab antibodies depicted in FIGS. 1A-1C. Binding to recombinant CD8a (filled square), CD8b (filled triangle), CD8ab heterodimer (filled circle) and an irrelevant antigen, ovalbumin (open triangles) was measured. xhCD8al (clone OKT8) and xhCD8a2 antibodies (clone SK1) were previously described.

FIGS. 3A-3C depict the binding preferences on various CD8+ immune cell types by the three types of anti-CD8ab antibodies depicted in FIGS. 2A-2C. Antibodies with CD8a epitopes depicted in FIG. 1A bind to both CD8ab+ T cells and CD8aa+NK cells as depicted in FIG. 3A. Antibodies with epitopes that span both CD8a and CD8b depicted in FIG. 1B preferentially bind to CD8ab+ T cells over CD8aa+NK cells as depicted in FIG. 3B. Antibodies with CD8b epitopes depicted in FIG. 1C preferentially bind to CD8ab+ T cells over CD8aa+NK cells as depicted in FIG. 3C.

FIGS. 4 & 5 depict the results of a flow cytometry assay to detect the binding of CD8ab antibodies to human PBMCs and distinguish the three types of CD8ab antibodies depicted in FIGS. 1A-1C. Binding of CD8ab antibodies to T cells (FIG. 4) and NK cells (FIG. 5) in hPBMCs was detected by staining with anti-human Fc antibody conjugated to APC. Anti-hFc was used to measure the binding of CD8 antibodies containing hFc. Mean fluorescence intensity (MFI) of staining with anti-hFc is used to denote binding. Binding of anti-hFc was determined in CD3+CD8a+CD8b+ cells (CD8+ T cells) and CD3-CD56+CD8a+ cells (CD8+NK cells).

FIG. 6 depicts the results of a flow cytometry assay measuring the binding of CD8ab antibodies binding CD8b epitopes, xhCD8v1 to xhCD8v5, to CD8+ T cells. Negative control antibody, binding HA antigen, was also included.

FIG. 7 depicts four different formats (formats A, B, C, and D) of fusion proteins comprising CD8ab antibodies of the present disclosure, in accordance with some embodiments.

FIGS. 8A-8C depict fusion proteins comprising the three types of CD8ab antibodies depicted in FIGS. 1A-1C and FIGS. 3A-3C and an IL-2Rbg-binding polypeptide. Preferential activation of CD8ab+ T cells over CD8aa+NK cells is shown.

FIG. 9 shows the results of an assay to determine the selectivity of fusion proteins comprising the CD8ab antibodies and an IL-2Rbg-binding polypeptide, IL2 ml. The expression of the proliferation marker Ki-67 in CD8+ T cells and NK cells from a hPBMC donor was measured by flow cytometry after five days of co-culture with the following fusion proteins: Ctrl Ab-IL2v, comprising a control antibody and a previously published IL-2 variant (Klein et al, Oncoimmunol. 2017; 6 (3); c1277306) (top panel); xhCD8al-IL-2 ml, comprising CD8 antibody, xhCD8a1, targeting CD8a epitope, and the mutant IL-2 polypeptide of the present disclosure, IL2 ml (bottom left panel); and xhCD8v1-IL2 ml, comprising CD8ab antibody of the present disclosure, xhCD8v1, and IL2 ml (bottom right panel). xhCD8al-IL-2 ml was of format C, while Ctrl Ab-IL2v and xhCD8v1-IL2 ml were of format A. Ki-67 expression was measured in CD8+T cells (filled circles) and NK cells (filled squares). NK cells were defined as CD3-CD56+. CD8 expression was measured by staining with CD8 antibody SK1.

FIGS. 10A-10C depict the expression of Ki-67 proliferation marker after five day co-culture of hPBMCs from three different donors with indicated fusion proteins. All fusion proteins contained the mutant IL-2 polypeptide, IL2 ml, and CD8ab antibodies of the present disclosure, xhCD8v1 to xhCD8v7 and were of format A. Ki-67 expression was measured in CD8+ T cells (solid lines) and NK cells (dashed lines).

FIGS. 11A-11D show the results of the phospho-STAT5 assay with hPBMCs from one donor cultured with the indicated fusion proteins, all of format C. Percent pSTAT5-expressing cells was depicted in the following hPBMC subsets: CD8+ T cells (FIG. 11A), NK cells (FIG. 11B), Treg cells (FIG. 11C), and CD4+ Foxp3-T cells (FIG. 11D). NK cells were identified as CD3-Perforin+. Treg cells were identified as CD4+ Foxp3+CD25+.

FIG. 12 shows the expression of Ki-67 proliferation marker after five day co-culture of hPBMCs from one donor with indicated fusion proteins. All fusion proteins contained the CD8ab antibody of the present disclosure, xhCD8v1, and various mutant IL-2 polypeptides of the present disclosure, IL2 ml to IL2m5, and were of format A. Ki-67 expression was measured in CD8+ T cells (left panel) and NK cells (right panel).

FIG. 13A and FIG. 13B show the results of the phospho-STAT5 assay with hPBMCs from one donor cultured with the indicated fusion proteins. Percent pSTAT5-expressing cells was depicted in the following hPBMC subsets: CD8+ T cells and NK cells. In FIG. 13A, all fusion proteins contained the CD8ab antibody of the present disclosure, xhCD8v1, and various mutant IL-2 polypeptides of the present disclosure, IL2 ml, IL2m2, and IL2m6 to IL2m10, and were of format A. In FIG. 13B, fusion proteins contained the CD8ab antibodies of the present disclosure, xhCD8v11 and xhCD8v12, and various mutant IL-2 polypeptides of the present disclosure, IL2m11, IL2m12, and IL2m13, and were of format A. NK cells were identified as CD3-Perforin+.

FIG. 14A shows the expression of Ki-67 proliferation marker after five day co-culture of hPBMCs from one donor with xhCD8v1-IL2 ml (left panel) and xhCD8v6-IL2 ml (right panel). Three different fusion protein formats as depicted in FIG. 7 were tested: format A, format B and format C, as indicated. Solid lines depict Ki-67 expression in CD8+ T cells and dashed lines represent Ki-67 expression in NK cells. Format A resulted in substantial increase in activity on CD8+ T cells over NK cells for both CD8ab antibodies tested suggesting this is the preferred format for fusion proteins containing CD8ab antibodies targeting epitopes between CD8a and CD8b (FIG. 8B) or CD8b epitopes (FIG. 8C). FIG. 14B depicts the binding (left panel) and induction of Ki67 (right panel) in CD8+ T cells from one donor with xhCD8v6-IL2 ml. Four different fusion protein formats as depicted in FIG. 7 were tested: format A, format B, format C, and format D as indicated. While format A is approximately 5-fold stronger than other format in cell binding, format A is 20- to 40-fold more potent than other formats in inducing Ki-67. FIG. 14C depicts the results of nine fusion molecules in format A and D as assessed for binding to CD8+ T cells, and induction of Ki67 in CD8+ T cells. Examples for binding and ki-67 curves for xhCD8v12-IL2m4 were shown in the left panel. For a given binder and mutein pair, the ratio of EC50s of format D over format A is shown for binding and Ki67 in the right panel. For all the molecules tested, format A showed a potency shift over format D of approximately 10-fold in binding EC50. However, when assessed for the activation of a further downstream proliferation marker Ki67, format A exhibits approximately 1000-fold increase in potency over format D in inducing Ki-67.

FIG. 15 shows the expression of Ki-67 proliferation marker after five day co-culture of hPBMCs with xhCD8v8-IL2 ml, a fusion protein of format A containing the mutant IL-2 polypeptide, IL2 ml, and CD8ab antibody of the present disclosure, xhCD8v8. Ki-67 expression was measured in CD8+ T cells (squares) and NK cells (triangles).

FIG. 16A depicts the expression of Ki-67 in tumor infiltrating lymphocytes (TILs) gated on CD8+ T cells (left panel) and NK cells (right panel). TILs were incubated with rhIL-2 or the indicated fusion proteins (format A) for five days before analysis. FIG. 16B depicts the number of CD8+ T cells, NK cells, and CD4+ T cells per well in each of the indicated conditions.

FIGS. 17A-17E show expression of Ki-67 proliferation marker after five day co-culture of hPBMCs with fusion proteins of format A containing a mutant IL-2 polypeptide (IL2 ml or IL2m4) and an CD8ab antibody of the present disclosure. CD8ab antibodies xhCD8v9-14 were tested. Ki-67 expression was measured in CD8+ T cells (squares) and NK cells (triangles).

FIG. 18A shows numbers of amino acid liabilities (e.g., putative N-linked glycosylation, deamination, or acid cleavage sites) and amino acid mismatches as compared to human germline of the indicated anti-CD8ab antibodies.

FIG. 18B shows alignments illustrating how CDRs (italics) from xhCD8v1 VH (top) and VK (bottom) were grafted onto VH1-69 and VK1-39 human germline frameworks, respectively. VH domain sequences (top) correspond to SEQ ID Nos: 58, 384, and 60, respectively (top to bottom). VL domain sequences (bottom) correspond to SEQ ID Nos: 59, 385, and 61, respectively (top to bottom). Underlined residues depict framework residues of human germline.

FIGS. 18C & 18D show alignments of VH (FIG. 18C) and VL (FIG. 18D) domains of the indicated anti-CD8ab antibodies of the present disclosure. VH domain sequences (FIG. 18C) correspond to SEQ ID Nos: 60, 62, 64, 66, 68, 245, 251, and 253, respectively (top to bottom). VL domain sequences (FIG. 18D) correspond to SEQ ID Nos: 61, 63, 65, 67, 69, 246, 252, and 254, respectively (top to bottom).

FIG. 19 shows the results of an ELISA assay used to identify glycosylation of the parental xhCD8v6 (black solid line with black squares), which is removed upon treatment with glycosidases (black dashed line with black squares). No glycosylation is detected on the xhCD8v11 (gray lines with open triangles). Fetuin is shown as a positive control.

### DETAILED DESCRIPTION

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the present disclosure include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per sc.

“Immune cells” as used here are cells of the immune system that react to organisms or other entities that are deemed foreign to the immune system of the host. They protect the host against foreign pathogens, organisms and diseases. Immune cells, also called leukocytes, are involved in both innate and adaptive and immune responses to fight pathogens. Innate immune responses occur immediately upon exposure to pathogens without additional priming or learning processes. Adaptive immune processes require initial priming, and subsequently create memory, which in turn leads to enhanced responsiveness during subsequent encounters with the same pathogen. Innate immune cells include, but are not limited to monocytes, macrophages, dendritic cells, innate lymphoid cells (ILCs) including natural killer (NK) cells, neutrophils, megakaryocytes, cosinophils and basophils. Adaptive immune cells include B and T lymphocytes/cells. T cells subsets include, but are not limited to, alpha beta CD4+ T (naïve CD4+, memory CD4+, effector memory CD4+, effector CD4+, regulatory CD4+), and alpha beta CD8+ T (naïve CD8+, memory CD8+, effector memory CD8+, effector CD8+). B cell subsets include, but is not limited to, naïve B, memory B, and plasma cells. NK T cells and T gamma delta (Tγδ) cells exhibit properties of both innate and adaptive lymphocytes.

“T cells” or “T lymphocytes” are immune cells that play a key role in the orchestration of immune responses in health and disease. Two major T cell subsets exist that have unique functions and properties: T cells that express the CD8 antigen (CD8+ T cells) are cytotoxic or killer T cells that can lyse target cells using the cytotoxic proteins such as granzymes and perforin; and T cells that express the CD4 antigen (CD4+ T cells) are helper T cells that are capable of regulating the function of many other immune cell types including that of CD8+ T cells, B cells, macrophages etc. Furthermore, CD4+ T cells are further subdivided into several subsets such as: T regulatory (Treg) cells that are capable of suppressing the immune response, and T helper 1 (Th1), T helper 2 (Th2), and T helper 17 (Th17) cells that regulate different types of immune responses by secreting immunomodulatory proteins such as cytokines. T cells recognize their targets via alpha beta T cell receptors that bind to unique antigen-specific motifs and this recognition mechanism is generally required in order to trigger their cytotoxic and cytokine-secreting functions. “Innate lymphocytes” can also exhibit properties of CD8+ and CD4⁺ T cells, such as the cytotoxic activity or the secretion of Th1, Th2, and Th17 cytokines. Some of these innate lymphocyte subsets include NK cells and ILC1, ILC2, and ILC3 cells; and innate-like T cells such as Tγδ cells; and NK T cells. Typically, these cells can rapidly respond to inflammatory stimuli from infected or injured tissues, such as immunomodulatory cytokines, but unlike alpha beta T cells, they can respond without the need to recognize antigen-specific patterns.

“Cytokine” is a form of immunomodulatory polypeptide that mediates cross-talk between initiating/primary cells and target/effector cells. It can function as a soluble form or cell-surface associated to bind the “cytokine receptor” on target immune cells to activate signaling. “Cytokine receptor” as used here is the polypeptide on the cell surface that activates intracellular signaling upon binding the cytokine on the extracellular cell surface. Cytokines includes, but are not limited to, chemokines, interferons, interleukins, lymphokines, and tumor necrosis factors. Cytokines are produced by a wide range of cells, including immune cells, endothelial cells, fibroblasts, and stromal cells. A given cytokine may be produced by more than one cell type. Cytokine are pleiotropic; since the receptors are expressed on multiple immune cell subsets, one cytokine can activate the signaling pathway in multiple cells. However, depending on the cell type, the signaling events for a cytokine can result in different downstream cellular events such as activation, proliferation, survival, apoptosis, effector function and secretion of other immunomodulatory proteins.

“Amino acid” as used here refers to naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Polypeptide” or “protein” as used here refers to a molecule where monomers (amino acids) are linearly linked to one another by peptide bonds (also known as amide bonds). The term “polypeptide” refers to any chain of two or more amino acids and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of A polypeptide may be derived from a natural biological source or produced by recombinant technology but is not necessarily translated from a designated nucleic acid sequence. It may be generated in any manner, including by chemical synthesis. Polypeptides normally have a defined three-dimensional structure, but they do not necessarily have such structure. A polypeptide of the present disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides with a defined three-dimensional structure are referred to as folded, and polypeptides which do not possess a defined three-dimensional structure, but rather can adopt many different conformations and are referred to as unfolded. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. The terms “polypeptide” and “protein” also refer to modified polypeptides/proteins wherein the post-expression modification is affected including without limitation glycosylation, acetylation, phosphorylation, amidation, derivatization by known protecting/blocking groups, proteolytic cleavage, or modification by non-naturally occurring amino acids.

“Residue” as used herein is meant a position in a protein and its associated amino acid identity. For example, Leu 234 (also referred to as Leu234 or L234) is a residue at position 234 in the human antibody IgG1.

“Wild-type” herein means an amino acid sequence or a nucleotide sequence that is found in nature, including allelic variations. A wild-type protein has an amino acid sequence or a nucleotide sequence that has not been intentionally modified.

“Substitution” or “mutation” refers to a change to the polypeptide backbone wherein an amino acid occurring naturally in the wild-type sequence of a polypeptide is substituted to another amino acid not naturally occurring at the same position in the said polypeptide. Preferably, a mutation or mutations are introduced to modify polypeptide's affinity to its receptor thereby altering its activity such that it becomes different from the affinity and activity of the wild-type cognate polypeptide. Mutations can also improve polypeptide's biophysical properties. Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

“CD8” as used here refers to any native human CD8. Unless otherwise indicated expressly or by context, references to “CD8” refer to CD8aa and/or CD8ab. The amino acid sequence of an exemplary human CD8b, the beta chain of human CD8, is described under UniProt P10966 (CD8B_HUMAN). “CD8a” refers to the alpha chain of human CD8 (e.g., as is described under UniProt P01732 (CD8A_HUMAN)). “CD8aa” refers to a homodimer of CD8a. “CD8ab” refers to a heterodimer of CD8a and CD8b. “CD8,” “CD8a,” “CD8b,” “CD8aa,” and “CD8ab” encompass unprocessed forms as well as mature forms that result from processing in the cell. “CD8,” “CD8a,” “CD8b,” “CD8aa,” and “CD8ab” also include but are not limited to naturally occurring variants, e.g. allelic or splice variants or variants.

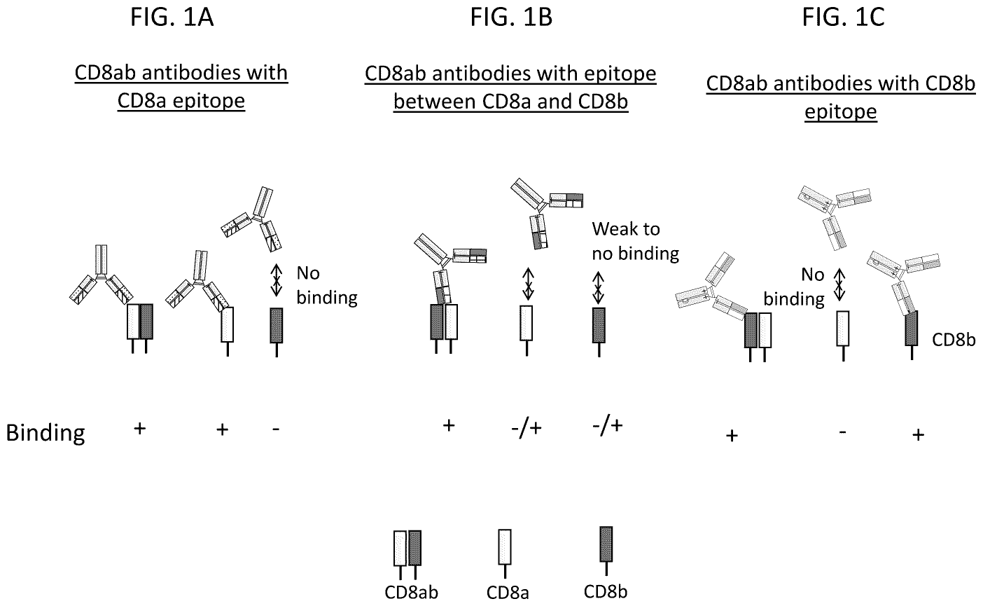

“Interleukin-2” or “IL-2” as used here refers to any native human IL-2, unless otherwise indicated. “IL-2” encompasses unprocessed IL-2 as well as “mature IL-2” which is a form of IL-2 that results from processing in the cell. The sequence of “mature IL-2” is depicted in FIG. 1A. One exemplary form of unprocessed human IL-2 comprises of an additional N-terminal amino acid signal peptide attached to mature IL-2. “IL-2” also includes but is not limited to naturally occurring variants of IL-2, e.g. allelic or splice variants or variants. The amino acid sequence of an exemplary human IL-2 is described under UniProt P60568 (IL2_HUMAN).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity can generally be represented by the dissociation constant (K_{D}), which is the ratio of dissociation and association rate constants (koff and kon, respectively). Thus, equivalent affinities may comprise different rate constants, as long as the ratio of the rate constants remains the same. Affinity can be measured by common methods known in the art, such as enzyme-linked immunosorbent assay (ELISA), surface plasmon resonance (SPR) technologies (e.g. BIAcore), BioLayer Interferometry (BLI) technologies (e.g. Octet) and other traditional binding assays (Heeley, Endocr Res 28, 217-229 (2002).

“Binding” or “specific binding” as used here, refers the ability of a polypeptide or an antigen binding molecule to selectively interact with the receptor for the polypeptide or target antigen, respectively, and this specific interaction can be distinguished from non-targeted or undesired or non-specific interactions. Examples of specific binding include but are not limited to IL-2 cytokine binding to its specific receptors (e.g. IL-2Rα, IL-2Rβ and IL-2Ry) and an antigen binding molecule binding to a specific antigen (e.g. CD8 or PD-1).

“Mutant IL-2 polypeptide” refers to IL-2 polypeptide that has reduced affinity to its receptor wherein such decreased affinity will result in reduced biological activity of the mutant. Reduction in affinity and thereby activity can be obtained by introducing a small number of amino acid mutations or substitutions. The mutant IL-2 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-2Rβγ. Desired activity may also include improved biophysical properties compared to the wild-type IL-2 polypeptide. Multiple modifications may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. Preferably, a mutation or mutations are introduced to the polypeptide to modify its activity.

The terms “antibody” and “immunoglobulin” are used interchangeably and herein are used in the broadest sense and encompasses various antibody structures, including but not limited to monoclonal antibodies (e.g., full length or intact monoclonal antibodies), polyclonal antibodies, multispecific antibodies (e.g. bispecific antibodies), antibody fragments and single domain antibody (as described in greater detail herein), so long as they exhibit the desired antigen binding activity.

Antibodies (immunoglobulins) refer to a protein having a structure substantially similar to a native antibody structure. “Native antibodies” refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000, Cellular and Mol, and Kindt et al., Kuby Immunology, 6th ed., W.H. Freeman and Co., page 91 (2007). Antibodies (immunoglobulins) are assigned to different classes, depending on the amino acid sequences of the heavy chain constant domains. There are five major classes of antibodies: α (IgA), δ (IgD), ∈(IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

“Fc” or “Fc region” or “Fc domain” as used herein refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. The “hinge” region usually extends from amino acid residue at about position 216 to amino acid residue at about position 230. The hinge region herein may be a native hinge domain or variant hinge domain. The “CH2 domain” of a human IgG Fc region usually extends from an amino acid residue at about position 231 to an amino acid residue at about position 340. The CH2 domain herein may be a native sequence CH2 domain or variant CH2 domain. The “CH3 domain” comprises the stretch of residues C-terminal to a CH2 domain in an Fc region, from an amino acid residue at about position 341 to an amino acid residue at about position 447 of an IgG. The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.). Human IgG Fc domains are of particular use in the present disclosure, and can be the Fc domain from human IgG1, IgG2 or IgG4.

A “variant Fc domain” or “Fc variant” or “variant Fc” contains amino acid modifications (e.g. substitution, addition, and deletion) as compared to a parental Fc domain. The term also includes naturally occurring allelic variants of the Fc region of an immunoglobulin. In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. For example, one or more amino acids can be deleted from the N-terminus or C-terminus of the Fc region of an immunoglobulin without substantial loss of biological function. Additionally, as discussed herein, the variant Fc domains herein still retain the ability to form a dimer with another Fc domain as measured using known techniques as described herein, such as non-denaturing gel electrophoresis.

“Fc gamma receptor”, “FcγR” or “Fc gamma R” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIla (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NAI and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “effector function” as used herein is meant a biochemical event that results from the interaction of an antibody Fc region with an Fc receptor or ligand, which vary with the antibody isotype. Effector functions include but are not limited to antibody-dependent cell-mediated cytotoxicity (ADCC), antibody-dependent cell-mediated phagocytosis (ADCP), complement-dependent cytotoxicity (CDC), cytokine secretion, immune complex-mediated antigen uptake by antigen presenting cells, down regulation of cell surface receptors (e.g. B cell receptor), and B cell activation. “Antibody-dependent cell-mediated cytotoxicity” or “ADCC” refer to a cell-mediated reaction in which nonspecific cytotoxic cells that express FcRs (such as Natural Killer (NK) cells, neutrophils, and macrophages) recognize bound antibody on a target cell and subsequently cause lysis of the target cell. ADCC is correlated with binding to FcγRIIIa; increased binding to FcγRIIIa leads to an increase in ADCC activity. To assess ADCC activity of a molecule of interest, an in vitro ADCC assay such as that described in U.S. Pat. No. 5,500,362 or 5,821,337 may be performed. “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

“Fc null” and “Fc null variant” are used interchangeably and used herein to describe a modified Fc which have reduced or abolished effector functions. Such Fc null or Fc null variant have reduced or abolished to FcγRs and/or complement receptors. Preferably, such Fc null or Fc null variant has abolished effector functions. Exemplary methods for the modification include but not limited to chemical alteration, amino acid residue substitution, insertion and deletions. Exemplary amino acid positions on Fc molecules where one or more modifications were introduced to decrease effector function of the resulting variant (numbering based on the EU numbering scheme) at position i) IgG1: C220, C226, C229, E233, L234, L235, G237, P238, S239 D265, S267, N297, L328, P331, K322, A327 and P329, ii) IgG2: V234, G237, D265, H268, N297, V309, A330, A331, K322 and iii) IgG4: L235, G237, D265 and E318. Exemplary Fc molecules having decreased effector function include those having one or more of the following substitutions: i) IgG1: N297A, N297Q, N297G, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G,E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted; ii) IgG2: A330S/A331S, V234A/G237A, V234A/G237A/D265A, D265A/A330S/A331S, V234A/G237A/D265A/A330S/A331S, and H268Q/V309L/A330S/A331S; iii) IgG4: L235A/G237A/E318A, D265A, L235A/G237A/D265A and L235A/G237A/D265A/E318A.

“Epitope” as used herein refers to a determinant capable of specific binding to the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding and other amino acid residues, which are not directly involved in the binding, such as amino acid residues which are effectively blocked by the antigen binding peptide (in other words, the amino acid residue is within the footprint of the antigen binding peptide). Epitopes may be either conformational or linear. An epitope typically includes at least 3, and more usually, at least 5 or 8-10 amino acids. Antibodies that recognize the same epitope can be verified in a simple immunoassay showing the ability of one antibody to block the binding of another antibody to a target antigen, for example “binning”.

“Linker” as used herein refers to a molecule that connect two polypeptide chains. Linker can be a polypeptide linker or a synthetic chemical linker (for example, see disclosed in Protein Engineering, 9 (3), 299-305, 1996). The length and sequence of the polypeptide linkers is not particularly limited and can be selected according to the purpose by those skilled in the art. Polypeptide linker comprises one or more amino acids. In some embodiments, the polypeptide linker is a peptide with a length of at least 5 amino acids, preferably with a length of 5 to 100, more preferably of 10 to 50 amino acids. In one embodiment, said peptide linker is G, S, GS, SG, SGG, GGS, and GSG (with G=glycine and S=serine). In another embodiment, said peptide linker is (GGGS)xGn (SEQ ID NO:74) or (GGGGS)xGn (SEQ ID NO:75) or (GGGGGS)xGn (SEQ ID NO:76) or S(GGGS)xGn (SEQ ID NO:386) or S(GGGGS)xGn (SEQ ID NO:387) or S(GGGGGS)xGn (SEQ ID NO:388), with x=1, 2, 3, 4, 5, 6, 7, 8, 9, 10, 11, or 12 and n=0, 1, 2 or 3. Preferably, the said linker is (GGGGS)xGn with x=2,3, or 4 and n=0 (SEQ ID NO:77); more preferably the said linker is (GGGGS)xGn with x=3 and n=0 (SEQ ID NO:78). In some embodiments, the linker comprises the sequence GGGGSGGGGSGGGGS (SEQ ID NO:79) or or SGGGGSGGGGSGGGGS (SEQ ID NO:389). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis [2-(succinimidoxycarbonyloxy)ethyl] sulfone (BSOCOES), and bis [2-(succinimidoxycarbonyloxy)ethyl] sulfone (sulfo-BSOCOES).

The term “polynucleotide” refers to an isolated nucleic acid molecule or construct, e.g. messenger RNA (mRNA), virally-derived RNA, or plasmid DNA (pDNA) encoding the polypeptides of the present disclosure. A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide. In some aspects, one or more vectors (particularly expression vectors) comprising such nucleic acids are provided. In one aspect, a method for making a polypeptide of the present disclosure is provided, wherein the methods comprises culturing a host cell comprising a nucleic acid encoding the polypeptide under conditions suitable for expression of the polypeptide and recovering the polypeptide from the host cell. “Recombinant” means the proteins are generated using recombinant nucleic acid techniques in exogeneous host cells. Recombinantly produced proteins expressed in host cells are considered isolated for the purpose of the present disclosure, as are native or recombinant proteins which have been separated, fractionated, or partially or substantially purified by any suitable technique.

“Isolated,” when used to describe the various polypeptides disclosed herein, means a polypeptide that has been identified and separated and/or recovered from a cell or cell culture from which it was expressed. Typically, an isolated polypeptide will be purified by at least one purification step. There is no required level of purity; “purification” or “purified” refers to increase of the target protein concentration relative to the concentration of contaminants in a composition as compared to the starting material. An “isolated protein,” as used herein refers to a target protein which is substantially free of other proteins having different binding specificities.

The terms “cancer” refers the physiological condition in mammals that is typically characterized by unregulated and abnormal cell growth with the potential to invade or spread to other parts of the body. Examples of cancer include but are not limited to, carcinoma, lymphoma, blastoma, sarcoma, and leukemia. More particular examples of such cancers include lung cancer, small-cell lung cancer, non-small cell lung (NSCL) cancer, bronchioloalviolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliay cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

### Antibodies and Antigen Binding Domains

Certain aspects of the present disclosure relate to antibodies, antibody fragments, and antigen binding domains that specifically bind human CD8b and/or human CD8ab. Any of the anti-CD8 antibodies of the present disclosure (e.g., that specifically bind human CD8b and/or human CD8ab) may find use in the fusion proteins, methods, and uses disclosed herein.

In some embodiments, the anti-CD8 antibody of the present disclosure specifically binds human CD8b and/or human CD8ab with at least 10-fold, at least 20-fold, at least 30-fold, at least 40-fold, at least 50-fold, at least 60-fold, at least 70-fold, at least 80-fold, at least 90-fold, at least 100-fold, or at least 200-fold higher affinity than its binding to human CD8a and/or human CD8aa, e.g., as expressed on natural killer (NK) cells (e.g., human NK cells). In some embodiments, the anti-CD8 antibody of the present disclosure specifically binds human CD8b and/or human CD8ab with at least 10-fold higher affinity than its binding to human CD8a and/or human CD8aa, e.g., as expressed on natural killer (NK) cells. In some embodiments, the human CD8b and/or human CD8ab are expressed on the surface of a human cell, e.g., a human T cell.

In some embodiments, the anti-CD8 antibody of the present disclosure specifically binds to a cell expressing a human CD8ab heterodimer on its surface (e.g., a human T cell) with an EC50 that is less than 1000 nM. In some embodiments, the anti-CD8 antibody of the present disclosure specifically binds to human CD8+ T cells.

In some embodiments, the anti-CD8 antibody of the present disclosure is a human antibody or antibody fragment. In some embodiments, a human antibody or antibody fragment comprises human-derived CDRs and framework sequences in the variable domain, e.g., as isolated from a human or generated using a library with human antibody sequences (e.g., CDR sequences). In some embodiments, the anti-CD8 antibody of the present disclosure is a humanized antibody or antibody fragment. In some embodiments, a humanized antibody or antibody fragment comprises non-human-derived CDRs (e.g., from a mouse, rabbit, goat, etc.) and human-derived framework sequences in the variable domain. In some embodiments, a human or humanized antibody further comprises a human Fc region. In some embodiments, the human Fc region further comprises one or more Fc mutations, e.g., as disclosed herein. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, δ, ε, γ, and μ, respectively. The subunit structures and three-dimensional configurations of different classes of immunoglobulins are well known.

Multiple definitions for the CDR sequences of antibody variable domains are known in the art. Unless otherwise specified, CDR sequences are described herein according to the definition of Kabat (see, e.g., Kabat et al., Sequences of Proteins of Immunological Interest, Fifth Edition, NIH Publication 91-3242, Bethesda MD (1991), vols. 1-3). However, other definitions are known and contemplated for use. For example, in some embodiments, CDR sequences can be described by the definition of Chothia (see, e.g., Chothia and Lesk, J. Mol. Biol. 196:901-917 (1987). Depending upon the specific CDR definition used, the precise framework sequences can also vary, but, as is known in the art, the first framework sequence (FW-1) refers to the sequence from the N-terminus of the VH or VL domain to the beginning of CDR-H1/-L1, the second framework sequence (FW-2) refers to the sequence from end of CDR-H1/-L1 to the beginning of CDR-H2/-L2, the third framework sequence (FW-3) refers to the sequence from end of CDR-H2/-L2 to the beginning of CDR-H3/-L3, and the fourth framework sequence (FW-4) refers to the sequence from end of CDR-H3/-L3 to the C-terminal boundary of the VH or VL domain.

In some embodiments, an anti-CD8 antibody of the present disclosure comprises a VH domain comprising a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3 and a VL domain comprising a CDR-L1 comprising the amino acid sequence of SEQ ID NO:4, a CDR-L2 comprising the amino acid sequence of SEQ ID NO: 5, and a CDR-L3 comprising the amino acid sequence of SEQ ID NO:6. In some embodiments, an anti-CD8 antibody of the present disclosure comprises 1, 2, or 3 heavy chain CDRs of antibody xhCD8v1 (e.g., as shown in Tables 1-3) and/or 1, 2, or 3 light chain CDRs of antibody xhCD8v1 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:58 and a VL domain comprising a CDR-L1, CDR-L2, and CDR-L3 from the sequence of SEQ ID NO: 59.

In some embodiments, an anti-CD8 antibody of the present disclosure comprises a VH domain comprising a CDR-H1 comprising the amino acid sequence of SEQ ID NO:177, a CDR-H2 comprising the amino acid sequence of SEQ ID NO: 178, and a CDR-H3 comprising the amino acid sequence of SEQ ID NO: 179 and a VL domain comprising a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182. In some embodiments, an anti-CD8 antibody of the present disclosure comprises 1, 2, or 3 heavy chain CDRs of antibody xhCD8v8 (e.g., as shown in Tables 1-3) and/or 1, 2, or 3 light chain CDRs of antibody xhCD8v8 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 185 and a VL domain comprising a CDR-L1, CDR-L2, and CDR-L3 from the sequence of SEQ ID NO:186.

In some embodiments, an anti-CD8 antibody of the present disclosure comprises a VH domain comprising a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO:15 and a VL domain comprising a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:18. In some embodiments, the VH domain comprises an amino acid sequence that is at least 90%, at least 95%, at least 99%, or 100% identical to the sequence of SEQ ID NO:62 and/or the VL domain comprises an amino acid sequence that is at least 90%, at least 95%, at least 99%, or 100% identical to the sequence of SEQ ID NO:63. In some embodiments, an anti-CD8 antibody of the present disclosure comprises 1, 2, or 3 heavy chain CDRs of antibody xhCD8v2 (e.g., as shown in Tables 1-3) and/or 1, 2, or 3 light chain CDRs of antibody xhCD8v2 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:62 and a VL domain comprising a CDR-L1, CDR-L2, and CDR-L3 from the sequence of SEQ ID NO:63.

In some embodiments, an anti-CD8 antibody of the present disclosure comprises a VH domain comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21 and a VL domain comprising a CDR-L1 comprising the amino acid sequence of SEQ ID NO:22, a CDR-L2 comprising the amino acid sequence of SEQ ID NO: 23, and a CDR-L3 comprising the amino acid sequence of SEQ ID NO:24. In some embodiments, the VH domain comprises an amino acid sequence that is at least 90%, at least 95%, at least 99%, or 100% identical to the sequence of SEQ ID NO:64 and/or the VL domain comprises an amino acid sequence that is at least 90%, at least 95%, at least 99%, or 100% identical to the sequence of SEQ ID NO:65. In some embodiments, an anti-CD8 antibody of the present disclosure comprises 1, 2, or 3 heavy chain CDRs of antibody xhCD8v3 (e.g., as shown in Tables 1-3) and/or 1, 2, or 3 light chain CDRs of antibody xhCD8v3 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:64 and a VL domain comprising a CDR-L1, CDR-L2, and CDR-L3 from the sequence of SEQ ID NO:65.

In some embodiments, an anti-CD8 antibody of the present disclosure comprises a VH domain comprising a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27 and a VL domain comprising a CDR-L1 comprising the amino acid sequence of SEQ ID NO:28, a CDR-L2 comprising the amino acid sequence of SEQ ID NO: 29, and a CDR-L3 comprising the amino acid sequence of SEQ ID NO:30. In some embodiments, the VH domain comprises an amino acid sequence that is at least 90%, at least 95%, at least 99%, or 100% identical to the sequence of SEQ ID NO:66 and/or the VL domain comprises an amino acid sequence that is at least 90%, at least 95%, at least 99%, or 100% identical to the sequence of SEQ ID NO:67. In some embodiments, an anti-CD8 antibody of the present disclosure comprises 1, 2, or 3 heavy chain CDRs of antibody xhCD8v4 (e.g., as shown in Tables 1-3) and/or 1, 2, or 3 light chain CDRs of antibody xhCD8v4 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:66 and a VL domain comprising a CDR-L1, CDR-L2, and CDR-L3 from the sequence of SEQ ID NO:67.

In some embodiments, an anti-CD8 antibody of the present disclosure comprises a VH domain comprising a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33 and a VL domain comprising a CDR-L1 comprising the amino acid sequence of SEQ ID NO:34, a CDR-L2 comprising the amino acid sequence of SEQ ID NO: 35, and a CDR-L3 comprising the amino acid sequence of SEQ ID NO:36. In some embodiments, the VH domain comprises an amino acid sequence that is at least 90%, at least 95%, at least 99%, or 100% identical to the sequence of SEQ ID NO:68 and/or the VL domain comprises an amino acid sequence that is at least 90%, at least 95%, at least 99%, or 100% identical to the sequence of SEQ ID NO:69. In some embodiments, an anti-CD8 antibody of the present disclosure comprises 1, 2, or 3 heavy chain CDRs of antibody xhCD8v5 (e.g., as shown in Tables 1-3) and/or 1, 2, or 3 light chain CDRs of antibody xhCD8v5 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:68 and a VL domain comprising a CDR-L1, CDR-L2, and CDR-L3 from the sequence of SEQ ID NO:69.

In some embodiments, an anti-CD8 antibody of the present disclosure comprises a VH domain comprising a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39 and a VL domain comprising a CDR-L1 comprising the amino acid sequence of SEQ ID NO:40, a CDR-L2 comprising the amino acid sequence of SEQ ID NO: 41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:70 and/or the VL domain comprises an amino acid sequence that is at least 90%, at least 95%, at least 99%, or 100% identical to the sequence of SEQ ID NO:71. In some embodiments, an anti-CD8 antibody of the present disclosure comprises 1, 2, or 3 heavy chain CDRs of antibody xhCD8v6 (e.g., as shown in Tables 1-3) and/or 1, 2, or 3 light chain CDRs of antibody xhCD8v6 (e.g., as shown in Tables 1-3). In some embodiments, the antibody is human. In some embodiments, an anti-CD8 antibody of the present disclosure comprises a VH domain comprising a CDR-H1, CDR-H2, and CDR-H3 from the sequence of SEQ ID NO:70 and a VL domain comprising a CDR-L1, CDR-L2, and CDR-L3 from the sequence of SEQ ID NO:71.

In some embodiments, an anti-CD8 antibody of the present disclosure comprises a VH domain comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45 and a VL domain comprising a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48. In some embodiments, the VH domain comprises an amino acid sequence that is at least 90%, at least 95%, at least 99%, or 100% identical to the sequence of SEQ ID NO:72 and/or the VL domain comprises an amino acid sequence that is at least 90%, at least 95%, at least 99%, or 100% identical to the sequence of SEQ ID NO:73. In some embodiments, an anti-CD8 antibody of the present disclosure comprises 1, 2, or 3 heavy chain CDRs of antibody xhCD8v7 (e.g., as shown in Tables 1-3) and/or 1, 2, or 3 light chain CDRs of antibody xhCD8v7 (e.g., as shown in Tables 1-3). In some embodiments, the antibody is human. In some embodiments, an anti-CD8 antibody of the present disclosure comprises a VH domain comprising a CDR-H1, CDR-H2, and CDR-H3 from the sequence of SEQ ID NO:72 and a VL domain comprising a CDR-L1, CDR-L2, and CDR-L3 from the sequence of SEQ ID NO:73.

In some embodiments, an anti-CD8 antibody of the present disclosure comprises a VH domain comprising a CDR-H1 comprising the amino acid sequence of X₁X₂AIS, wherein X₁ is S, K, G, N, R, D, T, or G, and wherein X₂ is Y, L, H, or F (SEQ ID NO:259), a CDR-H2 comprising the amino acid sequence of X₁X₂X₃PX₄X₅X₆X₇X₈X₉YX₁₀QKFX1G, wherein X₁ is Gor H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, Xio is A or N, and Xu is Q or T (SEQ ID NO:260), and a CDR-H3 comprising the amino acid sequence of X₁X₂X₃GX₄XLFX6X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:261) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227 and a VL domain comprising a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:245 and/or the VL domain comprises an amino acid sequence that is at least 90%, at least 95%, at least 99%, or 100% identical to the sequence of SEQ ID NO:246. In some embodiments, an anti-CD8 antibody of the present disclosure comprises 1, 2, or 3 heavy chain CDRs of antibody xhCD8v9 (e.g., as shown in Tables 1-3) and/or 1, 2, or 3 light chain CDRs of antibody xhCD8v9 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:245 and a VL domain comprising a CDR-L1, CDR-L2, and CDR-L3 from the sequence of SEQ ID NO:246. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233 and a VL domain comprising a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises an amino acid sequence that is at least 90%, at least 95%, at least 99%, or 100% identical to the sequence of SEQ ID NO:251 and/or the VL domain comprises an amino acid sequence that is at least 90%, at least 95%, at least 99%, or 100% identical to the sequence of SEQ ID NO:252. In some embodiments, the VH domain comprises the amino acid sequence of SEQ ID NO:251; and the VL domain comprises the amino acid sequence of SEQ ID NO:252. In some embodiments, an anti-CD8 antibody of the present disclosure comprises 1, 2, or 3 heavy chain CDRs of antibody xhCD8v12 (e.g., as shown in Tables 1-3) and/or 1, 2, or 3 light chain CDRs of antibody xhCD8v12 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:251 and a VL domain comprising a CDR-L1, CDR-L2, and CDR-L3 from the sequence of SEQ ID NO:252. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:253 and/or the VL domain comprises an amino acid sequence that is at least 90%, at least 95%, at least 99%, or 100% identical to the sequence of SEQ ID NO:254. In some embodiments, an anti-CD8 antibody of the present disclosure comprises 1, 2, or 3 heavy chain CDRs of antibody xhCD8v13 (e.g., as shown in Tables 1-3) and/or 1, 2, or 3 light chain CDRs of antibody xhCD8v13 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:253 and a VL domain comprising a CDR-L1, CDR-L2, and CDR-L3 from the sequence of SEQ ID NO:254. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of X₁YX₂MS, wherein X₁ is S, D, E, A, or Q and X₂ is A, G, or T (SEQ ID NO:268), a CDR-H2 comprising the amino acid sequence of DIX1X₂X₃GX₄X₅TX6 YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO: 269), and a CDR-H3 comprising the amino acid sequence of X₁X₂X₃YX+WX5X₆AX-DX8, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270) and a VL domain comprising a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain further comprises a FW-1 comprising the sequence EVQLVESGGGLVQPGGSLRLSCAASGFTFS (SEQ ID NO: 281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO: 283), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:247 and/or the VL domain comprises an amino acid sequence that is at least 90%, at least 95%, at least 99%, or 100% identical to the sequence of SEQ ID NO:248. In some embodiments, an anti-CD8 antibody of the present disclosure comprises 1, 2, or 3 heavy chain CDRs of antibody xhCD8v10 (e.g., as shown in Tables 1-3) and/or 1, 2, or 3 light chain CDRs of antibody xhCD8v10 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:247 and a VL domain comprising a CDR-L1, CDR-L2, and CDR-L3 from the sequence of SEQ ID NO:248. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:249 and/or the VL domain comprises an amino acid sequence that is at least 90%, at least 95%, at least 99%, or 100% identical to the sequence of SEQ ID NO:250. In some embodiments, the VH domain comprises the amino acid sequence of SEQ ID NO:249; and the VL domain comprises the amino acid sequence of SEQ ID NO:250. In some embodiments, an anti-CD8 antibody of the present disclosure comprises 1, 2, or 3 heavy chain CDRs of antibody xhCD8v11 (e.g., as shown in Tables 1-3) and/or 1, 2, or 3 light chain CDRs of antibody xhCD8v11 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:249 and a VL domain comprising a CDR-L1, CDR-L2, and CDR-L3 from the sequence of SEQ ID NO:250. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:255 and/or the VL domain comprises an amino acid sequence that is at least 90%, at least 95%, at least 99%, or 100% identical to the sequence of SEQ ID NO:256. In some embodiments, an anti-CD8 antibody of the present disclosure comprises 1, 2, or 3 heavy chain CDRs of antibody xhCD8v14 (e.g., as shown in Tables 1-3) and/or 1, 2, or 3 light chain CDRs of antibody xhCD8v14 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:255 and a VL domain comprising a CDR-L1, CDR-L2, and CDR-L3 from the sequence of SEQ ID NO:256. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:257 and/or the VL domain comprises an amino acid sequence that is at least 90%, at least 95%, at least 99%, or 100% identical to the sequence of SEQ ID NO:258. In some embodiments, an anti-CD8 antibody of the present disclosure comprises 1, 2, or 3 heavy chain CDRs of antibody xhCD8v15 (e.g., as shown in Tables 1-3) and/or 1, 2, or 3 light chain CDRs of antibody xhCD8v15 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:257 and a VL domain comprising a CDR-L1, CDR-L2, and CDR-L3 from the sequence of SEQ ID NO:258. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3 and a VL domain comprising a CDR-L1 comprising the amino acid sequence of SEQ ID NO:4, a CDR-L2 comprising the amino acid sequence of SEQ ID NO: 5, and a CDR-L3 comprising the amino acid sequence of SEQ ID NO:6. In some embodiments, an anti-CD8 antibody of the present disclosure comprises a VH domain comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15 and a VL domain comprising a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO:18. In some embodiments, an anti-CD8 antibody of the present disclosure comprises a VH domain comprising a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21 and a VL domain comprising a CDR-L1 comprising the amino acid sequence of SEQ ID NO:22, a CDR-L2 comprising the amino acid sequence of SEQ ID NO: 23, and a CDR-L3 comprising the amino acid sequence of SEQ ID NO:24. In some embodiments, an anti-CD8 antibody of the present disclosure comprises a VH domain comprising a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27 and a VL domain comprising a CDR-L1 comprising the amino acid sequence of SEQ ID NO:28, a CDR-L2 comprising the amino acid sequence of SEQ ID NO: 29, and a CDR-L3 comprising the amino acid sequence of SEQ ID NO:30. In some embodiments, an anti-CD8 antibody of the present disclosure comprises a VH domain comprising a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33 and a VL domain comprising a CDR-L1 comprising the amino acid sequence of SEQ ID NO:34, a CDR-L2 comprising the amino acid sequence of SEQ ID NO: 35, and a CDR-L3 comprising the amino acid sequence of SEQ ID NO:36. In some embodiments, an anti-CD8 antibody of the present disclosure comprises a VH domain comprising a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39 and a VL domain comprising a CDR-L1 comprising the amino acid sequence of SEQ ID NO:40, a CDR-L2 comprising the amino acid sequence of SEQ ID NO: 41, and a CDR-L3 comprising the amino acid sequence of SEQ ID NO:42. In some embodiments, an anti-CD8 antibody of the present disclosure comprises a VH domain comprising a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45 and a VL domain comprising a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48. In some embodiments, an anti-CD8 antibody of the present disclosure comprises a VH domain comprising a CDR-H1 comprising the amino acid sequence of SEQ ID NO: 183, a CDR-H2 comprising the amino acid sequence of SEQ ID NO:184, and a CDR-H3 comprising the amino acid sequence of SEQ ID NO:179 and a VL domain comprising a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO:182.

In some embodiments, an anti-CD8 antibody of the present disclosure comprises a VH domain comprising a CDR-H1 comprising the amino acid sequence of GX₁X₂FX3X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO:265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO:266), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:267) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO: 262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO: 263), and a CDR-L3 comprising the amino acid sequence of QX₁X₂X₃X₄XsPWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233 and a VL domain comprising a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233 and a VL domain comprising a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of GFTFX₁X₂Y, wherein X₁ is S, D, E, Q, S, or A and X₂ is S, D, E, A, or Q (SEQ ID NO:271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO:272), and a CDR-H3 comprising the amino acid sequence of X₁X₂X₃YX₄WX5X₆AX7DX8, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:273) and a VL domain comprising a CDR-L1 comprising the amino acid sequence of RASQSVSSNLA (SEQ ID NO: 40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain further comprises a FW-1 comprising the sequence EVOLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain further comprises a FW-1 comprising the sequence EVOLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain further comprises a FW-1 comprising the sequence EVOLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296). In some embodiments, the present disclosure provides an anti-CD8 antibody comprising a VH domain comprising CDR-H1, CDR-H2, and CDR-H3 sequences of a single antibody listed in Table 1 and a VL domain comprising CDR-L1, CDR-L2, and CDR-L3 sequences of the single antibody listed in Table 1. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides a fusion protein comprising an anti-CD8 antibody comprising a VH domain comprising CDR-H1, CDR-H2, and CDR-H3 sequences of the single antibody listed in Table 1 and a VL domain comprising CDR-L1, CDR-L2, and CDR-L3 sequences of a single antibody listed in Table 1. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides a fusion protein comprising an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains are from the same single antibody listed in Table 3). For example, the anti-CD8 antibody comprises the VH and VL of antibody xhCD8v1, xhCD8v1.1, xhCD8v2, xhCD8v3, xhCD8v4, xhCD8v5, xhCD8v6, xhCD8v7, xhCD8v8,xhCD8v9, xhCD8v10, xhCD8v11, xhCD8v12, xhCD8v13, xhCD8v14, or xhCD8v15 shown in Table 3. In some embodiments, the present disclosure provides a fusion protein comprising an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains arc from the same single antibody listed in Table 3). In some embodiments, the present disclosure provides an anti-CD8 antibody comprising a VH domain sequence and a VL domain sequence for a single antibody as listed in Table 3. In some embodiments, the present disclosure provides a fusion protein comprising an anti-CD8 antibody comprising a VH domain sequence and a VL domain sequence for a single antibody as listed in Table 3. For example, the anti-CD8 antibody of the fusion protein comprises the VH and VL of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, or xhCD8v15 shown in Table 3.

TABLE 1
Anti-CD8 antibody CDRs (Kabat)

| Name | CDR-H1 | CDR-H2 | CDR-H3 | CDR-L1 | CDR-L2 | CDR-L3 |
|---|---|---|---|---|---|---|
| xhCD8v1 | KYTMH | HFNPNND | DGLGLRL | GASENIY | GATNLAD | QNILDTP |
| | (SEQ ID | ETKYNQK | FAD | GALN | (SEQ ID | WT |
| | NO: 1) | FTG | (SEQ ID | (SEQ ID | NO: 5) | (SEQ ID |
| | | (SEQ ID | NO: 3) | NO: 4) | | NO: 6) |
| | | NO: 2) | | | | |
| xhCD8v1.1 | KYAIS | HFNPNND | DGLGLRL | RASENIYG | GATNLAD | QNILDTP |
| | (SEQ ID | ETKYNQK | FAD | ALN | (SEQ ID | WT |
| | NO: 7) | FQG | (SEQ ID | (SEQ ID | NO: 11) | (SEQ ID |
| | | (SEQ ID | NO: 9) | NO: 10) | | NO: 12) |
| | | NO: 8) | | | | |
| xhCD8v2 | NFAIS | GIIPGHAK | DGLGIRLF | RASQEIYG | GATNLQS | QDIYDAP |
| | (SEQ ID | ANYAQKF | AD | ALN | (SEQ ID | WT |
| | NO: 13) | QG | (SEQ ID | (SEQ ID | NO: 17) | (SEQ ID |
| | | (SEQ ID | NO: 15) | NO: 16) | | NO: 18) |
| | | NO: 14) | | | | |
| xhCD8v3 | KFAIS | GIIPGHAK | DGLGIRLF | RASQEIYG | GATNLQS | QDIYDAP |
| | (SEQ ID | ANYAQKF | AD | ALN | (SEQ ID | WT |
| | NO: 19) | QG | (SEQ ID | (SEQ ID | NO: 23) | (SEQ ID |
| | | (SEQ ID | NO: 21) | NO: 22) | | NO: 24) |
| | | NO: 20) | | | | |
| xhCD8v4 | KYAIS | GIIPGHAK | DGLGIRLF | RASQKIY | GATNLQS | QNTYDTP |
| | (SEQ ID | ANYAQKF | AD | GALN | (SEQ ID | WT |
| | NO: 25) | QG | (SEQ ID | (SEQ ID | NO: 29) | (SEQ ID |
| | | (SEQ ID | NO: 27) | NO: 28) | | NO: 30) |
| | | NO: 26) | | | | |
| xhCD8v5 | GHAIS | GIIPGHAK | DGLGIRLF | RASQKIY | GATNLQS | QNTYDTP |
| | (SEQ ID | ANYAQKF | AD | GALN | (SEQ ID | WT |
| | NO: 31) | QG | (SEQ ID | (SEQ ID | NO: 35) | (SEQ ID |
| | | (SEQ ID | NO: 33) | NO: 34) | | NO: 36) |
| | | NO: 32) | | | | |
| xhCD8v6 | DYGMS | DINWSGEI | SNSYRWD | RASQSVSS | GASSRAT | QQYGSSP |
| | (SEQ ID | TAYADSV | DALDI | NLA | (SEQ ID | PVT |
| | NO: 37) | KG | (SEQ ID | (SEQ ID | NO: 41) | (SEQ ID |
| | | (SEQ ID | NO: 39) | NO: 40) | | NO: 42) |
| | | NO: 38) | | | | |
| xhCD8v7 | DYAMH | VISYDGSN | DRIGWYD | RASHSVG | DASNRAT | QQRSNWP |
| | (SEQ ID | KYYADSV | YDAFDI | SNLA | (SEQ ID | PT |
| | NO: 43) | KG | (SEQ ID | (SEQ ID | NO: 47) | (SEQ ID |
| | | (SEQ ID | NO: 45) | NO: 46) | | NO: 48) |
| | | NO: 44) | | | | |
| xhCD8v8 | SYWMN | QIYPGDG | SGAAFSS | RASENIYS | AATNLAD | QHFWGTP |
| | (SEQ ID | DTNYNGK | YYAMDY | NLA (SEQ | (SEQ ID | WT (SEQ |
| | NO: 177) | FKG (SEQ | (SEQ ID | ID NO: 180) | NO: 181) | ID NO: 182) |
| | | ID NO: 178) | NO: 179) | | | |
| xhCD8v9 | SYAIS | GIIPGAAT | DAAGIRLF | RASQEIYG | GATNLQS | QSTYDAP |
| | (SEQ ID | ANYAQKF | AD | ALN | (SEQ ID | WT (SEQ |
| | NO: 225) | QG | (SEQ ID | (SEQ ID | NO: 17) | ID NO: 228) |
| | | (SEQ ID | NO: 227) | NO: 16) | | |
| | | NO: 226) | | | | |
| xhCD8v10 | SYAMS | DITYAGG | SNAYAW | RASQSVSS | GASSRAT | QQYGSSP |
| | (SEQ ID | STAYADS | DDALDI | NLA | (SEQ ID | PVT |
| | NO: 229) | VKG (SEQ | (SEQ ID | (SEQ ID | NO: 41) | (SEQ ID |
| | | ID NO: 230) | NO: 231) | NO: 40) | | NO: 42) |
| xhCD8v11 | SYAMS | DITYAGG | SNAYAW | RASQSVSS | GASSRAT | QQYGSSP |
| | (SEQ ID | STAYADS | DDALDI | NLA | (SEQ ID | PVT |
| | NO: 229) | VKG (SEQ | (SEQ ID | (SEQ ID | NO: 41) | (SEQ ID |
| | | ID NO: 230) | NO: 231) | NO: 40) | | NO: 42) |
| xhCD8v12 | SYAIS | GIIPGYAT | DAAGIRLF | RASQSIYG | GASNLQS | QSTYTAP |
| | (SEQ ID | ANYAQKF | AD (SEQ | ALN (SEQ | (SEQ ID | WT (SEQ |
| | NO: 225) | QG (SEQ | ID NO: 233) | ID NO: 234) | NO: 235) | ID NO: 236) |
| | | ID NO: 232) | | | | |
| xhCD8v13 | SYAIS | GIIPGYAT | DAAGIRLF | RASQEIYG | GATNLQS | QSTYDAP |
| | (SEQ ID | ANYAQKF | AD (SEQ | ALN | (SEQ ID | WT (SEQ |
| | NO: 225) | QG (SEQ | ID NO: 233) | (SEQ ID | NO: 17) | ID NO: 228) |
| | | ID NO: 232) | | NO: 16) | | |
| xhCD8v14 | SYAMS | DISYAGGS | SNAYAW | RASQSVSS | GASSRAT | QQYGSSP |
| | (SEQ ID | TAYADSV | DDALDI | NLA | (SEQ ID | PVT |
| | NO: 229) | KG (SEQ | (SEQ ID | (SEQ ID | NO: 41) | (SEQ ID |
| | | ID NO: 237) | NO: 231) | NO: 40) | | NO: 42) |
| xhCD8v15 | SYAMS | DISYAGGS | SNAYAW | RASQSVSS | GASSRAT | QQYGSSP |
| | (SEQ ID | TAYADSV | DDALDI | NLA | (SEQ ID | PVT |
| | NO: 229) | KG (SEQ | (SEQ ID | (SEQ ID | NO: 41) | (SEQ ID |
| | | ID NO: 237) | NO: 231) | NO: 40) | | NO: 42) |
| V9 family | X₁X₂AIS | X₁X₂X₃PX₄ | X₁X₂X₃GX₄ | X₁X₂SX₃X₄I | GX₁X₂X₃L | QX₁X₂X₃X₄ |
| | X₁ is S, K, | X₅X₆X₇X₈X₉ | X₅LFX₆X₇ | X₅GX₆LN | X₄X₅ | X₅PWT |
| | G, N, R, D, | YX₁₀QKF | X₁ is D or | X₁ is R or | X₁ is A or S, | X₁ is S, N, |
| | T, or G | X₁₁G | A, | G, | X₂ is T, S, | D, Q, A, or |
| | X₂ is Y, L, | X₁ is G or | X₂ is A, G, | X₂ is A or | E, Q, or D, | E, |
| | H, or F | H, X₂ is I or | E, R, Y, K, | T, | X₃ is N, R, | X₂ is T, I, or |
| | (SEQ ID | F, | N, Q, L, or | X₃ is Q or | A, E, or H, | S, |
| | NO: 259) | X₃ is I, N, | F, | E, | X₄ is Q or | X₃ is Y, L, |
| | | or M, | X₃ is A, L, | X₄ is E, N, | A, | or F, |
| | | X₄ is G, N, | P, or Y, | T, S, A, K, | X₅ is S or D | X₄ is D, G, |
| | | H, S, R, I, | X₄ is I or L, | D, G, R, or | (SEQ ID | T, E, Q, A, |
| | | or A, X₅ is | X₅ is R, A, | Q, | NO: 263) | or Y, |
| | | A, N, H, S, | Q, or S, | X₅ is Y or S, | | X₅ is A, T, |
| | | T, F, or Y, | X₆ is A or | X₆ is A or V | | R, S, K, or |
| | | X₆ is A, D, | D, | (SEQ ID | | Y |
| | | or G, | X₇ is D, E, | NO: 262) | | (SEQ ID |
| | | X₇ is T, E, | A, or S | | | NO: 264) |
| | | K, V, Q, or | (SEQ ID | | | |
| | | A, | NO: 261) | | | |
| | | X₈ is A or | | | | |
| | | T, X₉ is N | | | | |
| | | or K, X₁₀ is | | | | |
| | | A or N, X₁₁ | | | | |
| | | is Q or T | | | | |
| | | (SEQ ID | | | | |
| | | NO: 260) | | | | |
| V11 family | X₁YX₂MS | DIX₁X₂X₃G | X₁X₂X₃YX₄ | RASQSVSS | GASSRAT | QQYGSSP |
| | X₁ is S, D, | X₄X₅TX₆Y | WX₅X₆AX₇ | NLA | (SEQ ID | PVT |
| | E, A, or Q | ADSVKG | DX₈ | (SEQ ID | NO: 41) | (SEQ ID |
| | X₂ is A, G, | X₁ is T, N, | X₁ is S or A, | NO: 40) | | NO: 42) |
| | or T (SEQ | S, Q, E, H, | X₂ is N, H, | | | |
| | ID NO: 268) | R, or A, | A, D, L, Q, | | | |
| | | X₂ is Y, W, | Y, or R, | | | |
| | | F, or H, | X₃ is A, N, | | | |
| | | X₃ is A, S, | S, or G, | | | |
| | | Q, E, or T, | X₄ is A, V, | | | |
| | | X₄ is G or | R, E, or S, | | | |
| | | E, X₅ is S or | X₅ is D or S, | | | |
| | | I, | X₆ is D, N, | | | |
| | | X₆ is A or G | Q, E, S, T, | | | |
| | | (SEQ ID | or L, | | | |
| | | NO: 269) | X₇ is L, F, | | | |
| | | | or M, | | | |
| | | | X₈ is I, Y, | | | |
| | | | or V | | | |
| | | | (SEQ ID | | | |
| | | | NO: 270) | | | |

TABLE 2
Anti-CD8 antibody CDRs (Chothia)

| Name | CDR-H1 | CDR-H2 | CDR-H3 | CDR-L1 | CDR-L2 | CDR-L3 |
|---|---|---|---|---|---|---|
| xhCD8v1 | GYTFTKY | NPNNDE | DGLGLRL | GASENIY | GATNLAD | QNILDTP |
| | (SEQ ID | (SEQ ID | FAD | GALN | (SEQ ID | WT |
| | NO: 49) | NO: 50) | (SEQ ID | (SEQ ID | NO: 5) | (SEQ ID |
| | | | NO: 3) | NO: 4) | | NO: 6) |
| xhCD8v1.1 | GYTFTKY | NPNNDE | DGLGLRL | RASENIYG | GATNLAD | QNILDTP |
| | (SEQ ID | (SEQ ID | FAD | ALN | (SEQ ID | WT |
| | NO: 49) | NO: 50) | (SEQ ID | (SEQ ID | NO: 11) | (SEQ ID |
| | | | NO: 9) | NO: 10) | | NO: 12) |
| xhCD8v2 | GYRFHNF | IPGHAK | DGLGIRLF | RASQEIYG | GATNLQS | QDIYDAP |
| | (SEQ ID | (SEQ ID | AD | ALN | (SEQ ID | WT |
| | NO: 51) | NO: 52) | (SEQ ID | (SEQ ID | NO: 17) | (SEQ ID |
| | | | NO: 15) | NO: 16) | | NO: 18) |
| xhCD8v3 | GSRFYKF | IPGHAK | DGLGIRLF | RASQEIYG | GATNLQS | QDIYDAP |
| | (SEQ ID | (SEQ ID | AD | ALN | (SEQ ID | WT |
| | NO: 53) | NO: 52) | (SEQ ID | (SEQ ID | NO: 23) | (SEQ ID |
| | | | NO: 21) | NO: 22) | | NO: 24) |
| xhCD8v4 | GYTFTKY | IPGHAK | DGLGIRLF | RASQKIY | GATNLQS | QNTYDTP |
| | (SEQ ID | (SEQ ID | AD | GALN | (SEQ ID | WT |
| | NO: 49) | NO: 52) | (SEQ ID | (SEQ ID | NO: 29) | (SEQ ID |
| | | | NO: 27) | NO: 28) | | NO: 30) |
| xhCD8v5 | GSGFRGH | IPGHAK | DGLGIRLF | RASQKIY | GATNLQS | QNTYDTP |
| | (SEQ ID | (SEQ ID | AD | GALN | (SEQ ID | WT |
| | NO: 54) | NO: 52) | (SEQ ID | (SEQ ID | NO: 35) | (SEQ ID |
| | | | NO: 33) | NO: 34) | | NO: 36) |
| xhCD8v6 | GFTFDDY | NWSGEI | SNSYRWD | RASQSVSS | GASSRAT | QQYGSSP |
| | (SEQ ID | (SEQ ID | DALDI | NLA | (SEQ ID | PVT |
| | NO: 55) | NO: 56) | (SEQ ID | (SEQ ID | NO: 41) | (SEQ ID |
| | | | NO: 39) | NO: 40) | | NO: 42) |
| xhCD8v7 | GFTFDDY | SYDGSN | DRIGWYD | RASHSVG | DASNRAT | QQRSNWP |
| | (SEQ ID | (SEQ ID | YDAFDI | SNLA | (SEQ ID | PT |
| | NO: 55) | NO: 57) | (SEQ ID | (SEQ ID | NO: 47) | (SEQ ID |
| | | | NO: 45) | NO: 46) | | NO: 48) |
| xhCD8v8 | GYAFSSY | YPGDGD | SGAAFSS | RASENIYS | AATNLAD | QHFWGTP |
| | (SEQ ID | (SEQ ID | YYAMDY | NLA (SEQ | (SEQ ID | WT (SEQ |
| | NO: 183) | NO: 184) | (SEQ ID | ID NO: 180) | NO: 181) | ID NO: 182) |
| | | | NO: 179) | | | |
| xhCD8v9 | GGTFSSY | IPGAAT | DAAGIRLF | RASQEIYG | GATNLQS | QSTYDAP |
| | (SEQ ID | (SEQ ID | AD (SEQ | ALN | (SEQ ID | WT (SEQ |
| | NO: 238) | NO: 239) | ID NO: 233) | (SEQ ID | NO: 17) | ID NO: 228) |
| | | | | NO: 16) | | |
| xhCD8v10 | GFTFSSY | TYAGGS | SNAYAW | RASQSVSS | GASSRAT | QQYGSSP |
| | (SEQ ID | (SEQ ID | DDALDI | NLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v11 | GFTFSSY | TYAGGS | SNAYAW | RASQSVSS | GASSRAT | QQYGSSP |
| | (SEQ ID | (SEQ ID | DDALDI | NLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v12 | GGTFSSY | IPGYAT | DAAGIRLF | RASQSIYG | GASNLQS | QSTYTAP |
| | (SEQ ID | (SEQ ID | AD (SEQ | ALN (SEQ | (SEQ ID | WT (SEQ |
| | NO: 238) | NO: 243) | ID NO: 233) | ID NO: 234) | NO: 235) | ID NO: 236) |
| xhCD8v13 | GGTFSSY | IPGYAT | DAAGIRLF | RASQEIYG | GATNLQS | QSTYDAP |
| | (SEQ ID | (SEQ ID | AD (SEQ | ALN | (SEQ ID | WT (SEQ |
| | NO: 238) | NO: 243) | ID NO: 233) | (SEQ ID | NO: 17) | ID NO: 228) |
| | | | | NO: 16) | | |
| xhCD8v14 | GFTFSSY | SYAGGS | SNAYAW | RASQSVSS | GASSRAT | QQYGSSP |
| | (SEQ ID | (SEQ ID | DDALDI | NLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| xhCD8v15 | GFTFSSY | SYAGGS | SNAYAW | RASQSVSS | GASSRAT | QQYGSSP |
| | (SEQ ID | (SEQ ID | DDALDI | NLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| V9 family | GX₁X₂FX₃ | X₁PX₂X₃X₄ | X₁X₂X₃GX₄ | X₁X₂SX₃X₄I | GX₁X₂X₃L | QX₁X₂X₃X₄ |
| | X₄X₅ | X₅ | X₅LFX₆X₇ | X₅GX₆LN | X₄X₅ | X₅PWT |
| | X₁ is G, Y, | X₁ is I, N, | X₁ is D or | X₁ is R or | X₁ is A or S, | X₁ is S, N, |
| | S, or A, | or M, | A, | G, | X₂ is T, S, | D, Q, A, or |
| | X₂ is T, S, | X₂ is G, N, | X₂ is A, G, | X₂ is A or | E, Q, or D, | E, |
| | G, R, N, or | H, S, R, I, | E, R, Y, K, | T, | X₃ is N, R, | X₂ is T, I, or |
| | H, | or A, | N, Q, L, or | X₃ is Q or | A, E, or H, | S, |
| | X₃ is S, T, | X₃ is A, N, | F, | E, | X₄ is Q or | X₃ is Y, L, |
| | R, H, Y, G, | H, S, T, F, | X₃ is A, L, | X₄ is E, N, | A, | or F, |
| | or P, | or Y, | P, or Y, | T, S, A, K, | X₅ is S or D | X₄ is D, G, |
| | X₄ is S, K, | X₄ is A, D, | X₄ is I or L, | D, G, R, or | (SEQ ID | T, E, Q, A, |
| | G, N, R, D, | or G, | X₅ is R, A, | Q, | NO: 263) | or Y, |
| | T, or G, | X₅ is T, E, | Q, or S, | X₅ is Y or S, | | X₅ is A, T, |
| | X₅ is Y, L, | K, V, Q, or | X₆ is A or | X₆ is A or V | | R, S, K, or |
| | H, or F | A | D, | (SEQ ID | | Y |
| | (SEQ ID | (SEQ ID | X₇ is D, E, | NO: 262) | | (SEQ ID |
| | NO: 265) | NO: 266) | A, or S | | | NO: 264) |
| | | | (SEQ ID | | | |
| | | | NO: 267) | | | |
| V11 family | GFTFX₁X₂ | X₁X₂X₃GX₄ | X₁X₂X₃YX₄ | RASQSVSS | GASSRAT | QQYGSSP |
| | Y | X₅ | WX₅X₆AX₇ | NLA | (SEQ ID | PVT |
| | X₁ is S, D, | X₁ is T, N, | DX₈ | (SEQ ID | NO: 41) | (SEQ ID |
| | E, Q, S, or | S, Q, E, H, | X₁ is S or A, | NO: 40) | | NO: 42) |
| | A | R or A, | X₂ is N, H, | | | |
| | X₂ is S, D, | X₂ is Y, W, | A, D, L, Q, | | | |
| | E, A, or Q | F, or H, | Y, or R, | | | |
| | (SEQ ID | X₃ is A, S, | X₃ is A, N, | | | |
| | NO: 271) | Q, E, or T, | S, or G, | | | |
| | | X₄ is G or | X₄ is A, V, | | | |
| | | E, | R, E, or S, | | | |
| | | X₅ is S or I | X₅ is D or S, | | | |
| | | (SEQ ID | X₆ is D, N, | | | |
| | | NO: 272) | Q, E, S, T, | | | |
| | | | or L, | | | |
| | | | X₇ is L, F, | | | |
| | | | or M, | | | |
| | | | X₈ is I, Y, | | | |
| | | | or V | | | |
| | | | (SEQ ID | | | |
| | | | NO: 273) | | | |

TABLE 3
Anti-CD8 antibody variable domain sequences

| Name | VH | VL |
|---|---|---|
| xhCD8v1 | QVHLQQSGPELVKPGASVKMSCKTS | DIQMTQSPASLSASVGETVTITCGAS |
| | GYTFTKYTMHWVKQGHEESLEWIG | ENIYGALNWYQRKQGKSPQLLIFGA |
| | HFNPNNDETKYNQKFTGKATLTVDK | TNLADGVSSRFSGSGSDRQYSLKISS |
| | SSTTAYMELRSLTSDDSALYYCARD | LHPDDVATYYCQNILDTPWTFGGGT |
| | GLGLRLFADWGQGTLITVSA | KLEIK |
| | (SEQ ID NO: 58) | (SEQ ID NO: 59) |
| xhCD8v1.1 | QVQLVQSGAEVKKPGSSVKVSCKAS | DIQMTQSPSSLSASVGDRVTITCRASE |
| | GYTFTKYAISWVRQAPGQGLEWMG | NIYGALNWYQQKPGKAPKLLIYGAT |
| | HFNPNNDETKYNQKFQGRVTITADE | NLADGVPSRFSGSGSGTDFTLTISSLQ |
| | STSTAYMELSSLRSEDTAVYYCARD | PEDFATYYCQNILDTPWTFGGGTKLE |
| | GLGLRLFADWGQGTLVTVSS | IK |
| | (SEQ ID NO: 60) | (SEQ ID NO: 61) |
| xhCD8v2 | QVQLVQSGAEVKKPGSSVKVSCKAS | DIQMTQSPSSLSASVGDRVTITCRAS |
| | GYRFHNFAISWVRQAPGQGLEWMG | QEIYGALNWYQQKPGKAPKLLIYGA |
| | GIIPGHAKANYAQKFQGRVTITADES | TNLQSGVPSRFSGSGSGTDFTLTISSL |
| | TSTAYMELSSLRSEDTAVYYCARDG | QPEDFATYYCQDIYDAPWTFGGGTK |
| | LGIRLFADWGQGTLVTVSS | VEIK |
| | (SEQ ID NO: 62) | (SEQ ID NO: 63) |
| xhCD8v3 | QVQLVQSGAEVKKPGSSVKVSCKAS | DIQMTQSPSSLSASVGDRVTITCRAS |
| | GSRFYKFAISWVRQAPGQGLEWMG | QEIYGALNWYQQKPGKAPKLLIYGA |
| | GIIPGHAKANYAQKFQGRVTITADES | TNLQSGVPSRFSGSGSGTDFTLTISSL |
| | TSTAYMELSSLRSEDTAVYYCARDG | QPEDFATYYCQDIYDAPWTFGGGTK |
| | LGIRLFADWGQGTLVTVSS | VEIK |
| | (SEQ ID NO: 64) | (SEQ ID NO: 65) |
| xhCD8v4 | QVQLVQSGAEVKKPGSSVKVSCKAS | DIQMTQSPSSLSASVGDRVTITCRAS |
| | GYTFTKYAISWVRQAPGQGLEWMG | QKIYGALNWYQQKPGKAPKLLIYGA |
| | GIIPGHAKANYAQKFQGRVTITADES | TNLQSGVPSRFSGSGSGTDFTLTISSL |
| | TSTAYMELSSLRSEDTAVYYCARDG | QPEDFATYYCQNTYDTPWTFGGGTK |
| | LGIRLFADWGQGTLVTVSS | VEIK |
| | (SEQ ID NO: 66) | (SEQ ID NO: 67) |
| xhCD8v5 | QVQLVQSGAEVKKPGSSVKVSCKAS | DIQMTQSPSSLSASVGDRVTITCRAS |
| | GSGFRGHAISWVRQAPGQGLEWMG | QKIYGALNWYQQKPGKAPKLLIYGA |
| | GIIPGHAKANYAQKFQGRVTITADES | TNLQSGVPSRFSGSGSGTDFTLTISSL |
| | TSTAYMELSSLRSEDTAVYYCARDG | QPEDFATYYCQNTYDTPWTFGGGTK |
| | LGIRLFADWGQGTLVTVSS | VEIK |
| | (SEQ ID NO: 68) | (SEQ ID NO: 69) |
| xhCD8v6 | EVQLVESGGGAVRPGGSLRLSCAAS | EIVLTQSPATLSVSPGERATLSCRASQ |
| | GFTFDDYGMSWVRQAPGKGLEWVS | SVSSNLAWYQQKPGQAPRLLIYGAS |
| | DINWSGEITAYADSVKGRFTISRDNA | SRATGIPDRFSGSGSGTDFTLTISRLEP |
| | KNSLYLQMNSLRAEDTAVYYCARSN | EDFAVYYCQQYGSSPPVTFGQGTKV |
| | SYRWDDALDIWGQGTMVTVSS | EIK |
| | (SEQ ID NO: 70) | (SEQ ID NO: 71) |
| xhCD8v7 | EVQLVESGGGLVQPGRSLRLSCAAS | EIVLTQSPATLSVTPGEGATLSCRASH |
| | GFTFDDYAMHWVRQAPGKGLEWV | SVGSNLAWYQQKPGQAPRLLIYDAS |
| | AVISYDGSNKYYADSVKGRFTISRDN | NRATGIPARFSGSGSGTDFTLTISSLE |
| | SKNTLYLQMNSLRAEDTAVYYCAK | PEDLAVYYCQQRSNWPPTFGQGTRL |
| | DRIGWYDYDAFDIWGQGTMVTVSS | EIK |
| | (SEQ ID NO: 72) | (SEQ ID NO: 73) |
| xhCD8v8 | QVQLQQSGAELVRPGSSVKISCKASG | DIQMTQSPASLSVSVGETVTITCRASE |
| | YAFSSYWMNWVKQRPGQGLEWIGQ | NIYSNLAWYQQKQGKSPQLLVYAAT |
| | IYPGDGDTNYNGKFKGKATLTADKS | NLADGVPSRFSGSGSGTQYSLKINSL |
| | SSTAYMQLSSLTSEDSAVYFCARSGA | QSEDFGSYYCQHFWGTPWTFGGGTK |
| | AFSSYYAMDYWGQGTSVTVSS (SEQ | LEIK (SEQ ID NO: 186) |
| | ID NO: 185) | |
| xhCD8v9 | QVQLVQSGAEVKKPGSSVKVSCKAS | DIQMTQSPSSLSASVGDRVTITCRAS |
| | GGTFSSYAISWVRQAPGQGLEWMG | QEIYGALNWYQQKPGKAPKLLIYGA |
| | GIIPGAATANYAQKFQGRVTITADES | TNLQSGVPSRFSGSGSGTDFTLTISSL |
| | TSTAYMELSSLRSEDTAVYYCARDA | QPEDFATYYCQSTYDAPWTFGGGTK |
| | AGIRLFADWGQGTLVTVSS (SEQ ID | VEIK (SEQ ID NO: 246) |
| | NO: 245) | |
| xhCD8v10 | EVQLVESGGGLVQPGGSLRLSCAAS | EIVLTQSPGTLSLSPGERATLSCRASQ |
| | GFTFSSYAMSWVRQAPGKGLEWVS | SVSSNLAWYQQKPGQAPRLLIYGAS |
| | DITYAGGSTAYADSVKGRFTISRDNA | SRATGIPDRFSGSGSGTDFTLTISRLEP |
| | KNSLYLQMNSLRAEDTAVYYCARSN | EDFAVYYCQQYGSSPPVTFGQGTKV |
| | AYAWDDALDIWGQGTMVTVSS | EIK (SEQ ID NO: 248) |
| | (SEQ ID NO: 247) | |
| xhCD8v11 | EVQLVESGGGLVQPGGSLRLSCAAS | EIVLTQSPGTLSLSPGERATLSCRASQ |
| | GFTFSSYAMSWVRQAPGKGLEWVS | SVSSNLAWYQQKPGQAPRLLIYGAS |
| | DITYAGGSTAYADSVKGRFTISRDNA | SRATGIPDRFSGSGSGTDFTLTISRLEP |
| | KNSLYLQMNSLRAEDTAVYYCARSN | EDFAVYYCQQYGSSPPVTFGQGTKV |
| | AYAWDDALDIWGQGTLVTVSS (SEQ | EIK (SEQ ID NO: 250) |
| | ID NO: 249) | |
| xhCD8v12 | QVQLVQSGAEVKKPGSSVKVSCKAS | DIQMTQSPSSLSASVGDRVTITCRAS |
| | GGTFSSYAISWVRQAPGQGLEWMG | QSIYGALNWYQQKPGKAPKLLIYGA |
| | GIIPGYATANYAQKFQGRVTITADES | SNLQSGVPSRFSGSGSGTDFTLTISSL |
| | TSTAYMELSSLRSEDTAVYYCARDA | QPEDFATYYCQSTYTAPWTFGGGTK |
| | AGIRLFADWGQGTLVTVSS (SEQ ID | VEIK (SEQ ID NO: 252) |
| | NO: 251) | |
| xhCD8v13 | QVQLVQSGAEVKKPGSSVKVSCKAS | DIQMTQSPSSLSASVGDRVTITCRAS |
| | GGTFSSYAISWVRQAPGQGLEWMG | QEIYGALNWYQQKPGKAPKLLIYGA |
| | GIIPGYATANYAQKFQGRVTITADES | TNLQSGVPSRFSGSGSGTDFTLTISSL |
| | TSTAYMELSSLRSEDTAVYYCARDA | QPEDFATYYCQSTYDAPWTFGGGTK |
| | AGIRLFADWGQGTLVTVSS (SEQ ID | VEIK (SEQ ID NO: 254) |
| | NO: 253) | |
| xhCD8v14 | EVQLVESGGGLVQPGGSLRLSCAAS | EIVLTQSPGTLSLSPGERATLSCRASQ |
| | GFTFSSYAMSWVRQAPGKGLEWVS | SVSSNLAWYQQKPGQAPRLLIYGAS |
| | DISYAGGSTAYADSVKGRFTISRDNA | SRATGIPDRFSGSGSGTDFTLTISRLEP |
| | KNSLYLQMNSLRAEDTAVYYCARSN | EDFAVYYCQQYGSSPPVTFGQGTKV |
| | AYAWDDALDIWGQGTMVTVSS | EIK (SEQ ID NO: 256) |
| | (SEQ ID NO: 255) | |
| xhCD8v15 | EVQLVESGGGLVQPGGSLRLSCAAS | EIVLTQSPGTLSLSPGERATLSCRASQ |
| | GFTFSSYAMSWVRQAPGKGLEWVS | SVSSNLAWYQQKPGQAPRLLIYGAS |
| | DISYAGGSTAYADSVKGRFTISRDNA | SRATGIPDRFSGSGSGTDFTLTISRLEP |
| | KNSLYLQMNSLRAEDTAVYYCARSN | EDFAVYYCQQYGSSPPVTFGQGTKV |
| | AYAWDDALDIWGQGTLVTVSS (SEQ | EIK (SEQ ID NO: 258) |
| | ID NO: 257) | |

### Fusion Proteins

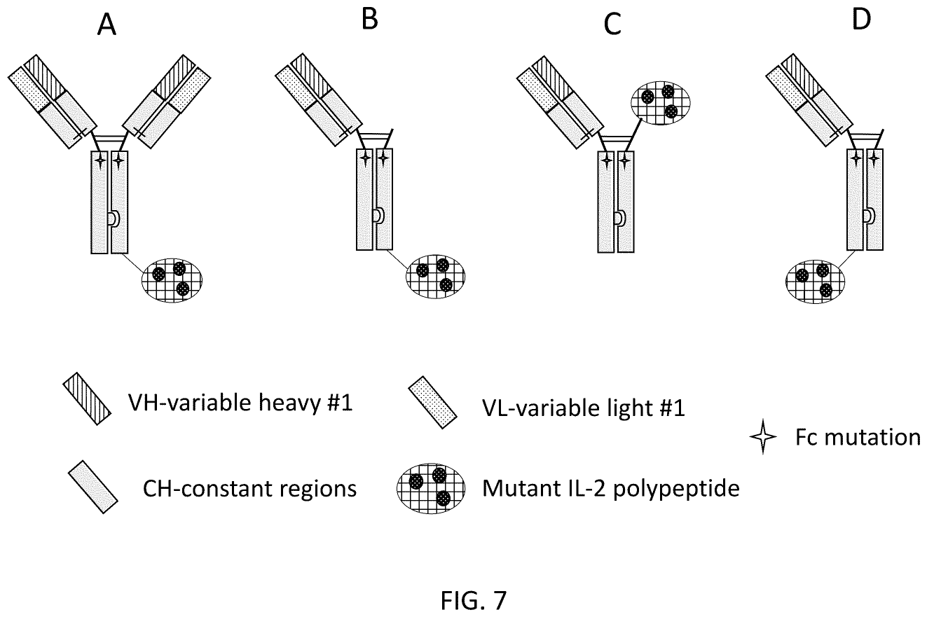

Further provided herein are fusion proteins comprising any one of the anti-CD8 antibodies, or antigen binding domains, or antibody fragments disclosed herein. In some embodiments, a fusion protein of the present disclosure comprises a first moiety comprising a human or humanized antibody or antigen-binding fragment thereof that specifically binds CD8b and/or CD8ab (e.g., any one of the anti-CD8 antibodies described supra) and a second moiety comprising a cytokine, chemokine, or growth factor. In some embodiments, the first moiety is fused to the second moiety directly. In some embodiments, the first moiety is fused to the second moiety via a linker. Exemplary and non-limiting illustrations of fusion proteins of the present disclosure are depicted in FIG. 7.

### Fusion Protein Formats

In some embodiments, the first moiety comprises an antibody (e.g., an anti-CD8 antibody of the present disclosure). In some embodiments, the first moiety comprises an antibody fragment (e.g., an anti-CD8 antibody fragment of the present disclosure). In some embodiments, the first moiety comprises a single chain antibody or single chain variable fragment (scFv). In some embodiments, the first moiety comprises a VHH antibody. In some embodiments, the first moiety comprises one or two antibody heavy chain polypeptides and one or two antibody light chain polypeptides (e.g., of an anti-CD8 antibody of the present disclosure). In some embodiments, the first moiety comprises the 3 heavy chain CDRs and/or 3 light chain CDRs of a single anti-CD8 antibody of the present disclosure, e.g., as shown in Tables 1-3. In some embodiments, the first moiety comprises the VH and/or VL domain(s) of a single anti-CD8 antibody of the present disclosure, e.g., as shown in Table 3. In some embodiments, the first moiety further comprises one or two human IgG Fc domains. In some embodiments, the one or two human IgG Fc domains are IgG1, IgG2, IgG3 or IgG4 Fc domains. In some embodiments, the one or two human IgG Fc domains do not have the C-terminus lysine residue. In some embodiments, the one or two human IgG Fc domains comprise amino acid modifications (such as substitutions, deletions, additions, etc.). In some embodiments, the Fc domain modifications promote heterodimeric formation (e.g., as shown in Table 4). In some embodiments, the one or two Fc domains comprise Fc gamma-null mutations.

In some embodiments, the first moiety comprises two antibody heavy chain polypeptides comprising a structure according to formula [I], from N-terminus to C-terminus:

VH—CH1-hinge-CH2-CH3 [I]

and two antibody light chain polypeptides comprising a structure according to formula [II], from N-terminus to C-terminus:

VL-CL [II]

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the N-terminus of the second moiety is fused to the C-terminus of one of the two CH3 domains (see, e.g., format A in FIG. 7).

In some embodiments, the first moiety comprises a first antibody heavy chain polypeptide comprising a structure according to formula [I], from N-terminus to C-terminus:

VH—CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], from N-terminus to C-terminus:

hinge-CH2-CH3 [III],

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the N-terminus of the second moiety is fused to the C-terminus of the CH3 domain of the second antibody heavy chain polypeptide (see, e.g., format B in FIG. 7). In some embodiments, the N-terminus of the second moiety is fused to the C-terminus of the CH3 domain of the first antibody heavy chain polypeptide (see, e.g., format D in FIG. 7).

In some embodiments, the first moiety comprises a first antibody heavy chain polypeptide comprising a structure according to formula [I], from N-terminus to C-terminus:

VH—CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], from N-terminus to C-terminus:

hinge-CH2-CH3 [III],

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the C-terminus of the second moiety is fused to the N-terminus of the hinge domain of the second antibody heavy chain polypeptide (see, e.g., format C in FIG. 7).

In some embodiments, an anti-CD8 antibody of the present disclosure is a multispecific (e.g., bispecific) antibody or antibody fragment. For example, in some embodiments, the multispecific antibody (e.g., bispecific antibody) comprises a first antigen binding domain that binds to CD8 (e.g., as described supra) and a second antigen binding domain that binds a target of interest. In some embodiments, a bispecific antibody can be generated via fusion of an additional binding site to either the heavy or light chain of an immunoglobulin. Examples of the additional binding site include but not limited to variable regions, scFv, Fab, VHH, and peptide.

In some embodiments, In some embodiments, the recombinant bispecific antibodies disclosed herein can be very roughly classified in two categories, namely i) formats resulting from the combination of variable regions only and ii) formats combining variable regions with Fc domains. Representatives of the first category are tandem scFv (taFv), diabodies (Db), DART, single-chain diabodies (scDbs), Fab-Fc, tandem Fab, Dual variable region Fab and tandem dAb/VHH. The two variable regions can be linked together via covalent bonds or non-covalent interaction.

Noncovalent interaction may involve the use of heterodimerization modules such as leucine zipper, dock-and-lock methods of using regulatory subunit of cAMP-dependent protein kinase (PKA) and the anchoring domains of A kinase anchor proteins (AKAPs) or knob-into-holes CH3 domain (U.S. Pat. Nos. 5,731,168; 7,695,936; Ridgway et al., Prot Eng 9, 617-621 (1996) and Carter, J Immunol Meth 248, 7-15 (2001)) to pair up the variable regions.

In some embodiments, bispecific antibodies are generated on the natural immunoglobulin architecture containing two pairs of heavy chain and light chain combination with each pair having distinct binding specificity. Homodimerization of the two heavy chains in an IgG is mediated by the CH3 interaction. To promote heterodimeric formation, genetic modifications are introduced to the two respective CH3 regions. There heterodimerization mutations often involve steric repulsion, charge steering interaction, or interchain disulfide bond formation. Exemplary and non-limiting Fc modifications to promote heterodimerization include the following:

TABLE 4
Exemplary Fc modifications to promote heterodimerization.

| Strategy | CH3 domain 1 | CH3 domain 2 | References |
|---|---|---|---|
| knobs-into-holes 1 | T366Y | Y407T | Brinkmann & |
| knobs-into-holes 2 | T366W | T366S- | Kontermann, MAbs. |
| | | L368A- | 2017 February-March; 9(2): |
| | | Y407V | 182-212; |
| knobs-into-holes 3 | S354C, | Y349C, | Atwell et al, J Mol |
| | T366W | T366S, | Biol 1997; 270: 26-35; |
| | | L368A, | Merchant et |
| | | Y407V | al, Nat Biotechnol 1998; |
| knobs-into-holes 4 | Y349C, | S354C, | 16: 677-681 |
| | T366W | T366S, | |
| | | L368A, | |
| | | Y407V | |
| HA-TF | S364H, | Y349T, | Moore et al, MAbs 2011; |
| | F405A | T394F | 3: 546-557 |
| ZW1 | T350V, | T350V, | Von Kreudenstein et al, |
| | L351Y, | T366L, | MAbs 2013; 5: 646-54 |
| | F405A, | K392L, | |
| | Y407V | T394W | |
| CH3 charge pairs (DD-KK) | K392D, | E356K, | Gunasekaran et al, J Biol |
| | K409D | D399K | Chem 2010; 285: 19637- |
| | | | 46 |
| IgG1 hinge/CH3 charge pairs (EEE- | IgG1: D221E, | IgG1: D221R, | Strop Pet al, J Mol |
| RRR) | P228E, L368E | P228R, | Biol 2012; 420: 204-19 |
| | | K409R | |
| IgG2 hinge/CH3 charge pairs (EEE- | IgG2: C223E, | IgG2: C223R, | |
| RRRR) | P228E, L368E | E225R, | |
| | | P228R, | |
| | | K409R | |
| EW-RVT | K360E, | Q347R, | Choi et al, Mol Cancer |
| | K409W, | D399V, | Ther 2013; 12: 2748-59 |
| | | F405T | |
| EW-RVT_{S-S} | K360E, | Q347R, | Choi et al, Mol |
| | K409W, | D399V, | Immunol 2015; 65: 377- |
| | Y349C | F405T, S354C | 83 |
| Biclonic | 366K (+351K) | 351D or E or | Geuijen et al, ournal of |
| | | D at 349, 368, | Clinical Oncology 2014; |
| | | 349, or 349 + | 32: suppl: 560 |
| | | 355 | |
| DuoBody (L-R) 1 | F405L | K409R | Labrijn et al, Nat |
| DuoBody (L-R) 2 | F405L-R409K | WT (R409) | Protoc 2014; 9: 2450-63; |
| | | | Labrijn et al, PNAS |
| | | | 2013; 110(13): 5145-50 |
| SEEDbody | IgG/A | IgG/A | Davis et al, Protein Eng |
| | chimera | chimera | Des |
| | | | Sel 2010; 23: 195-202 |
| BEAT | residues from | residues from | Moretti et al, BMC |
| | TCRα | TCRβ | Proceedings 2013; |
| | interface | interface | 7(Suppl 6): O9 |
| Mixed interface (MI) heterodimers | IgG—CH3 | igA/D/M CH3 | Skegro et al, J Biol |
| | variants | variants or | Chem. 2017 292(23): |
| | | IgM CH4 | 9745-9759. |
| | | variants | |
| XmAb | E357Q-S364K | L368D-K370S | Moore et al, Methods. |
| | | | 2019 |
| | | | 154: 38-50 |
| DEKK Fc | L351D-L368E | L351K- | De Nardis et al., J Biol |
| | | T366K | Chem. 2017; 292(35): |
| | | | 14706-14717. |
| Charge pair | E356K or | K370E, | Igawa T, Tsunoda H. |
| | E357K or | K409D, | WO2006106905. 2006. |
| | D399K | K439E | |
| KKA-DDW | D356K- | K392D- | Zhou et al, |
| | D399K- | K409D- | WO2014079000A1 |
| | Y407A | T366W | |
| Charge pair | L368E-Y407E | E357K- | Labrijn et al, Nat |
| | | D399K | Review Drug Discovery |
| | | | 2019; 18, 585-608 |
| Knob-hole-electrostatic | S354C- | Y349C- | Wei et al., Oncotarget. |
| | T366W- | T366S- | 2017; 8(31): 51037- |
| | K409A | L368A-Y407- | 51049 |
| | | F405K | |
| KA | F405K | K409A | Wei et al., Oncotarget. |
| | | | 2017; 8(31): 51037- |
| | | | 51049 |
| PPV-TPP | P395K- | T394D- | Wenjun Zhang |
| | P396K- | P395D- | US10538595B2 |
| | V397K, | P396D, | |
| | P395K- | T394C- | |
| | P396K- | P395D-P396D | |
| | V397C or | or T394E- | |
| | P395R- | P395E-P396E | |
| | P396R-V397R | | |

In some embodiments, bispecific antibody can be generated by post-production assembly from half-antibodies, thereby solving the issues of heavy and light chain mispairing. These antibodies often contain modification to favor heterodimerization of half-antibodies. Exemplary systems include but not limited to the knob-into-hole, IgG1 (EEE-RRR), IgG2 (EEE -RRRR) (Strop et al. J Mol Biol (2012)) and DuoBody (F405L-K409R), listed in Table 5. In such case, half-antibody is individually produced in separate cell line and purified. The purified antibodies were then subjected to mild reduction to obtain half-antibodies, which were then assembled into bispecific antibodies. Heterodimeric bispecific antibody was then purified from the mixture using conventional purifications methods.

In some embodiments, strategies on bispecific antibody generation that do not rely on the preferential chain pairing can also be employed. These strategies typically involve introducing genetic modification on the antibody in such a manner that the heterodimer will have distinct biochemical or biophysical properties from the homodimers; thus the post-assembled or expressed heterodimer can be selectively purified from the homodimers. One example was to introduce H435R/Y436F in IgG1 CH3 domain to abolish the Fc binding to protein A resin and then co-express the H435R/Y436F variant with a wildtype Fc. The resulting homodimeric antibodies containing two copies of H435R/Y436F cannot bind to the Protein A column, while heterodimeric antibody comprising one copy of H435R/Y436F mutation will have a decreased affinity for protein A as compared to the strong interaction from homodimeric wildtype antibody (Tustian et al Mabs 2016). Other examples include kappa/lambda antibody (Fischer et al., Nature Communication 2015) and introduction of differential charges (E357Q, S267K or N208D/Q295E/N384D/Q418E/N421D) on the respective chains (US 2018/0142040 A1; (Strop et al. J Mol Biol (2012)).

In some embodiments, bispecific antibody can be generated via fusion of an additional binding site to either the heavy or light chain of an immunoglobulin. Examples of the additional binding site include but not limited to variable regions, scFv, Fab, VHH, and peptide.

### Fusion Protein Fc Regions

In some embodiments, an antibody or fusion protein of the present disclosure comprises an Fc region. In some embodiments, the Fc region comprises one or more mutations that reduce or eliminate FcγR binding and/or effector function. In some embodiments, the Fc region (e.g., an IgG1 Fc region) comprises a substitution at one or more of the following positions: C220, C226, C229, E233, L234, L235, G237, P238, S239 D265, S267, N297, L328, P331, K322, A327 and P329. In some embodiments, the Fc region (e.g., an IgG2 Fc region) comprises a substitution at one or more of the following positions: V234, G237, D265, H268, N297, V309, A330, A331, K322. In some embodiments, the Fc region (e.g., an IgG4 Fc region) comprises a substitution at one or more of the following positions: L235, G237, D265 and E318. In some embodiments, the Fc region (e.g., an IgG1 Fc region) comprises one or more of the following mutations or groups of mutations: N297A, N297Q, N297G, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G,E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted. In some embodiments, the Fc region (e.g., an IgG2 Fc region) comprises one or more of the following mutations or groups of mutations: A330S/A331S, V234A/G237A, V234A/G237A/D265A, D265A/A330S/A331S, V234A/G237A/D265A/A330S/A331S, and H268Q/V309L/A330S/A331S. In some embodiments, the Fc region (e.g., an IgG4 Fc region) comprises one or more of the following mutations or groups of mutations: L235A/G237A/E318A, D265A, L235A/G237A/D265A and L235A/G237A/D265A/E318A. In some embodiments, the Fc region comprises one, two, three, or all of the following mutations: L234A, L235A, G237A, and K322A, numbering according to EU index. In some embodiments, the Fc region comprises one, two, or all of the following mutations: L234A, L235A, and G237A, numbering according to EU index.

In some embodiments, said first and second Fc domains of the fusion protein contain one or more of the following Fc mutations to decrease effector function according to EU numbering: L234A, L235A, G237A, and K322A. In some embodiments, said first and second Fc domains of the fusion protein contain the following Fc mutations to decrease effector function according to EU numbering: L234A, L235A, and G237A. In some embodiments, said first and second Fc domains of the fusion protein contain the following Fc mutations to decrease effector function according to EU numbering: L234A, L235A, G237A, and K322A. In some embodiments, said first and second Fc domains of the fusion protein contain the following amino acid substitutions to facilitate heterodimeric formation: Y349C/T366W (knob) and S354C, T366S, L368A and Y407V (hole).

In some embodiments, the heterodimeric mutations and/or mutations to modify Fc gamma receptor binding resulted in reduction of Fc stability. Therefore, additional mutation(s) was added to the Fc region to increase its stability. For example, one or more pairs of disulfide bonds such as A287C and L306C, V259C and L306C, R292C and V302C, and V323C and 1332C are introduced into the Fc region. Another example is to introduce S228P to IgG4 based bispecific antibodies to stabilize the hinge disulfide. Additional example includes introducing K338I, A339K, and K340S mutations to enhance Fc stability and aggregation resistance (Gao et al, 2019 Mol Pharm. 2019; 16:3647).

### Fusion Protein Cytokines

In some embodiments, the second moiety induces activation of CD8+ T cells. In some embodiments, the second moiety comprises a polypeptide that induces signaling via IL2Rβγ. For example, in some embodiments, the second moiety comprises an IL-15 polypeptide (e.g., a human IL-15 polypeptide or derivative thereof) or a neoleukin. In some embodiments, the second moiety comprises an IL-21 polypeptide (e.g., a human IL-21 polypeptide or derivative thereof).

In some embodiments, the second moiety comprises an IL-2 polypeptide (e.g., a human IL-2 polypeptide or derivative thereof).

In some embodiments, the mutant IL-2 polypeptide has a binding affinity to IL-2Rα that is reduced by 50% or more, compared to binding affinity of a wild-type IL-2 polypeptide comprising the sequence of SEQ ID: 81 for IL-2Rα. In some embodiments, the mutant IL-2 polypeptide has a binding affinity to IL-2Rβ that is reduced by 50% or more, compared to binding affinity of a wild-type IL-2 polypeptide comprising the sequence of SEQ ID: 81 for IL-2Rβ. In some embodiments, the mutant IL-2 polypeptide has a binding affinity to IL-2Rγ that is reduced by 50% or more, compared to binding affinity of a wild-type IL-2 polypeptide comprising the sequence of SEQ ID: 81 for IL-2Ry. Differences in binding affinity of wild-type and disclosed mutant polypeptide for IL-2Rα and IL-2Rß can be measured, e.g., in standard surface plasmon resonance (SPR) assays that measure affinity of protein-protein interactions familiar to those skilled in the art. Differences in binding affinity of wild-type and disclosed mutant polypeptide for IL-2Rγ cannot reliably be measured by SPR assays as the affinity of wild-type IL-2 polypeptide for IL-2Rγ is very low. Instead, their reduced affinity to IL-2Rγ can be deduced by performing an in vitro assay that measures pSTAT5 and compares the activity of IL-2 polypeptides with and without the IL-2Rγ affinity-reducing substitution on IL-2R-expressing cells. Exemplary sequences for IL-2Rα, IL-2Rβ, and IL-2Rγ are provided below.

IL-2Rα:
(SEQ ID NO: 82)
MDSYLLMWGLLTFIMVPGCQAELCDDDPPEIPHATFKAMAYKEGTMLNCE
CKRGFRRIKSGSLYMLCTGNSSHSSWDNQCQCTSSATRNTTKQVTPQPEE
QKERKTTEMQSPMQPVDQASLPGHCREPPPWENEATERIYHFVVGQMVYY
QCVQGYRALHRGPAESVCKMTHGKTRWTQPQLICTGEMETSQFPGEEKPQ
ASPEGRPESETSCLVTTTDFQIQTEMAATMETSIFTTEYQVAVAGCVFLL
ISVLLLSGLTWQRRQRKSRRTI
IL-2Rβ:
(SEQ ID NO: 83)
MAAPALSWRLPLLILLLPLATSWASAAVNGTSQFTCFYNSRANISCVWSQ
DGALQDTSCQVHAWPDRRRWNQTCELLPVSQASWACNLILGAPDSQKLTT
VDIVTLRVLCREGVRWRVMAIQDFKPFENLRLMAPISLQVVHVETHRCNI
SWEISQASHYFERHLEFEARTLSPGHTWEEAPLLTLKQKQEWICLETLTP
DTQYEFQVRVKPLQGEFTTWSPWSQPLAFRTKPAALGKDTIPWLGHLLVG
LSGAFGFIILVYLLINCRNTGPWLKKVLKCNTPDPSKFFSQLSSEHGGDV
QKWLSSPFPSSSFSPGGLAPEISPLEVLERDKVTQLLLQQDKVPEPASLS
SNHSLTSCFTNQGYFFFHLPDALEIEACQVYFTYDPYSEEDPDEGVAGAP
TGSSPQPLQPLSGEDDAYCTFPSRDDLLLFSPSLLGGPSPPSTAPGGSGA
GEERMPPSLQERVPRDWDPQPLGPPTPGVPDLVDFQPPPELVLREAGEEV
PDAGPREGVSFPWSRPPGQGEFRALNARLPLNTDAYLSLQELQGQDPTHL
V
IL-2Rγ:
(SEQ ID NO: 84)
MLKPSLPFTSLLFLQLPLLGVGLNTTILTPNGNEDTTADFFLTTMPTDSL
SVSTLPLPEVQCFVFNVEYMNCTWNSSSEPQPTNLTLHYWYKNSDNDKVQ
KCSHYLFSEEITSGCQLQKKEIHLYQTFVVQLQDPREPRRQATQMLKLQN
LVIPWAPENLTLHKLSESQLELNWNNRFLNHCLEHLVQYRTDWDHSWTEQ
SVDYRHKFSLPSVDGQKRYTFRVRSRFNPLCGSAQHWSEWSHPIEWGSNT
SKENPFLFALEAVVISVGSMGLIISLLCVYFWLERTMPRIPTLKNLEDLV
TEYHGNFSAWSGVSKGLAESLQPDYSERLCLVSEIPPKGGALGEGPGASP
CNQHSPYWAPPCYTLKPET

In some embodiments, the IL-2 polypeptide is a mutant IL-2 polypeptide comprising one or more mutations relative to a human IL-2 polypeptide comprising the sequence of APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO:81). In some embodiments, the mutant IL-2 polypeptides of the present disclosure have one or more, two or more, or three or more affinity-reducing amino acid substitutions relative to the wild-type mature IL-2 polypeptide, e.g., having the amino acid sequence of SEQ ID NO:81. In some embodiments, one or more, two or more, or three or more substituted residues, are selected from the following group: Q11, H16, L18, L19, D20, D84, S87, Q22, R38, F42, K43, Y45, E62, P65, E68, V69, L72, D84, S87, N88, V91, 192, T123, Q126, S127, 1129, and S130. Decreased affinity to IL-2Rx may be obtained by substituting one or more of the following residues in the sequence of the wild-type mature IL-2 polypeptide: R38, F42, K43, Y45, E62, P65, E68, V69, and L72. Decreased affinity to IL-2Rβ may be obtained by substituting one or more of the following residues: E15, H16, L19, D20, D84, S87, N88, V91, and 192. Decreased affinity to IL-2Rγ may be obtained by substituting one or more of the following residues in the sequence of the wild-type mature IL-2 polypeptide: Q11, L18, Q22, T123, Q126, S127, 1129, and $130.

In some embodiments, the mutant IL-2 polypeptide comprises an F42A or F42K amino acid substitution relative to the wild-type mature IL-2 amino acid sequence. In some embodiments, the mutant IL-2 polypeptide comprises an F42A or F42K amino acid substitution and an R38A, R38D, R38E, E62Q, E68A, E68Q, E68K, or E68R amino acid substitution relative to the wild-type mature IL-2 amino acid sequence. For example, in some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence, e.g., as shown in SEQ ID NO:81. In some embodiments, the mutant IL-2 polypeptide comprises R38E and F42A amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V9IT, V91E, 192A, E95S, E95A, E95R, T123A, T123E, T123K, T123Q, Q126A, Q126S, Q126T, Q126E, S127A, S127E, S127K, or S127Q amino acid substitution relative to the wild-type IL-2 amino acid sequence. In some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence and an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V91T, V91E, 192A, E95S, E95A, E95R, T123A, T123E, T123K, T123Q, Q126A, Q126S, Q126T, Q126E, S127A, S127E, S127K, or S127Q amino acid substitution relative to the wild-type mature IL-2 amino acid sequence, e.g., as shown in SEQ ID NO:81. For example, in some embodiments, the mutant IL-2 polypeptide comprises R38E, F42A, and H16E amino acid substitutions relative to the wild-type IL-2 amino acid sequence. In some embodiments, the mutant IL-2 polypeptide comprises R38E, F42A, and H16D amino acid substitutions relative to the wild-type IL-2 amino acid sequence. In some embodiments, the mutant IL-2 polypeptide comprises R38E, F42A, and D84K amino acid substitutions relative to the wild-type IL-2 amino acid sequence. In some embodiments, the mutant IL-2 polypeptide comprises R38E, F42A, and D84R amino acid substitutions relative to the wild-type IL-2 amino acid sequence. In some embodiments, the mutant IL-2 polypeptide comprises R38E, F42A, and N88S amino acid substitutions relative to the wild-type IL-2 amino acid sequence. In some embodiments, the mutant IL-2 polypeptide comprises R38E, F42A, and N88A amino acid substitutions relative to the wild-type IL-2 amino acid sequence. In some embodiments, the mutant IL-2 polypeptide comprises R38E, F42A, and N88G amino acid substitutions relative to the wild-type IL-2 amino acid sequence. In some embodiments, the mutant IL-2 polypeptide comprises R38E, F42A, and N88R amino acid substitutions relative to the wild-type IL-2 amino acid sequence. In some embodiments, the mutant IL-2 polypeptide comprises R38E, F42A, and N88T amino acid substitutions relative to the wild-type IL-2 amino acid sequence. In some embodiments, the mutant IL-2 polypeptide comprises R38E, F42A, and N88D amino acid substitutions relative to the wild-type IL-2 amino acid sequence. In some embodiments, the mutant IL-2 polypeptide comprises R38E, F42A, and V91E amino acid substitutions relative to the wild-type IL-2 amino acid sequence. In some embodiments, the mutant IL-2 polypeptide comprises R38E, F42A, and Q126S amino acid substitutions relative to the wild-type IL-2 amino acid sequence. In some embodiments, the mutant IL-2 polypeptide comprises the amino acid sequence of SEQ ID NO:81 with one of the following sets of amino acid substitutions (relative to the sequence of SEQ ID NO: 81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88T, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, N88G, and C125A; R38A, F42K, N88R, and C125A; R38A, F42K, N88T, and C125A; R38A, F42K, N88D, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the IL-2 polypeptide comprises the sequence of SEQ ID NO:81 with one, two, three, four, or five amino acid substitutions relative to SEQ ID NO:81, and wherein the one, two, three, four, or five substitution(s) comprise substitution(s) at positions of SEQ ID NO:81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130. In some embodiments, the IL-2 polypeptide comprises the sequence of SEQ ID NO:81 with one of the following sets of amino acid substitutions (relative to the sequence of SEQ ID NO:81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R.

In some embodiments, the IL-2 polypeptide comprises the sequence of SEQ ID NO: 81 with a further amino acid substitution relative to SEQ ID NO:81 at position C125. In some embodiments, the IL-2 polypeptide comprises the sequence of SEQ ID NO:81 with one of the following sets of amino acid substitutions (relative to the sequence of SEQ ID NO:81): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A;F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISAINVIVLELKGSETTFMCEYADETATIVEFLNR WITFAQSIISTLT (SEQ ID NO:80). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:85). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:86). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCQSIISTLT (SEQ ID NO:87). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISSINVIVLELKGSETTFMCEYADETATIVEFLNR WITFCQSIISTLT (SEQ ID NO:89). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISAINVIVLELKGSETTFMCEYADETATIVEFLNR WITFCQSIISTLT (SEQ ID NO:90). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINEIVLELKGSETTFMCEYADETATIVEFLNR WITFCQSIISTLT (SEQ ID NO:91). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRHLISNINVIVLELKGSETTFMCEYADETATIVEFLNR WITFCQSIISTLT (SEQ ID NO:92). In some embodiments, the mutant IL-2 polypeptide comprises the amino acid sequence of APTSSSTKKTQLQLEDLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:93). In some embodiments, the mutant IL-2 polypeptide comprises the amino acid sequence of APTSSSTKKTQLQLEELLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:94). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFCSSIISTLT (SEQ ID NO:95). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISSINVIVLELKGSETTFMCEYADETATIVEFLNR WITFCQSIISTLT (SEQ ID NO:96). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISAINVIVLELKGSETTFMCEYADETATIVEFLNR WITFCQSIISTLT (SEQ ID NO:97). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINEIVLELKGSETTFMCEYADETATIVEFLNR WITFCQSIISTLT (SEQ ID NO:98). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRHLISNINVIVLELKGSETTFMCEYADETATIVEFLNR WITFCQSIISTLT (SEQ ID NO:99). In some embodiments, the mutant IL-2 polypeptide comprises the amino acid sequence of APTSSSTKKTQLQLEDLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:100). In some embodiments, the mutant IL-2 polypeptide comprises the amino acid sequence of APTSSSTKKTQLQLEELLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:101). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFCSSIISTLT (SEQ ID NO:102). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISSINVIVLELKGSETTFMCEYADETATIVEFLNR WITFCQSIISTLT (SEQ ID NO:103). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISAINVIVLELKGSETTFMCEYADETATIVEFLNR WITFCQSIISTLT (SEQ ID NO:104). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINEIVLELKGSETTFMCEYADETATIVEFLNR WITFCQSIISTLT (SEQ ID NO:105). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRHLISNINVIVLELKGSETTFMCEYADETATIVEFLNR WITFCQSIISTLT (SEQ ID NO:106). In some embodiments, the mutant IL-2 polypeptide comprises the amino acid sequence of APTSSSTKKTQLQLEDLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCQSIISTLT (SEQ ID NO:107). In some embodiments, the mutant IL-2 polypeptide comprises the amino acid sequence of APTSSSTKKTQLQLEELLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCQSIISTLT (SEQ ID NO:108). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCSSIISTLT (SEQ ID NO:109). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISSINVIVLELKGSETTFMCEYADETATIVEFLNR WITFCQSIISTLT (SEQ ID NO:110). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISAINVIVLELKGSETTFMCEYADETATIVEFLNR WITFCQSIISTLT (SEQ ID NO:111). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINEIVLELKGSETTFMCEYADETATIVEFLNR WITFCQSIISTLT (SEQ ID NO:112). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRHLISNINVIVLELKGSETTFMCEYADETATIVEFLNR WITFCQSIISTLT (SEQ ID NO:113). In some embodiments, the mutant IL-2 polypeptide comprises the amino acid sequence of APTSSSTKKTQLQLEDLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCQSIISTLT (SEQ ID NO:114). In some embodiments, the mutant IL-2 polypeptide comprises the amino acid sequence of APTSSSTKKTQLQLEELLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCQSIISTLT (SEQ ID NO:115). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCSSIISTLT (SEQ ID NO:116). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:117). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:118). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFAQSIISTLT (SEQ ID NO:119). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFAQSIISTLT (SEQ ID NO:120). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISSINVIVLELKGSETTFMCEYADETATIVEFLNR WITFAQSIISTLT (SEQ ID NO:121). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISAINVIVLELKGSETTFMCEYADETATIVEFLNR WITFAQSIISTLT (SEQ ID NO:122). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINEIVLELKGSETTFMCEYADETATIVEFLNR WITFAQSIISTLT (SEQ ID NO:123). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRHLISNINVIVLELKGSETTFMCEYADETATIVEFLNR WITFAQSIISTLT (SEQ ID NO:124). In some embodiments, the mutant IL-2 polypeptide comprises the amino acid sequence of APTSSSTKKTQLQLEDLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:125). In some embodiments, the mutant IL-2 polypeptide comprises the amino acid sequence of APTSSSTKKTQLQLEELLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:126). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFASSIISTLT (SEQ ID NO:127). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISSINVIVLELKGSETTFMCEYADETATIVEFLNR WITFAQSIISTLT (SEQ ID NO:128). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISAINVIVLELKGSETTFMCEYADETATIVEFLNR WITFAQSIISTLT (SEQ ID NO:129). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINEIVLELKGSETTFMCEYADETATIVEFLNR WITFAQSIISTLT (SEQ ID NO:130). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRHLISNINVIVLELKGSETTFMCEYADETATIVEFLNR WITFAQSIISTLT (SEQ ID NO:131). In some embodiments, the mutant IL-2 polypeptide comprises the amino acid sequence of APTSSSTKKTQLQLEDLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:132). In some embodiments, the mutant IL-2 polypeptide comprises the amino acid sequence of APTSSSTKKTQLQLEELLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:133). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFASSIISTLT (SEQ ID NO:134). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISSINVIVLELKGSETTFMCEYADETATIVEFLNR WITFAQSIISTLT (SEQ ID NO:135). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISAINVIVLELKGSETTFMCEYADETATIVEFLNR WITFAQSIISTLT (SEQ ID NO:136). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINEIVLELKGSETTFMCEYADETATIVEFLNR WITFAQSIISTLT (SEQ ID NO:137). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRHLISNINVIVLELKGSETTFMCEYADETATIVEFLNR WITFAQSIISTLT (SEQ ID NO:138). In some embodiments, the mutant IL-2 polypeptide comprises the amino acid sequence of APTSSSTKKTQLQLEDLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFAQSIISTLT (SEQ ID NO:139). In some embodiments, the mutant IL-2 polypeptide comprises the amino acid sequence of APTSSSTKKTQLQLEELLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFAQSIISTLT (SEQ ID NO:140). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFASSIISTLT (SEQ ID NO:141). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISSINVIVLELKGSETTFMCEYADETATIVEFLNR WITFAQSIISTLT (SEQ ID NO:142). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISAINVIVLELKGSETTFMCEYADETATIVEFLNR WITFAQSIISTLT (SEQ ID NO:143). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINEIVLELKGSETTFMCEYADETATIVEFLNR WITFAQSIISTLT (SEQ ID NO:144). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRHLISNINVIVLELKGSETTFMCEYADETATIVEFLNR WITFAQSIISTLT (SEQ ID NO:145). In some embodiments, the mutant IL-2 polypeptide comprises the amino acid sequence of APTSSSTKKTQLQLEDLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFAQSIISTLT (SEQ ID NO:146). In some embodiments, the mutant IL-2 polypeptide comprises the amino acid sequence of APTSSSTKKTQLQLEELLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFAQSIISTLT (SEQ ID NO:147). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFASSIISTLT (SEQ ID NO:148). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISSINVIVLELKGSETTFMCEYADETATIVEFLNR WITFAQSIISTLT (SEQ ID NO:149). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISAINVIVLELKGSETTFMCEYADETATIVEFLNR WITFAQSIISTLT (SEQ ID NO:150). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISNINEIVLELKGSETTFMCEYADETATIVEFLNR WITFAQSIISTLT (SEQ ID NO:151). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRHLISNINVIVLELKGSETTFMCEYADETATIVEFLNR WITFAQSIISTLT (SEQ ID NO:152). In some embodiments, the mutant IL-2 polypeptide comprises the amino acid sequence of APTSSSTKKTQLQLEDLLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:153). In some embodiments, the mutant IL-2 polypeptide comprises the amino acid sequence of APTSSSTKKTQLQLEELLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:154). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFASSIISTLT (SEQ ID NO:155). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISGINVIVLELKGSETTFMCEYADETATIVEFLNR WITFCQSIISTLT (SEQ ID NO:190). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRKLISNINVIVLELKGSETTFMCEYADETATIVEFLNR WITFCQSIISTLT (SEQ ID NO:191). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRRLISNINVIVLELKGSETTFMCEYADETATIVEFLNR WITFCQSIISTLT (SEQ ID NO:192). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISGINVIVLELKGSETTFMCEYADETATIVEFLNR WITFCQSIISTLT (SEQ ID NO:193). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRKLISNINVIVLELKGSETTFMCEYADETATIVEFLNR WITFCQSIISTLT (SEQ ID NO:194). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRRLISNINVIVLELKGSETTFMCEYADETATIVEFLNR WITFCQSIISTLT (SEQ ID NO:195). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISGINVIVLELKGSETTFMCEYADETATIVEFLNR WITFCQSIISTLT (SEQ ID NO:196). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRKLISNINVIVLELKGSETTFMCEYADETATIVEFLNR WITFCQSIISTLT (SEQ ID NO:197). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRRLISNINVIVLELKGSETTFMCEYADETATIVEFLNR WITFCQSIISTLT (SEQ ID NO:198). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISGINVIVLELKGSETTFMCEYADETATIVEFLNR WITFCQSIISTLT (SEQ ID NO:199). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRKLISNINVIVLELKGSETTFMCEYADETATIVEFLNR WITFCQSIISTLT (SEQ ID NO:200). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRRLISNINVIVLELKGSETTFMCEYADETATIVEFLNR WITFCQSIISTLT (SEQ ID NO:201). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISGINVIVLELKGSETTFMCEYADETATIVEFLNR WITFAQSIISTLT (SEQ ID NO:202). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRKLISNINVIVLELKGSETTFMCEYADETATIVEFLNR WITFAQSIISTLT (SEQ ID NO:203). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRRLISNINVIVLELKGSETTFMCEYADETATIVEFLNR WITFAQSIISTLT (SEQ ID NO:204). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISGINVIVLELKGSETTFMCEYADETATIVEFLNR WITFAQSIISTLT (SEQ ID NO:205). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRKLISNINVIVLELKGSETTFMCEYADETATIVEFLNR WITFAQSIISTLT (SEQ ID NO:206). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRRLISNINVIVLELKGSETTFMCEYADETATIVEFLNR WITFAQSIISTLT (SEQ ID NO:207). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISGINVIVLELKGSETTFMCEYADETATIVEFLNR WITFAQSIISTLT (SEQ ID NO:208). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRKLISNINVIVLELKGSETTFMCEYADETATIVEFLNR WITFAQSIISTLT (SEQ ID NO:209). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRRLISNINVIVLELKGSETTFMCEYADETATIVEFLNR WITFAQSIISTLT (SEQ ID NO:210). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISGINVIVLELKGSETTFMCEYADETATIVEFLNR WITFAQSIISTLT (SEQ ID NO:211). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRKLISNINVIVLELKGSETTFMCEYADETATIVEFLNR WITFAQSIISTLT (SEQ ID NO:212). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRRLISNINVIVLELKGSETTFMCEYADETATIVEFLNR WITFAQSIISTLT (SEQ ID NO:213). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISGINVIVLELKGSETTFMCEYADETATIVEFLNR WITFAQSIISTLT (SEQ ID NO:214). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRKLISNINVIVLELKGSETTFMCEYADETATIVEFLNR WITFAQSIISTLT (SEQ ID NO:215). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRRLISNINVIVLELKGSETTFMCEYADETATIVEFLNR WITFAQSIISTLT (SEQ ID NO:216). In some embodiments, the mutant IL-2 polypeptide comprises the amino acid sequence of APTSSSTKKTQLQLEELLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:297). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISDINVIVLELKGSETTFMCEYADETATIVEFLNR WITFAQSIISTLT (SEQ ID NO:354). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISTINVIVLELKGSETTFMCEYADETATIVEFLNR WITFAQSIISTLT (SEQ ID NO:355). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISRINVIVLELKGSETTFMCEYADETATIVEFLNR WITFAQSIISTLT (SEQ ID NO:356). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISRINVIVLELKGSETTFMCEYADETATIVEFLNR WITFCQSIISTLT (SEQ ID NO:357). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISTINVIVLELKGSETTFMCEYADETATIVEFLNR WITFCQSIISTLT (SEQ ID NO:358). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISDINVIVLELKGSETTFMCEYADETATIVEFLNR WITFCQSIISTLT (SEQ ID NO:359). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISRINVIVLELKGSETTFMCEYADETATIVEFLNR WITFCQSIISTLT (SEQ ID NO:360). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISTINVIVLELKGSETTFMCEYADETATIVEFLNR WITFCQSIISTLT (SEQ ID NO:361). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISDINVIVLELKGSETTFMCEYADETATIVEFLNR WITFCQSIISTLT (SEQ ID NO:362). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISRINVIVLELKGSETTFMCEYADETATIVEFLNR WITFCQSIISTLT (SEQ ID NO:363). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISTINVIVLELKGSETTFMCEYADETATIVEFLNR WITFCQSIISTLT (SEQ ID NO:364). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISDINVIVLELKGSETTFMCEYADETATIVEFLNR WITFCQSIISTLT (SEQ ID NO:365). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISRINVIVLELKGSETTFMCEYADETATIVEFLNR WITFCQSIISTLT (SEQ ID NO:366). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISTINVIVLELKGSETTFMCEYADETATIVEFLNR WITFCQSIISTLT (SEQ ID NO:367). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISDINVIVLELKGSETTFMCEYADETATIVEFLNR WITFCQSIISTLT (SEQ ID NO:368). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISRINVIVLELKGSETTFMCEYADETATIVEFLNR WITFAQSIISTLT (SEQ ID NO:369). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISTINVIVLELKGSETTFMCEYADETATIVEFLNR WITFAQSIISTLT (SEQ ID NO:370). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISDINVIVLELKGSETTFMCEYADETATIVEFLNR WITFAQSIISTLT (SEQ ID NO:371). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISRINVIVLELKGSETTFMCEYADETATIVEFLNR WITFAQSIISTLT (SEQ ID NO:372). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISTINVIVLELKGSETTFMCEYADETATIVEFLNR WITFAQSIISTLT (SEQ ID NO:373). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISDINVIVLELKGSETTFMCEYADETATIVEFLNR WITFAQSIISTLT (SEQ ID NO:374). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISRINVIVLELKGSETTFMCEYADETATIVEFLNR WITFAQSIISTLT (SEQ ID NO:375). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISTINVIVLELKGSETTFMCEYADETATIVEFLNR WITFAQSIISTLT (SEQ ID NO:376). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISDINVIVLELKGSETTFMCEYADETATIVEFLNR WITFAQSIISTLT (SEQ ID NO:377). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISRINVIVLELKGSETTFMCEYADETATIVEFLNR WITFAQSIISTLT (SEQ ID NO:378). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISTINVIVLELKGSETTFMCEYADETATIVEFLNR WITFAQSIISTLT (SEQ ID NO:379). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISDINVIVLELKGSETTFMCEYADETATIVEFLNR WITFAQSIISTLT (SEQ ID NO:380). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISRINVIVLELKGSETTFMCEYADETATIVEFLNR WITFAQSIISTLT (SEQ ID NO:381). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISTINVIVLELKGSETTFMCEYADETATIVEFLNR WITFAQSIISTLT (SEQ ID NO:382). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISDINVIVLELKGSETTFMCEYADETATIVEFLNR WITFAQSIISTLT (SEQ ID NO:383). In some embodiments, the mutant IL-2 polypeptide comprises the amino acid sequence of an IL-2 polypeptide listed in Table 7.

TABLE 7
Exemplary IL-2 polypeptide sequences

| | | SEQ |
|---|---|---|
| IL-2 | | ID |
| ID | Sequence | NO |
| m1 | APTSSSTKKTQLQLEHLLLDLQMILNGINNYKNPKLTEMLTAKFYMPKKATEL | 80 |
| | KHLQCLEEELKPLEEVLNLAQSKNFHLRPRDLISAINVIVLELKGSETTFMCEYA | |
| | DETATIVEFLNRWITFAQSIISTLT | |
| m2 | APTSSSTKKTQLQLEHLLLDLQMILNGINNYKNPKLTEMLTAKFYMPKKATEL | 121 |
| | KHLQCLEEELKPLEEVLNLAQSKNFHLRPRDLISSINVIVLELKGSETTFMCEYA | |
| | DETATIVEFLNRWITFAQSIISTLT | |
| m3 | APTSSSTKKTQLQLEDLLLDLQMILNGINNYKNPKLTEMLTAKFYMPKKATEL | 125 |
| | KHLQCLEEELKPLEEVLNLAQSKNFHLRPRDLISNINVIVLELKGSETTFMCEYA | |
| | DETATIVEFLNRWITFAQSIISTLT | |
| m4 | APTSSSTKKTQLQLEELLLDLQMILNGINNYKNPKLTEMLTAKFYMPKKATELK | 297 |
| | HLQCLEEELKPLEEVLNLAQSKNFHLRPRDLISNINVIVLELKGSETTFMCEYAD | |
| | ETATIVEFLNRWITFAQSIISTLT | |
| m5 | APTSSSTKKTQLQLEHLLLDLQMILNGINNYKNPKLTEMLTAKFYMPKKATEL | 202 |
| | KHLQCLEEELKPLEEVLNLAQSKNFHLRPRDLISGINVIVLELKGSETTFMCEYA | |
| | DETATIVEFLNRWITFAQSIISTLT | |
| m6 | APTSSSTKKTQLQLEHLLLDLQMILNGINNYKNPKLTEMLTAKFYMPKKATEL | 117 |
| | KHLQCLEEELKPLEEVLNLAQSKNFHLRPRDLISNINVIVLELKGSETTFMCEYA | |
| | DETATIVEFLNRWITFAQSIISTLT | |
| m7 | APTSSSTKKTQLQLEHLLLDLQMILNGINNYKNPKLTEMLTAKFYMPKKATEL | 123 |
| | KHLQCLEEELKPLEEVLNLAQSKNFHLRPRDLISNINEIVLELKGSETTFMCEYA | |
| | DETATIVEFLNRWITFAQSIISTLT | |
| m8 | APTSSSTKKTQLQLEHLLLDLQMILNGINNYKNPKLTEMLTAKFYMPKKATEL | 124 |
| | KHLQCLEEELKPLEEVLNLAQSKNFHLRPRHLISNINVIVLELKGSETTFMCEYA | |
| | DETATIVEFLNRWITFAQSIISTLT | |
| m9 | APTSSSTKKTQLQLEHLLLDLQMILNGINNYKNPKLTEMLTAKFYMPKKATEL | 203 |
| | KHLQCLEEELKPLEEVLNLAQSKNFHLRPRKLISNINVIVLELKGSETTFMCEYA | |
| | DETATIVEFLNRWITFAQSIISTLT | |
| m10 | APTSSSTKKTQLQLEHLLLDLQMILNGINNYKNPKLTEMLTAKFYMPKKATEL | 204 |
| | KHLQCLEEELKPLEEVLNLAQSKNFHLRPRRLISNINVIVLELKGSETTFMCEYA | |
| | DETATIVEFLNRWITFAQSIISTLT | |
| m11 | APTSSSTKKTQLQLEHLLLDLQMILNGINNYKNPKLTEMLTAKFYMPKKATEL | 354 |
| | KHLQCLEEELKPLEEVLNLAQSKNFHLRPRDLISDINVIVLELKGSETTFMCEYA | |
| | DETATIVEFLNRWITFAQSIISTLT | |
| m12 | APTSSSTKKTQLQLEHLLLDLQMILNGINNYKNPKLTEMLTAKFYMPKKATEL | 355 |
| | KHLQCLEEELKPLEEVLNLAQSKNFHLRPRDLISTINVIVLELKGSETTFMCEYA | |
| | DETATIVEFLNRWITFAQSIISTLT | |
| m13 | APTSSSTKKTQLQLEHLLLDLQMILNGINNYKNPKLTEMLTAKFYMPKKATEL | 356 |
| | KHLQCLEEELKPLEEVLNLAQSKNFHLRPRDLISRINVIVLELKGSETTFMCEYA | |
| | DETATIVEFLNRWITFAQSIISTLT | |

In some embodiments, the mutant IL-2 polypeptides of the present disclosure also contain other modifications, including but not limited to mutations and deletions, that provide additional advantages such as improved biophysical properties. Improved biophysical properties include but are not limited to improved thermostability, aggregation propensity, acid reversibility, viscosity, and production in a mammalian or bacterial or yeast cell. For example, residue C125 may be replaced with a neutral amino acid such as serine, alanine, threonine or valine; and N terminal Al residue could be deleted, both of which were described in U.S. Pat. No. 4,518,584. Mutant IL-2 polypeptides may also include a mutation of the residue M104, such as M104A, as described in U.S. Pat. No. 5,206,344. Thus, in certain embodiments the mutant IL-2 polypeptide of the present disclosure comprises the amino acid substitution C125A. In other embodiments, one, two, or three N-terminal residues are deleted.

### Fusion Protein Linkers

In some embodiments, a fusion protein of the present disclosure comprises a linker. In some embodiments, the linker is a chemical linker (for example, see disclosed in Protein Engineering, 9 (3), 299-305, 1996). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis [2-(succinimidoxycarbonyloxy)ethyl] sulfone (BSOCOES), and bis [2-(succinimidoxycarbonyloxy)ethyl] sulfone (sulfo-BSOCOES).

In some embodiments, the linker is an amino acid- or peptide-based linker. In some embodiments, the polypeptide linker is a peptide with a length of at least 5 amino acids, preferably with a length of 5 to 100, more preferably of 10 to 50 amino acids. In one embodiment, said peptide linker is G, S, GS, SG, SGG, GGS, and GSG (with G=glycine and S=serine). In some embodiments, the linker comprises the sequence (GGGS)xGn (SEQ ID NO: 74), (GGGGS)xGn (SEQ ID NO:75), (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3. In some embodiments, the linker comprises the sequence GGGGSGGGGSGGGGS (SEQ ID NO:79) or SGGGGSGGGGSGGGGS (SEQ ID NO:389).

### Fusion Protein Properties

In some embodiments, the fusion protein induces activation of cells expressing a human CD8ab heterodimer with at least 2-fold, at least 5-fold, or at least 10-fold higher potency than activation of cells expressing a human CD8aa homodimer. In some embodiments, the fusion protein induces activation of CD8+ T cells with at least 2-fold, at least 5-fold, or at least 10-fold higher potency than activation of NK cells. In some embodiments, potency of activation is measured by EC50, as assessed by cell proliferation. Exemplary assays are further described infra.

Preferential activity of the targeted IL-2 fusion proteins comprising the mutant IL-2 polypeptides on antigen-expressing cells is demonstrated in assays that contain antigen-expressing and antigen-non expressing cells that also express IL-2Rβγ or IL-2Rαβγ. One such assay is an in vitro assay that measures STAT5 (pSTAT5)phosphorylation and/or expression of the proliferation marker Ki-67 in human immune cells, such as human peripheral blood and/or tumor-infiltrating immune cells upon exposure to IL-2 polypeptides. In one format of the assay, the activity of the targeted IL-2 fusion protein is measured on antigen-expressing and non-expressing cells to demonstrate the selectivity on antigen-expressing cells. In another format of the assay, the activity of the targeted IL-2 fusion protein comprising the mutant IL-2 polypeptide on antigen-expressing cells is compared to that of the untargeted IL-2 fusion protein comprising the same mutant IL-2 polypeptide and a control antibody not recognizing any antigens on antigen-expressing cells to demonstrate the magnitude of rescue in signaling of the mutant IL-2 polypeptide when fused to an antigen binding molecule.

In some embodiments, the fusion protein of the present disclosure containing CD8b antigen binding molecules activates CD8b+IL-2Rβ+ cells over CD8b-IL-2Rβ+ cells by at least 10-fold, at least 50-fold, or at least 100-fold. In some embodiments, said fusion protein activates CD8b+IL-2Rβ+ cells more than 50-fold, 100 fold, or 200 fold compared to a fusion molecule comprising the said IL-2 mutant polypeptide and a control antibody not binding to any antigens expressed on said cells. Said cell activation by the IL-2 fusion protein is determined by measuring the expression of pSTAT5 or the cell proliferation marker Ki67 in said cells following the treatment with said IL-2 fusion protein.

In some embodiments, a fusion protein of the present disclosure displays one or more of the following: binds human CD8 and does not block an interaction of CD8 with MHC class I; and activates CD8+ T cells with at least 10-fold, 25-fold, 50-fold, 100-fold, 250-fold, 500-fold, or 1000-fold greater potency, e.g., as compared to activation of NK cells. In some embodiments, whether an anti-CD8 antibody or fusion protein of the present disclosure blocks the interaction of CD8 with MHC class I can be assayed, e.g., by assaying activation status of CD8+ T cells (e.g., upon antigen stimulation) in the presence or absence of the anti-CD8 antibody or fusion protein. In some embodiments, activation of CD8+ T cells and/or NK cells can be measured, e.g., by assaying one or more markers (e.g., proportion of treated cells expressing one or more markers) of proliferation (e.g., Ki67), IL-2Rβ/γ downstream signaling, and/or STAT5 downstream signaling.

### Exemplary Fusion Proteins

In some embodiments, a fusion protein of the present disclosure comprises one, two, or all three polypeptides shown for a single fusion protein in Table 5. In some embodiments, a fusion protein of the present disclosure comprises two light chains, a heavy chain comprising an IL-2 fusion, and a heavy chain not comprising an IL-2 fusion for a single fusion protein in Table 5. As is known in the art, the C-terminal lysine of some antibody heavy chain species may be cleaved off in some fraction of molecules. Therefore, in some embodiments, a fusion protein of the present disclosure comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos: 158, 161, 164, 167, 170, 173, 176, 189, 300, 304, 308, 312, 326, 320, 324, 328, 332, 336, 340, 344, 348, or 352 for the respective fusion protein. In some embodiments, a fusion protein of the present disclosure comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos: 217-224, 301, 305, 309, 313, 317, 321, 325, 329, 333, 337, 341, 345, 349, or 353 for the respective fusion protein. In some embodiments, the present disclosure provides a plurality of fusion proteins of the present disclosure (e.g., in a mixture), wherein each fusion protein of the plurality comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos: 158, 161, 164, 167, 170, 173, 176, 189, 300, 304, 308, 312, 326, 320, 324, 328, 332, 336, 340, 344, 348, or 352 for the respective fusion protein, or the sequence of SEQ ID Nos: 217-224, 301, 305, 309, 313, 317, 321, 325, 329, 333, 337, 341, 345, 349, or 353 for the respective fusion protein, or the plurality comprises a mixture of species representing cleaved (e.g., comprising the sequence of SEQ ID Nos: 158, 161, 164, 167, 170, 173, 176, 189, 300, 304, 308, 312, 326, 320, 324, 328, 332, 336, 340, 344, 348, or 352 for the respective fusion protein) and uncleaved (e.g., comprising the sequence of SEQ ID Nos: 217-224, 301, 305, 309, 313, 317, 321, 325, 329, 333, 337, 341, 345, 349, or 353 for the respective fusion protein) species.

For example, in some embodiments, a fusion protein of the present disclosure comprises one or two light chains comprising the amino acid sequence of SEQ ID NO:156, a heavy chain comprising the amino acid sequence of SEQ ID NO: 157, and a heavy chain comprising the amino acid sequence of SEQ ID NO:158. In some embodiments, a fusion protein of the present disclosure comprises one or two light chains comprising the amino acid sequence of SEQ ID NO: 159, a heavy chain comprising the amino acid sequence of SEQ ID NO: 160, and a heavy chain comprising the amino acid sequence of SEQ ID NO:161. In some embodiments, a fusion protein of the present disclosure comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164. In some embodiments, a fusion protein of the present disclosure comprises one or two light chains comprising the amino acid sequence of SEQ ID NO: 165, a heavy chain comprising the amino acid sequence of SEQ ID NO: 166, and a heavy chain comprising the amino acid sequence of SEQ ID NO:167. In some embodiments, a fusion protein of the present disclosure comprises one or two light chains comprising the amino acid sequence of SEQ ID NO:168, a heavy chain comprising the amino acid sequence of SEQ ID NO: 169, and a heavy chain comprising the amino acid sequence of SEQ ID NO:170. In some embodiments, a fusion protein of the present disclosure comprises one or two light chains comprising the amino acid sequence of SEQ ID NO: 171, a heavy chain comprising the amino acid sequence of SEQ ID NO:172, and a heavy chain comprising the amino acid sequence of SEQ ID NO: 173. In some embodiments, a fusion protein of the present disclosure comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO:176. In some embodiments, a fusion protein of the present disclosure comprises one or two light chains comprising the amino acid sequence of SEQ ID NO:187, a heavy chain comprising the amino acid sequence of SEQ ID NO: 188, and a heavy chain comprising the amino acid sequence of SEQ ID NO:189. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:300. In some embodiments, a fusion protein of the present disclosure comprises one or two light chains comprising the amino acid sequence of SEQ ID NO: 302, a heavy chain comprising the amino acid sequence of SEQ ID NO:303, and a heavy chain comprising the amino acid sequence of SEQ ID NO:304. In some embodiments, a fusion protein of the present disclosure comprises one or two light chains comprising the amino acid sequence of SEQ ID NO:306, a heavy chain comprising the amino acid sequence of SEQ ID NO: 307, and a heavy chain comprising the amino acid sequence of SEQ ID NO:308. In some embodiments, a fusion protein of the present disclosure comprises one or two light chains comprising the amino acid sequence of SEQ ID NO:310, a heavy chain comprising the amino acid sequence of SEQ ID NO:311, and a heavy chain comprising the amino acid sequence of SEQ ID NO:312. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:316. In some embodiments, a fusion protein of the present disclosure comprises one or two light chains comprising the amino acid sequence of SEQ ID NO: 318, a heavy chain comprising the amino acid sequence of SEQ ID NO:319, and a heavy chain comprising the amino acid sequence of SEQ ID NO:320. In some embodiments, a fusion protein of the present disclosure comprises one or two light chains comprising the amino acid sequence of SEQ ID NO:322, a heavy chain comprising the amino acid sequence of SEQ ID NO: 323, and a heavy chain comprising the amino acid sequence of SEQ ID NO:324. In some embodiments, a fusion protein of the present disclosure comprises one or two light chains comprising the amino acid sequence of SEQ ID NO:326, a heavy chain comprising the amino acid sequence of SEQ ID NO:327, and a heavy chain comprising the amino acid sequence of SEQ ID NO:328. In some embodiments, a fusion protein of the present disclosure comprises one or two light chains comprising the amino acid sequence of SEQ ID NO:334, a heavy chain comprising the amino acid sequence of SEQ ID NO:335, and a heavy chain comprising the amino acid sequence of SEQ ID NO:336. In some embodiments, a fusion protein of the present disclosure comprises one or two light chains comprising the amino acid sequence of SEQ ID NO: 338, a heavy chain comprising the amino acid sequence of SEQ ID NO:339, and a heavy chain comprising the amino acid sequence of SEQ ID NO:340. In some embodiments, a fusion protein of the present disclosure comprises one or two light chains comprising the amino acid sequence of SEQ ID NO:342, a heavy chain comprising the amino acid sequence of SEQ ID NO: 343, and a heavy chain comprising the amino acid sequence of SEQ ID NO:344. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:348. In some embodiments, a fusion protein of the present disclosure comprises one or two light chains comprising the amino acid sequence of SEQ ID NO:350, a heavy chain comprising the amino acid sequence of SEQ ID NO:351, and a heavy chain comprising the amino acid sequence of SEQ ID NO:352.

In some embodiments, a fusion protein of the present disclosure comprises one or two light chains comprising the amino acid sequence of SEQ ID NO:156, a heavy chain comprising the amino acid sequence of SEQ ID NO:157, and a heavy chain comprising the amino acid sequence of SEQ ID NO:217. In some embodiments, a fusion protein of the present disclosure comprises one or two light chains comprising the amino acid sequence of SEQ ID NO:159, a heavy chain comprising the amino acid sequence of SEQ ID NO: 160, and a heavy chain comprising the amino acid sequence of SEQ ID NO:218. In some embodiments, a fusion protein of the present disclosure comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO:219. In some embodiments, a fusion protein of the present disclosure comprises one or two light chains comprising the amino acid sequence of SEQ ID NO:165, a heavy chain comprising the amino acid sequence of SEQ ID NO: 166, and a heavy chain comprising the amino acid sequence of SEQ ID NO:220. In some embodiments, a fusion protein of the present disclosure comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO:221. In some embodiments, a fusion protein of the present disclosure comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO:222. In some embodiments, a fusion protein of the present disclosure comprises one or two light chains comprising the amino acid sequence of SEQ ID NO: 174, a heavy chain comprising the amino acid sequence of SEQ ID NO:175, and a heavy chain comprising the amino acid sequence of SEQ ID NO:223. In some embodiments, a fusion protein of the present disclosure comprises one or two light chains comprising the amino acid sequence of SEQ ID NO: 187, a heavy chain comprising the amino acid sequence of SEQ ID NO: 188, and a heavy chain comprising the amino acid sequence of SEQ ID NO:224. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:301. In some embodiments, a fusion protein of the present disclosure comprises one or two light chains comprising the amino acid sequence of SEQ ID NO:302, a heavy chain comprising the amino acid sequence of SEQ ID NO:303, and a heavy chain comprising the amino acid sequence of SEQ ID NO:305. In some embodiments, a fusion protein of the present disclosure comprises one or two light chains comprising the amino acid sequence of SEQ ID NO: 306, a heavy chain comprising the amino acid sequence of SEQ ID NO:307, and a heavy chain comprising the amino acid sequence of SEQ ID NO:309. In some embodiments, a fusion protein of the present disclosure comprises one or two light chains comprising the amino acid sequence of SEQ ID NO:310, a heavy chain comprising the amino acid sequence of SEQ ID NO: 311, and a heavy chain comprising the amino acid sequence of SEQ ID NO:313. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:317. In some embodiments, a fusion protein of the present disclosure comprises one or two light chains comprising the amino acid sequence of SEQ ID NO:318, a heavy chain comprising the amino acid sequence of SEQ ID NO:319, and a heavy chain comprising the amino acid sequence of SEQ ID NO:321. In some embodiments, a fusion protein of the present disclosure comprises one or two light chains comprising the amino acid sequence of SEQ ID NO: 322, a heavy chain comprising the amino acid sequence of SEQ ID NO:323, and a heavy chain comprising the amino acid sequence of SEQ ID NO:325. In some embodiments, a fusion protein of the present disclosure comprises one or two light chains comprising the amino acid sequence of SEQ ID NO:326, a heavy chain comprising the amino acid sequence of SEQ ID NO: 327, and a heavy chain comprising the amino acid sequence of SEQ ID NO:329. In some embodiments, a fusion protein of the present disclosure comprises one or two light chains comprising the amino acid sequence of SEQ ID NO:330, a heavy chain comprising the amino acid sequence of SEQ ID NO:331, and a heavy chain comprising the amino acid sequence of SEQ ID NO:333. In some embodiments, a fusion protein of the present disclosure comprises one or two light chains comprising the amino acid sequence of SEQ ID NO:334, a heavy chain comprising the amino acid sequence of SEQ ID NO:335, and a heavy chain comprising the amino acid sequence of SEQ ID NO:337. In some embodiments, a fusion protein of the present disclosure comprises one or two light chains comprising the amino acid sequence of SEQ ID NO: 338, a heavy chain comprising the amino acid sequence of SEQ ID NO:339, and a heavy chain comprising the amino acid sequence of SEQ ID NO:341. In some embodiments, a fusion protein of the present disclosure comprises one or two light chains comprising the amino acid sequence of SEQ ID NO:342, a heavy chain comprising the amino acid sequence of SEQ ID NO: 343, and a heavy chain comprising the amino acid sequence of SEQ ID NO:345. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:349. In some embodiments, a fusion protein of the present disclosure comprises one or two light chains comprising the amino acid sequence of SEQ ID NO:350, a heavy chain comprising the amino acid sequence of SEQ ID NO:351, and a heavy chain comprising the amino acid sequence of SEQ ID NO:353.

In some embodiments, the present disclosure provides a mixture of fusion protein species, wherein each species comprises one or two light chains comprising the amino acid sequence of SEQ ID NO:156, a heavy chain comprising the amino acid sequence of SEQ ID NO: 157, and a heavy chain comprising the amino acid sequence of SEQ ID NO: 158 or 217. In some embodiments, the present disclosure provides a mixture of fusion protein species, wherein each species comprises one or two light chains comprising the amino acid sequence of SEQ ID NO: 159, a heavy chain comprising the amino acid sequence of SEQ ID NO: 160, and a heavy chain comprising the amino acid sequence of SEQ ID NO: 161 or 218. In some embodiments, the present disclosure provides a mixture of fusion protein species, wherein each species comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO:164 or 219. In some embodiments, the present disclosure provides a mixture of fusion protein species, wherein each species comprises one or two light chains comprising the amino acid sequence of SEQ ID NO:165, a heavy chain comprising the amino acid sequence of SEQ ID NO:166, and a heavy chain comprising the amino acid sequence of SEQ ID NO:167 or 220. In some embodiments, the present disclosure provides a mixture of fusion protein species, wherein each species comprises one or two light chains comprising the amino acid sequence of SEQ ID NO:168, a heavy chain comprising the amino acid sequence of SEQ ID NO: 169, and a heavy chain comprising the amino acid sequence of SEQ ID NO:170 or 221. In some embodiments, the present disclosure provides a mixture of fusion protein species, wherein each species comprises one or two light chains comprising the amino acid sequence of SEQ ID NO: 171, a heavy chain comprising the amino acid sequence of SEQ ID NO:172, and a heavy chain comprising the amino acid sequence of SEQ ID NO:173 or 222. In some embodiments, the present disclosure provides a mixture of fusion protein species, wherein each species comprises one or two light chains comprising the amino acid sequence of SEQ ID NO: 174, a heavy chain comprising the amino acid sequence of SEQ ID NO:175, and a heavy chain comprising the amino acid sequence of SEQ ID NO:176 or 223. In some embodiments, the present disclosure provides a mixture of fusion protein species, wherein each species comprises one or two light chains comprising the amino acid sequence of SEQ ID NO: 187, a heavy chain comprising the amino acid sequence of SEQ ID NO:188, and a heavy chain comprising the amino acid sequence of SEQ ID NO: 189 or 224. In some embodiments, the present disclosure provides a mixture of fusion protein species, wherein each species comprises one or two light chains comprising the amino acid sequence of SEQ ID NO:298, a heavy chain comprising the amino acid sequence of SEQ ID NO:299, and a heavy chain comprising the amino acid sequence of SEQ ID NO:300 or 301. In some embodiments, the present disclosure provides a mixture of fusion protein species, wherein each species comprises one or two light chains comprising the amino acid sequence of SEQ ID NO:302, a heavy chain comprising the amino acid sequence of SEQ ID NO:303, and a heavy chain comprising the amino acid sequence of SEQ ID NO:304 or 305. In some embodiments, the present disclosure provides a mixture of fusion protein species, wherein each species comprises one or two light chains comprising the amino acid sequence of SEQ ID NO:306, a heavy chain comprising the amino acid sequence of SEQ ID NO:307, and a heavy chain comprising the amino acid sequence of SEQ ID NO:308 or 309. In some embodiments, the present disclosure provides a mixture of fusion protein species, wherein each species comprises one or two light chains comprising the amino acid sequence of SEQ ID NO:310, a heavy chain comprising the amino acid sequence of SEQ ID NO:311, and a heavy chain comprising the amino acid sequence of SEQ ID NO:312 or 313. In some embodiments, the present disclosure provides a mixture of fusion protein species, wherein each species comprises one or two light chains comprising the amino acid sequence of SEQ ID NO:314, a heavy chain comprising the amino acid sequence of SEQ ID NO:315, and a heavy chain comprising the amino acid sequence of SEQ ID NO:316 or 317. In some embodiments, the present disclosure provides a mixture of fusion protein species, wherein each species comprises one or two light chains comprising the amino acid sequence of SEQ ID NO: 318, a heavy chain comprising the amino acid sequence of SEQ ID NO:319, and a heavy chain comprising the amino acid sequence of SEQ ID NO:320 or 321. In some embodiments, the present disclosure provides a mixture of fusion protein species, wherein each species comprises one or two light chains comprising the amino acid sequence of SEQ ID NO:322, a heavy chain comprising the amino acid sequence of SEQ ID NO:323, and a heavy chain comprising the amino acid sequence of SEQ ID NO:324 or 325. In some embodiments, the present disclosure provides a mixture of fusion protein species, wherein each species comprises one or two light chains comprising the amino acid sequence of SEQ ID NO:326, a heavy chain comprising the amino acid sequence of SEQ ID NO:327, and a heavy chain comprising the amino acid sequence of SEQ ID NO:328 or 329. In some embodiments, the present disclosure provides a mixture of fusion protein species, wherein each species comprises one or two light chains comprising the amino acid sequence of SEQ ID NO:330, a heavy chain comprising the amino acid sequence of SEQ ID NO:331, and a heavy chain comprising the amino acid sequence of SEQ ID NO:332 or 333. In some embodiments, the present disclosure provides a mixture of fusion protein species, wherein each species comprises one or two light chains comprising the amino acid sequence of SEQ ID NO:334, a heavy chain comprising the amino acid sequence of SEQ ID NO:335, and a heavy chain comprising the amino acid sequence of SEQ ID NO:336 or 337. In some embodiments, the present disclosure provides a mixture of fusion protein species, wherein each species comprises one or two light chains comprising the amino acid sequence of SEQ ID NO:338, a heavy chain comprising the amino acid sequence of SEQ ID NO:339, and a heavy chain comprising the amino acid sequence of SEQ ID NO:340 or 341. In some embodiments, the present disclosure provides a mixture of fusion protein species, wherein each species comprises one or two light chains comprising the amino acid sequence of SEQ ID NO: 342, a heavy chain comprising the amino acid sequence of SEQ ID NO:343, and a heavy chain comprising the amino acid sequence of SEQ ID NO:344 or 345. In some embodiments, the present disclosure provides a mixture of fusion protein species, wherein each species comprises one or two light chains comprising the amino acid sequence of SEQ ID NO:346, a heavy chain comprising the amino acid sequence of SEQ ID NO:347, and a heavy chain comprising the amino acid sequence of SEQ ID NO:348 or 349. In some embodiments, the present disclosure provides a mixture of fusion protein species, wherein each species comprises one or two light chains comprising the amino acid sequence of SEQ ID NO:350, a heavy chain comprising the amino acid sequence of SEQ ID NO:351, and a heavy chain comprising the amino acid sequence of SEQ ID NO:352 or 353.

In some embodiments, the present disclosure provides a fusion protein comprising two heavy chain sequences and two light chain sequences of a single fusion protein listed in Table 5, wherein one of the heavy chain sequences has an IL2 fusion and the other heavy chain sequence is without an IL2 fusion, and wherein the two light chain sequences are identical. In some embodiments, the heavy chain sequence without an IL2 fusion comprises a lysine at the C terminus. In some embodiments, the fusion protein is of format A shown in FIG. 7. For example, in some embodiments, the fusion protein comprises four polypeptide chains, wherein (1) the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:336, and the fourth polypeptide chain comprises the amino acid sequence of SEQ ID NO:334; (2) the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:337, and the fourth polypeptide chain comprises the amino acid sequence of SEQ ID NO:334; (3) the first polypeptide chain comprises the amino acid sequence of SEQ ID NO:338, the second polypeptide chain comprises the amino acid sequence of SEQ ID NO: 339, the third polypeptide chain comprises the amino acid sequence of SEQ ID NO:340, and the fourth polypeptide chain comprises the amino acid sequence of SEQ ID NO:338; or (4) the first polypeptide chain comprises the amino acid sequence of SEQ ID NO:338, the second polypeptide chain comprises the amino acid sequence of SEQ ID NO:339, the third polypeptide chain comprises the amino acid sequence of SEQ ID NO:341, and the fourth polypeptide chain comprises the amino acid sequence of SEQ ID NO:338.

TABLE 5
Anti-CD8:IL2 fusion protein sequences

| | | | | Heavy chain |
|---|---|---|---|---|
| | | | | sequence |
| | | Heavy chain | Heavy chain | (without |
| | | sequence | sequence | IL2 fusion) |
| | Light chain | (with IL2 | (without | plus |
| Name | sequence | fusion) | IL2 fusion) | C-term lysine |
| xh | DIQMTQSPAS | QVHLQQSGPE | QVHLQQSGPE | QVHLQQSGPE |
| CD8v1- | LSASVGETVT | LVKPGASVKM | LVKPGASVKM | LVKPGASVKM |
| IL-2m1 | ITCGASENIY | SCKTSGYTFT | SCKTSGYTFT | SCKTSGYTFT |
| | GALNWYQRKQ | KYTMHWVKQG | KYTMHWVKQG | KYTMHWVKQG |
| | GKSPQLLIFG | HEESLEWIGH | HEESLEWIGH | HEESLEWIGH |
| | ATNLADGVSS | FNPNNDETKY | FNPNNDETKY | FNPNNDETKY |
| | RFSGSGSDRQ | NQKFTGKATL | NQKFTGKATL | NQKFTGKATL |
| | YSLKISSLHP | TVDKSSTTAY | TVDKSSTTAY | TVDKSSTTAY |
| | DDVATYYCQN | MELRSLTSDD | MELRSLTSDD | MELRSLTSDD |
| | ILDTPWTFGG | SALYYCARDG | SALYYCARDG | SALYYCARDG |
| | GTKLEIKRTV | LGLRLFADWG | LGLRLFADWG | LGLRLFADWG |
| | AAPSVFIFPP | QGTLVTVSSA | QGTLVTVSSA | QGTLVTVSSA |
| | SDEQLKSGTA | STKGPSVFPL | STKGPSVFPL | STKGPSVFPL |
| | SVVCLLNNFY | APSSKSTSGG | APSSKSTSGG | APSSKSTSGG |
| | PREAKVQWKV | TAALGCLVKD | TAALGCLVKD | TAALGCLVKD |
| | DNALQSGNSQ | YFPEPVTVSW | YFPEPVTVSW | YFPEPVTVSW |
| | ESVTEQDSKD | NSGALTSGVH | NSGALTSGVH | NSGALTSGVH |
| | STYSLSSTLT | TFPAVLQSSG | TFPAVLQSSG | TFPAVLQSSG |
| | LSKADYEKHK | LYSLSSVVTV | LYSLSSVVTV | LYSLSSVVTV |
| | VYACEVTHQG | PSSSLGTQTY | PSSSLGTQTY | PSSSLGTQTY |
| | LSSPVTKSFN | ICNVNHKPSN | ICNVNHKPSN | ICNVNHKPSN |
| | RGEC | TKVDKKVEPK | TKVDKKVEPK | TKVDKKVEPK |
| | (SEQ ID | SCDKTHTCPP | SCDKTHTCPP | SCDKTHTCPP |
| | NO: 156) | CPAPEAAGAP | CPAPEAAGAP | CPAPEAAGAP |
| | | SVFLFPPKPK | SVFLFPPKPK | SVFLFPPKPK |
| | | DTLMISRTPE | DTLMISRTPE | DTLMISRTPE |
| | | VTCVVVDVSH | VTCVVVDVSH | VTCVVVDVSH |
| | | EDPEVKFNWY | EDPEVKFNWY | EDPEVKFNWY |
| | | VDGVEVHNAK | VDGVEVHNAK | VDGVEVHNAK |
| | | TKPREEQYNS | TKPREEQYNS | TKPREEQYNS |
| | | TYRVVSVLTV | TYRVVSVLTV | TYRVVSVLTV |
| | | LHQDWLNGKE | LHQDWLNGKE | LHQDWLNGKE |
| | | YKCKVSNKAL | YKCKVSNKAL | YKCKVSNKAL |
| | | PAPIEKTISK | PAPIEKTISK | PAPIEKTISK |
| | | AKGQPREPQV | AKGQPREPQV | AKGQPREPQV |
| | | YTLPPCREEM | CTLPPSREEM | CTLPPSREEM |
| | | TKNQVSLSCA | TKNQVSLWCL | TKNQVSLWCL |
| | | VKGFYPSDIA | VKGFYPSDIA | VKGFYPSDIA |
| | | VEWESNGQPE | VEWESNGQPE | VEWESNGQPE |
| | | NNYKTTPPVL | NNYKTTPPVL | NNYKTTPPVL |
| | | DSDGSFFLVS | DSDGSFFLYS | DSDGSFFLYS |
| | | KLTVDKSRWQ | KLTVDKSRWQ | KLTVDKSRWQ |
| | | QGNVFSCSVM | QGNVFSCSVM | QGNVFSCSVM |
| | | HEALHNHYTQ | HEALHNHYTQ | HEALHNHYTQ |
| | | KSLSLSPGSG | KSLSLSPG | KSLSLSPGK |
| | | GGGSGGGGSG | (SEQ ID | (SEQ ID |
| | | GGGSAPTSSS | NO: 158) | NO: 217) |
| | | TKKTQLQLEH | | |
| | | LLLDLQMILN | | |
| | | GINNYKNPKL | | |
| | | TEMLTAKFYM | | |
| | | PKKATELKHL | | |
| | | QCLEEELKPL | | |
| | | EEVLNLAQSK | | |
| | | NFHLRPRDLI | | |
| | | SAINVIVLEL | | |
| | | KGSETTFMCE | | |
| | | YADETATIVE | | |
| | | FLNRWITFAQ | | |
| | | SIISTLT | | |
| | | (SEQ ID | | |
| | | NO: 157) | | |
| xh | DIQMTQSPSS | QVQLVQSGAE | QVQLVQSGAE | QVQLVQSGAE |
| CD8v2- | LSASVGDRVT | VKKPGSSVKV | VKKPGSSVKV | VKKPGSSVKV |
| IL-2m1 | ITCRASQEIY | SCKASGYRFH | SCKASGYRFH | SCKASGYRFH |
| | GALNWYQQKP | NFAISWVRQA | NFAISWVRQA | NFAISWVRQA |
| | GKAPKLLIYG | PGQGLEWMGG | PGQGLEWMGG | PGQGLEWMGG |
| | ATNLQSGVPS | IIPGHAKANY | IIPGHAKANY | IIPGIIAKAN |
| | RFSGSGSGTD | AQKFQGRVTI | AQKFQGRVTI | YAQKFQGRVT |
| | FTLTISSLQP | TADESTSTAY | TADESTSTAY | ITADESTSTA |
| | EDFATYYCQD | MELSSLRSED | MELSSLRSED | YMELSSLRSE |
| | IYDAPWTFGG | TAVYYCARDG | TAVYYCARDG | DTAVYYCARD |
| | GTKVEIKRTV | LGIRLFADWG | LGIRLFADWG | GLGIRLFADW |
| | AAPSVFIFPP | QGTLVTVSSA | QGTLVTVSSA | GQGTLVTVSS |
| | SDEQLKSGTA | STKGPSVFPL | STKGPSVFPL | ASTKGPSVFP |
| | SVVCLLNNFY | APSSKSTSGG | APSSKSTSGG | LAPSSKSTSG |
| | PREAKVQWKV | TAALGCLVKDY | TAALGCLVKD | GTAALGCLVK |
| | DNALQSGNSQ | FPEPVTVSWN | YFPEPVTVSW | DYFPEPVTVS |
| | FSVTFQDSKD | SGALTSGVHT | NSGALTSGVH | WNSGALTSGV |
| | STYSLSSTLT | FPAVLQSSGL | TFPAVLQSSG | HTFPAVLQSS |
| | LSKADYEKHK | YSLSSVVTVP | LYSLSSVVTV | GLYSLSSVVT |
| | VYACEVTHQG | SSSLGTQTYI | PSSSLGTQTY | VPSSSLGTQT |
| | LSSPVTKSFN | CNVNHKPSNT | ICNVNHKPSN | YICNVNHKPS |
| | RGEC | KVDKKVEPKS | TKVDKKVEPK | NTKVDKKVEP |
| | (SFQ ID | CDKTHTCPPC | SCDKTHTCPP | KSCDKTHTCP |
| | NO: 159) | PAPEAAGAPS | CPAPEAAGAP | PCPAPEAAGA |
| | | VFLFPPKPKD | SVFLFPPKPK | PSVFLFPPKP |
| | | TLMISRTPEV | DTLMISRTPE | KDTLMISRTP |
| | | TCVVVDVSHE | VTCVVVDVSH | EVTCVVVDVS |
| | | DPEVKFNWYV | EDPEVKFNWY | HEDPEVKFNW |
| | | DGVEVHNAKT | VDGVEVHNAK | YVDGVEVHNA |
| | | KPREEQYNST | TKPREEQYNS | KTKPREEQYN |
| | | YRVVSVLTVL | TYRVVSVLTV | STYRVVSVLT |
| | | HQDWLNGKEY | LHQDWLNGKE | VLHQDWLNGK |
| | | KCKVSNKALP | YKCKVSNKAL | EYKCKVSNKA |
| | | APIEKTISKA | PAPIEKTISK | LPAPIEKTIS |
| | | KGQPREPQVY | AKGQPREPQV | KAKGQPREPQ |
| | | TLPPCREEMT | CTLPPSREEM | VCTLPPSREE |
| | | KNQVSLSCAV | TKNQVSLWCL | MTKNQVSLWC |
| | | KGFYPSDIAV | VKGFYPSDIA | LVKGFYPSDI |
| | | EWESNGQPEN | VEWESNGQPE | AVEWESNGQP |
| | | NYKTTPPVLD | NNYKTTPPVL | ENNYKTTPPV |
| | | SDGSFFLVSK | DSDGSFFLYS | LDSDGSFFLY |
| | | LTVDKSRWQQ | KLTVDKSRWQ | SKLTVDKSRW |
| | | GNVFSCSVMH | QGNVFSCSVM | QQGNVFSCSV |
| | | EALHNHYTQK | HEALHNHYTQ | MHEALHNHYT |
| | | SLSLSPGSGG | KSLSLSPG | QKSLSLSPGK |
| | | GGSGGGGSG | (SEQ ID | (SEQ ID |
| | | GGGSAPTSSS | NO: 161) | NO: 218) |
| | | TKKTQLQLEH | | |
| | | LLLDLQMILN | | |
| | | GINNYKNPKL | | |
| | | TEMLTAKFYM | | |
| | | PKKATELKHL | | |
| | | QCLEEELKPL | | |
| | | EEVLNLAQSK | | |
| | | NFHLRPRDLI | | |
| | | SAINVIVLEL | | |
| | | KGSETTFMCE | | |
| | | YADETATIVE | | |
| | | FLNRWITFAQ | | |
| | | SIISTLT | | |
| | | (SEQ ID | | |
| | | NO: 160) | | |
| xh | DIQMTQSPSS | QVQLVQSGAE | QVQLVQSGAE | QVQLVQSGAE |
| CD8v3- | LSASVGDRVT | VKKPGSSVKV | VKKPGSSVKV | VKKPGSSVKV |
| IL-2m1 | ITCRASQEIY | SCKASGSRFY | SCKASGSRFY | SCKASGSRFY |
| | GALNWYQQKP | KFAISWVRQA | KFAISWVRQA | KFAISWVRQA |
| | GKAPKLLIYG | PGQGLEWMGG | PGQGLEWMGG | PGQGLEWMGG |
| | ATNLQSGVPS | IIPGHAKANY | IIPGHAKANY | IIPGHAKANY |
| | RFSGSGSGTD | AQKFQGRVTI | AQKFQGRVTI | AQKFQGRVTI |
| | FTLTISSLQP | TADESTSTAY | TADESTSTAY | TADESTSTAY |
| | EDFATYYCQD | MELSSLRSED | MELSSLRSED | MELSSLRSED |
| | IYDAPWTFGG | TAVYYCARDG | TAVYYCARDG | TAVYYCARDG |
| | GTKVEIKRTV | LGIRLFADWG | LGIRLFADWG | LGIRLFADWG |
| | AAPSVFIFPP | QGTLVTVSSA | QGTLVTVSSA | QGTLVTVSSA |
| | SDEQLKSGTA | STKGPSVFPL | STKGPSVFPL | STKGPSVFPL |
| | SVVCLLNNFY | APSSKSTSGG | APSSKSTSGG | APSSKSTSGG |
| | PREAKVQWKV | TAALGCLVKD | TAALGCLVKD | TAALGCLVKD |
| | DNALQSGNSQ | YFPEPVTVSW | YFPEPVTVSW | YFPEPVTVSW |
| | ESVTEQDSKD | NSGALTSGVI | NSGALTSGVH | NSGALTSGVH |
| | STYSLSSTLT | ITFPAVLQSS | TFPAVLQSSG | TFPAVLQSSG |
| | LSKADYEKHK | GLYSLSSVVT | LYSLSSVVTV | LYSLSSVVTV |
| | VYACEVTHQG | VPSSSLGTQT | PSSSLGTQTY | PSSSLGTQTY |
| | LSSPVTKSFN | YICNVNHKPS | CNVNHKPSNT | ICNVNHKPSN |
| | RGEC | NTKVDKKVEP | KVDKKVEPKS | TKVDKKVEPK |
| | (SEQ ID | KSCDKTHTCP | CDKTHTCPPC | SCDKTHTCPP |
| | NO: 162) | PCPAPEAAGA | PAPEAAGAPS | CPAPEAAGAP |
| | | PSVFLFPPKP | VFLFPPKPKD | SVFLFPPKPK |
| | | KDTLMISRTP | TLMISRTPEV | DTLMISRTPE |
| | | EVTCVVVDVS | TCVVVDVSHE | VTCVVVDVSH |
| | | HEDPEVKFNW | DPEVKFNWYV | EDPEVKFNWY |
| | | YVDGVEVHNA | DGVEVHNAKT | VDGVEVHNAK |
| | | KTKPREEQYN | KPREEQYNST | TKPREEQYNS |
| | | STYRVVSVLT | YRVVSVLTVL | TYRVVSVLTV |
| | | VLHQDWLNGK | HQDWLNGKEY | LHQDWLNGKE |
| | | EYKCKVSNKA | KCKVSNKALP | YKCKVSNKAL |
| | | LPAPIEKTIS | APIEKTISKA | PAPIEKTISK |
| | | KAKGQPREPQ | KGQPREPQVC | AKGQPREPQV |
| | | VYTLPPCREE | TLPPSREEMT | CTLPPSREEM |
| | | MTKNQVSLSC | KNQVSLWCLV | TKNQVSLWCL |
| | | AVKGFYPSDI | KGFYPSDIAV | VKGFYPSDIA |
| | | AVEWESNGQP | EWESNGQPEN | VEWESNGQPE |
| | | ENNYKTTPPV | NYKTTPPVLD | NNYKTTPPVL |
| | | LDSDGSFFLV | SDGSFFLYSK | DSDGSFFLYS |
| | | SKLTVDKSRW | LTVDKSRWQQ | KLTVDKSRWQ |
| | | QQGNVFSCSV | GNVFSCSVMH | QGNVFSCSVM |
| | | MHEALHNHYT | EALHNHYTQK | HEALHNHYTQ |
| | | QKSLSLSPGS | SLSLSPG | KSLSLSPGK |
| | | GGGGSGGGGS | (SEQ ID | (SEQ ID |
| | | GGGGSAPTSS | NO: 164) | NO: 219) |
| | | STKKTQLQLE | | |
| | | HLLLDLQMIL | | |
| | | NGINNYKNPK | | |
| | | LTEMLTAKFY | | |
| | | MPKKATELKH | | |
| | | LQCLEEELKP | | |
| | | LEEVLNLAQS | | |
| | | KNFHLRPRDL | | |
| | | ISAINVIVLE | | |
| | | LKGSETTFMC | | |
| | | EYADETATIV | | |
| | | EFLNRWITFA | | |
| | | QSIISTLT | | |
| | | (SEQ ID | | |
| | | NO: 163) | | |
| xh | DIQMTQSPSS | QVQLVQSGAE | QVQLVQSGAE | QVQLVQSGAE |
| CD8v4- | LSASVGDRVT | VKKPGSSVKV | VKKPGSSVKV | VKKPGSSVKV |
| IL-2m1 | ITCRASQKIY | SCKASGYTFT | SCKASGYTFT | SCKASGYTFT |
| | GALNWYQQKP | KYAISWVRQA | KYAISWVRQA | KYAISWVRQA |
| | GKAPKLLIYG | PGQGLEWMGG | PGQGLEWMGG | PGQGLEWMGG |
| | ATNLQSGVPS | IIPGHAKANY | IIPGHAKANY | IIPGHAKANY |
| | RFSGSGSGTD | AQKFQGRVTI | AQKFQGRVTI | AQKFQGRVTI |
| | FTLTISSLQP | TADESTSTAY | TADESTSTAY | TADESTSTAY |
| | EDFATYYCQN | MELSSLRSED | MELSSLRSED | MELSSLRSED |
| | TYDTPWTFGG | TAVYYCARDG | TAVYYCARDG | TAVYYCARDG |
| | GTKVEIKRTV | LGIRLFADWG | LGIRLFADWG | LGIRLFADWG |
| | AAPSVFIFPP | QGTLVTVSSA | QGTLVTVSSA | QGTLVTVSSA |
| | SDEQLKSGTA | STKGPSVFPL | STKGPSVFPL | STKGPSVFPL |
| | SVVCLLNNFY | APSSKSTSGG | APSSKSTSGG | APSSKSTSGG |
| | PREAKVQWKV | TAALGCLVKD | TAALGCLVKD | TAALGCLVKD |
| | DNALQSGNSQ | YFPEPVTVSW | YFPEPVTVSW | YFPEPVTVSW |
| | ESVTEQDSKD | NSGALTSGVH | NSGALTSGVH | NSGALTSGVH |
| | STYSLSSTLT | TFPAVLQSSG | TFPAVLQSSG | TFPAVLQSSG |
| | LSKADYEKHK | LYSLSSVVTV | LYSLSSVVTV | LYSLSSVVTV |
| | VYACEVTIIQ | PSSSLGTQTY | PSSSLGTQTY | PSSSLGTQTY |
| | GLSSPVTKSF | ICNVNHKPSN | ICNVNIIKPSN | ICNVNHKPSN |
| | NRGEC | TKVDKKVEPK | TKVDKKVEPK | TKVDKKVEPK |
| | (SEQ ID | SCDKTHTCPP | SCDKTHTCPP | SCDKTHTCPP |
| | NO: 165) | CPAPEAAGAP | CPAPEAAGAP | CPAPEAAGAP |
| | | SVFLFPPKPK | SVFLFPPKPK | SVFLFPPKPK |
| | | DTLMISRTPE | DTLMISRTPE | DTLMISRTPE |
| | | VTCVVVD | VTCVVVDVSH | VTCVVVDVSH |
| | | VSIIEDP | EDPEVKFNWY | EDPEVKFNWY |
| | | EVKFNWYVDG | VDGVEVHNAK | VDGVEVHNAK |
| | | VEVHNAKTKP | TKPREEQYNS | TKPREEQYNS |
| | | REEQYNSTYR | TYRVVSVLTV | TYRVVSVLTV |
| | | VVSVLTVLHQ | LHQDWLNGKE | LHQDWLNGKE |
| | | DWLNGKEYKC | YKCKVSNKAL | YKCKVSNKAL |
| | | KVSNKALPAP | PAPIEKTISK | PAPIEKTISK |
| | | IEKTISKAKG | AKGQPREPQV | AKGQPREPQV |
| | | QPREPQVYTL | CTLPPSREEM | CTLPPSREEM |
| | | PPCREEMTKN | TKNQVSLWCL | TKNQVSLWCL |
| | | QVSLSCAVKG | VKGFYPSDIA | VKGFYPSDIA |
| | | FYPSDIAVEW | VEWESNGQPE | VEWESNGQPE |
| | | ESNGQPENNY | NNYKTTPPVL | NNYKTTPPVL |
| | | KTTPPVLDSD | DSDGSFFLYS | DSDGSFFLYS |
| | | GSFFLVSKLT | KLTVDKSRWQ | KLTVDKSRWQ |
| | | VDKSRWQQGN | QGNVFSCSVM | QGNVFSCSVM |
| | | VFSCSVMHEA | HEALHNHYTQ | HEALHNHYTQ |
| | | LHNHYTQKSL | KSLSLSPG | KSLSLSPGK |
| | | SLSPGSGGGG | (SEQ ID | (SEQ ID |
| | | SGGGGSGGGG | NO: 167) | NO: 220) |
| | | SAPTSSSTKK | | |
| | | TQLQLEHLLL | | |
| | | DLQMILNGIN | | |
| | | NYKNPKLTEM | | |
| | | LTAKFYMPKK | | |
| | | ATELKHLQCL | | |
| | | EEELKPLEEV | | |
| | | LNLAQSKNFH | | |
| | | LRPRDLISAI | | |
| | | NVIVLELKGS | | |
| | | ETTFMCEYAD | | |
| | | ETATIVEFLN | | |
| | | RWITFAQSII | | |
| | | STLT | | |
| | | (SEQ ID | | |
| | | NO: 166) | | |
| xh | DIQMTQSPSS | QVQLVQSGAE | QVQLVQSGAE | QVQLVQSGAE |
| CD8v5- | LSASVGDRVT | VKKPGSS | VKKPGSSVKV | VKKPGSSVKV |
| IL-2m1 | ITCRASQKIY | VKVSCKASG | SCKASGSGFR | SCKASGSGFR |
| | GALNWYQQKP | SGFRGHA | GHAISWVRQA | GHAISWVRQA |
| | CKAPKLLI | ISWVRQAPGQ | PGQGLEWMGG | PGQGLEWMGG |
| | YGATNLQ | GLEWMGGIIP | IIPGHAKANY | IIPGHAKANY |
| | SGVPSR | GHAKANYAQK | AQKFQGRVTI | AQKFQGRVTI |
| | FSGSGSGTDF | FQGRVTITAD | TADESTSTAY | TADESTSTAY |
| | TLTISSLQPE | ESTSTAYMEL | MELSSLRSED | MELSSLRSED |
| | DFATYYCQNT | SSLRSEDTAV | TAVYYCARDG | TAVYYCARDG |
| | YDTPWTFGGG | YYCARDGLGI | LGIRLFADWG | LGIRLFADWG |
| | TKVEIKRTVA | RLFADWGQGT | QGTLVTVSSA | QGTLVTVSSA |
| | APSVFIFPPS | LVTVSSASTK | STKGPSVFPL | STKGPSVFPL |
| | DEQLKSGTAS | GPSVFPLAPS | APSSKSTSGG | APSSKSTSGG |
| | VVCLLNNFYP | SKSTSGGTAA | TAALGCLVKD | TAALGCLVKD |
| | REAKVQWKVD | LGCLVKDYFP | YFPEPVTVSW | YFPEPVTVSW |
| | NALQSGNSQE | EPVTVSWNSG | NSGALTSGVH | NSGALTSGVH |
| | SVTEQDSKDS | ALTSGVHTFP | TFPAVLQSSG | TFPAVLQSSG |
| | TYSLSSTLTL | AVLQSSGLYS | LYSLSSVVTV | LYSLSSVVTV |
| | SKADYEKHKV | LSSVVTVPSS | PSSSLGTQTY | PSSSLGTQTY |
| | YACEVTHQGL | SLGTQTYICN | ICNVNHKPSN | ICNVNHKPSN |
| | SSPVTKSFNR | VNHKPSNTKV | TKVDKKVEPK | TKVDKKVEPK |
| | GEC | DKKVEPKSCD | SCDKTHTCPP | SCDKTHTCPP |
| | (SEQ ID | KTHTCPPCPA | CPAPEAAGAP | CPAPEAAGAP |
| | NO: 168) | PEAAGAPSVF | SVFLFPPKPK | SVFLFPPKPK |
| | | LFPPKPKDTL | DTLMISRTPE | DTLMISRTPE |
| | | MISRTPEVTC | VTCVVVDVSI | VTCVVVDVSH |
| | | VVVDVSHEDP | IEDPEVKFNW | EDPEVKFNWY |
| | | EVKFNWYVDG | YVDGVEVHNA | VDGVEVHNAK |
| | | VEVHNAKTKP | KTKPREEQYN | TKPREEQYNS |
| | | REEQYNSTYR | STYRVVSVLT | TYRVVSVLTV |
| | | VVSVLTVLHQ | VLHQDWLNGK | LHQDWLNGKE |
| | | DWLNGKEYKC | EYKCKVSNKA | YKCKVSNKAL |
| | | KVSNKALPAP | LPAPIEKTIS | PAPIEKTISK |
| | | IEKTISKAKG | KAKGQPREPQ | AKGQPREPQV |
| | | QPREPQVYTL | VCTLPPSREE | CTLPPSREEM |
| | | PPCREEMTKN | MTKNQVSLWC | TKNQVSLWCL |
| | | QVSLSCAVKG | LVKGFYPSDI | VKGFYPSDIA |
| | | FYPSDIAVEW | AVEWESNGQP | VEWESNGQPE |
| | | ESNGQPENNY | ENNYKTTPPV | NNYKTTPPVL |
| | | KTTPPVLDSD | LDSDGSFFLY | DSDGSFFLYS |
| | | GSFFLVSKLT | SKLTVDKSRW | KLTVDKSRWQ |
| | | VDKSRWQQGN | QQGNVFSCSV | QGNVFSCSVM |
| | | VFSCSVMHEA | MHEALHNHYT | HEALHNHYTQ |
| | | LHNHYTQKSL | QKSLSLSPG | KSLSLSPGK |
| | | SLSPGSGGGG | (SEQ ID | (SEQ ID |
| | | SGGGGSGGGG | NO: 170) | NO: 221) |
| | | SAPTSSSTKK | | |
| | | TQLQLEHLLL | | |
| | | DLQMILNGIN | | |
| | | NYKNPKLTEM | | |
| | | LTAKFYMPKK | | |
| | | ATELKHLQCL | | |
| | | EEELKPLEEV | | |
| | | LNLAQSKNFH | | |
| | | LRPRDLISAI | | |
| | | NVIVLELKGS | | |
| | | ETTFMCEYAD | | |
| | | ETATIVEFLN | | |
| | | RWITFAQSII | | |
| | | STLT | | |
| | | (SEQ ID | | |
| | | NO: 169) | | |
| xh | EIVLTQSPAT | EVQLVESGGG | EVQLVESGGG | EVQLVESGGG |
| CD8v6- | LSVSPGERAT | AVRPGGSLRL | AVRPGGSLRL | AVRPGGSLRL |
| IL-2m1 | LSCRASQSVS | SCAASGFTFD | SCAASGFTFD | SCAASGFTFD |
| | SNLAWYQQKP | DYGMSWVRQA | DYGMSWVRQA | DYGMSWVRQA |
| | GQAP | PGKGLEWVSD | PGKGL | P |
| | RLLIYGASSR | INWSGEI | EWVSDINWSG | GKGLEWVSDI |
| | ATGIPDRFSG | TAYADSVKGR | EITAYADSVK | NWSGEITAYA |
| | SGSGTDFTLT | FTISRDNAKN | GRFTISRDNA | DSVKGRFTIS |
| | ISRLEPEDFA | SLYLQMNSLR | KNSLYLQMNS | RDNAKNSLYL |
| | VYYCQQYGSS | AEDTAVYYCA | LRAEDTAVYY | QMNSLRAEDT |
| | PPVTFGQGTK | RSNSYRWDDA | CARSNSYRWD | AVYYCARSNS |
| | VEIKRTVAAP | LDIWGQGTMV | DALDIWGQGT | YRWDDALDIW |
| | SVFIFPPSDE | TVSSASTKGP | MVTVSSASTK | GQGTMVTVSS |
| | QLKSGTASVV | SVFPLAPSSK | GPSVFPLAPS | ASTKGPSVFP |
| | CLLNNFYPRE | STSGGTAALG | SKSTSGGTAA | LAPSSKSTSG |
| | AKVQWKVDNA | CLVKDYFPEP | LGCLVKDYFP | GTAALGCLVK |
| | LQSGNSQESV | VTVSWNSGAL | EPVTVSWNSG | DYFPEPVTVS |
| | TEQDSKDSTY | TSGVHTFPAV | ALTSGVHTFP | WNSGALTSGV |
| | SLSSTLTLSK | LQSSGLYSLS | AVLQSSGLYS | HTFPAVLQSS |
| | ADYEKHKVYA | SVVTVPSSSL | LSSVVTVPSS | GLYSLSSVVT |
| | CEVTHQGLSS | GTQTYICNVN | SLGTQTYICN | VPSSSLGTQT |
| | PVTKSFNRGE | HKPSNTKVDK | VNHKPSNTKV | YICNVNHKPS |
| | C | KVEPKSCDKT | DKKVEPKSCD | NTKVDKKVEP |
| | (SEQ ID | HTCPPCPAPE | KTHTCPPCPA | KSCDKTHTCP |
| | NO: 171) | AAGAPSVFLF | PEAAGAPSVF | PCPAPEAAGA |
| | | PPKPKDTLMI | LFPPKPKDTL | PSVFLFPPKP |
| | | SRTPEVTCVV | MISRTPEVTC | KDTLMISRTP |
| | | VDVSHEDPEV | VVVDVSHEDP | EVTCVVVDVS |
| | | KFNWYVDGVE | EVKFNWYVDG | HEDPEVKFNW |
| | | VHNAKTKPRE | VEVHNAKTKP | YVDGVEVHNA |
| | | EQYNSTYRVV | REEQYNSTYR | KTKPREEQYN |
| | | SVLTVLHQDW | VVSVLTVLHQ | STYRVVSVLT |
| | | LNGKEYKCKV | DWLNGKEYKC | VLHQDWLNGK |
| | | SNKALPAPIE | KVSNKALPAP | EYKCKVSNKA |
| | | KTISKAKGQP | IEKTISKAKG | LPAPIEKTIS |
| | | REPQVYTLPP | QPREPQVCTL | KAKGQPREPQ |
| | | CREEMTKNQV | PPSREEMTKN | VCTLPPSREE |
| | | SLSCAVKGFY | QVSLWCLVKG | MTKNQVSLWC |
| | | PSDIAVEWES | FYPSDIAVEW | LVKGFYPSDI |
| | | NGQPENNYKT | ESNGQPENNY | AVEWESNGQP |
| | | TPPVLDSDGS | KTTPPVLDSD | ENNYKTTPPV |
| | | FFLVSKLTVD | GSFFLYSKLT | LDSDGSFFLY |
| | | KSRWQQGNVF | VDKSRWQQGN | SKLTVDKSRW |
| | | SCSVMHEALH | VFSCSVMHEA | QQGNVFSCSV |
| | | NHYTQKSLSL | LHNHYTQKSL | MHEALHNHYT |
| | | SPGSGGGGSG | SLSPG | QKSLSLSPGK |
| | | GGGSGGGGSA | (SEQ ID | (SEQ ID |
| | | PTSSSTKKTQ | NO: 173) | NO: 222) |
| | | LQLEHLLLDL | | |
| | | QMILNGINNY | | |
| | | KNPKLTEMLT | | |
| | | AKFYMPKKAT | | |
| | | ELKHLQCLEE | | |
| | | ELKPLEEVLN | | |
| | | LAQSKNFHLR | | |
| | | PRDLISAINV | | |
| | | IVLELKGSET | | |
| | | TFMCEYADET | | |
| | | ATIVEFLNRW | | |
| | | ITFAQSIIST | | |
| | | LT | | |
| | | (SEQ ID | | |
| | | NO: 172) | | |
| xh | EIVLTQSPAT | EVQLVESGGG | EVQLVESGGG | EVQLVESGGG |
| CD8v7- | LSVTPGEGAT | LVQPGRSLRL | LVQPGRSLRL | LVQPGRSLRL |
| IL-2m1 | LSCRASHSVG | SCAASGFTFD | SCAASGFTFD | SCAASGFTFD |
| | SNLAWYQQKP | DYAMHWVRQA | DYAMHWVRQA | DYAMHWVRQA |
| | GQAPRLLIYD | PGKGLEWVAV | PGKGLEWVAV | PGKGLEWVAV |
| | ASNRATGIPA | ISYDGSNKYY | ISYDGSNKYY | ISYDGSNKYY |
| | RFSGSGSGTD | ADSVKGRFTI | ADSVKGRFTI | ADSVKGRFTI |
| | FTLTISSLEP | SRDNSKNTLY | SRDNSKNTLY | SRDNSKNTLY |
| | EDLAVYYCQQ | LQMNSLRAED | LQMNSLRAED | LQMNSLRAED |
| | RSNWPPTFGQ | TAVYYCAKDR | TAVYYCAKDR | TAVYYCAKDR |
| | GTRLEIKRTV | IGWYDYDAFD | IGWYDYDAFD | IGWYDYDAFD |
| | AAPSVFIFPP | IWGQGTMVTV | IWGQGTMVTV | IWGQGTMVTV |
| | SDEQLKSGTA | SSASTKGPSV | SSASTKGPSV | SSASTKGPSV |
| | SVVCLLNNFY | FPLAPSSKST | FPLAPSSKST | FPLAPSSKST |
| | PREAKVQWKV | SGGTAALGCL | SGGTAALGCL | SGGTAALGCL |
| | DNALQSGNSQ | VKDYFPEPVT | VKDYFPEPVT | VKDYFPEPVT |
| | ESVTEQDSKD | VSWNSGALTS | VSWNSGALTS | VSWNSGALTS |
| | STYSLSSTLT | GVHTFPAVLQ | GVHTFPAVLQ | GVHTFPAVLQ |
| | LSKADYEKHK | SSGLYSLSSV | SSGLYSLSSV | SSGLYSLSSV |
| | VYACEVTHQG | VTVPSSSLGT | VTVPSSSLGT | VTVPSSSLGT |
| | LSSPVTKSFN | QTYICNVNHK | QTYICNVNHK | QTYICNVNHK |
| | RGEC | PSNTKVDKKV | PSNTKVDKKV | PSNTKVDKKV |
| | (SEQ ID | EPKSCDKTHT | EPKSCDKTHT | EPKSCDKTHT |
| | NO: 174) | CPPCPAPEAA | CPPCPAPEAA | CPPCPAPEAA |
| | | GAPSVFLFPP | GAPSVFLFPP | GAPSVFLFPP |
| | | KPKDTLMISR | KPKDTLMISR | KPKDTLMISR |
| | | TPEVTCVVVD | TPEVTCVVVD | TPEVTCVVVD |
| | | VSHEDPEVKF | VSHEDPEVKF | VSHEDPEVKF |
| | | NWYVDGVEVH | NWYVDGVEVH | NWYVDGVEVH |
| | | NAKTKPREEQ | NAKTKPREEQ | NAKTKPREEQ |
| | | YNSTYRVVSV | YNSTYRVVSV | YNSTYRVVSV |
| | | LTVLHQDWLN | LTVLHQDWLN | LTVLHQDWLN |
| | | GKEYKCKVSN | GKEYKCKVSN | GKEYKCKVSN |
| | | KALPAPIEKT | KALPAPIEKT | KALPAPIEKT |
| | | ISKAKGQPRE | ISKAKGQPRE | ISKAKGQPRE |
| | | PQVYTLPPCR | PQVCTLPPSR | PQVCTLPPSR |
| | | EEMTKNQVSL | EEMTKNQVSL | EEMTKNQVSL |
| | | SCAVKGFYPS | WCLVKGFYPS | WCLVKGFYPS |
| | | DIAVEWESNG | DIAVEWESNG | DIAVEWESNG |
| | | QPENNYKTTP | QPENNYKTTP | QPENNYKTTP |
| | | PVLDSDGSFF | PVLDSDGSFF | PVLDSDGSFF |
| | | LVSKLTVDKS | LYSKLTVDKS | LYSKLTVDKS |
| | | RWQQGNVFSC | RWQQGNVFSC | RWQQGNVFSC |
| | | SVMHEALHNH | SVMHEALHNH | SVMHEALHNH |
| | | YTQKSLSLSP | YTQKSLSLSP | YTQKSLSLSP |
| | | GSGGGGSGGG | G | GK |
| | | GSGGGGSAPT | (SEQ ID | (SEQ ID |
| | | SSSTKKTQLQ | NO: 176) | NO: 223) |
| | | LEHLLLDLQM | | |
| | | ILNGINNYKN | | |
| | | PKLTEMLTAK | | |
| | | FYMPKKATEL | | |
| | | KHLQCLEEEL | | |
| | | KPLEEVLNLA | | |
| | | QSKNFHLRPR | | |
| | | DLISAINVIV | | |
| | | LELKGSETTF | | |
| | | MCEYADETAT | | |
| | | IVEFLNRWIT | | |
| | | FAQSIISTLT | | |
| | | (SEQ ID | | |
| | | NO: 175) | | |
| xh | DIQMTQSPAS | QVQLQQSGAE | QVQLQQSGAE | QVQLQQSGAE |
| CD8v8- | LSVSVGETVT | LVRPGSSVKI | LVRPGSSVKI | LVRPGSSVKI |
| IL-2m1 | ITCRASENIY | SCKASGYAFS | SCKASGYAFS | SCKASGYAFS |
| | SNLAWYQQKQ | SYWMNWVKQR | SYWMNWVKQR | SYWMNWVKQR |
| | GKSPQLLVYA | PGQGLEWIGQ | PGQGLEWIGQ | PGQGLEWIGQ |
| | ATNLADGVPS | IYPGDGDTNY | IYPGDGDTNY | IYPGDGDTNY |
| | RFSGSGSGTQ | NGKFKGKATL | NGKFKGKATL | NGKFKGKATL |
| | YSLKINSLQS | TADKSSSTAY | TADKSSSTAY | TADKSSSTAY |
| | EDFGSYYCQH | MQLSSLTSED | MQLSSLTSED | MQLSSLTSED |
| | FWGTPWTFGG | SAVYFCARSG | SAVYFCARSG | SAVYFCARSG |
| | GTKLEIKRTV | AAFSSYYAMD | AAFSSYYAMD | AAFSSYYAMD |
| | AAPSVFIFPP | YWGQGTSVTV | YWGQGTSVTV | YWGQGTSVTV |
| | SDEQLKSGTA | SSASTKGPSV | SSASTKGPSV | SSASTKGPSV |
| | SVVCLLNNFY | FPLAPSSKST | FPLAPSSKST | FPLAPSSKST |
| | PREAKVQWKV | SGGTAALGCL | SGGTAALGCL | SGGTAA |
| | DNAL | VKDYFPEPVT | VKDYFPEPVT | LGCLVKDYFP |
| | QSGNSQESVT | VSWNSGALTS | VSW | EPVTVSWNSG |
| | EQDSKDSTYS | GVHTFPAVLQ | NSGALTSGVH | ALTSGVHTFP |
| | LSSTLTLSKA | SSGLYSLSSV | TFPAVLQSSG | AVLQSSGLYS |
| | DYEKHKVYAC | VTVPSSSLGT | LYSLSSVVTV | LSSVVTVPSS |
| | EVTHQGLSSP | QTYICNV | PSSSLGTQTY | SLGTQTYICN |
| | VTKSFNRGEC | NHKPSNTKVD | ICNVNHKPSN | VNHKPSNTKV |
| | (SEQ ID | KKVEPKSCDK | TKVDKKVEPK | DKKVEPKSCD |
| | NO: 187) | THTCPPCPAP | SCDKTHTCPP | KTHTCPPCPA |
| | | EAAGAPSVFL | CPAPEAAGAP | PEAAGAPSVF |
| | | FPPKPKDTLM | SVFLFPPKPK | LFPPKPKDTL |
| | | ISRTPEVTCV | DTLMISRTPE | MISRTPEVTC |
| | | VVDVSHEDPE | VTCVVVDVSH | VVVDVSHEDP |
| | | VKFNWYVDGV | EDPEVKFNWY | EVKFNWYVDG |
| | | EVHNAKTKPR | VDGVEVHNAK | VEVHNAKTKP |
| | | EEQYNSTYRV | TKPREEQYNS | REEQYNSTYR |
| | | VSVLTVLHQD | TYRVVSVLTV | VVSVLTVLHQ |
| | | WLNGKEYKCK | LHQDWLNGKE | DWLNGKEYKC |
| | | VSNKALPAPI | YKCKVSNKAL | KVSNKALPAP |
| | | EKTISKAKGQ | PAPIEKTISK | IEKTISKAKG |
| | | PREPQVYTLP | AKGQPREPQV | QPREPQVCTL |
| | | PCREEMTKNQ | CTLPPSREEM | PPSREEMTKN |
| | | VSLSCAVKGF | TKNQVSLWCL | QVSLWCLVKG |
| | | YPSDIAVEWE | VKGFYPSDIA | FYPSDIAVEW |
| | | SNGQPENNYK | VEWESNGQPE | ESNGQPENNY |
| | | TTPPVLDSDG | NNYKTTPPVL | KTTPPVLDSD |
| | | SFFLVSKLTV | DSDGSFFLYS | GSFFLYSKLT |
| | | DKSRWQQGNV | KLTVDKSRWQ | VDKSRWQQGN |
| | | FSCSVMHEAL | QGNVFSCSVM | VFSCSVMHEA |
| | | HNHYTQKSLS | HEALHNHYTQ | LHNHYTQKSL |
| | | LSPGSGGGGS | KSLSLSPG | SLSPGK |
| | | GGGGSGGGGS | (SEQ ID | (SEQ ID |
| | | APTSSSTKKT | NO: 189) | NO: 224) |
| | | QLQLEHLLLD | | |
| | | LQMILNGINN | | |
| | | YKNPKLTEML | | |
| | | TAKFYMPKKA | | |
| | | TELKHLQCLE | | |
| | | EELKPLEEVL | | |
| | | NLAQSKNFHL | | |
| | | RPRDLISAIN | | |
| | | VIVLELKGSE | | |
| | | TTFMCEYADE | | |
| | | TATIVEFLNR | | |
| | | WITFAQSIIS | | |
| | | TLT | | |
| | | (SEQ ID | | |
| | | NO: 188) | | |
| xh | DIQMTQSPSS | QVQLVQSGAE | QVQLVQSGAE | QVQLVQSGAE |
| CD8v9- | LSASVGDRVT | VKKPGSSVKV | VKKPGSSVKV | VKKPGSSVKV |
| IL-2m1 | ITCRASQEIY | SCKASGGTFS | SCKASGGTFS | SCKASGGTFS |
| | GALNWYQQKP | SYAISWVRQA | SYAISWVRQA | SYAISWVRQA |
| | GKAPKLLIYG | PGQGLEWMGG | PGQGLEWMGG | PGQGLEWMGG |
| | ATNLQSGVPS | IIPGAATANY | IIPGAATANY | IIPGAATANY |
| | RFSGSGSGTD | AQKFQGRVTI | AQKFQGRVTI | AQKFQGRVTI |
| | FTLTISSLQP | TADESTSTAY | TADESTSTAY | TADESTSTAY |
| | EDFATYYCQS | MELSSLRSED | MELSSLRSED | MELSSLRSED |
| | TYDAPWTFGG | TAVYYCARDA | TAVYYCARDA | TAVYYCARDA |
| | GTKVEIKRTV | AGIRLFADWG | AGIRLFADWG | AGIRLFADWG |
| | AAPSVFIFPP | QGTLVTVSSA | QGTLVTVSSA | QGTLVTVSSA |
| | SDEQLKSGTA | STKGPSVFPL | STKGPSVFPL | STKGPSVFPL |
| | SVVCLLNNFY | APSSKSTSGG | APSSKSTSGG | APSSKSTSGG |
| | PREAKVQWKV | TAALGCLVKD | TAALGCLVKD | TAALGCLVKD |
| | DNALQSGNSQ | YFPEPVTVSW | YFPEPVTVSW | YFPEPVTVSW |
| | ESVTEQDSKD | NSGALTSGVH | NSGALTSGVH | NSGALTSGVH |
| | STYSLSSTLT | TFPAVLQSSG | TFPAVLQSSG | TFPAVLQSSG |
| | LSKADYEKHK | LYSLSSVVTV | LYSLSSVVTV | LYSLSSVVTV |
| | VYACEVTHQG | PSSSLGTQTY | PSSSLGTQTY | PSSSLGTQTY |
| | LSSPVTKSFN | ICNVNHKPSN | ICNVNHKPSN | ICNVNHKPSN |
| | RGEC | TKVDKKVEPK | TKVDKKVEPK | TKVDKKVEPK |
| | (SEQ ID | SCDKTHTCPP | SCDKTHTCPP | SCDKTHTCPP |
| | NO: 298) | CPAPEAAGAP | CPAPEAAGAP | CPAPEAAGAP |
| | | SVFLFPPKPK | SVFLFPPKPK | SVFLFPPKPK |
| | | DTLMISRTPE | DTLMISRTPE | DTLMISRTPE |
| | | VTCVVVDVSH | VTCVVVDVSH | VTCVVVDVSH |
| | | EDPEVKFNWY | EDPEVKFNWY | EDPEVKFNWY |
| | | VDGVEVHNAK | VDGVEVHNAK | VDGVEVHNAK |
| | | TKPREEQYNS | TKPREEQYNS | TKPREEQYNS |
| | | TYRVVSVLTV | TYRVVSVLTV | TYRVVSVLTV |
| | | LHQDWLNGKE | LHQDWLNGKE | LHQDWLNGKE |
| | | YKCKVSNKAL | YKCKVSNKAL | YKCKVSNKAL |
| | | PAPIEKTISK | PAPIEKTISK | PAPIEKTISK |
| | | AKGQPREPQV | AKGQPREPQV | AKGQPREPQV |
| | | YTLPPCREEM | CTLPPSREEM | CTLPPSREEM |
| | | TKNQVSLSCA | TKNQVSLWCL | TKNQVSLWCL |
| | | VKGFYPSDIA | VKGFYPSDIA | VKGFYPSDIA |
| | | VEWESNGQPE | VEWESNGQPE | VEWESNGQPE |
| | | NNYKTTPPVL | NNYKTTPPVL | NNYKTTPPVL |
| | | DSDGSFFLVS | DSDGSFFLYS | DSDGSFFLYS |
| | | KLTVDKSRWQ | KLTVDKSRWQ | KLTVDKSRWQ |
| | | QGNVFSCSVM | QGNVFSCSVM | QGNVFSCSVM |
| | | HEALHNHYTQ | HEALHNHYTQ | HEALHNHYTQ |
| | | KSLSLSPGSG | KSLSLSPG | KSLSLSPGK |
| | | GGGSGGGGSG | (SEQ ID | (SEQ ID |
| | | GGGSAPTSSS | NO: 300) | NO: 301) |
| | | TKKTQLQLEH | | |
| | | LLLDLQMILN | | |
| | | GINNYKNPKL | | |
| | | TEMLTAKFYM | | |
| | | PKKATELKHL | | |
| | | QCLEEELKPL | | |
| | | EEVLNLAQSK | | |
| | | NFHLRPRDLI | | |
| | | SAINVIVLEL | | |
| | | KGSETTFMCE | | |
| | | YADETATIVE | | |
| | | FLNRWITFAQ | | |
| | | SIISTLT | | |
| | | (SEQ ID | | |
| | | NO: 299) | | |
| xh | EIVLTQSPGT | EVQLVESGGG | EVQLVESGGG | EVQLVESGGG |
| CD8v10- | LSLSPGERAT | LVQPGGSLRL | LVQPGGSLRL | LVQPGGSLRL |
| IL-2m1 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLLIYG | PGKGLEWVSD | PGKGLEWVSD | PGKGLEWVSD |
| | ASSRATGIPD | ITYAGGSTAY | ITYAGGSTAY | ITYAGGSTAY |
| | RFSGSGSGTD | ADSVKGRFTI | ADSVKGRFTI | ADSVKGRFTI |
| | ETLTISRLEP | SRDNAKNSLY | SRDNAKNSLY | SRDNAKNSLY |
| | EDFAVYYCQQ | LQMNSLRAED | LQMNSLRAED | LQMNSLRAED |
| | YGSSPPVTFG | TAVYYCARSN | TAVYYCARSN | TAVYYCARSN |
| | QGTKVEIKRT | AYAWDDALDI | AYAWDDALDI | AYAWDDALDI |
| | VAAPSVFIFP | WGQGTMVTVS | WGQGTMVTVS | WGQGTMVTVS |
| | PSDEQLKSGT | SASTKGPSVF | SASTKGPSVF | SASTKGPSVF |
| | ASVVCLLNNF | PLAPSSKSTS | PLAPSSKSTS | PLAPSSKSTS |
| | YPREAKVQWK | GGTAALGCLV | GGTAALGCLV | GGTAALGCLV |
| | VDNALQSGNS | KDYFPEPVTV | KDYFPEPVTV | KDYFPEPVTV |
| | QESVTEQDSK | SWNSGALTSG | SWNSGALTSG | SWNSGALTSG |
| | DSTYSLSSTL | VHTFPAVLQS | VHTFPAVLQS | VHTFPAVLQS |
| | TLSKADYEKH | SGLYSLSSVV | SGLYSLSSVV | SGLYSLSSVV |
| | KVYACEVTHQ | TVPSSSLGTQ | TVPSSSLGTQ | TVPSSSLGTQ |
| | GLSSPVTKSF | TYICNVNHKP | TYICNVNHKP | TYICNVNHKP |
| | NRGEC | SNTKVDKKVE | SNTKVDKKVE | SNTKVDKKVE |
| | (SEQ ID | PKSCDKTHTC | PKSCDKTHTC | PKSCDKTHTC |
| | NO: 302) | PPCPAPEAAG | PPCPAPEAAG | PPCPAPEAAG |
| | | APSVFLFPPK | APSVFLFPPK | APSVFLFPPK |
| | | PKDTLMISRT | PKDTLMISRT | PKDTLMISRT |
| | | PEVTCVVVDV | PEVTCVVVDV | PEVTCVVVDV |
| | | SHEDPEVKFN | SHEDPEVKFN | SHEDPEVKFN |
| | | WYVDGVEVHN | WYVDGVEVHN | WYVDGVEVHN |
| | | AKTKPREEQY | AKTKPREEQY | AKTKPREEQY |
| | | NSTYRVVSVL | NSTYRVVSVL | NSTYRVVSVL |
| | | TVLHQDWLNG | TVLHQDWLNG | TVLHQDWLNG |
| | | KEYKCKVSNK | KEYKCKVSNK | KEYKCKVSNK |
| | | ALPAPIEKTI | ALPAPIEKTI | ALPAPIEKTI |
| | | SKAKGQPREP | SKAKGQPREP | SKAKGQPREP |
| | | QVYTLPPCRE | QVCTLPPSRE | QVCTLPPSRE |
| | | EMTKNQVSLS | EMTKNQVSLW | EMTKNQVSLW |
| | | CAVKGFYPSD | CLVKGFYPSD | CLVKGFYPS |
| | | IAVEWESNGQ | IAVEWESNGQ | DIAVEW |
| | | PENNYKTTPP | PENNYKTTPP | ESNGQPENNY |
| | | VLDSDGSFFL | VLDSDGSFFL | KTTPPVLDSD |
| | | VSKLTVDKSR | YSKLTVDKSR | GSFFLYSKLT |
| | | WQQGNVFSCS | WQQGNVFSCS | VDKSRWQQGN |
| | | VMHEALHNHY | VMHEALHNHY | VFSCSVMHEA |
| | | TQKSLSLSPG | TQKSLSLSPG | LHNHYT |
| | | SGGGGSG | (SEQ ID | QKSLSLSPGK |
| | | GGGSGG | NO: 304) | (SEQ ID |
| | | GGSAPTSSST | | NO: 305) |
| | | KKTQLQLEHL | | |
| | | LLDLQMILNG | | |
| | | INNYKNPKLT | | |
| | | EMLTAKFYMP | | |
| | | KKATELKHLQ | | |
| | | CLEEELKPLE | | |
| | | EVLNLAQSKN | | |
| | | FHLRPRDLIS | | |
| | | AINVIVLELK | | |
| | | GSETTFMCEY | | |
| | | ADETATIVEF | | |
| | | LNRWITFAQS | | |
| | | IISTLT | | |
| | | (SEQ ID | | |
| | | NO: 303) | | |
| xh | EIVLTQSPGT | EVQLVESGGG | EVQLVESGGG | EVQLVESGGG |
| CD8v11- | LSLSPGERAT | LVQPGGSLRL | LVQPGGSLRL | LVQPGGSLRL |
| IL-2m1 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLLIYG | PGKGLEWVSD | PGKGLEWVSD | PGKGLEWVSD |
| | ASSRATGIPD | ITYAGGSTAY | ITYAGGSTAY | ITYAGGSTAY |
| | RFSGSGSGTD | ADSVKGRFTI | ADSVKGRFTI | ADSVKGRFTI |
| | FTLTISRLEP | SRDNAKNSLY | SRDNAKNSLY | SRDNAKNSLY |
| | EDFAVYYCQQ | LQMNSLRAED | LQMNSLRAED | LQMNSLRAED |
| | YGSSPPVTFG | TAVYYCARSN | TAVYYCARSN | TAVYYCARSN |
| | QGTKVEIKRT | AYAWDDALDI | AYAWDDALDI | AYAWDDALDI |
| | VAAPSVFIFP | WGQGTLVTVS | WGQGTLVTVS | WGQGTLVTVS |
| | PSDEQLKSGT | SASTKGPSVF | SASTKGPSVF | SASTKGPSVF |
| | ASVVCLLNNF | PLAPSSKSTS | PLAPSSKSTS | PLAPSSKSTS |
| | YPREAKVQWK | GGTAALGCLV | GGTAALGCLV | GGTAALGCLV |
| | VDNALQSGNS | KDYFPEPVTV | KDYFPEPVTV | KDYFPEPVTV |
| | QESVTEQDSK | SWNSGALTSG | SWNSGALTSG | SWNSGALTSG |
| | DSTYSLSSTL | VHTFPAVLQS | VHTFPAVLQS | VHTFPAVLQS |
| | TLSKADYEKH | SGLYSLSSVV | SGLYSLSSVV | SGLYSLSSVV |
| | KVYACEVTHQ | TVPSSSLGTQ | TVPSSSLGTQ | TVPSSSLGTQ |
| | GLSSPVTKSF | TYICNVNHKP | TYICNVNHKP | TYICNVNHKP |
| | NRGEC | SNTKVDKKVE | SNTKVDKKVE | SNTKVDKKVE |
| | (SEQ ID | PKSCDKTHTC | PKSCDKTHTC | PKSCDKTHTC |
| | NO: 306) | PPCPAPEAAG | PPCPAPEAAG | PPCPAPEAAG |
| | | APSVFLFPPK | APSVFLFPPK | APSVFLFPPK |
| | | PKDTLMISRT | PKDTLMISRT | PKDTLMISRT |
| | | PEVTCVVVDV | PEVTCVVVDV | PEVTCVVVDV |
| | | SHEDPEVKFN | SHEDPEVKFN | SHEDPEVKFN |
| | | WYVDGVEVHN | WYVDGVEVHN | WYVDGVEVHN |
| | | AKTKPREEQY | AKTKPREEQY | AKTKPREEQY |
| | | NSTYRVVSVL | NSTYRVVSVL | NSTYRVVSVL |
| | | TVLHQDWLNG | TVLHQDWLNG | TVLHQDWLNG |
| | | KEYKCKVSNK | KEYKCKVSNK | KEYKCKVSNK |
| | | ALPAPIEKTI | ALPAPIEKTI | ALPAPIEKTI |
| | | SKAKGQPREP | SKAKGQPREP | SKAKGQPREP |
| | | QVYTLPPCRE | QVCTLPPSRE | QVCTLPPSRE |
| | | EMTKNQVSLS | EMTKNQVSLW | EMTKNQVSLW |
| | | CAVKGFYPSD | CLVKGFYPSD | CLVKCFYPSD |
| | | IAVEWESNGQ | IAVEWESNGQ | IAVEWESNGQ |
| | | PENNYKTTPP | PENNYKTTPP | PENNYKTTPP |
| | | VLDSDGSFFL | VLDSDGSFFL | VLDSDGSFFL |
| | | VSKLTVDKSR | YSKLTVDKSR | YSKLTVDKSR |
| | | WQQGNVFSCS | WQQGNVFSCS | WQQGNVFSCS |
| | | VMHEALHNHY | VMHEALHNHY | VMHEALHNHY |
| | | TQKSLSLSPG | TQKSLSLSPG | TQKSLSLSPG |
| | | SGGGGSGGGG | (SEQ ID | K |
| | | SGGGGSAPTS | NO: 308) | (SEQ ID |
| | | SSTKKTQLQL | | NO: 309) |
| | | EHLLLDLQMI | | |
| | | LNGINNYKNP | | |
| | | KLTEMLTAKF | | |
| | | YMPKKATELK | | |
| | | HLQCLEEELK | | |
| | | PLEEVLNLAQ | | |
| | | SKNFHLRPRD | | |
| | | LISAINVIVL | | |
| | | ELKGSETTFM | | |
| | | CEYADETATI | | |
| | | VEFLNRWITF | | |
| | | AQSIISTLT | | |
| | | (SEQ ID | | |
| | | NO: 307) | | |
| xh | DIQMTQSPSS | QVQLVQSGAE | QVQLVQSGAE | QVQLVQSGAE |
| CD8v1- | LSASVGDRVT | VKKPGSSVKV | VKKPGSSVKV | VKKPGSSVKV |
| IL-2m1 | ITCRASQSIY | SCKASGGTFS | SCKASGGTFS | SCKASGGTFS |
| | GALNWYQQKP | SYAISWVRQA | SYAISWVRQA | SYAISWVRQA |
| | GKAPKLLIYG | PGQGLEWMGG | PGQGLEWMGG | PGQGLEWMGG |
| | ASNLQSGVPS | IIPGYATANY | IIPGYATANY | IIPGYATANY |
| | RFSGSGSGTD | AQKEQGRVTI | AQKFQGRVTI | AQKFQGRVTI |
| | FTLTISSLQP | TADESTSTAY | TADESTSTAY | TADESTSTAY |
| | EDFATYYCQS | MELSSLRSED | MELSSLRSED | MELSSLRSED |
| | TYTAPWTFGG | TAVYYCARDA | TAVYYCARDA | TAVYYCARDA |
| | GTKVEIKRTV | AGIRLFADWG | AGIRLFADWG | AGIRLFADWG |
| | AAPSVFIFPP | QGTLVTVSSA | QGTLVTVSSA | QGTLVTVSSA |
| | SDEQLKSGTA | STKGPSVFPL | STKGPSVFPL | STKGPSVFPL |
| | SVVCLLNNFY | APSSKSTSGG | APSSKSTSGG | APSSKSTSGG |
| | PREAKVQWKV | TAALGCLVKD | TAALGCLVKD | TAALGCLVKD |
| | DNALQSGNSQ | YFPEPVTVSW | YFPEPVTVSW | YFPEPVTVSW |
| | ESVTEQDSKD | NSGALTSGVH | NSGALTSGVH | NSGALTSGVH |
| | STYSLSSTLT | TFPAVLQSSG | TFPAVLQSSG | TEPAVLQSSG |
| | LSKADYEKHK | LYSLSSVVTV | LYSLSSVVTV | LYSLSSVVTV |
| | VYACEVTHQG | PSSSLGTQTY | PSSSLGTQTY | PSSSLGTQTY |
| | LSSPVTKSFN | ICNVNHKPSN | ICNVNHKPSN | ICNVNHKPSN |
| | RGEC | TKVDKKVEPK | TKVDKKVEPK | TKVDKKVEPK |
| | (SEQ ID | SCDKTHTCPP | SCDKTHTCPP | SCDKTHTCPP |
| | NO: 310) | CPAPEAAGAP | CPAPEAAGAP | CPAPEAAGAP |
| | | SVFLFPPKPK | SVFLFPPKPK | SVFLFPPKPK |
| | | DTLMISRTPE | DTLMISRTPE | DTLMISRTPE |
| | | VTCVVVDVSH | VTCVVVDVSH | VTCVVVDVSH |
| | | EDPEVKENWY | EDPEVKFNWY | EDPEVKFNWY |
| | | VDGVEVHNAK | VDGVEVHNAK | VDGVEVHNAK |
| | | TKPREEQYNS | TKPREEQYNS | TKPREEQYNS |
| | | TYRVVSVLTV | TYRVVSVLTV | TYRVVSVLTV |
| | | LHQDWLNGKE | LHQDWLNGKE | LHQDWLNGKE |
| | | YKCKVSNKAL | YKCKVSNKAL | YKCKVSNKAL |
| | | PAPIEKTISK | PAPIEKTISK | PAPIEKTISK |
| | | AKGQPREPQV | AKGQPREPQV | AKGQPREPQV |
| | | YTLPPCREEM | CTLPPSREEM | CTLPPSREEM |
| | | TKNQVSLSCA | TKNQVSLWCL | TKNQVSLWCL |
| | | VKGFYPSDIA | VKGFYPSDIA | VKGFYPSDIA |
| | | VEWESNGQPE | VEWESNGQPE | VEWESNGQPE |
| | | NNYKTTPPVL | NNYKTTPPVL | NNYKTTPPVL |
| | | DSDGSFFLVS | DSDGSFFLYS | DSDGSFFLYS |
| | | KLTVDKSRWQ | KLTVDKSRWQ | KLTVDKSRWQ |
| | | QGNVFSCSVM | QGNVFSCSVM | QGNVFSCSVM |
| | | HEALHNHYTQ | HEALHNHYTQ | HEALHNHYTQ |
| | | KSLSLSPGSG | KSLSLSPG | KSLSLSPGK |
| | | GGGSGGGGSG | (SEQ ID | (SEQ ID |
| | | GGGSAPTSSS | NO: 312) | NO: 313) |
| | | TKKTQLQLEH | | |
| | | LLLDLQMILN | | |
| | | GINNYKNPKL | | |
| | | TEMLTAKFYM | | |
| | | PKKATELKHL | | |
| | | QCLEEELKPL | | |
| | | EEVLNLAQSK | | |
| | | NFHLRPRDLI | | |
| | | SAINVIVLEL | | |
| | | KGSETTFMCE | | |
| | | YADETATIVE | | |
| | | FLNRWITFAQ | | |
| | | SIISTLT | | |
| | | (SEQ ID | | |
| | | NO: 311) | | |
| xh | DIQMTQSPSS | QVQLVQSGAE | QVQLVQSGAE | QVQLVQSGAE |
| CD8v13- | LSASVGDRVT | VKKPGSSVKV | VKKPGSSVKV | VKKPGSSVKV |
| IL-2m1 | ITCRASQEIY | SCKASGGTFS | SCKASGGTFS | SCKASGGTFS |
| | GALNWYQQKP | SYAISWVRQA | SYAISWVRQA | SYAISWVRQA |
| | GKAPKLLIYG | PGQGLEWMGG | PGQGLEWMGG | PGQGLEWMGG |
| | ATNLQSGVPS | IIPGYATANY | IIPGYATANYA | IIPGYATANY |
| | RFSGSGSGTD | AQKEQGRVTI | QKFQGRVTIT | AQKFQGRVTI |
| | FTLTISSLQP | TADESTSTAY | ADESTSTAYM | TADESTSTAY |
| | EDFATYYCQS | MELSSLRSED | ELSSLRSEDT | MELSSLRSED |
| | TYDAPWTFGG | TAVYYCARDA | AVYYCARDAA | TAVYYCARDA |
| | GTKVEIKRTV | AGIRLFADWG | GIRLFADWGQ | AGIRLFADWG |
| | AAPSVFIFPP | QGTLVTVSSA | GTLVTVSSAS | QGTLVTVSSA |
| | SDEQLKSGTA | STKGPSVFPL | TKGPSVFPLA | STKGPSVFPL |
| | SVVCLLNNFY | APSSKSTSGG | PSSKSTSGGT | APSSKSTSGG |
| | PREAKVQWKV | TAALGCLVKD | AALGCLVKDY | TAALGCLVKD |
| | DNALQSGNSQ | YFPEPVTVSW | FPEPVTVSWN | YFPEPVTVSW |
| | ESVTEQDSKD | NSGALTSGVH | SGALTSGVHT | NSGALTSGVH |
| | STYSLSSTLT | TFPAVLQSSG | FPAVLQSSGL | TEPAVLQSSG |
| | LSKADYEKHK | LYSLSSVVTV | YSLSSVVTVP | LYSLSSVVTV |
| | VYACEVTHQG | PSSSLGTQTY | SSSLGTQTYI | PSSSLGTQTY |
| | LSSPVTKSFN | ICNVNHKPSN | CNVNHKPSNT | ICNVNHKPSN |
| | RGEC | TKVDKKVEPK | KVDKKVEPKS | TKVDKKVEPK |
| | (SEQ ID | SCDKTHTCPP | CDKTHTCPPC | SCDKTHTCPP |
| | NO: 314) | CPAPEAAGAP | PAPEAAGAPS | CPAPEAAGAP |
| | | SVFLFPPKPK | VFLFPPKPKD | SVFLFPPKPK |
| | | DTLMISRTPE | TLMISRTPEV | DTLMISRTPE |
| | | VTCVVVDVSI | TCVVVDVSHE | VTCVVVDVSH |
| | | IEDPEVKFNW | DPEVKFNWYV | EDPEVKFNWY |
| | | YVDGVEVHNA | DGVEVHNAKT | VDGVEVHNAK |
| | | KTKPREEQYN | KPREEQYNST | TKPREEQYNS |
| | | STYRVVSVLT | YRVVSVLTVL | TYRVVSVLTV |
| | | VLHQDWLNGK | HQDWLNGKEY | LHQDWLNGKE |
| | | EYKCKVSNKA | KCKVSNKALP | YKCKVSNKAL |
| | | LPAPIEKTIS | APIEKTISKA | PAPIEKTISK |
| | | KAKGQPREPQ | KGQPREPQVC | AKGQPREPQV |
| | | VYTLPPCREE | TLPPSREEMT | CTLPPSREEM |
| | | MTKNQVSLSC | KNQVSLWCLV | TKNQVSLWCL |
| | | AVKGFYPSDI | KGFYPSDIAV | VKGFYPSDIA |
| | | AVEWESNGQP | EWESNGQPEN | VEWESNGQPE |
| | | ENNYKTTPPV | NYKTTPPVLD | NNYKTTPPVL |
| | | LDSDGSFFLV | SDGSFFLYSK | DSDGSFFLYS |
| | | SKLTVDKSRW | LTVDKSRWQQ | KLTVDKSRWQ |
| | | QQGNVFSCSV | GNVFSCSVMH | QGNVFSCSVM |
| | | MHEALHNHYT | EALHNHYTQK | HEALHNHYTQ |
| | | QKSLSLSPGS | SLSLSPG | KSLSLSPGK |
| | | GGGGSGGGGS | (SEQ ID | (SEQ ID |
| | | GGGGSAPTSS | NO: 316) | NO: 317) |
| | | STKKTQLQLE | | |
| | | HLLLDLQMIL | | |
| | | NGINNYKNPK | | |
| | | LTEMLTAKFY | | |
| | | MPKKATELKH | | |
| | | LQCLEEELKP | | |
| | | LEEVLNLAQS | | |
| | | KNFHLRPRDL | | |
| | | ISAINVIVLE | | |
| | | LKGSETTFMC | | |
| | | EYADETATIV | | |
| | | EFLNRWITFA | | |
| | | QSIISTLT | | |
| | | (SEQ ID | | |
| | | NO: 315) | | |
| xh | EIVLTQSPGT | EVQLVESGGG | EVQLVESGGG | EVQLVESGGG |
| CD8v14- | LSLSPGERAT | LVQPGGSLRL | LVQPGGSLRL | LVQPGGSLRL |
| IL-2m1 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLLIYG | PGKGLEWVSD | PGKGLEWVSD | PGKGLEWVSD |
| | ASSRATGIPD | ISYAGGSTAY | ISYAGGSTAY | ISYAGGSTAY |
| | RFSGSGSGTD | ADSVKGRFTI | ADSVKGRFTI | ADSVKGRFTI |
| | FTLTISRLEP | SRDNAKNSLY | SRDNAKNSLY | SRDNAKNSLY |
| | EDFAVYYCQQ | LQMNSLRAED | LQMNSLRAED | LQMNSLRAED |
| | YGSSPPVTFG | TAVYYCARSN | TAVYYCARSN | TAVYYCARSN |
| | QGTKVEIKRT | AYAWDDALDI | AYAWDDALDI | AYAWDDALDI |
| | VAAPSVFIFP | WGQGTMVTVS | WGQGTMVTVS | WGQGTMVTVS |
| | PSDEQLKSGT | SASTKGPSVF | SASTKGPSVF | SASTKGPSVF |
| | ASVVCLLNNF | PLAPSSKSTS | PLAPSSKSTS | PLAPSSKSTS |
| | YPREAKVQWK | GGTAALGCLV | GGTAALGCLV | GGTAALGCLV |
| | VDNALQSGNS | KDYFPEPVTV | KDYFPEPVTV | KDYFPEPVTV |
| | QESVTEQDSK | SWNSGALTSG | SWNSGALTSG | SWNSGALTSG |
| | DSTYSLSSTL | VHTFPAVLQS | VHTFPAVLQS | VHTFPAVLQS |
| | TLSKADYEKH | SGLYSLSSVV | SGLYSLSSVV | SGLYSLSSVV |
| | KVYACEVTHQ | TVPSSSLGTQ | TVPSSSLGTQ | TVPSSSLGTQ |
| | GLSSPVTKSF | TYICNVNHKP | TYICNVNHKP | TYICNVNHKP |
| | NRGEC | SNTKVDKKVE | SNTKVDKKVE | SNTKVDKKVE |
| | (SEQ ID | PKSCDKTHTC | PKSCDKTHTC | PKSCDKTHTC |
| | NO: 18) | PPCPAPEAAG | PPCPAPEAAG | PPCPAPEAAG |
| | | APSVFLFPPK | APSVFLFPPK | APSVFLFPPK |
| | | PKDTLMISRT | PKDTLMISRT | PKDTLMISRT |
| | | PEVTCVVVDV | PEVTCVVVDV | PEVTCVVVDV |
| | | SHEDPEVKFN | SHEDPEVKFN | SHEDPEVKFN |
| | | WYVDGVEVHN | WYVDGVEVHN | WYVDGVEVHN |
| | | AKTKPREEQY | AKTKPREEQY | AKTKPREEQY |
| | | NSTYRVVSVL | NSTYRVVSVL | NSTYRVVSVL |
| | | TVLHQDWLNG | TVLHQDWLNG | TVLHQDWLNG |
| | | KEYKCKVSNK | KEYKCKVSNK | KEYKCKVSNK |
| | | ALPAPIEKTI | ALPAPIEKTI | ALPAPIEKTI |
| | | SKAKGQPREP | SKAKGQPREP | SKAKGQPREP |
| | | QVYTLPPCRE | QVCTLPPSRE | QVCTLPPSRE |
| | | EMTKNQVSLS | EMTKNQVSLW | EMTKNQVSLW |
| | | CAVKGFYPSD | CLVKGFYPSD | CLVKGFYPSD |
| | | IAVEWESNGQ | IAVEWESNGQ | IAVEWESNGQ |
| | | PENNYKTTPP | PENNYKTTPP | PENNYKTTPP |
| | | VLDSDGSFFL | VLDSDGSFFL | VLDSDGSFFL |
| | | VSKLTVDKSR | YSKLTVDKSR | YSKLTVDKSR |
| | | WQQGNVFSCS | WQQGNVFSCS | WQQGNVFSCS |
| | | VMHEALHNHY | VMHEALHNHY | VMHEALHNHY |
| | | TQKSLSLSPG | TQKSLSLSPG | TQKSLSLSPG |
| | | SGGGGSGGGG | (SEQ ID | K |
| | | SGGGGSAPTS | NO: 320) | (SEQ ID |
| | | SSTKKTQLQL | | NO: 321) |
| | | EHLLLDLQMI | | |
| | | LNGINNYKNP | | |
| | | KLTEMLTAKF | | |
| | | YMPKKATELK | | |
| | | HLQCLEEELK | | |
| | | PLEEVLNLAQ | | |
| | | SKNFHLRPRD | | |
| | | LISAINVIVL | | |
| | | ELKGSETTEM | | |
| | | CEYADETATI | | |
| | | VEFLNRWITF | | |
| | | AQSIISTLT | | |
| | | (SEQ ID | | |
| | | NO: 319) | | |
| xh | EIVLTQSPGT | EVQLVESGGG | EVQLVESGGG | EVQLVESGGG |
| CD8v15- | LSLSPGERAT | LVQPGGSLRL | LVQPGGSLRL | LVQPGGSLRL |
| IL-2m1 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLAPSV | PGKGLEWVSD | PGKGLESSAS | PGLFADWGQG |
| | FIFPPSDEQL | ISYAGGSTAY | TKGPSVFPLA | TLVTVSSAST |
| | KSGTASVVCL | KDYFPEPVTV | PSSKSTSGGT | KGPSVFPLAP |
| | LNNFYPREAK | SWNSGALTSG | AALGCLVKDY | SSKSTSGGTA |
| | VQWKVDNALQ | VHTFPAVLQS | FPEPVTVSWN | ALGCLVKDYF |
| | SGNSQESVTE | SGLYSLSSVV | SGALTSGVHT | PEPVTVSWNS |
| | QDSKDSTYSL | TVPSSSLGTQ | FPAVLQSSGL | GALTSGVHTF |
| | SSTLTLSKAD | TYICNVNHKP | YSLSSVVTVP | PAVLQSSGLY |
| | YEKHKVYACE | SNTKVDKKVE | SSSLGTQTYI | SLSSVVTVPS |
| | VTHQGLSSPV | PKSCDKTHTC | CNVNHKPSNT | SSLGTQTYIC |
| | TKSFNRGEC | PPCPAPEAAG | KVDKKVEPKS | NVNHKPSNTK |
| | (SEQ ID | APSVFLFPPK | CDKTHTCPPC | VDKKVEPKSC |
| | NO: 326) | PKDTLMISRT | PAPEAAGAPS | DKTHTCPPCP |
| | | PEVTCVVVDV | VFLFPPKPKD | APEAAGAPSV |
| | | SHEDPEVKFN | TLMISRTPEV | FLFPPKPKDT |
| | | WYVDGVEVHN | TCVVVDVSHE | LMISRTPEVT |
| | | AKTKPREEQY | DPEVKFNWYV | CVVVDVSHED |
| | | NSTYRVVSVL | DGVEVHNAKT | PEVKFNWYVD |
| | | TVLHQDWLNG | KPREEQYNST | GVEVHNAKTK |
| | | KEYKCKVSNK | YRVVSVLTVL | PREEQYNSTY |
| | | ALPAPIEKTI | HQDWLNGKEY | RVVSVLTVLH |
| | | SKAKGQPREP | KCKVSNKALP | QDWLNGKEYK |
| | | QVYTLPPCRE | APIEKTISKA | CKVSNKALPA |
| | | EMTKNQVSLS | KGQPREPQVC | PIEKTISKAK |
| | | CAVKGFYPSD | TLPPSREEMT | GQPREPQVCT |
| | | IAVEWESNGQ | KNQVSLWCLV | LPPSREEMTK |
| | | PENNYKTTPP | KGFYPSDIAV | NQVSLWCLVK |
| | | VLDSDGSFFL | EWESNGQPEN | GFYPSDIAVE |
| | | VSKLTVDKSR | NYKTTPPVLD | WESNGQPENN |
| | | WQQGNVFSCS | SDGSFFLYSK | YKTTPPVLDS |
| | | VMHEALHNHY | LTVDKSRWQQ | DGSFFLYSKL |
| | | TQKSLSLSPG | GNVFSCSVMH | TVDKSRWQQG |
| | | SGGGGSGGGG | EALHNHYTQK | NVFSCSVMHE |
| | | SGGGGSAPTS | SLSLSPG | ALHNHYTQKS |
| | | SSTKKTQLQL | (SEQ ID | LSLSPGK |
| | | EELLLDLQMI | NO: 328) | (SEQ ID |
| | | LNGINNYKNP | | NO: 329) |
| | | KLTEMLTAKF | | |
| | | YMPKKATELK | | |
| | | HLQCLEEELK | | |
| | | PLEEVLNLAQ | | |
| | | SKNFHLRPRD | | |
| | | LISNINVIVL | | |
| | | ELKGSETTFM | | |
| | | CEYADETATI | | |
| | | VEFLNRWITF | | |
| | | AQSIISTLT | | |
| | | (SEQ ID | | |
| | | NO: 327) | | |
| xh | EIVLTQSPGT | EVQLVESGGG | EVQLVESGGG | EVQLVESGGG |
| CD8v10- | LSLSPGERAT | LVQPGGSLRL | LVQPGGSLRL | LVQPGGSLRL |
| IL-2m4 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLLIYG | PGKGLEWVSD | PGKGLEWVSD | PGKGLEWVSD |
| | ASSRATGIPD | ITYAGGSTAY | ITYAGGSTAY | ITYAGGSTAY |
| | RFSGSGSGTD | ADSVKGRFTI | ADSVKGRFTI | ADSVKGRFTI |
| | FTLTISRLEP | SRDNAKNSLY | SRDNAKNSLY | SRDNAKNSLY |
| | EDFAVYYCQQ | LQMNSLRAED | LQMNSLRAED | LQMNSLRAED |
| | YGSSPPVTFG | TAVYYCARSN | TAVYYCARSN | TAVYYCARSN |
| | QGTKVEIKRT | AYAWDDALDI | AYAWDDALDI | AYAWDDALDI |
| | VAAPSVFIFP | WGQGTMVTVS | WGQGTMVTVS | WGQGTMVTVS |
| | PSDEQLKSGT | SASTKGPSVF | SASTKGPSVF | SASTKGPSVF |
| | ASVVCLLNNF | PLAPSSKSTS | PLAPSSKSTS | PLAPSSKSTS |
| | YPREAKVQWK | GGTAALGCLV | GGTAALGCLV | GGTAALGCLV |
| | VDNALQSGNS | KDYFPEPVTV | KDYFPEPVTV | KDYFPEPVTV |
| | QESVTEQDSK | SWNSGALTSG | SWNSGALTSG | SWNSGALTSG |
| | DSTYSLSSTL | VHTFPAVLQS | VHTFPAVLQS | VHTFPAVLQS |
| | TLSKADYEKH | SGLYSLSSVV | SGLYSLSSVV | SGLYSLSSVV |
| | KVYACEVTHQ | TVPSSSLGTQ | TVPSSSLGTQ | TVPSSSLGTQ |
| | GLSSPVTKSF | TYICNVNHKP | TYICNVNHKP | TYICNVNHKP |
| | NRGEC | SNTKVDKKVE | SNTKVDKKVE | SNTKVDKKVE |
| | (SEQ ID | PKSCDKTHTC | PKSCDKTHTC | PKSCDKTHTC |
| | NO: 330) | PPCPAPEAAG | PPCPAPEAAG | PPCPAPEAAG |
| | | APSVFLFPPK | APSVFLFPPK | APSVFLFPPK |
| | | PKDTLMISRT | PKDTLMISRT | PKDTLMISRT |
| | | PEVTCVVVDV | PEVTCVVVDV | PEVTCVVVDV |
| | | SHEDPEVKFN | SHEDPEVKFN | SHEDPEVKFN |
| | | WYVDGVEVHN | WYVDGVEVHN | WYVDGVEVHN |
| | | AKTKPREEQY | AKTKPREEQY | AKTKPREEQY |
| | | NSTYRVVSVL | NSTYRVVSVL | NSTYRVVSVL |
| | | TVLHQDWLNG | TVLHQDWLNG | TVLHQDWLNG |
| | | KEYKCKVSNK | KEYKCKVSNK | KEYKCKVSNK |
| | | ALPAPIEKTI | ALPAPIEKTI | ALPAPIEKTI |
| | | SKAKGQPREP | SKAKGQPREP | SKAKGQPREP |
| | | QVYTLPPCRE | QVCTLPPSRE | QVCTLPPSRE |
| | | EMTKNQVSLS | EMTKNQVSLW | EMTKNQVSLW |
| | | CAVKGFYPSD | CLVKGFYPSD | CLVKGFYPSD |
| | | IAVEWESNGQ | IAVEWESNGQ | IAVEWESNGQ |
| | | PENNYKTTPP | PENNYKTTPP | PENNYKTTPP |
| | | VLDSDGSFFL | VLDSDGSFFL | VLDSDGSFFL |
| | | VSKLTVDKSR | YSKLTVDKSR | YSKLTVDKSR |
| | | WQQGNVFSCS | WQQGNVFSCS | WQQGNVFSCS |
| | | VMHEALHNHY | VMHEALHNHY | VMHEALHNHY |
| | | TQKSLSLSPG | TQKSLSLSPG | TQKSLSLSPG |
| | | SGGGGSGGGG | (SEQ ID | K |
| | | SGGGGSAPTS | NO: 332) | (SEQ ID |
| | | SSTKKTQLQL | | NO: 333) |
| | | EELLLDLQMI | | |
| | | LNGINNYKNP | | |
| | | KLTEMLTAKF | | |
| | | YMPKKATELK | | |
| | | HLQCLEEELK | | |
| | | PLEEVLNLAQ | | |
| | | SKNFHLRPRD | | |
| | | LISNINVIVL | | |
| | | ELKGSETTFM | | |
| | | CEYADETATI | | |
| | | VEFLNRWITF | | |
| | | AQSIISTLT | | |
| | | (SEQ ID | | |
| | | NO: 331) | | |
| xh | EIVLTQSPGT | EVQLVESGGG | EVQLVESGGG | EVQLVESGGG |
| CD8v11- | LSLSPGERAT | LVQPGGSLRL | LVQPGGSLRL | LVQPGGSLRL |
| IL-2m4 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLLIYG | PGKGLEWVSD | PGKGLEWVSD | PGKGLEWVSD |
| | ASSRATGIPD | ITYAGGSTAY | ITYAGGSTAY | ITYAGGSTAY |
| | RFSGSGSGTD | ADSVKGRFTI | ADSVKGRFTI | ADSVKGRFTI |
| | FTLTISRLEP | SRDNAKNSLY | SRDNAKNSLY | SRDNAKNSLY |
| | EDFAVYYCQQ | LQMNSLRAED | LQMNSLRAED | LQMNSLRAED |
| | YGSSPPVTFG | TAVYYCARSN | TAVYYCARSN | TAVYYCARSN |
| | QGTKVEIKRT | AYAWDDALDI | AYAWDDALDI | AYAWDDALDI |
| | VAAPSVFIFP | WGQGTLVTVS | WGQGTLVTVS | WGQGTLVTVS |
| | PSDEQLKSGT | SASTKGPSVF | SASTKGPSVF | SASTKGPSVF |
| | ASVVCLLNNF | PLAPSSKSTS | PLAPSSKSTS | PLAPSSKSTS |
| | YPREAKVQWK | GGTAALGCLV | GGTAALGCLV | GGTAALGCL |
| | VDNALQSGNS | KDYFPEPVTV | KDYFPEPVTV | VKDYFPEP |
| | QESVTEQDSK | SWNSGALTSG | SWNSGALTSG | VTVSWNSGAL |
| | DSTYSLSSTL | VHTFPAVLQS | VHTFPAVLQS | TSGVHTFPAV |
| | TLSKADYEKH | SGLYSLSSVV | SGLYSLSSVV | LQSSGLYSLS |
| | KVYACEVTHQ | TVPSSSLGTQ | TVPSSSLGTQ | SVVTVPSSSL |
| | GLSSPVTKSF | TYICNVNHKP | TYICNVNHKP | GTQTYICNVN |
| | NRGEC | SNTKVDKKVE | SNTKVDKKVE | HKPSNTKVDK |
| | (SEQ ID | PKSCDKTHTC | PKSCDKTHTC | KVEPKSCDKT |
| | NO: 334) | PPCPAPEAAG | PPCPAPEAAG | HTCPPCPAPE |
| | | APSVFLFPPK | APSVFLFPPK | AAGAPSVFLF |
| | | PKDTLMISRT | PKDTLMISRT | PPKPKDTLMI |
| | | PEVTCVVVDV | PEVTCVVVDV | SRTPEVTCVV |
| | | SHEDPEVKFN | SHEDPEVKFN | VDVSHEDPEV |
| | | WYVDGVEVHN | WYVDGVEVHN | KFNWYVDGVE |
| | | AKTKPREEQY | AKTKPREEQY | VHNAKTKPRE |
| | | NSTYRVVSVL | NSTYRVVSVL | EQYNSTYRVV |
| | | TVLHQDWLNG | TVLHQDWLNG | SVLTVLHQDW |
| | | KEYKCKVSNK | KEYKCKVSNK | LNGKEYKCKV |
| | | ALPAPIEKTI | ALPAPIEKTI | SNKALPAPIE |
| | | SKAKGQPREP | SKAKGQPREP | KTISKAKGQP |
| | | QVYTLPPCRE | QVCTLPPSRE | REPQVCTLPP |
| | | EMTKNQVSLS | EMTKNQVSLW | SREEMTKNQV |
| | | CAVKGFYPSD | CLVKGFYPSD | SLWCLVKGFY |
| | | IAVEWESNGQ | IAVEWESNGQ | PSDIAVEWES |
| | | PENNYKTTPP | PENNYKTTPP | NGQPENNYKT |
| | | VLDSDGSFFL | VLDSDGSFFL | TPPVLDSDGS |
| | | VSKLTVDKSR | YSKLTVDKSR | FFLYSKLTVD |
| | | WQQGNVFSCS | WQQGNVFSCS | KSRWQQGNVF |
| | | VMHEALHNHY | VMHEALHNHY | SCSVMHEALH |
| | | TQKSLSLSPG | TQKSLSLSPG | NHYTQKSLSL |
| | | SGGGGSGGGG | (SEQ ID | SPGK |
| | | SGGGGSAPTS | NO: 336) | (SEQ ID |
| | | SSTKKTQLQL | | NO: 337) |
| | | EELLLDLQMI | | |
| | | LNGINNYKNP | | |
| | | KLTEMLTAKF | | |
| | | YMPKKATELK | | |
| | | HLQCLEEELK | | |
| | | PLEEVLNLAQ | | |
| | | SKNFHLRPRD | | |
| | | LISNINVIVL | | |
| | | ELKGSETTFM | | |
| | | CEYADETATI | | |
| | | VEFLNRWITF | | |
| | | AQSIISTLT | | |
| | | (SEQ ID | | |
| | | NO: 335) | | |
| xh | DIQMTQSPSS | QVQLVQSGAE | QVQLVQSGAE | QVQLVQSGAE |
| CD8v12- | LSASVGDRVT | VKKPGSSVKV | VKKPGSSVKV | VKKPGSSVKV |
| IL-2m4 | ITCRASQSIY | SCKASGGTFS | SCKASGGTFS | SCKASGGTFS |
| | GALNWYQQKP | SYAISWVRQA | SYAISWVRQA | SYAISWVRQA |
| | GKAPKLLIYG | PGQGLEWMGG | PGQGLEWMGG | PGQGLEWMGG |
| | ASNLQSGVPS | IIPGYATANY | IIPGYATANY | IIPGYATANY |
| | RFSGSGSGTD | AQKFQGRVTI | AQKFQGRVTI | AQKFQGRVTI |
| | FTLTISSLQP | TADESTSTAY | TADESTSTAY | TADESTSTAY |
| | EDFATYYCQS | MELSSLRSED | MELSSLRSED | MELSSLRSED |
| | TYTAPWTFGG | TAVYYCARDA | TAVYYCARDA | TAVYYCARDA |
| | GTKVEIKRTV | AGIRLFADWG | AGIRLFADWG | AGIRLFADWG |
| | AAPSVFIFPP | QGTLVTVSSA | QGTLVTVSSA | QGTLVTVSSA |
| | SDEQLKSGTA | STKGPSVFPL | STKGPSVFPL | STKGPSVFPL |
| | SVVCLLNNFY | APSSKSTSGG | APSSKSTSGG | APSSKSTSGG |
| | PREAKVQWKV | TAALGCLVKD | TAALGCLVKD | TAALGCLVKD |
| | DNALQSGNSQ | YFPEPVTVSW | YFPEPVTVSW | YFPEPVTVSW |
| | ESVTEQDSKD | NSGALTSGVH | NSGALTSGVH | NSGALTSGVH |
| | STYSLSSTLT | TFPAVLQSSG | TFPAVLQSSG | TFPAVLQSSG |
| | LSKADYEKHK | LYSLSSVVTV | LYSLSSVVTV | LYSLSSVVTV |
| | VYACEVTHQG | PSSSLGTQTY | PSSSLGTQTY | PSSSLGTQTY |
| | LSSPVTKSFN | ICNVNHKPSN | ICNVNHKPSN | ICNVNHKPSN |
| | RGEC | TKVDKKVEPK | TKVDKKVEPK | TKVDKKVEPK |
| | (SEQ ID | SCDKTHTCPP | SCDKTHTCPP | SCDKTHTCPP |
| | NO: 338) | CPAPEAAGAP | CPAPEAAGAP | CPAPEAAGAP |
| | | SVFLFPPKPK | SVFLFPPKPK | SVFLFPPKPK |
| | | DTLMISRTPE | DTLMISRTPE | DTLMISRTPE |
| | | VTCVVVDVSH | VTCVVVDVSH | VTCVVVDVSH |
| | | EDPEVKFNWY | EDPEVKFNWY | EDPEVKFNWY |
| | | VDGVEVHNAK | VDGVEVHNAK | VDGVEVHNAK |
| | | TKPREEQYNS | TKPREEQYNS | TKPREEQYNS |
| | | TYRVVSVLTV | TYRVVSVLTV | TYRVVSVLTV |
| | | LHQDWLNGKE | LHQDWLNGKE | LHQDWLNGKE |
| | | YKCKVSNKAL | YKCKVSNKAL | YKCKVSNKAL |
| | | PAPIEKTISK | PAPIEKTISK | PAPIEKTISK |
| | | AKGQPREPQV | AKGQPREPQV | AKGQPREPQV |
| | | YTLPPCREEM | CTLPPSREEM | CTLPPSREEM |
| | | TKNQVSLSCA | TKNQVSLWCL | TKNQVSLWCL |
| | | VKGFYPSDIA | VKGFYPSDIA | VKGFYPSDIA |
| | | VEWESNGQPE | VEWESNGQPE | VEWESNGQPE |
| | | NNYKTTPPVL | NNYKTTPPVL | NNYKTTPPVL |
| | | DSDGSFFLVS | DSDGSFFLYS | DSDGSFFLYS |
| | | KLTVDKSRWQ | KLTVDKSRWQ | KLTVDKSRWQ |
| | | QGNVFSCSVM | QGNVFSCSVM | QGNVFSCSVM |
| | | HEALHNHYTQ | HEALHNHYTQ | HEALHNHYTQ |
| | | KSLSLSPGSG | KSLSLSPG | KSLSLSPGK |
| | | GGGSGGGGSG | (SEQ ID | (SEQ ID |
| | | GGGSAPTSSS | NO: 340) | NO: 341) |
| | | TKKTQLQLEE | | |
| | | LLLDLQMILN | | |
| | | GINNYKNPKL | | |
| | | TEMLTAKFYM | | |
| | | PKKATELKHL | | |
| | | QCLEEELKPL | | |
| | | EEVLNLAQSK | | |
| | | NFHLRPRDLI | | |
| | | SNINVIVLEL | | |
| | | KGSETTFMCE | | |
| | | YADETATIVE | | |
| | | FLNRWITFAQ | | |
| | | SIISTLT | | |
| | | (SEQ ID | | |
| | | NO: 339) | | |
| xh | DIQMTQSPSS | QVQLVQSGAE | QVQLVQSGAE | QVQLVQSGAE |
| CD8v13- | LSASVGDRVT | VKKPGSSVKV | VKKPGSSVKV | VKKPGSSVKV |
| IL-2m4 | ITCRASQEIY | SCKASGGTFS | SCKASGGTFS | SCKASGGTFS |
| | GALNWYQQKP | SYAISWVRQA | SYAISWVRQA | SYAISWVRQA |
| | GKAPKLLIYG | PGQGLEWMGG | PGQGLEWMGG | PGQGLEWMGG |
| | ATNLQSGVPS | IIPGYATANY | IIPGYATANY | IIPGYATANY |
| | RFSGSGSGTD | AQKFQGRVTI | AQKFQGRVTI | AQKFQGRVTI |
| | FTLTISSLQP | TADESTSTAY | TADESTSTAY | TADESTSTAY |
| | EDFATYYCQS | MELSSLRSED | MELSSLRSED | MELSSLRSED |
| | TYDAPWTEGG | TAVYYCARDA | TAVYYCARDA | TAVYYCARDA |
| | GTKVEIKRTV | AGIRLFADWG | AGIRLFADWG | AGIRLFADWG |
| | AAPSVFIFPP | QGTLVTVSSA | QGTLVTVSSA | QGTLVTVSSA |
| | SDEQLKSGTA | STKGPSVFPL | STKGPSVFPL | STKGPSVFPL |
| | SVVCLLNNFY | APSSKSTSGG | APSSKSTSGG | APSSKSTSGG |
| | PREAKVQWKV | TAALGCLVKD | TAALGCLVKD | TAALGCLVKD |
| | DNALQSGNSQ | YFPEPVTVSW | YFPEPVTVSW | YFPEPVTVSW |
| | ESVTFQDSKD | NSGALTSGVH | NSGALTSGVH | NSGALTSGVH |
| | STYSLSSTLT | TFPAVLQSSG | TFPAVLQSSG | TFPAVLQSSG |
| | LSKADYEKHK | LYSLSSVVTV | LYSLSSVVTV | LYSLSSVVTV |
| | VYACEVTHQG | PSSSLGTQTY | PSSSLGTQTY | PSSSLGTQTY |
| | LSSPVTKSFN | ICNVNHKPSN | ICNVNHKPSN | ICNVNHKPSN |
| | RGEC | TKVDKKVEPK | TKVDKKVEPK | TKVDKKVEPK |
| | (SEQ ID | SCDKTHTCPP | SCDKTHTCPP | SCDKTHTCPP |
| | NO: 342) | CPAPEAAGAP | CPAPEAAGAP | CPAPEAAGAP |
| | | SVFLFPPKPK | SVFLFPPKPK | SVFLFPPKPK |
| | | DTLMISRTPE | DTLMISRTPE | DTLMISRTPE |
| | | VTCVVVDVSH | VTCVVVDVSH | VTCVVVDVSH |
| | | EDPEVKFNWY | EDPEVKFNWY | EDPEVKFNWY |
| | | VDGVEVHNAK | VDGVEVHNAK | VDGVEVHNAK |
| | | TKPREEQYNS | TKPREEQYNS | TKPREEQYNS |
| | | TYRVVSVLTV | TYRVVSVLTV | TYRVVSVLTV |
| | | LHQDWLNGKE | LHQDWLNGKE | LHQDWLNGKE |
| | | YKCKVSNKAL | YKCKVSNKAL | YKCKVSNKAL |
| | | PAPIEKTISK | PAPIEKTISK | PAPIEKTISK |
| | | AKGQPREPQV | AKGQPREPQV | AKGQPREPQV |
| | | YTLPPCREEM | CTLPPSREEM | CTLPPSREEM |
| | | TKNQVSLSCA | TKNQVSLWCL | TKNQVSLWCL |
| | | VKGFYPSDIA | VKGFYPSDIA | VKGFYPSDIA |
| | | VEWESNGQPE | VEWESNGQPE | VEWESNGQPE |
| | | NNYKTTPPVL | NNYKTTPPVL | NNYKTTPPVL |
| | | DSDGSFFLVS | DSDGSFFLYS | DSDGSFFLYS |
| | | KLTVDKSRWQ | KLTVDKSRWQ | KLTVDKSRWQ |
| | | QGNVFSCSVM | QGNVFSCSVM | QGNVFSCSVM |
| | | HEALHNHYTQ | HEALHNHYTQ | HEALHNHYTQ |
| | | KSLSLSPGSG | KSLSLSPG | KSLSLSPGK |
| | | GGGSGGGGSG | (SEQ ID | (SEQ ID |
| | | GGGSAPTSSS | NO: 344) | NO: 345) |
| | | TKKTQLQLEE | | |
| | | LLLDLQMILN | | |
| | | GINNYKNPKL | | |
| | | TEMLTAKFYM | | |
| | | PKKATELKHL | | |
| | | QCLEEELKPL | | |
| | | EEVLNLAQSK | | |
| | | NFHLRPRDLI | | |
| | | SNINVIVLEL | | |
| | | KGSETTFMCE | | |
| | | YADETATIVE | | |
| | | FLNRWITFAQ | | |
| | | SIISTLT | | |
| | | (SEQ ID | | |
| | | NO: 343) | | |
| xh | EIVLTQSPGT | EVQLVESGGG | EVQLVESGGG | EVQLVESGGG |
| CD8v14- | LSLSPGERAT | LVQPGGSLRL | LVQPGGSLRL | LVQPGGSLRL |
| IL-2m4 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLLIYG | PGKGLEWVSD | PGKGLEWVSD | PGKGLEWVSD |
| | ASSRATGIPD | ISYAGGSTAY | ISYAGGSTAY | ISYAGGSTAY |
| | RFSGSGSGTD | ADSVKGRFTI | ADSVKGRFTI | ADSVKGRFTI |
| | FTLTISRLEP | SRDNAKNSLY | SRDNAKNSLY | SRDNAKNSLY |
| | EDFAVYYCQQ | LQMNSLRAED | LQMNSLRAED | LQMNSLRAED |
| | YGSSPPVTFG | TAVYYCARSN | TAVYYCARSN | TAVYYCARSN |
| | QGTKVEIKRT | AYAWDDALDI | AYAWDDALDI | AYAWDDALDI |
| | VAAPSVFIFP | WGQGTMVTVS | WGQGTMVTVS | WGQGTMVTVS |
| | PSDEQLKSGT | SASTKGPSVF | SASTKGPSVF | SASTKGPSVF |
| | ASVVCLLNNF | PLAPSSKSTS | PLAPSSKSTS | PLAPSSKSTS |
| | YPREAKVQWK | GGTAALGCLV | GGTAALGCLV | GGTAALGCLV |
| | VDNALQSGNS | KDYFPEPVTV | KDYFPEPVTV | KDYFPEPVTV |
| | QESVTEQDSK | SWNSGALTSG | SWNSGALTSG | SWNSGALTSG |
| | DSTYSLSSTL | VHTFPAVLQS | VIITFPAVLQ | VHTFPAVLQS |
| | TLSKADYEKH | SGLYSLSSVV | SSGLYSLSSV | SGLYSLSSVV |
| | KVYACEVTHQ | TVPSSSLGTQ | VTVPSSSLGT | TVPSSSLGTQ |
| | GLSSPVTKSF | TYICNVNII | QTYICNVNHK | TYICNVNHKP |
| | NRGEC | KPSNTKVDK | PSNTKVDKKV | SNTKVDKKVE |
| | (SEQ ID | KVEPKSCDKT | EPKSCDKTHT | PKSCDKTHTC |
| | NO: 346) | HTCPPCPAPE | CPPCPAPEAA | PPCPAPEAAG |
| | | AAGAPSVFLF | GAPSVFLFPP | APSVFLFPPK |
| | | PPKPKDTLMI | KPKDTLMISR | PKDTLMISRT |
| | | SRTPEVTCVV | TPEVTCVVVD | PEVTCVVVDV |
| | | VDVSHEDPEV | VSHEDPEVKF | SHEDPEVKFN |
| | | KFNWYVDGVE | NWYVDGVEVH | WYVDGVEVHN |
| | | VHNAKTKPRE | NAKTKPREEQ | AKTKPREEQY |
| | | EQYNSTYRVV | YNSTYRVVSV | NSTYRVVSVL |
| | | SVLTVLHQDW | LTVLHQDWLN | TVLHQDWLNG |
| | | LNGKEYKCKV | GKEYKCKVSN | KEYKCKVSNK |
| | | SNKALPAPIE | KALPAPIEKT | ALPAPIEKTI |
| | | KTISKAKGQP | ISKAKGQPRE | SKAKGQPREP |
| | | REPQVYTLPP | PQVCTLPPSR | QVCTLPPSRE |
| | | CREEMTKNQV | EEMTKNQVSL | EMTKNQVSLW |
| | | SLSCAVKGFY | WCLVKGFYPS | CLVKGFYPSD |
| | | PSDIAVEWES | DIAVEWESNG | IAVEWESNGQ |
| | | NGQPENNYKT | QPENNYKTTP | PENNYKTTPP |
| | | TPPVLDSDGS | PVLDSDGSFF | VLDSDGSFFL |
| | | FFLVSKLTVD | LYSKLTVDKS | YSKLTVDKSR |
| | | KSRWQQGNVF | RWQQGNVFSC | WQQGNVFSCS |
| | | SCSVMHEALH | SVMHEALHNH | VMHEALHNHY |
| | | NHYTQKSLSL | YTQKSLSLSP | TQKSLSLSPG |
| | | SPGSGGGGSG | G | K |
| | | GGGSGGGGSA | (SEQ ID | (SEQ ID |
| | | PTSSSTKKTQ | NO: 348) | NO: 349) |
| | | LQLEELLLDL | | |
| | | QMILNGINNY | | |
| | | KNPKLTEMLT | | |
| | | AKFYMPKKAT | | |
| | | ELKHLQCLEE | | |
| | | ELKPLEEVLN | | |
| | | LAQSKNFHLR | | |
| | | PRDLISNINV | | |
| | | IVLELKGSET | | |
| | | TFMCEYADET | | |
| | | ATIVEFLNRW | | |
| | | ITFAQSIIST | | |
| | | LT | | |
| | | (SEQ ID | | |
| | | NO: 347) | | |
| xh | EIVLTQSPGT | EVQLVESGGG | EVQLVESGGG | EVQLVESGGG |
| CD8v15- | LSLSPGERAT | LVQPGGSLRL | LVQPGGSLRL | LVQPGGSLRL |
| IL-2m4 | LSCRASQSVS | SCAASGFTFS | SCAASGFTFS | SCAASGFTFS |
| | SNLAWYQQKP | SYAMSWVRQA | SYAMSWVRQA | SYAMSWVRQA |
| | GQAPRLLIYG | PGKGLEWVSD | PGKGLEWVSD | PGKGLEWVSD |
| | ASSRATGIPD | ISYAGGSTAY | ISYAGGSTAY | ISYAGGSTAY |
| | RFSGSGSGTD | ADSVKGRFTI | ADSVKGRFTI | ADSVKGRFTI |
| | FTLTISRLEP | SRDNAKNSLY | SRDNAKNSLY | SRDNAKNSLY |
| | EDFAVYYCQQ | LQMNSLRAED | LQMNSLRAED | LQMNSLRAED |
| | YGSSPPVTFG | TAVYYCARSN | TAVYYCARSN | TAVYYCARSN |
| | QGTKVEIKRT | AYAWDDALDI | AYAWDDALDI | AYAWDDALDI |
| | VAAPSVFIFP | WGQGTLVTVS | WGQGTLVTVS | WGQGTLVTVS |
| | PSDEQLKSGT | SASTKGPSVF | SASTKGPSVF | SASTKGPSVF |
| | ASVVCLLNNF | PLAPSSKSTS | PLAPSSKSTS | PLAPSSKSTS |
| | YPREAKVQWK | GGTAALGCLV | GGTAALGCLV | GGTAALGCLV |
| | VDNALQSGNS | KDYFPEPVTV | KDYFPEPVTV | KDYFPEPVTV |
| | QESVTEQDSK | SWNSGALTSG | SWNSGALTSG | SWNSGALTSG |
| | DSTYSLSSTL | VHTFPAVLQS | VHTFPAVLQS | VHTFPAVLQS |
| | TLSKADYEKH | SGLYSLSSVV | SGLYSLSSVV | SGLYSLSSVV |
| | KVYACEVTHQ | TVPSSSLGTQ | TVPSSSLGTQ | TVPSSSLGTQ |
| | GLSSPVTKSF | TYICNVNHKP | TYICNVNHKP | TYICNVNHKP |
| | NRGEC | SNTKVDKKVE | SNTKVDKKVE | SNTKVDKKVE |
| | (SEQ ID | PKSCDKTHTC | PKSCDKTHTC | PKSCDKTHTC |
| | NO: 350) | PPCPAPEAAG | PPCPAPEAAG | PPCPAPEAAG |
| | | APSVFLFPPK | APSVFLFPPK | APSVFLFPPK |
| | | PKDTLMISRT | PKDTLMISRT | PKDTLMISRT |
| | | PEVTCVVVDV | PEVTCVVVDV | PEVTCVVVDV |
| | | SHEDPEVKFN | SHEDPEVKFN | SHEDPEVKFN |
| | | WYVDGVEVHN | WYVDGVEVHN | WYVDGVEVHN |
| | | AKTKPREEQY | AKTKPREEQY | AKTKPREEQY |
| | | NSTYRVVSVL | NSTYRVVSVL | NSTYRVVSVL |
| | | TVLHQDWLNG | TVLHQDWLNG | TVLHQDWLNG |
| | | KEYKCKVSNK | KEYKCKVSNK | KEYKCKVSNK |
| | | ALPAPIEKTI | ALPAPIEKTI | ALPAPIEKTI |
| | | SKAKGQPREP | SKAKGQPREP | SKAKGQPREP |
| | | QVYTLPPCRE | QVCTLPPSRE | QVCTLPPSRE |
| | | EMTKNQVSLS | EMTKNQVSLW | EMTKNQVSLW |
| | | CAVKGFYPSD | CLVKGFYPSD | CLVKGFYPSD |
| | | IAVEWESNGQ | IAVEWESNGQ | IAVEWESNGQ |
| | | PENNYKTTPP | PENNYKTTPP | PENNYKTTPP |
| | | VLDSDGSFFL | VLDSDGSFFL | VLDSDGSFFL |
| | | VSKLTVDKSR | YSKLTVDKSR | YSKLTVDKSR |
| | | WQQGNVFSCS | WQQGNVFSCS | WQQGNVFSCS |
| | | VMHEALHNHY | VMHEALHNHY | VMHEALHNHY |
| | | TQKSLSLSPG | TQKSLSLSPG | TQKSLSLSPG |
| | | SGGGGSGGGG | (SEQ ID | K |
| | | SGGGGSAPTS | NO: 352) | (SEQ ID |
| | | SSTKKTQLQL | | NO: 353) |
| | | EELLLDLQMI | | |
| | | LNGINNYKNP | | |
| | | KLTEMLTAKF | | |
| | | YMPKKATELK | | |
| | | HLQCLEEELK | | |
| | | PLEEVLNLAQ | | |
| | | SKNFHLRPRD | | |
| | | LISNINVIVL | | |
| | | ELKGSETTFM | | |
| | | CEYADETATI | | |
| | | VEFLNRWITF | | |
| | | AQSIISTLT | | |
| | | (SEQ ID | | |
| | | NO: 351) | | |

### Production of Antibodies and Fusion Proteins

Further provided herein are polynucleotides (e.g., isolated polynucleotides) encoding any of the antibodies, antibody fragments, and fusion proteins described herein. Further provided herein are vectors (e.g., expression vectors) encoding any of the antibodies, antibody fragments, and fusion proteins described herein.

Further provided herein are host cells (e.g., isolated host cells or host cell lines) comprising any of the polynucleotides or vectors described herein.

Further provided herein are methods of producing any of the antibodies, antibody fragments, and fusion proteins described herein. In some embodiments, the methods comprise culturing a host cell of the present disclosure under conditions suitable for production of the antibody, antibody fragment, or fusion protein. In some embodiments, the methods further comprise recovering the antibody, antibody fragment, or fusion protein.

Antibodies, antibody fragments, and fusion proteins may be produced using recombinant methods, e.g., as exemplified infra. In some embodiments, nucleic acid encoding the antibody/fusion protein can be isolated and inserted into a replicable vector for further cloning or for expression. DNA encoding the antibody/fusion protein may be readily isolated and sequenced using conventional procedures (e.g., via oligonucleotide probes capable of binding specifically to genes encoding the heavy and light chains of the antibody/fragment). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody/fusion protein can be produced intracellularly, in the periplasmic space, or directly secreted into the medium. If the antibody/fragment is produced intracellularly, the particulate debris, either host cells or lysed fragments, are removed, for example, by centrifugation or ultrafiltration. Where the antibody/fusion protein is secreted into the medium, supernatants from such expression systems are generally first concentrated using a commercially available protein concentration filter.

### Pharmaceutical Compositions

In some embodiments, a fusion protein of the present disclosure is part of a pharmaceutical composition, e.g., including the fusion protein and one or more pharmaceutically acceptable carriers. Pharmaceutical compositions and formulations as described herein can be prepared by mixing the active ingredients (such as a fusion protein) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In some embodiments, a fusion protein of the present disclosure is lyophilized.

### Methods and Kits

Certain aspects of the present disclosure relate to methods of treating cancer or chronic infection. In some embodiments, the methods comprise administering an effective amount of a fusion protein or antibody, or a pharmaceutical composition comprising the fusion protein or antibody and a pharmaceutically acceptable carrier, to a patient. In some embodiments, the patient in need of said treatment has been diagnosed with cancer.

In some embodiments, the fusion protein or composition is administered in combination with a T cell therapy, cancer vaccine, chemotherapeutic agent, or immune checkpoint inhibitor (ICI). In some embodiments, the chemotherapeutic agent is a kinase inhibitor, antimetabolite, cytotoxin or cytostatic agent, anti-hormonal agent, platinum-based chemotherapeutic agent, methyltransferase inhibitor, antibody, or anti-cancer peptide. In some embodiments, the immune checkpoint inhibitor targets PD-L1, PD-1, CTLA-4, CEACAM, LAIRI, CD160, 2B4, CD80, CD86, CD276, VTCNI, HVEM, KIR, A2AR, MHC class I, MHC class II, GALS, adenosine, TGFR, OX40, CD137, CD40, CD47, TREM1, TREM2, HLA-G, CCR4, CCR8, CD39, CD73, IDO, CSFIR, TIM-3, BTLA, VISTA, LAG-3, TIGIT, IDO, MICA/B, LILRβ4, SIGLEC-15, or arginase, including without limitation an inhibitor of PD-1 (e.g., an anti-PD-1 antibody), PD-L1 (e.g., an anti-PD-L1 antibody), or CTLA-4 (e.g., an anti-CTLA-4 antibody).

Examples of anti-PD-1 antibodies include, without limitation, pembrolizumab, nivolumab, cemiplimab, zimberelimab (Arcus), sasanlimab (Pfizer), JTX-4014, spartalizumab (PDR001; Novartis), camrelizumab (SHR1210; Jiangsu HengRui Medicine), sintilimab (IBI308; Innovent and Eli Lilly), tislelizumab (BGB-A317), toripalimab (JS 001), dostarlimab (TSR-042, WBP-285), INCMGA00012 (MGA012), AMP-224, and AMP-514 (MEDI0680). Examples of anti-PD-L1 antibodies include, without limitation, atezolizumab, avelumab, durvalumab, KN035, and CK-301 (Checkpoint Therapeutics). Examples of PD-L1 inhibitors (non-antibody based) include, without limitation, AUNP12, CA-170, and BMS-986189. Examples of anti-CTLA-4 antibodies include, without limitation, ipilimumab, tremelimumab, BMS-986218, BMS-986249, BMS-986288, HBM4003, ONC-392, KN044, ADG116, ADU-1604, AGEN1181, AGEN1884, MK-1308, and REGN4659.

Examples of T cell therapies include, without limitation, CD4+ or CD8+ T cell-based therapies, adoptive T cell therapies, chimeric antigen receptor (CAR)-based T cell therapies, tumor-infiltrating lymphocyte (TIL)-based therapies, autologous T cell therapies, allogeneic T cell therapies, and therapies with T cells bearing a transduced TCR. Exemplary cancer vaccines include, without limitation, dendritic cell vaccines, vaccines comprising one or more polynucleotides encoding one or more cancer antigens, and vaccines comprising one or more cancer antigenic peptides.

Certain aspects of the present disclosure relate to methods of expanding T cells, e.g., ex vivo. In some embodiments, the methods comprise contacting one or more T cells, e.g., ex vivo with an effective amount of the antibody or fusion protein of the present disclosure. In some embodiments, the one or more T cells are tumor infiltrating lymphocytes (TILs). In some embodiments, the methods further comprise isolating tumor infiltrating lymphocytes (TILs) from a tumor or tumor specimen.

Certain aspects of the present disclosure relate to kits or articles of manufacture comprising any of the antibodies, antibody fragments, or fusion proteins disclosed herein. In some embodiments, the article of manufacture comprises a container and a label or package insert on or associated with the container. In some embodiments, the kit or article of manufacture further comprises instructions for using the antibody or fusion protein according to any of the methods disclosed herein, e.g., for treating cancer or chronic infection or expanding T cells, e.g., ex vivo.

Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). At least one active agent in the composition is an antibody or fusion protein as described herein. The label or package insert indicates that the composition is used for treating the particular condition. The label or package insert will further comprise instructions for administering the antibody or fusion protein composition to the subject. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials considered from a commercial and user standpoint, including other buffers, diluents, filters, needles, and syringes.

### EXAMPLES

### Example 1: Generation and characterization of antibodies that bind to human CD8ab

### Materials and Methods

### Recombinant DNA Techniques

Techniques involving recombinant DNA manipulation were previously described in Sambrook et al., Molecular cloning: A laboratory manual; Cold Spring Harbor Laboratory Press, Cold Spring Harbor, N.Y., 1989. All reagents were used according to the manufacturer's instructions. DNA sequences were determined by double strand sequencing.

### Gene Synthesis

Desired gene segments were either generated by PCR using appropriate templates or synthesized at Genewiz (South Plainfield, NJ), Integrated DNA Technologies (Coralville, IA) or GeneScript (Piscataway, NJ) from synthetic oligonucleotides. The gene segments were cloned into the expression vectors using either Gibson assembly® method or using restriction digest followed by ligation. DNA was purified from transformed bacteria and concentration was determined by UV visible spectroscopy. DNA sequencing was used to confirm the DNA sequences of the subcloned gene fragments.

### Isolation of Antibodies

Antibodies binding to CD8 antigens were generated using either humanization of mouse antibodies or in vitro phage display system.

For humanization, complementarity-determining regions (CDRs) of mouse residues were grafted into selected human framework(s) which exhibit close sequence similarity to the parental mouse framework and good stability. The resulting CDR-grafted antibodies were further humanized to remove any unnecessary non-human mutations.

For in vitro display method, a non-immune human single chain Fv phage library generated from naïve B cells was panned for 5 to 6 rounds to isolate antibodies against the CD8 antigens. After the panning, individual phage clones that exhibited specific binding to target antigen over non-specific antigens in ELISA were identified. DNA fragments of heavy and light chain V-domain of the specific binders were subsequently cloned and sequenced. Parental clones of xhCD8v6 and xhCD8v7 were isolated from a human antibody phage library. xhCD8v1 was a mouse monoclonal antibody against CD8b and xhCD8v1.1 was a humanized version of xCD8v1.

### In Vitro Affinity Maturation of Human Antibodies

Affinity maturation library was generated by either shuffling the light chain of the parental clone against light chain isolated from healthy donor naïve B cell or by mutating selected CDR residues. The resulting library was displayed as scFv on the surface of either phage or yeast. Three to five rounds of screening with decreasing concentration of soluble CD8 antigen were performed. The resulting selected clones were subsequently cloned and sequenced. xhCD8v6 and xhCD8v7 are the resulting clones from affinity maturation. xhCD8v2, xhCD8v3, xhCD8v4, and xhCD8v5 are affinity matured antibodies derived from xhCD8v1.1.

### Cloning of Antibody Constructs

General information regarding the nucleotide sequences of human immunoglobulins light and heavy chains is given in: IMGT® (the international ImMunoGeneTics information system®) from Lefranc et al. IMGT®, the international ImMunoGeneTics information system® 25 years on. Nucleic Acids Res. 2015 January;43. The DNA fragments of heavy and light chain V-domains were inserted in frame into the human IgG1 and CK containing mammalian expression vector.

### Preparation of Antibody

DNA sequence verified constructs containing the heavy and light chain of an antibody were transfected into Expi 293 cells using polyethylenimine (PEI). After 5 days of culture, supernatant was collected and incubated with Protein A resin for >2 hr at room temperature. Next, the resin was used multiple times with PBS, and then the antibody was eluted off the resin with 20 mM sodium citrate, pH 3.6. The eluted fractions were pooled and further purified by size exclusion chromatography (Superdex 200, GE Healthcare) in PBS.

### Binding Affinity Determination by Surface Plasmon Resonance (SPR) for CD8ab Antigen

Recombinant human CD8ab protein was generated. Recombinant CD8ab, comprised of the extracellular domains of CD8a, fused to an acidic leucine zipper and an 8× Histidine tag, and CD8b, fused to a basic leucine zipper and a Strep-tag II, was expressed in secreted form from HEK293 cells and purified by IMAC, size exclusion, and Streptavidin affinity column chromatography. Antibody affinity to CD8 at 37° C. was determined by surface plasmon resonance using a Biacore T200 (Cytiva). Antibodies were first captured on an anti-hIgG-Fc CM5 or CM4 surface prepared using the Human Antibody Capture Kit (Cytiva). Soluble CD8ab antigen, diluted in HBS-EP+buffer at four or more concentrations spanning 0.1× to 10× the K_{D}, was flowed over the surface-captured anti-CD8 mAbs for 1-2 minutes. Dissociation was monitored for 5-10 minutes, and the anti-hIgG-Fc surface was regenerated with 3M MgCl2 before recapturing mAb in each subsequent cycle. Binding data were analyzed by Biacore T200 Evaluation Software version 3.2 using a 1:1 binding model.

### Results

Table 1 shows the binding kinetics of isolated anti-CD8ab antibodies (xhCD8v1 to xhCD8v7) to recombinant human CD8ab protein as determined by surface plasmon resonance.

TABLE 1

| Antibody | | | | |
|---|---|---|---|---|
| Name | Antibody Type | kₐ (1/MS) | k_{d} (1/s) | K_{D} (nM) |
| xhCD8v1 | Mouse monoclonal | 9.22E+05 | 0.0183 | 19.8 |
| xhCD8v2 | Affinity matured based on | 4.83E+05 | 0.02005 | 41.6 |
| | humanized xhCD8v1.1 | | | |
| xhCD8v3 | Affinity matured based on | 5.24E+05 | 0.03787 | 72.3 |
| | humanized xhCD8v1.1 | | | |
| xhCD8v4 | Affinity matured based on | 3.60E+05 | 0.0493 | 137.0 |
| | humanized xhCD8v1.1 | | | |
| xhCD8v5 | Affinity matured based on | 3.57E+05 | 0.04189 | 117.4 |
| | humanized xhCD8v1.1 | | | |
| xhCD8v6 | Affinity matured based on | 1.62E+06 | 5.71E−04 | 0.35 |
| | phage/yeast library clones | | | |
| xhCD8v7 | Affinity matured based on | 8.55E+05 | 9.22E−04 | 1.08 |
| | phage/yeast library clones | | | |

As depicted in Table 1, the isolated CD8ab antibodies had a range of affinities varying from 0.35 nM to 137 nM.

As depicted in FIGS. 1A-1C, three types of antibodies that bind to CD8ab antigen (CD8ab antibodies) could be identified: 1) antibodies binding CD8a antigen alone but not CD8b antigen (FIG. 1A), these antibodies target epitopes present on the CD8a molecule; 2) antibodies binding weakly or not binding to CD8a alone and CD8b alone antigens (FIG. 1B), these antibodies bind epitopes that are between CD8a and CD8b on the CD8ab heterodimer; 3) antibodies binding to CD8b alone but not CD8a alone antigens (FIG. 1C), these antibodies target epitopes on the CD8b molecule.

Binding specificity of the antibodies was determined by ELISA. 2 μg/mL of recombinant CD8ab, CD8a (Sino biological), CD8b (Sino biological) and ovalbumin (Sigma) or Erb2 (human epidermal growth factor receptor 2) (Sino biological) in PBS were coated onto the maxisorp plate overnight at 4° C. Ovalbumin or Erb2 was used as negative control. Plate was then blocked with casein blocking solution (Thermo Scientific) for Ihr at room temperature. After blocking, plate was washed with wash buffer (PBS/0.05% Tween-20). Next, dilution of antibody from 30 nM to 0.0009 nM in PBS/0.5% BSA/0.05% Tween-20 was added onto the plate and incubated for 1-2 hr at room temperature. After incubation, plate was washed, and incubated with anti-human IgG (Fc specific)-HRP conjugate or anti-mouse IgG (Fc specific)-HRP conjugate (Jackson Immunoresearch) in PBS/0.5% BSA/0.05% Tween-20 for Ihr. Binding was detected by adding TMB substrate (SeraCare) to the plate, followed by stopping solution of 0.1M HCl. Binding absorbance was read using GloMax® Discover Microplate Reader (Promega).

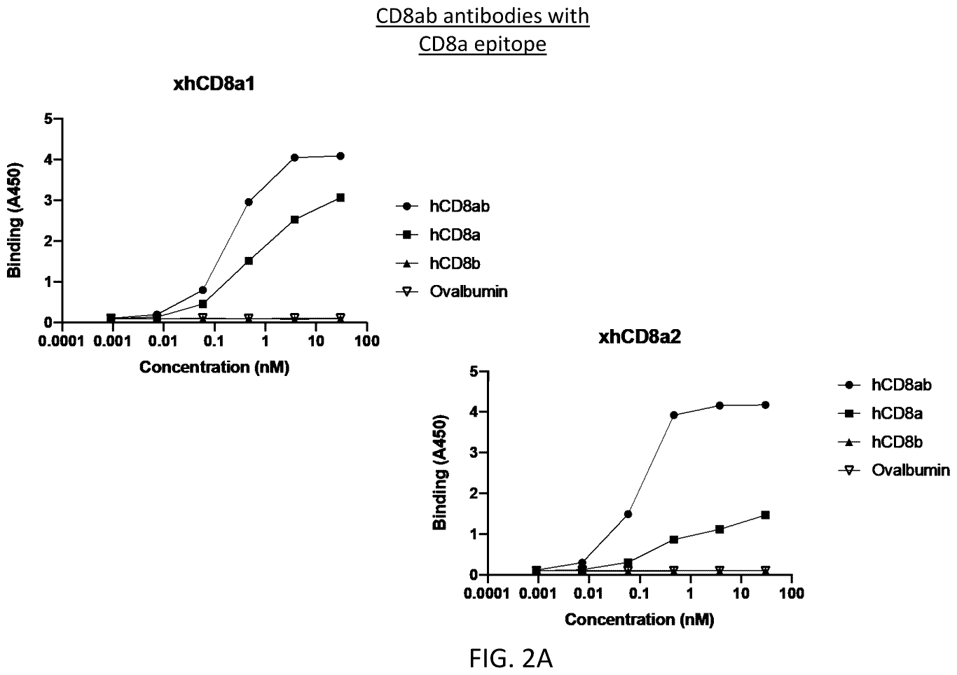

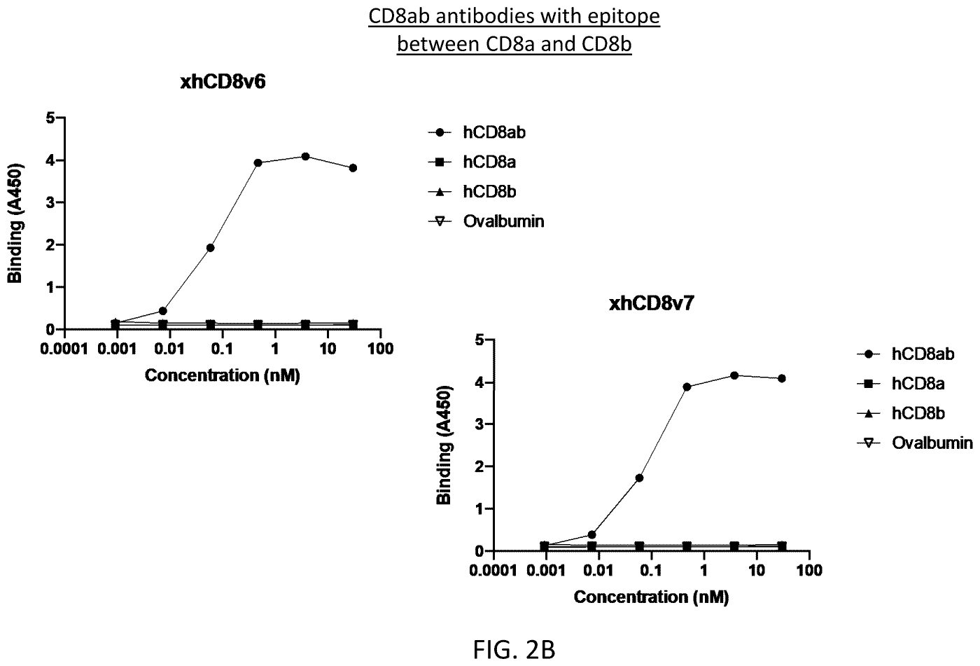

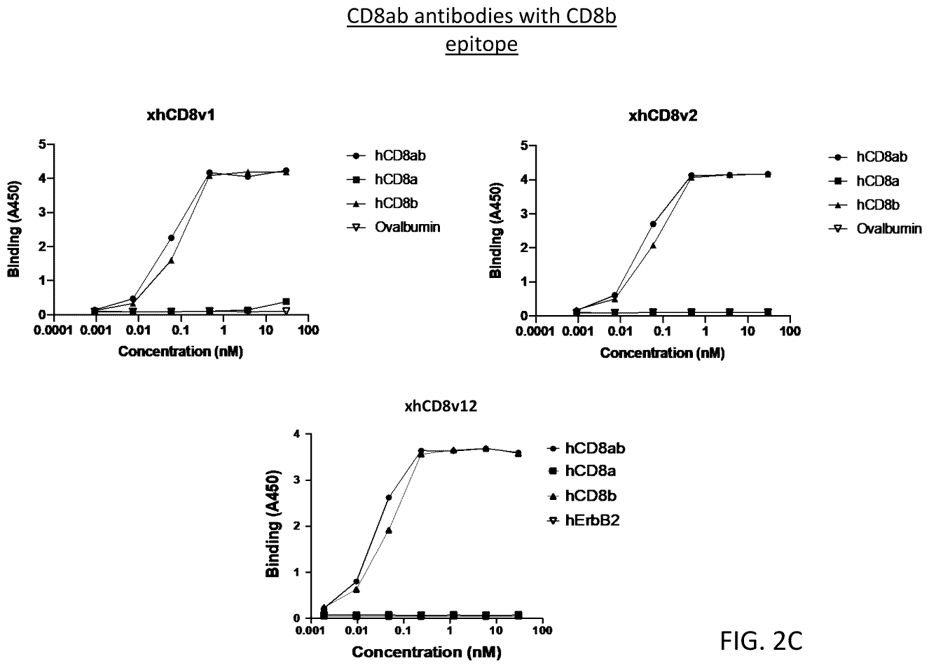

FIG. 2A shows two examples of antibodies with CD8a epitopes: xhCD8al (clone OKT8, Invitrogen) and xhCD8a2 (clone SK1, Biolegend), which both bind CD8a alone but not CD8b alone. Anti-CD8ab antibodies xhCD8v6 and xhCD8v7 both bind epitopes between CD8a and CD8b and do not bind to CD8a alone and CD8b alone (FIG. 2B). Mouse monoclonal antibody xhCD8v1 and its humanized variants, xhCD8v2 to xhCD8v5, xhCD8v9, xhCD8v12 and xhCD8v13 all bind to CD8b alone but not CD8 alone and therefore bind an epitope on the CD8b molecule (FIG. 2C).

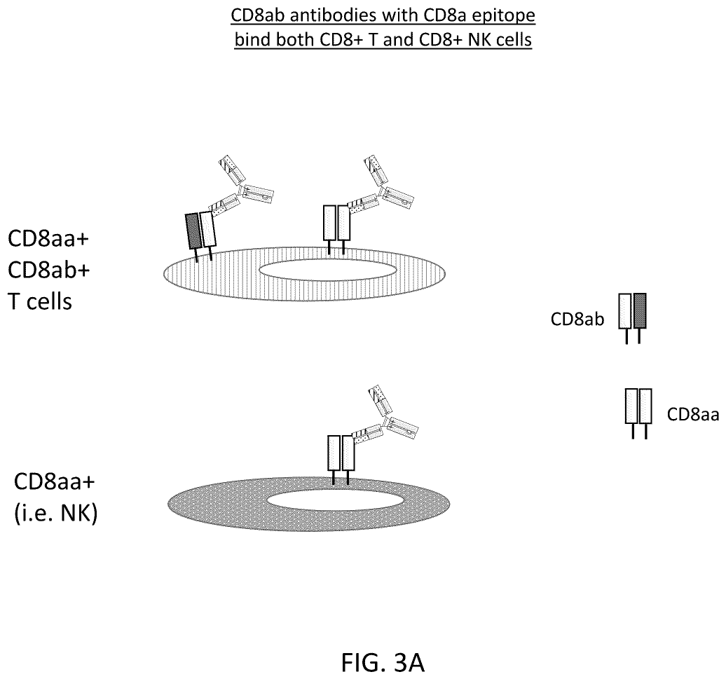

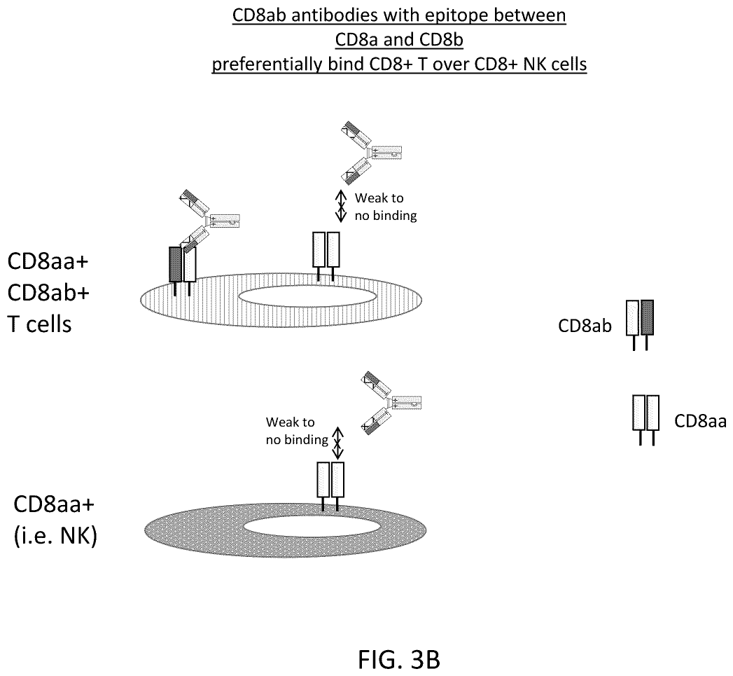

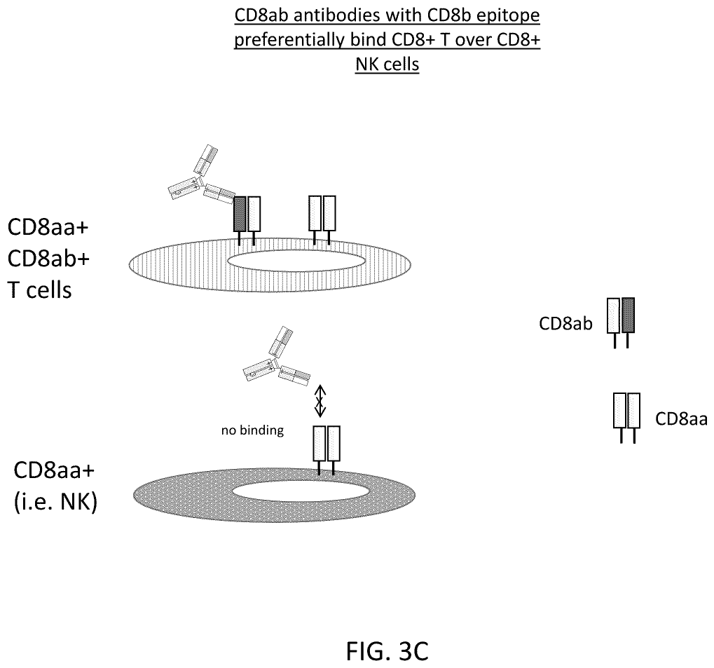

As depicted in FIGS. 3A-3C, the binding epitope for CD8ab antibodies determines their specificity for CD8+ T cells over CD8+NK cells. Certain immune cells, such as NK cells, express only CD8aa homodimers and will be recognized by antibodies depicted in FIG. 3A. T cells typically express both CD8aa homodimers and CD8ab heterodimers and will be recognized by all three types of antibodies depicted in FIGS. 3A-3C. However, antibodies depicted in FIG. 3B and FIG. 3C will only bind to CD8+ T cells and not CD8+NK cells and are therefore useful for distinguishing these two cell types.

The binding of CD8ab antibodies on cells was determined by flow cytometry. Freshly isolated PBMCs were incubated with CD8ab antibodies for 2 hours at 4° C. Cells were then stained with antibodies against surface markers CD3 (UCHT1), CD4 (RPA-T4), CD8a (SK1), CD56 (HCD56), and anti-human Fc antibody (HP6017). Anti-human Fc antibody was used to measure the binding of CD8ab antibodies containing hFc. Stained cells were washed and analyzed by flow cytometry and mean fluorescence intensity (MFI) of staining with anti-hFc was used to denote binding.

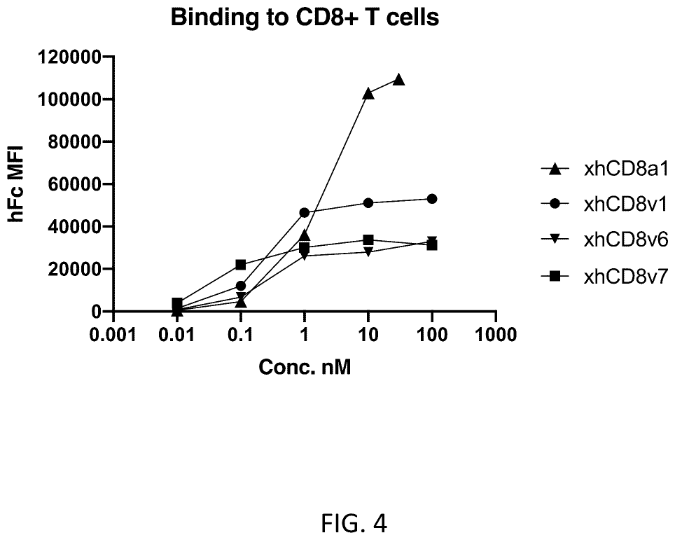

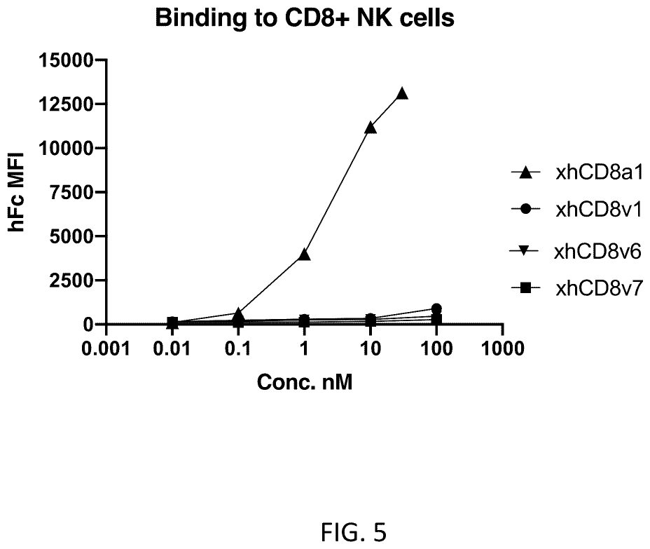

As depicted in FIGS. 4 & 5, xhCD8al antibody that recognizes an epitope on CD8a bound both CD8+ T cells and CD8+NK cells. CD8ab antibodies that recognized CD8ab epitopes or CD8b epitopes selectively bound to CD8+ T cells over CD8+NK cells.

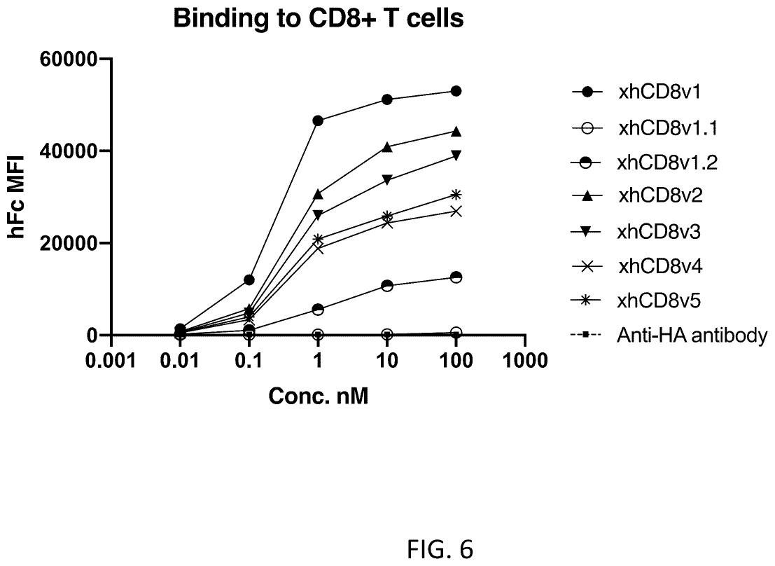

Next, anti-CD8 antibody xhCD8v1 was humanized and affinity matured. Surprisingly, when xhCD8v1 was grafted onto human framework to generate xhCD8v1.1, the binding to CD8+ T cells was lost (FIG. 6).

Affinity maturation of xhCD8v1.1 resulted in several new variants with increased binding to CD8+ T cells, xhCD8v2, xhCD8v3, xhCD8v4, and xhCD8v5.

### Example 2: Fusion Proteins Targeting CD8ab Heterodimer but not CD8aa Homodimer Selectively Activate Human CD8+ T Cells Over CD8+NK Cells

### Materials and Methods

### Cloning of Fusion Constructs

Antibody constructs were cloned as described in Example 1. The IL-2 portions of the constructs were cloned in frame with the heavy chain using a (G4S) 3 15-mer linker between the C-terminus of the IgG heavy chain and the N-terminus of IL-2. The C-terminal lysine residue of the IgG heavy chain was eliminated after fusing the IL-2 portion. To generate the construct in which a single IL-2 gene was fused to a full IgG, two heavy chain plasmids needed to be constructed and transfected for heterodimerization facilitated by a knob-into-hole modification in the IgG CH3 domains. The “hole” heavy chain connected to the IL-2 portion carried the Y349C, T366S, L368A and Y407V mutations in the CH3 domain, whereas the unfused “knob” heavy chain carried the S354C and T366W mutations in the CH3 domain (EU numbering). To abolish FcγR binding/effector function and prevent FcR co-activation, the following mutations were introduced into the CH2 domain of each of the IgG heavy chains: L234A/L235A/G237A (EU numbering). The expression of the antibody-IL-2 fusion constructs was driven by an CMV promoter and transcription terminated by a synthetic polyA signal sequence located downstream of the coding sequence.

Preparation of Fusion Proteins with IL-2 Polypeptides

Constructs encoding fusion proteins with IL-2 polypeptides as used in the examples were produced by co-transfecting exponentially growing Expi293 cells with the mammalian expression vectors using polyethylenimine (PEI). Supernatants were collected after 4-5 days of culture. IL-2 fusion constructs were first purified by affinity chromatography using a protein A matrix. The protein A column was equilibrated and washed in phosphate-buffered saline (PBS). The fusion constructs were eluted with 20 mM sodium citrate, 50 mM sodium chloride, pH 3.6. The eluted fractions were pooled and dialyzed into 10 mM MES, 25 mM sodium chloride pH 6. The proteins were further purified using ion-exchange chromatograph (Mono-S, GE Healthcare) to purify the heterodimers over the homodimers. After loading the protein, the column was washed with 10 mM MES 25 mM sodium chloride pH 6. The protein was then eluted with increasing gradient of sodium chloride from 25 mM up to 500 mM in 10 mM MES pH 6 buffer. The major eluent peak corresponding to the heterodimer was collected and concentrated. The purified protein was then polished by size exclusion chromatography (Superdex 200, GE Healthcare) in PBS.

The protein concentration of purified IL-2 fusion constructs was determined by measuring the optical density (OD) at 280 nm, using the molar extinction coefficient calculated on the basis of the amino acid sequence. Purity, integrity and monomeric state of the fusion constructs were analyzed by SDS-PAGE in the presence and absence of a reducing agent (5 mM 1,4-dithiothreitol) and stained with Coomassie blue (SimpleBlue™ SafeStain, Invitrogen). The NuPAGE® Pre-Cast gel system (Invitrogen) was used according to the manufacturer's instructions (4-20% Tris-glycine gels or 3-12% Bis-Tris). The aggregate content of immunoconjugate samples was analyzed using a Superdex 200 10/300 GL analytical size-exclusion column (GE Healthcare).

pSTAT5 and Ki-67 Assays to Measure Activation by IL-2 Fusion Proteins

Activity of IL-2 fusion proteins was determined in an assay with human PBMCs measuring the phosphorylation of STAT5. PBMCs were isolated from blood of healthy donors using Ficoll-Paque Plus (GE Healthcare) and red blood cells were lysed using ACK lysis buffer (Gibco) according to manufacturer's instructions. Typically, PBMCs were resuspended in serum-free RPMI1640 media at 20×10⁶ cells/ml and aliquoted into 96-well U-bottom plates (50 μl per well). IL-2 fusion proteins and control proteins, such as recombinant human IL-2 and control fusion proteins, were diluted to desired concentrations and added to wells (50 μl added as 2× stimulus). Incubation was typically performed for 30 min at 37° C., after which it was stopped with 100 μl pre-warmed 8% PFA (4% final) for 10 min at room temperature. Cells were washed 3× with wash buffer (2% FBS in PBS). Cells were permeabilized in pre-chilled Phosflow Perm buffer III (BD Biosciences) according to manufacturer's protocol and stored at −20° C. overnight. The next day cells were washed 3× with wash buffer and stained for 30-45 min at 4° C. with antibodies against: CD3 (UCHT1, BD Biosciences), CD8a (SK1, Biolegend; RPA-T8, Biolegend), CD4 (RPA-T4, Biolegend), and CD25 (M-A251, Biolegend), perforin (clone 8G9, BD Biosciences), Foxp3 (clone 259D, Biolegend), pSTAT5 [pY694] (clone 47, BD Biosciences). Cells were then analyzed on a flow cytometer. Data were expressed as percent pSTAT5 positive, and in some cases as pSTAT5 mean fluorescence intensity (MFI), and imported into GraphPad Prism.

To measure cellular changes induced by IL-2 fusion proteins further downstream from pSTAT5 such as proliferation, a flow cytometry assay was used to detect the expression of the intracellular proliferation marker Ki-67. Briefly, PBMCs were isolated as described above and resuspended at 1×10⁶ cells/ml serum-supplemented RPMI1640 (10% FBS) for 4 to 6 days or serum free AIM V media (Gibco). Cells were plated into 96-well U-bottom plates (150 μl per well) and incubated with 150 μl of IL-2 fusion proteins for five days at 37° C. On final day, cell surface staining was first performed by adding antibodies against: CD3 (UCHT1, BD Biosciences), CD8a (SK1, Biolegend), CD4 (RPA-T4, Biolegend), CD56 (HCD56, Biolegend) and CD25 (M-A251, Biolegend). Cells were washed 3× with wash buffer and intracellular staining performed using the Foxp3/Transcription Factor Staining Buffer Set (Thermo Fisher Scientific) according to manufacturer's protocol. Briefly, 1× Fix/Perm buffer was added to cells for 45 min at 4° C. in the dark. Cells were washed 3× with 1× Perm Wash buffer and stained for 45 min at 4° C. in dark with antibodies against intracellular markers Ki-67 (B56, BD Biosciences) and Foxp3 (259D, Biolegend). Cells were then washed 3× with Perm Wash buffer and analyzed on the flow cytometer.

### Results

Fusion proteins comprising the CD8ab antibodies and IL-2 polypeptides of the present disclosure were made in one of four formats (formats A, B,C, and D shown in FIG. 7).

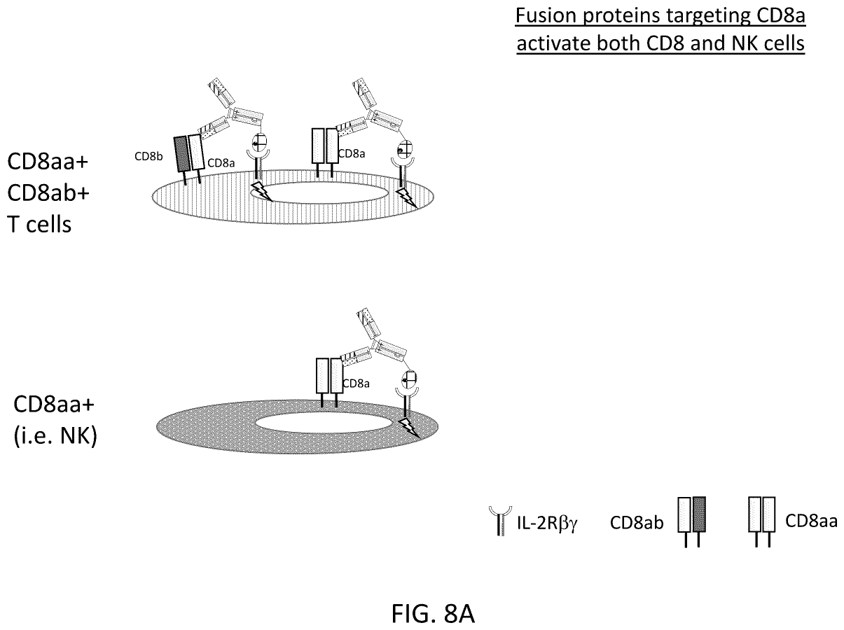

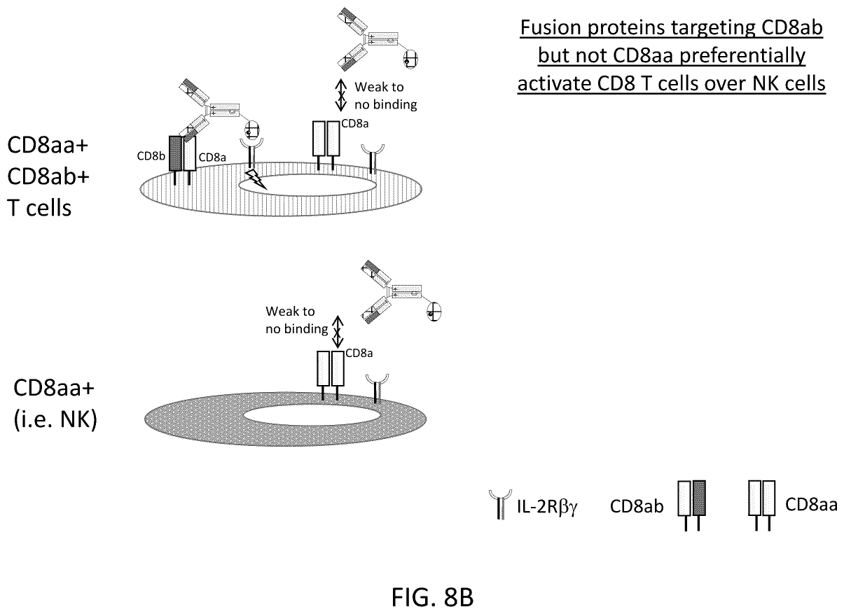

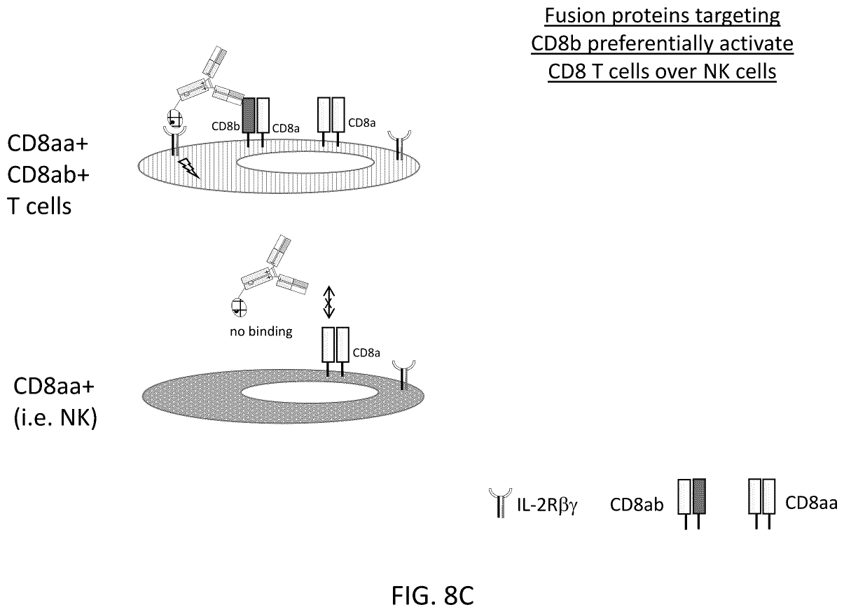

Fusion proteins comprising CD8ab antibodies targeting CD8a activate IL-2RBY signaling on both CD8+ T cells and CD8+NK cells (FIG. 8A). Fusion proteins comprising CD8ab antibodies targeting epitopes between CD8a and CD8b preferentially activate IL-2RBY signaling on CD8+ T cells over CD8+NK cells (FIG. 8B). Fusion proteins comprising CD8ab antibodies targeting CD8b preferentially activate IL-2Rβγ signaling on CD8+ T cells over CD8+NK cells (FIG. 8C).

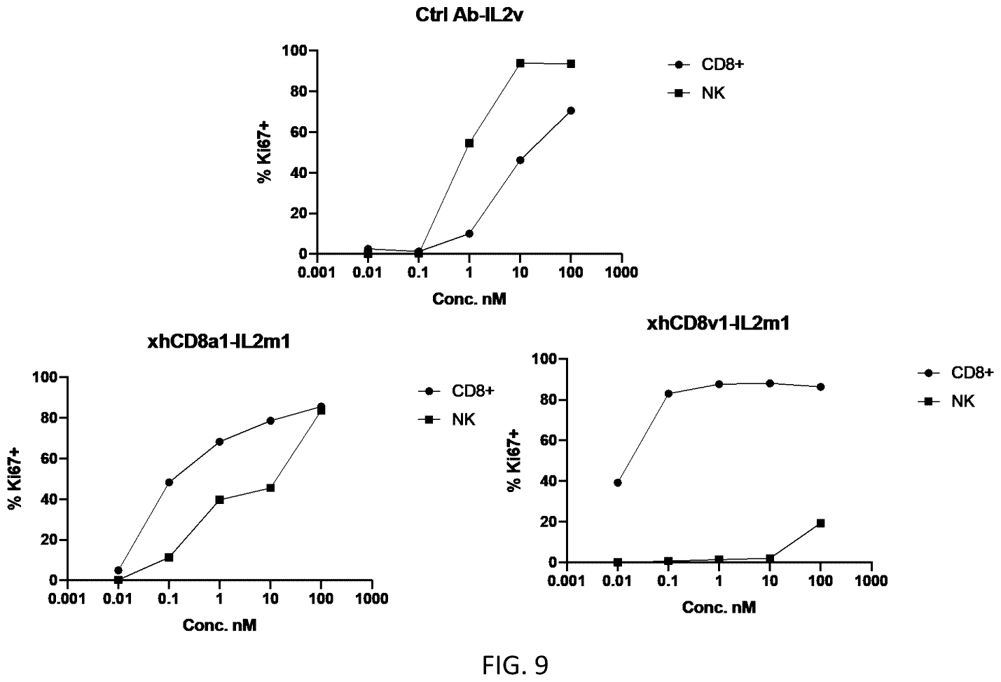

Because large proportion of NK cells can express CD8, fusion proteins comprising CD8ab antibodies not binding to CD8a epitopes also preferentially activated CD8+ T cells over total NK cells. In FIG. 9, the selectivity of different fusion proteins for human CD8+ T cells and NK cells was determined by measuring Ki-67 expression as described in Example 1. For a fusion protein comprising the control antibody and a previously published IL-2 variant (IL2v), NK cells were preferentially activated over CD8+ T cells. For a fusion protein comprising the CD8ab antibody targeting CD8a (xhCD8al) and IL-2 mutant polypeptide, IL2 ml, CD8+ T cells were preferentially activated over NK cells however only with a preference of ˜ 10x. Much stronger preference in activating CD8+ T cells was obtained by fusion proteins comprising CD8ab antibody targeting CD8b (xhCD8v1) and IL-2 mutant polypeptide, IL2 ml (>1000×).

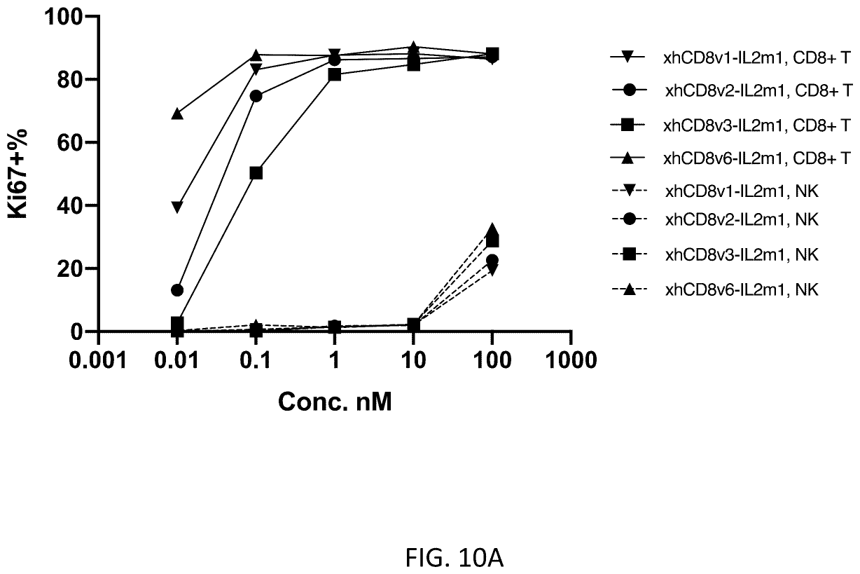

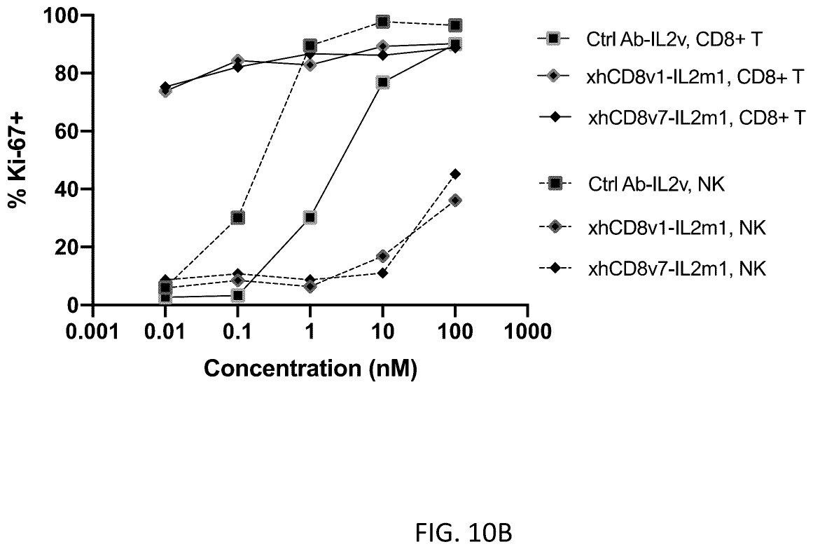

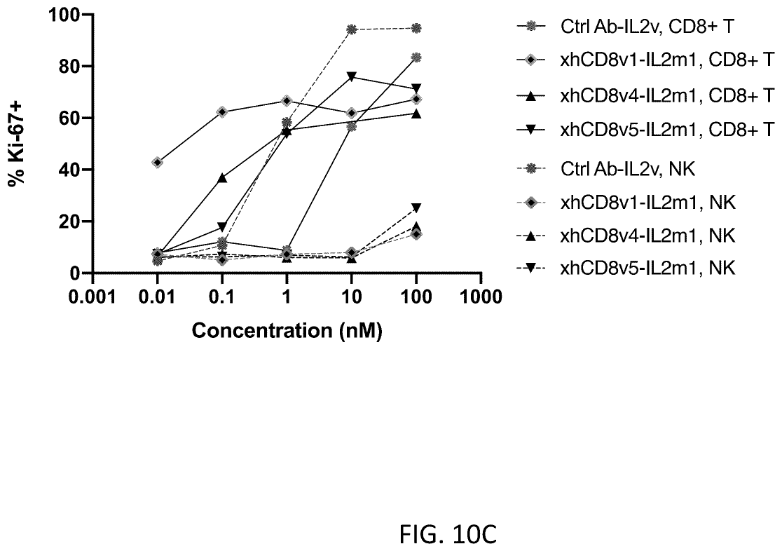

In additional examples shown in FIGS. 10A-10C, strong preference for activating Ki-67 proliferation marker on CD8+ T cells over NK cells was shown by other CD8ab antibodies targeting either epitopes between CD8a and CD8b, such as xhCD8v6 (FIG. 10A), and xhCD8v7 (FIG. 10B) or CD8b alone epitopes, such as xhCD8v2 to xhCD8v5 (FIG. 10A and FIG. 10C). For each of the tested fusion proteins comprising CD8ab antibodies, the preference for CD8 T cells over NK cells was >100× using Ki-67 assay as a readout.

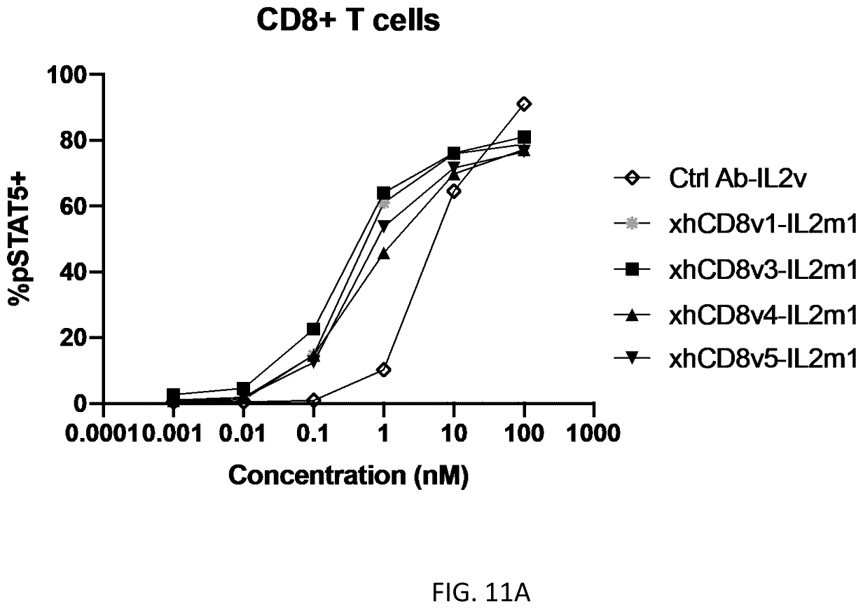

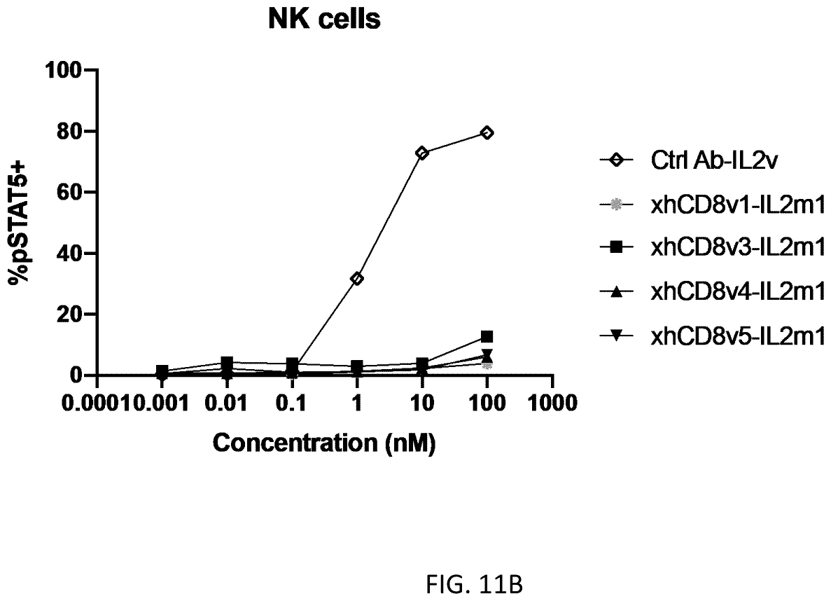

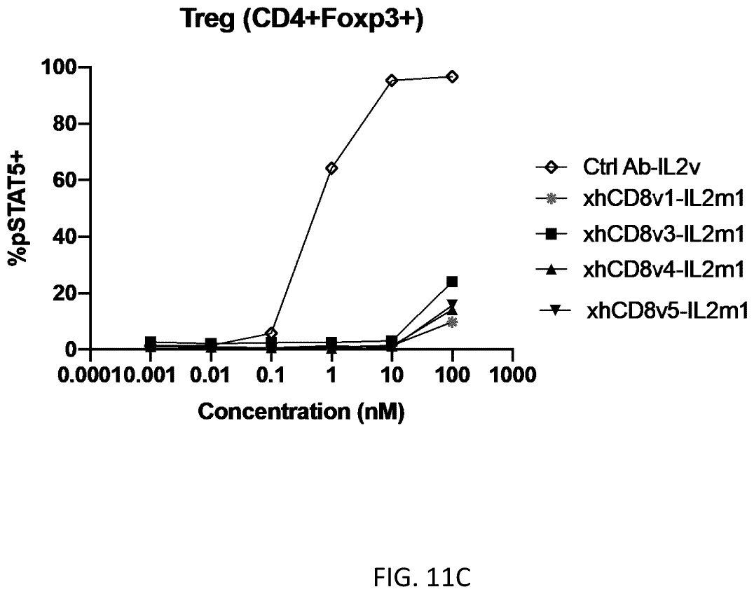

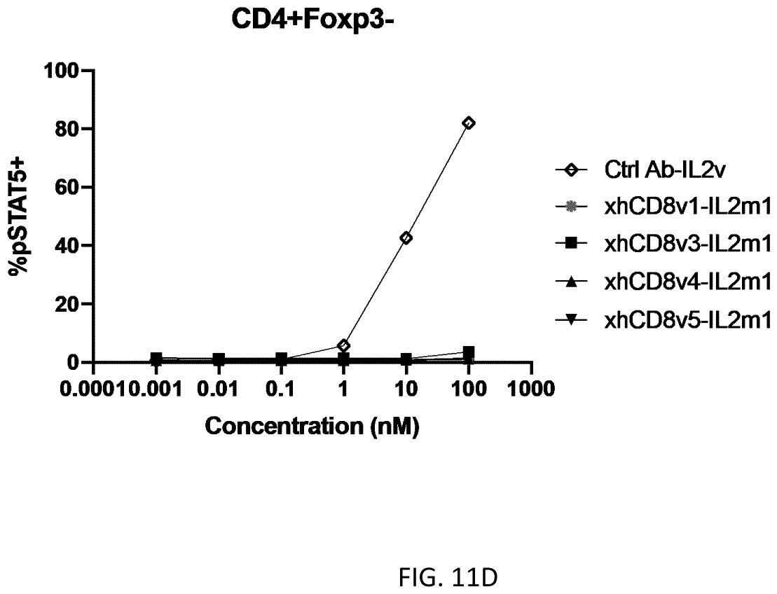

Similar preference for CD8+ T cells over NK cells was also demonstrated using pSTAT5 assay as shown in FIGS. 11A-11D. STAT5 activation was measured in different lymphocyte subsets from one human PBMC donor. Fusion protein Ctrl-IL2v indiscriminately activated CD8+ T cells, NK cells, Treg cells and T effector (CD4+Foxp3-) cells while fusion proteins comprising CD8ab antibodies of the present disclosure and mutant IL-2 polypeptide selectively activated CD8+ T cells over NK cells by at least 100×.

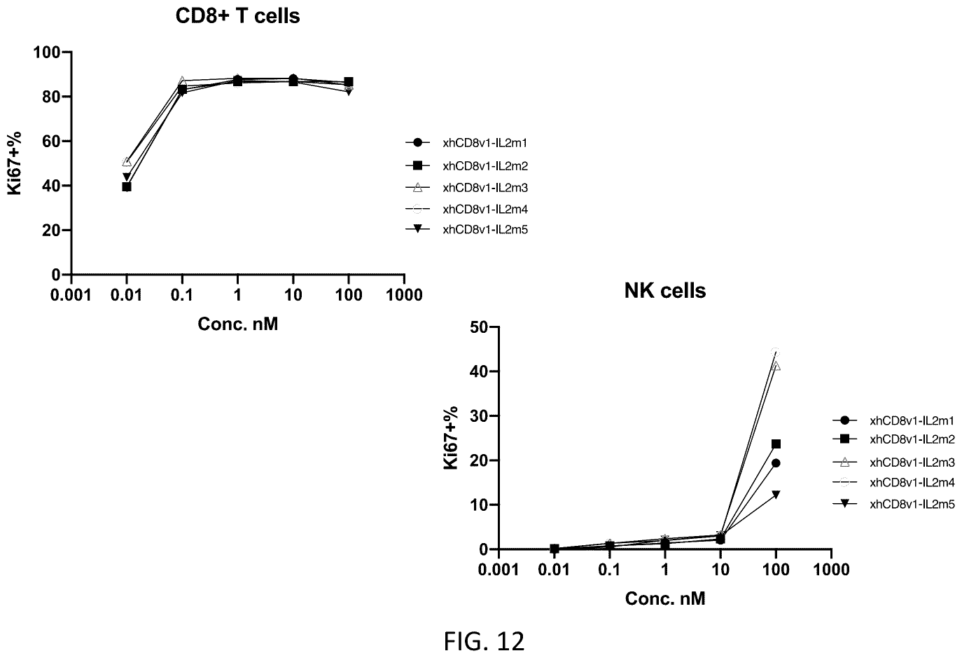

Ki67 activation in CD8+ T cell and NK cells was tested after incubation of hPBMCs from another donor with fusion proteins comprising the same xhCD8vl antibody and one of the following IL-2 polypeptides: IL2 ml, IL2m2, IL2m3, IL2m4 or IL2m5. IL-2 polypeptide sequences are shown in Table 7. As depicted in FIG. 12, all five fusion proteins achieved similar CD8+ T cell activation (EC₅₀˜0.01 nM), preferentially to NK cells with the following ranking for EC₅₀ for NK cells: IL2m3, IL2m4<IL2 ml, IL2m2<IL2m5.

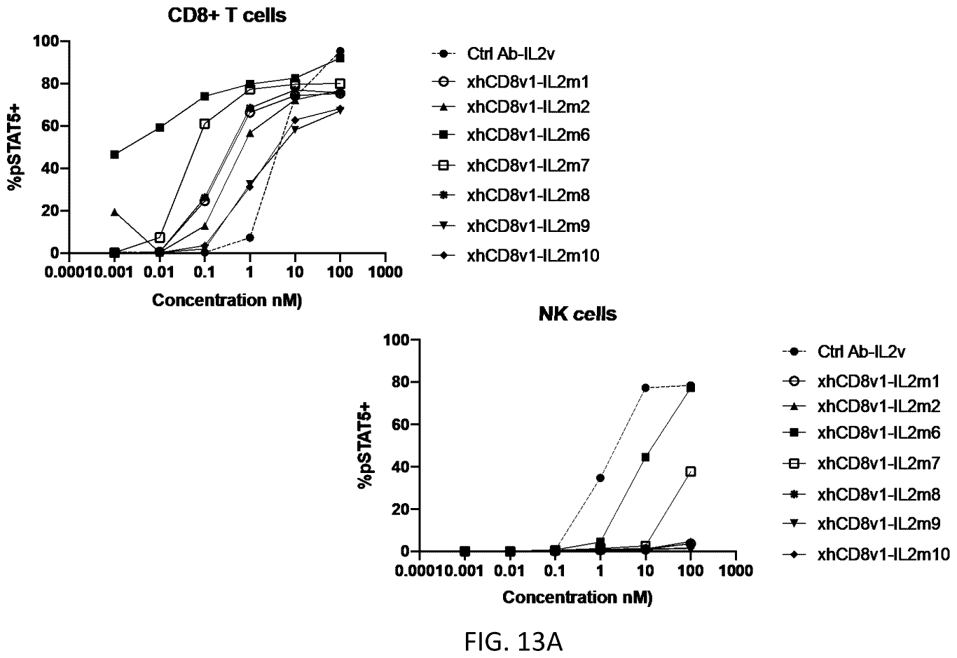

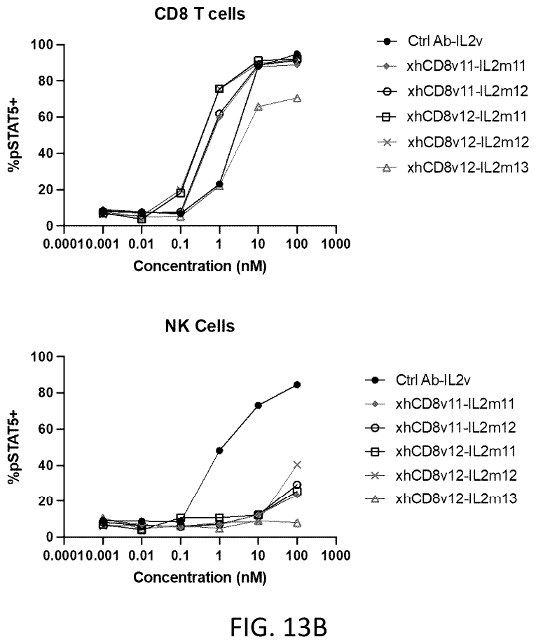

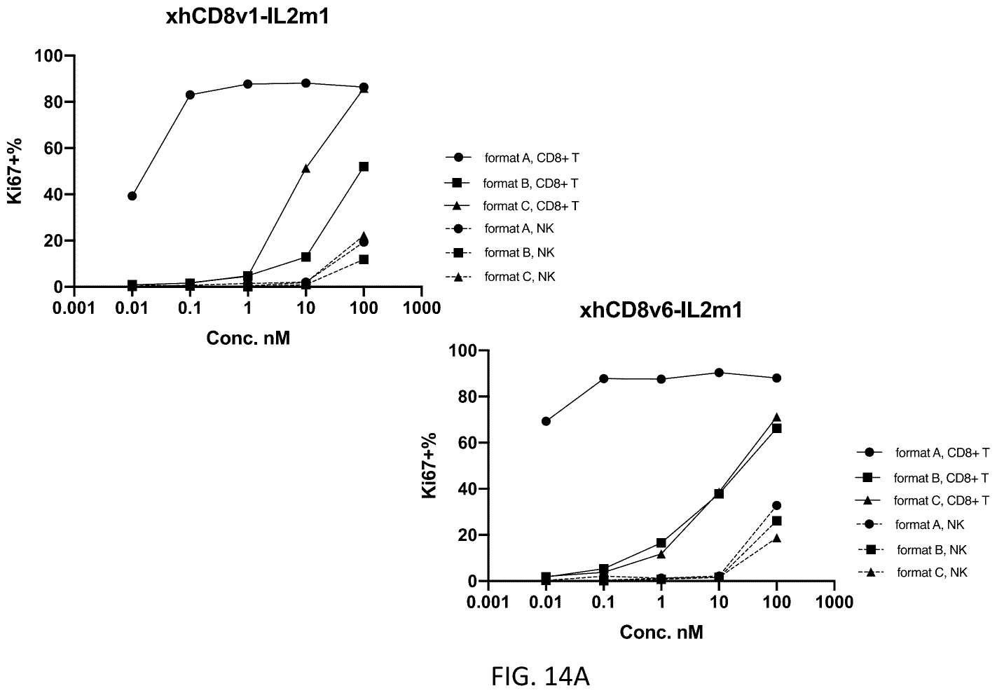

FIG. 13A shows STAT5 activation of CD8+ T cells and NK cells by fusion proteins containing the same xhCD8vl antibody and one of the following IL-2 polypeptides: IL2 ml, IL2m2, IL2m6, IL2m7, IL2m8, IL2m9 or IL2m10 (solid line) in comparison to the Ctrl Ab-IL2v (dashed line). All fusion proteins comprising xhCD8vl activated CD8+ T cells with lower EC50 than Ctrl Ab-IL2v and with the following ranking for EC50: IL2m6<IL2m7<IL2 ml, IL2m8<IL2m2<IL2m9, IL2m10. All fusion proteins comprising xhCD8v1 activated NK cells with higher EC50 than Ctrl Ab-IL2v. Except for xhCD8v1-IL2m6 and xhCD8v1-IL2m7, EC50 of STAT5 activation for NK cells was >100 nM. FIG. 13B shows the STAT5 activation of CD8+ T cells and NK cells by fusion proteins containing either xhCD8v11 or xhCD8v 12 antibody and one of the following IL-2 polypeptides: IL2m11, IL2m12, or IL2m13 in comparison to the Ctrl Ab-IL2v. All fusion proteins comprising either xhCD8v11 or xhCD8v 12 activated CD8+ T cells with lower EC50 than NK cells, and with the following ranking for EC50: IL2m12, IL2m11<IL2m13. All fusion proteins comprising xhCD8v11 or xhCD8v12 antibody activated NK cells with higher EC50 than Ctrl Ab-IL2v. EC50 of STAT5 activation for NK cells was >100 nM.

### Example 3: Effect of Fusion Protein Format on CD8+ T Cell Activation

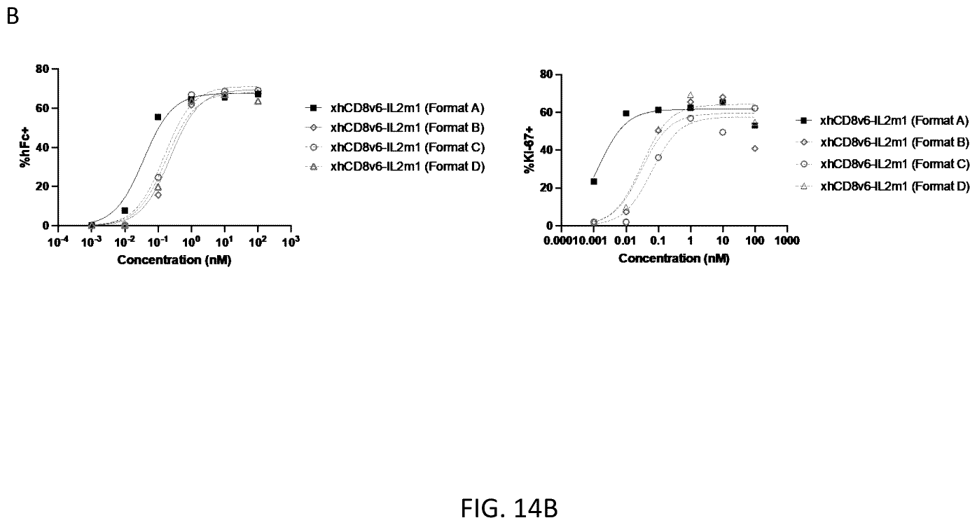

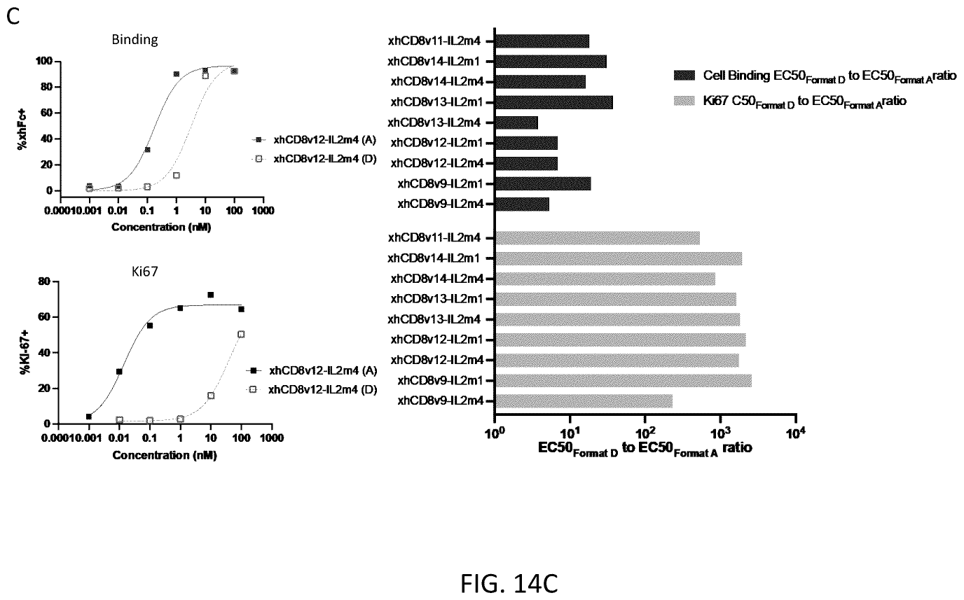

FIG. 14A depicts the impact of fusion protein format on preferential CD8+ T cell activation. Activation of CD8+ T cells (solid lines) and NK cells (dotted lines) by xhCD8v1-IL2 ml (left panel) and xhCD8v6-IL2 ml (right panel) in 3 different formats (A, B, C, see FIG. 7) as indicated in FIG. 14A legend are shown. Although all three formats activated CD8+ T cells preferentially over NK cells, format A resulted in the largest preference compared to format B and C. This suggests that format A is optimal for fusion proteins with CD8ab antibodies targeting epitopes between CD8a and CD8b or CD8b alone.

FIG. 14B depicts the impact of fusion protein format on preferential CD8+ T cell activation. The binding and the ability to induce Ki67 for xhCD8v6-IL2 ml in all four formats shown in FIGS. 7 (A, B, C, & D) were evaluated. Format A showed about 5-fold higher affinity in cell binding compared to other formats; this is likely due to the avidity-driven binding enhancement by format A. Surprisingly, compared to binding fold change, format A (black solid line) showed greater extent of Ki67 induction, ˜20- to 40-fold, over formats B, C, and D (grey dashed lines). Results here suggested that format A can preferentially and disproportionately induce a greater level of Ki-67 activation compared to other formats. FIG. 14C depicts the results of fusion molecules in format A and D as assessed for binding to CD8+ T cells, and induction of Ki67 in CD8+ T cells for a wide range of xhCD8 binders fused to IL2 ml and IL2m4. Due to the enhanced avidity mediated by bivalent binding, format A showed about 10-fold decrease in binding EC50s over format D for all fusions. Unexpectedly, format A showed a potency increase over format D of approximately 200-to over 1,000-fold in proliferation biomarker Ki-67 activation, beyond the 10-fold increase observed for binding. Therefore, results here supported that format A is more superior in inducing downstream proliferation, and hence the preferred format for fusion proteins containing CD8ab antibodies targeting epitopes between CD8a and CD8b (FIG. 8B) or CD8b epitopes (FIG. 8C).

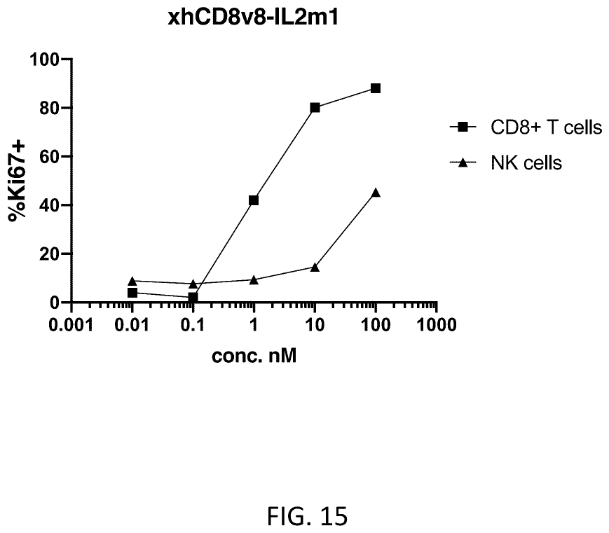

FIG. 15 depicts greater than 10 fold preferential Ki67 activation in CD8 T cells (filled squares) over NK cells (filled triangles) by the xCD8v8-IL2 ml fusion protein containing a CD8ab antibody xCD8v8.

### Example 4: Fusion Proteins of the Present Disclosure Selectively Activate and Enrich CD8+ T Cells in TILs from Human Cancer Patients

Single cell suspensions containing TILs were isolated from a tumor biopsy taken from a renal cell carcinoma patient using the Human Tumor Dissociation Kit (Miltenyi) according to manufacturer's protocol. Single cell suspensions were resuspended in complete RPMI media and plated at 1×10⁵ cells/well with the indicated fusion proteins in a total of 300 μl per well. After five days the cultures were analyzed for the expression of Ki-67 in various subsets and total cell counts per well determined by flow cytometry.

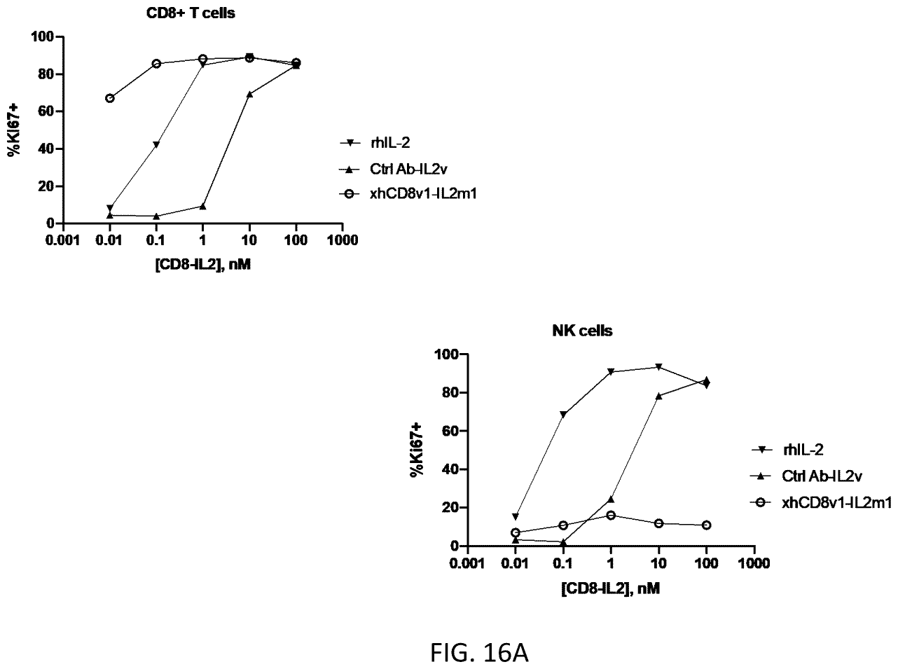

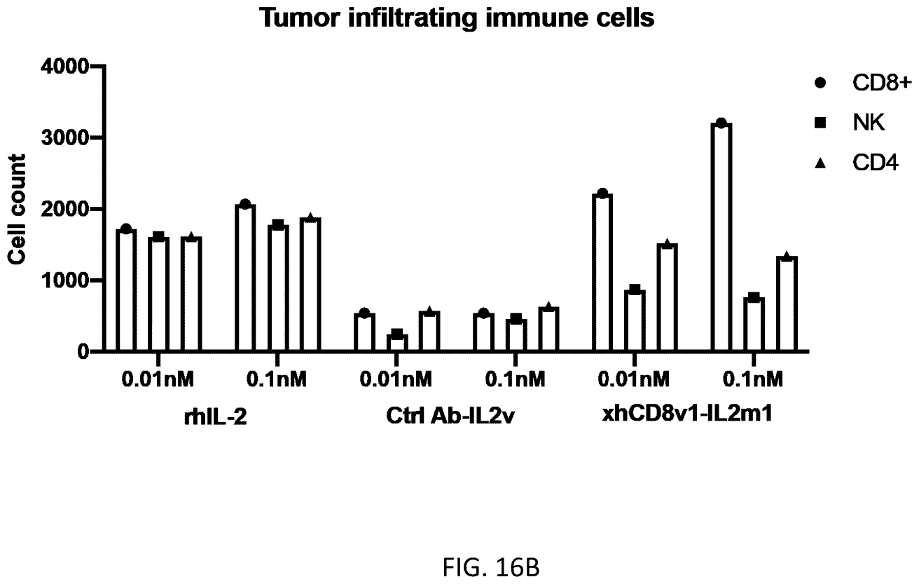

Ability of rhIL-2, Ctrl Ab-IL2v and xhCD8v1-IL2 ml to activate Ki-67 in CD8+ T cells and NK cells in isolated single cell suspensions from renal cell carcinoma was determined (FIG. 16A). Results showed that xhCD8v1-IL2 ml more potently activated Ki-67 in CD8+ T cells than rhIL-2 and Ctrl-IL2v with no activation of Ki-67 in NK cells. Furthermore, CD8+ T cell counts were preferentially increased compared to NK cell and CD4+ T cell counts (FIG. 16B).

By extension of these findings, one application of fusion proteins with CD8ab antibodies targeting is to enhance selective CD8+ T cell expansion in TILs isolated from cancer patients for the purpose of reinjecting them into patients for treatment.

Another application is to use fusion proteins with CD8ab antibodies in combination with TIL therapy or other T cell therapy, in particular TCR-T cell therapy with MHC class I-restricted T cells.

### Example 5: Testing Additional CD8 Antibodies Targeting CD8ab Heterodimer for Selective Activation of Human CD8+ T Cells Over CD8+NK Cells

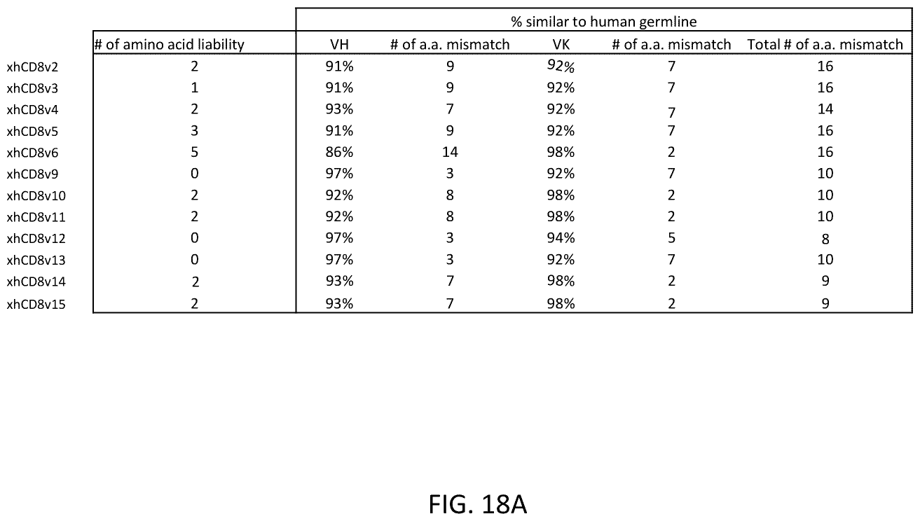

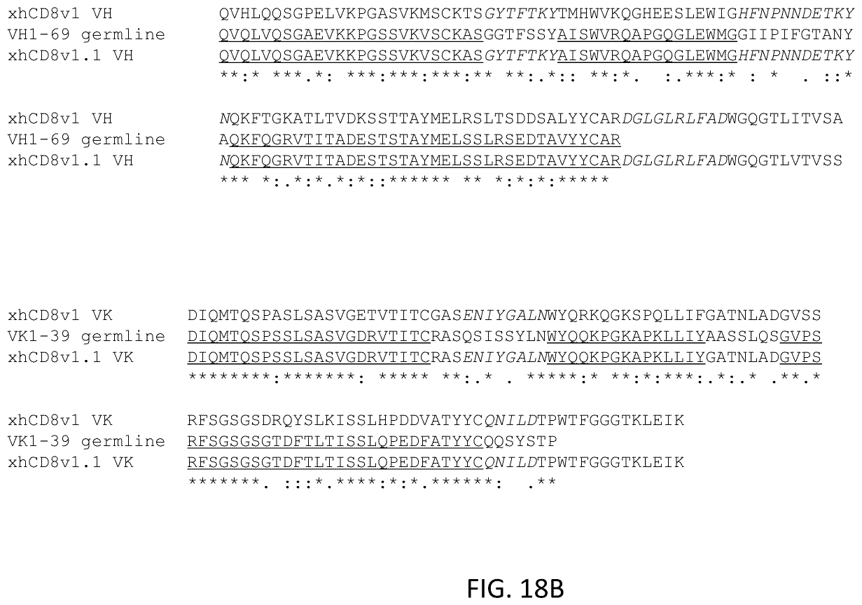

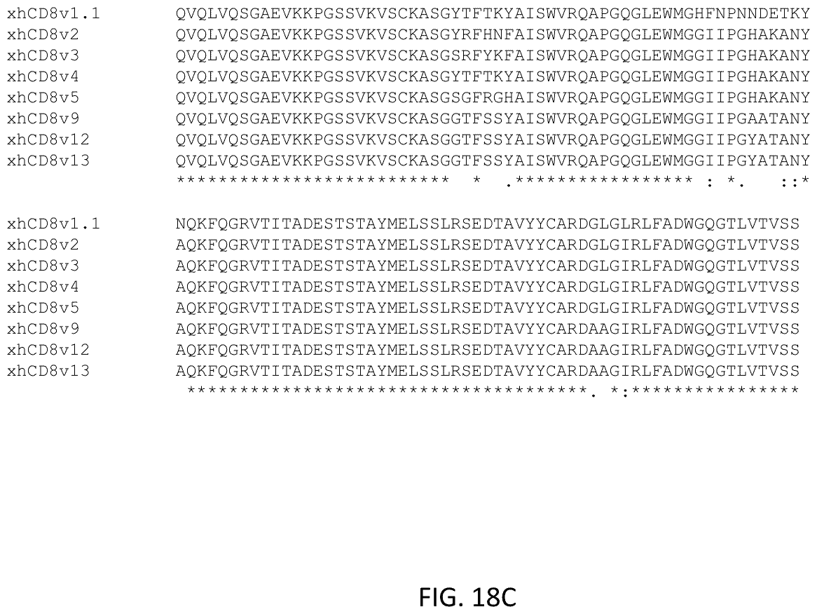

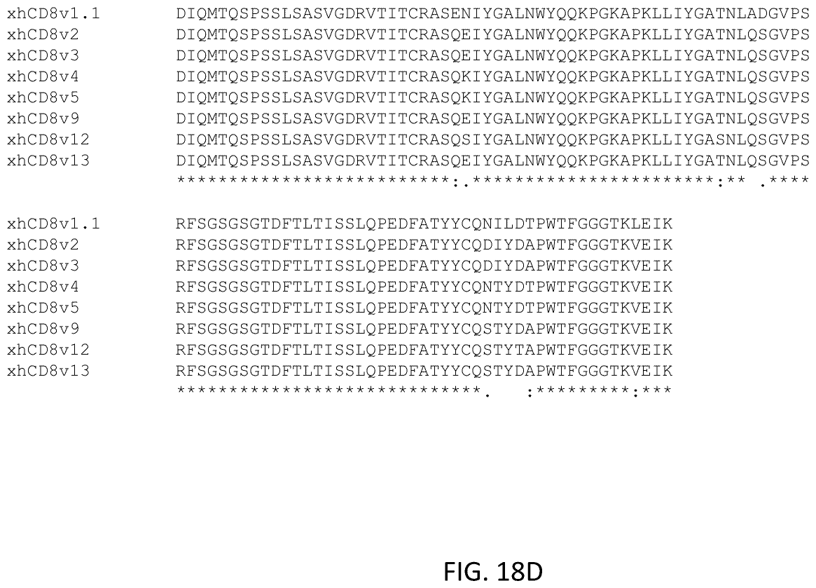

Additional CD8ab antibodies xhCD8v9-v15 were generated and tested for binding to recombinant CD8ab as described in Example 1. These antibodies were designed as improved versions (e.g., v9, v12, and v13 based on v2, and v10, v11, v14, and v15 based on v6) that (1) reduce the number of potential amino acid liabilities (e.g., putative N-linked glycosylation, deamination, or acid cleavage sites), potentially improving manufacturability; and (2) reduce the number of amino acid mismatches to human germline, potentially reducing immunogenicity in vivo (FIG. 18A). As noted above, when anti-CD8 antibody xhCD8v1 was grafted onto human framework to generate xhCD8v1.1 (as depicted in FIG. 18B), the binding to CD8+ T cells was surprisingly lost (FIG. 6). Further substitutions were needed in order to restore binding in the context of a humanized antibody, and yet further substitutions were introduced in order to reduce potential liabilities and/or mismatches to human germline (FIGS. 18C & 18D).

As shown in Table 6, all additional antibodies xhCD8v9-v15 showed high-affinity binding to CD8ab.

TABLE 6
Biosensor data of new CD8ab antibody binding.

| | | | K_{D} | K_{D} |
|---|---|---|---|---|
| | ka (1/Ms) | kd (1/s) | (M) | (nM) |
| xhCD8v8 | — | — | 2.36E− | 236.0* |
| | | | 07* | |
| xhCD8v9 | 2.25E+05 | 2.73E−02 | 1.21E− | 121.0 |
| | | | 07 | |
| xhCD8v10 | 1.06E+05 | 3.86E−03 | 3.64E− | 36.4 |
| | | | 08 | |
| xhCD8v11 | 9.55E+04 | 3.61E−03 | 3.78E− | 37.8 |
| | | | 08 | |
| xhCD8v12 | 2.59E+05 | 2.71E−02 | 1.05E− | 105.0 |
| | | | 07 | |
| xhCD8v13 | 3.68E+05 | 3.01E−02 | 8.18E− | 81.8 |
| | | | 08 | |
| xhCD8v14 | 1.62E+05 | 8.55E−03 | 5.29E− | 52.9 |
| | | | 08 | |
| xhCD8v15 | 1.86E+05 | 1.75E−02 | 9.43E− | 94.3 |
| | | | 08 | |

*determined by steady state fit

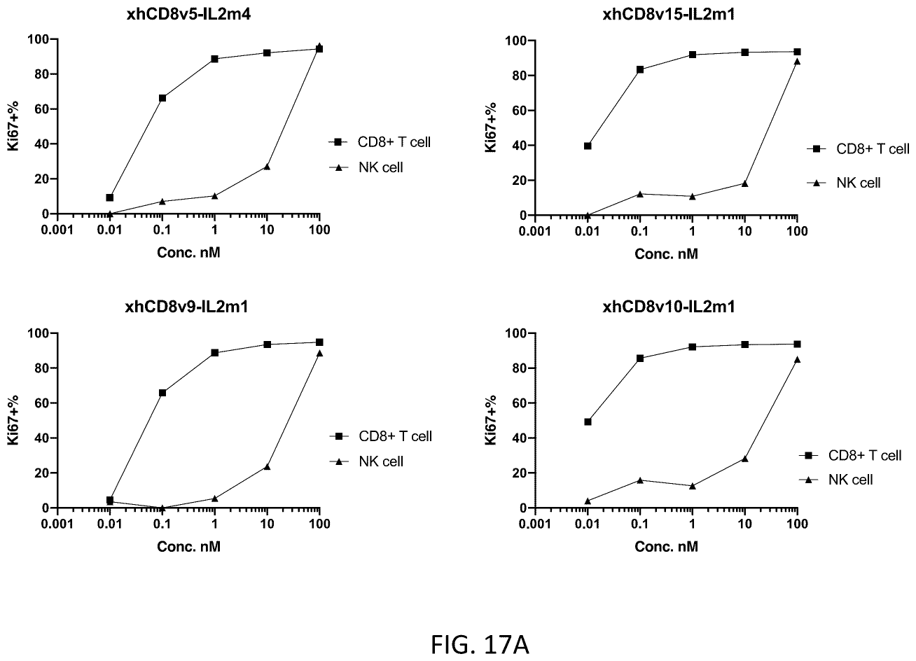

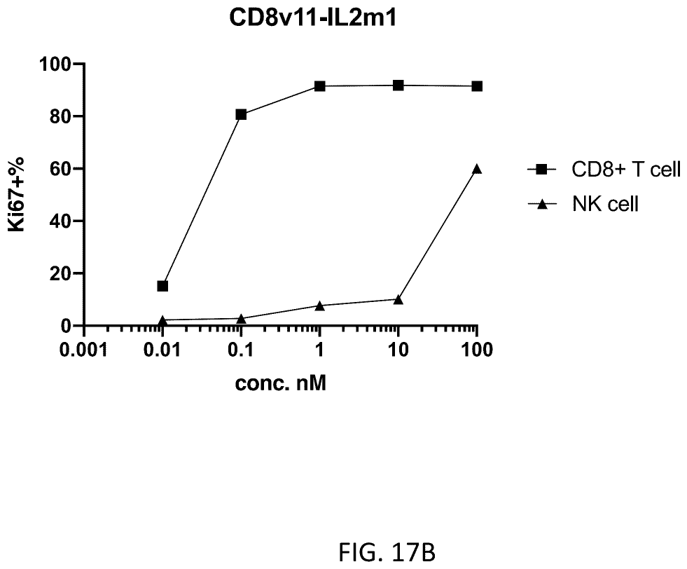

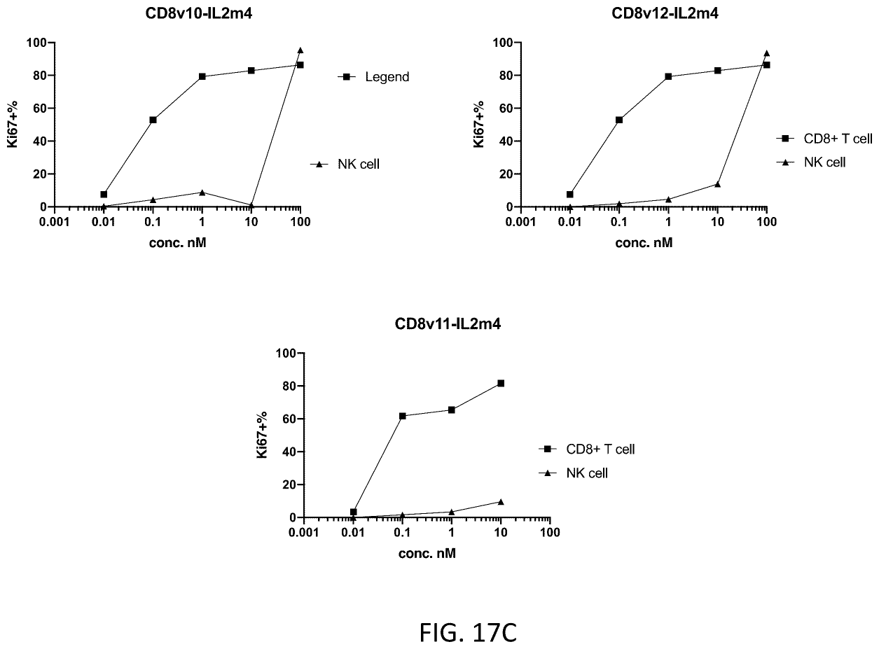

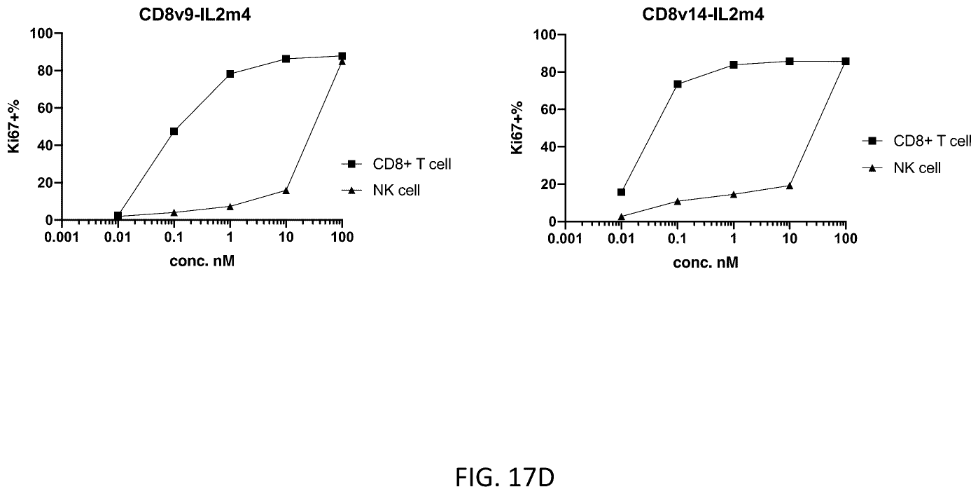

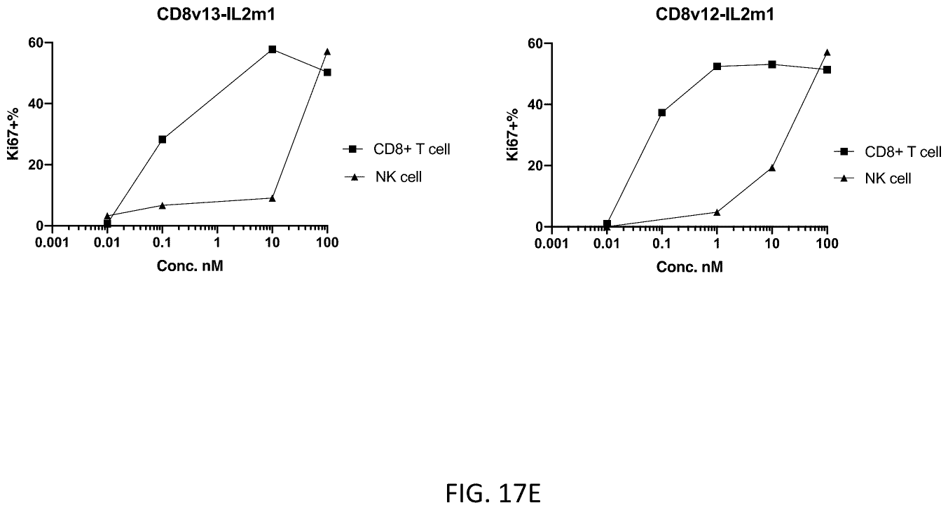

These antibodies were also tested as fusion proteins in format A (as shown in FIG. 7) with a mutant IL-2 polypeptide (IL2 ml or IL2m4) for ability to selectively activate human CD8+ T cells over CD8+NK cells, as described above in Example 2. As shown in FIGS. 17A-17E, these antibodies were able to promote selective activation of human CD8+ T cells over CD8+NK cells when used in the context of a fusion protein with a mutant IL-2 polypeptide.

### Example 6: Thermal Stability Assessment of xhCD8v2-Related Family Clones

### Material and Methods

### Forced Degradation Assay

To assess molecular stability and aggregation propensity, purified antibodies at a concentration of 1 mg/mL in PBS were stressed at temperatures from 4° C. to 64° C. for 24 hours. Samples were filtered and analyzed by analytical size exclusion chromatography on a Superdex 200 5/150 GL (Cytiva). The percent monomer and monomer area were calculated using OpenLab ChemStation (Agilent Technologies). Experiments were performed in duplicate.

### Thermal Stability Measurements

To determine thermal stability characteristics, intrinsic differential scanning fluorimetry (DSF) and static light scattering (SLS) were measured on a UNit instrument (Unchained labs). Purified antibodies at a concentration of 1 mg/mL in PBS were subject to a thermal ramp from 25° C.-95° C. at a rate of 1° C./min. UV spectra were recorded for DSF and SLS was recorded at 266 nm. Samples were performed in quadruplicate. Melting temperature (Tm), and aggregation temperature (Tagg) were analyzed and calculated by UNcle Analysis Software V3.2 (Unchained labs).

### Results

Improved Thermal Stability of xhCD8v9, xhCD8v12 and xhCD8v13 Compared to Parental xhCD8v2

Parental antibody xhCD8v2 and its derivative xhCD8v9, xhCD8v12 and xhCD8v13 were subjected to forced degradation and thermal stability assays. Results for the forced degradation assay, depicted as the % recovery of monomer (the ratio of the monomer area at 64° C. to 4° C.) for the different antibodies, are shown in Table 7. xhCD8v9, xhCD8v12 and xhCD8v13 antibodies have greater % recovery compared to parental antibody xhCD8v2. Thermal stability measurements, shown in in Table 7, revealed that xhCD8v9, xhCD8v12 and xhCD8v13 antibodies have higher Tm and Tagg values than that of parental antibody xhCD8v2. Taken together, these results demonstrate that xhCD8v9, xhCD8v12 and xhCD8v13 molecules have improved thermal stability over the parental antibody xhCD8v2 and therefore, xhCD8v9, xhCD8v12 and xhCD8v13 are more desirable as therapeutic agents.

TABLE 7
Results of Forced Degradation and Thermostability Assays

| | Forced | Thermostability | |
|---|---|---|---|
| | Degradation | | Tagg |
| Construct | % Recovery | Tm (° C.) | (° C.) |
| xhCD8v2 | 6.3 | 67.4 ± 0.2 | 66.4 ± 0.4 |
| xhCD8v9 | 85.5 | 68.9 ± 0.1 | 74.7 ± 0.1 |
| xhCD8v12 | 78.5 | 68.8 ± 0.2 | 70.9 ± 0.8 |
| xhCD8v13 | 76.7 | 69.2 ± 0.2 | 73.7 ± 0.3 |

### Example 7: Assessment of Glycan Occupancy on Parental xhCD8v6 and its Derviatives

### Materials and Methods

### Deglycosylation of Antibodies

To remove both N-linked and O-linked glycosylation, 10 μg of antibody was mixed with 1 μl of 10× GlycoBuffer 2 (New England Biolabs), 1 μl Protein Deglycosylation Mix II (New England Biolabs) and brought to 10 μl with water. Reactions were incubated overnight at room temperate.

### ELISA for Detection of Glycosylation of the P3F4-Related Molecule

Presence of N-linked glycosylation was determined by ELISA. Briefly, 25 μL of protein from 0.5 to 10 μg/mL was coated onto a 384-well Nunc MaxiSorp plate (Thermo Fisher Scientific) and incubated at 37° C. for 1 hour. Proteins were removed and the plate was washed with PBS+0.05% Tween-20 (PBST). Wells were filled with carbohydrate-free blocking solution (Vector Labs) and incubated at room temperature for 30 mins. The blocking solution was removed, and the wells washed with PBST. 25 μL of 5 μg/mL biotinylated wheat germ agglutinin (Vector Labs), which binds to glycoconjugates, in PBS was added and incubated at room temperature for 30 mins. It was then removed, and the plate was washed again with PBST. 25 μL of the detection reagent, VECTASTAIN Elite ABC-HRP Reagent, Peroxidase, R.T.U. (Vector Labs), was added and allowed to incubate for 30 mins at room temperature. The reagent was removed, and wells were washed with PBST. Wells were developed using 25 L of KPL SureBlue TMB Microwell Substrate (SeraCare) for 5-7 mins and quenched with 25 μL of 0.1 M HCl. The absorbance at 450 nm was recorded on a SpectraMax iD5 plate reader (Molecular Devices). Fetuin, a glycoprotein containing sialylated N-linked and O-linked glycans, was used as a positive control. All experiments were performed with n=8.

### Results

Removal of the N-Linked Glycosylation Site on xhCD8v6 Improves Homogeneity

xCD8v6 possesses a putative N-linked glycosylation motif in the CDR-H2 region. This putative N-linked glycosylation motif was removed in the derivatives of xhCD8v6, namely xhCD8v10, xhCD8v11, xhCD8v14, and xhCD8v15. Presence of variable amount of glycans in the CDR region decreases homogeneity of the final product and could affect binding capability.

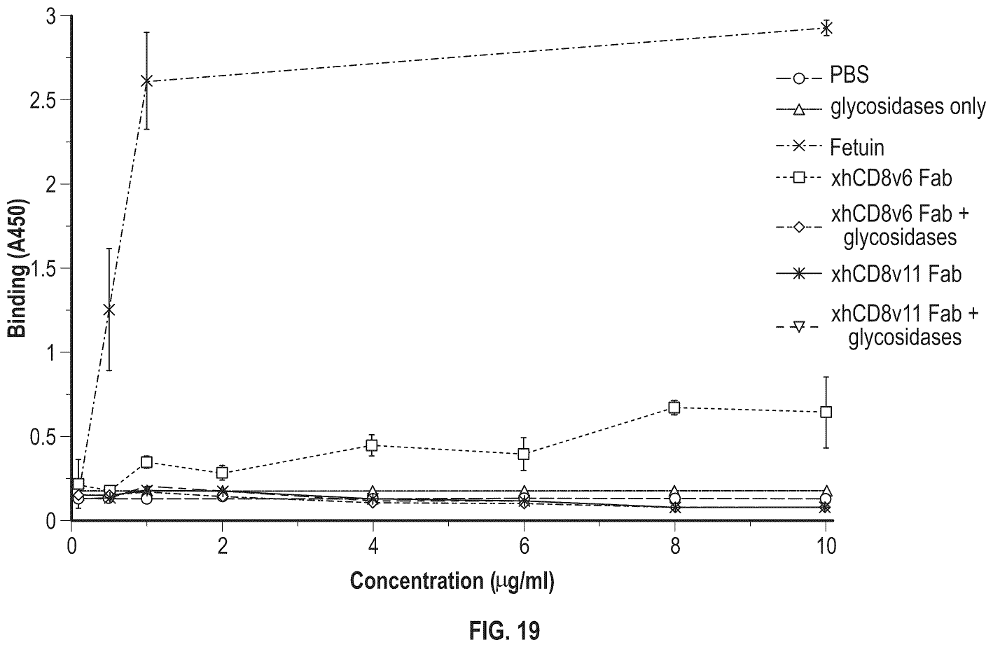

To probe for the presence of glycans, an ELISA using wheat germ agglutinin (WGA), which binds to N-acetylglucosamine, was performed. Fabs of parental xhCD8v6 and xhCD8v11, which served as the negative control, were assayed to examine only the variable domains. Fetuin was used as a positive control. As shown in FIG. 19, WGA binding was detected for the parental xhCD8v6, but not xhCD8v11. Upon treatment with deglycosidases, there was no binding of WGA to deglycosylated xhCD8v6, confirming the presence of glycan on xhCD8v6. Results here suggested that xhCD8v10, xhCD8v11, xhCD8v14, and xhCD8v15, which do not possess the putative N-linked glycosylation motif, are better and more homogenous therapeutics agents compared to parental xCD8v6.

1-53. (canceled)

54. A fusion protein, comprising:
(a) a first moiety comprising a human antibody or antigen-binding fragment thereof that specifically binds CD8b, wherein the antibody or fragment comprises a heavy chain variable (VH) domain and a light chain variable (VL) domain, wherein:
the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; and
(b) a second moiety comprising an IL-2 polypeptide;
wherein the first moiety is fused to the second moiety directly or via a linker.

55. (canceled)

56. The fusion protein of claim 54, wherein the IL-2 polypeptide induces activation of CD8+ T cells.

57. The fusion protein of claim 56, wherein the human antibody or antigen-binding fragment thereof of the first moiety further specifically binds CD8ab, and wherein the fusion protein induces activation of cells expressing a human CD8ab heterodimer with at least 10-fold higher potency than activation of cells expressing a human CD8aa homodimer.

58. The fusion protein of claim 56, wherein the fusion protein induces activation of CD8+ T cells with at least 10-fold higher potency than activation of NK cells.

59. The fusion protein of claim 58, wherein potency of activation is measured by EC50, as assessed by cell proliferation.

60. The fusion protein of claim 54, wherein the first moiety comprises two antibody heavy chain polypeptides comprising a structure according to formula [I], from N-terminus to C-terminus:
VH—CH1-hinge-CH2-CH3 [I]
and two antibody light chain polypeptides comprising a structure according to formula [II], from N-terminus to C-terminus:
VL-CL [II]
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the second moiety is fused to the C-terminus of one of the two CH3 domains.

61. The fusion protein of claim 54, wherein the first moiety comprises a first antibody heavy chain polypeptide comprising a structure according to formula [I], from N-terminus to C-terminus:
VH—CH1-hinge-CH2-CH3 [I],
an antibody light chain polypeptide comprising a structure according to formula [II], from N-terminus to C-terminus:
VL-CL [II],
and a second antibody heavy chain polypeptide comprising a structure according to formula [III], from N-terminus to C-terminus:
hinge-CH2-CH3 [III],
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the second moiety is fused to the C-terminus of the CH3 domain of the second antibody heavy chain polypeptide.

62. The fusion protein of claim 54, wherein the first moiety comprises a first antibody heavy chain polypeptide comprising a structure according to formula [I], from N-terminus to C-terminus:
VH—CH1-hinge-CH2-CH3 [I],
an antibody light chain polypeptide comprising a structure according to formula [II], from N-terminus to C-terminus:
VL-CL [II],
and a second antibody heavy chain polypeptide comprising a structure according to formula [III], from N-terminus to C-terminus:
hinge-CH2-CH3 [III],
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the C-terminus of the second moiety is fused to the N-terminus of the hinge domain of the second antibody heavy chain polypeptide.

63. The fusion protein of claim 54, wherein the first moiety comprises a first antibody heavy chain polypeptide comprising a structure according to formula [I], from N-terminus to C-terminus:
VH—CH1-hinge-CH2-CH3 [I],
an antibody light chain polypeptide comprising a structure according to formula [II], from N-terminus to C-terminus:
VL-CL [II],
and a second antibody heavy chain polypeptide comprising a structure according to formula [III], from N-terminus to C-terminus:
hinge-CH2-CH3 [III],
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the second moiety is fused to the C-terminus of the CH3 domain of the first antibody heavy chain polypeptide.

64-65. (canceled)

66. The fusion protein of claim 60, wherein:
the VH domain of both antibody heavy chain polypeptides comprises an amino acid sequence that is at least 90% identical to the sequence of SEQ ID NO:249, and wherein the VL domain of both antibody light chain polypeptides comprises an amino acid sequence that is at least 90% identical to the sequence of SEQ ID NO:250.

67-68. (canceled)

69. The fusion protein of claim 61, wherein:
the VH domain comprises the amino acid sequence of SEQ ID NO:249, and the VL domain comprises the amino acid sequence of SEQ ID NO:250.

70. The fusion protein of claim 60, wherein one or both of the antibody heavy chain polypeptides comprise(s) the following amino acid substitutions: L234A, L235A, and G237A, numbering according to EU index.

71. The fusion protein of claim 60, wherein a first of the antibody heavy chain polypeptides comprises amino acid substitutions Y349C and T366W, and a second of the antibody heavy chain polypeptides comprises amino acid substitutions S354C, T366S, L368A and Y407V, numbering according to EU index.

72. The fusion protein of claim 54, wherein the first moiety comprises one or two antibody heavy chain polypeptides and one or two antibody light chain polypeptides.

73-75. (canceled)

76. The fusion protein of claim 54, wherein the IL-2 polypeptide is a mutant IL-2 polypeptide comprising one or more mutations relative to a human IL-2 polypeptide comprising the sequence of
(SEQ ID NO: 81)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT.

77. The fusion protein of claim 76, wherein the mutant IL-2 polypeptide has a binding affinity to IL-2Rα that is reduced by 50% or more, compared to binding affinity of a wild-type IL-2 polypeptide comprising the sequence of SEQ ID: 81 for IL-2Rα.

78. The fusion protein of claim 77, wherein the mutant IL-2 polypeptide has a binding affinity to IL-2Rβ that is reduced by 50% or more, compared to binding affinity of a wild-type IL-2 polypeptide comprising the sequence of SEQ ID: 81 for IL-2Rβ; and/or
wherein the mutant IL-2 polypeptide has a binding affinity to IL-2Rγ that is reduced by 50% or more, compared to binding affinity of a wild-type IL-2 polypeptide comprising the sequence of SEQ ID: 81 for IL-2Ry.

79. The fusion protein of claim 54, wherein the IL-2 polypeptide comprises the sequence of SEQ ID NO:81 with one, two, three, four, or five amino acid substitutions relative to SEQ ID NO:81, and wherein the one, two, three, four, or five substitution(s) comprise substitution(s) at positions of SEQ ID NO:81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130.

80. The fusion protein of claim 79, wherein the IL-2 polypeptide comprises the sequence of SEQ ID NO:81 with one of the following sets of amino acid substitutions: R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R.

81. The fusion protein of claim 79, wherein the IL-2 polypeptide comprises the sequence of SEQ ID NO:81 with a further amino acid substitution relative to SEQ ID NO:81 at position C125.

82. The fusion protein of claim 81, wherein the IL-2 polypeptide comprises the sequence of SEQ ID NO:81 with one of the following sets of amino acid substitutions: R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A;F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

83. The fusion protein of claim 54, wherein the IL-2 polypeptide comprises the sequence
(SEQ ID NO: 80)
APTSSSTKKTQLQLEHLLLDLQMILNGINNYKNPKLTEMLTAKFYMPKKAT
ELKHLQCLEEELKPLEEVLNLAQSKNFHLRPRDLISAINVIVLELKGSETT
FMCEYADETATIVEFLNRWITFAQSIISTLT
or the sequence
(SEQ ID NO: 297)
APTSSSTKKTQLQLEELLLDLQMILNGINNYKNPKLTEMLTAKFYMPKKAT
ELKHLQCLEEELKPLEEVLNLAQSKNFHLRPRDLISNINVIVLELKGSETT
FMCEYADETATIVEFLNRWITFAQSIISTLT.

84. The fusion protein of claim 54, wherein the IL-2 polypeptide comprises a sequence selected from the group consisting of SEQ ID NOs: 80, 85-155, 190-216, 297, and 354-383.

85-86. (canceled)

87. The fusion protein of claim 60, wherein one or both of the antibody Fc domains comprise(s) human IgG1 Fc domains with the following amino acid substitutions: L234A, L235A, and G237A, numbering according to EU index.

88. The fusion protein of claim 60, wherein a first of the two Fc domains comprises a human IgG1 Fc domain with amino acid substitutions Y349C and T366W, and a second of the two Fc domain comprises a human IgG1 Fc domain with amino acid substitutions S354C, T366S, L368A and Y407V, numbering according to EU index.

89. The fusion protein of claim 54, wherein the linker comprises the sequence (GGGS)xGn (SEQ ID NO:74), (GGGGS)xGn (SEQ ID NO:75), or (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3.

90. The fusion protein of claim 54, wherein the linker comprises the sequence GGGGSGGGGSGGGGS (SEQ ID NO:79) or SGGGGSGGGGSGGGGS (SEQ ID NO: 389).

91. The fusion protein of claim 54, wherein the fusion protein comprises:
one or two light chains comprising the amino acid sequence of SEQ ID NO:306, a heavy chain comprising the amino acid sequence of SEQ ID NO:307, and a heavy chain comprising the amino acid sequence of SEQ ID NO:308; or
one or two light chains comprising the amino acid sequence of SEQ ID NO:334, a heavy chain comprising the amino acid sequence of SEQ ID NO:335, and a heavy chain comprising the amino acid sequence of SEQ ID NO:336.

92. A fusion protein comprising a first moiety that binds to a human CD8b and a second moiety comprising an IL2 polypeptide, wherein the fusion protein comprises four polypeptide chains, wherein:
the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:336, and the fourth polypeptide chain comprises the amino acid sequence of SEQ ID NO:334; or
the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:337, and the fourth polypeptide chain comprises the amino acid sequence of SEQ ID NO:334.

93. One or more polynucleotides encoding the fusion protein according to claim 54.

94. One or more vectors comprising the one or more polynucleotides of claim 93.

95. (canceled)

96. An isolated host cell comprising the one or more polynucleotides of claim 93.

97. A method of producing a fusion protein, comprising culturing the host cell of claim 96 under conditions suitable for production of the fusion protein.

98. (canceled)

99. A pharmaceutical composition comprising the fusion protein according to claim 54 and a pharmaceutically acceptable carrier.

100-105. (canceled)

106. A method of treating infection comprising administering to an individual in need thereof an effective amount of the composition of claim 99.

107. The method of claim 106, wherein the infection is a viral infection.

108-110. (canceled)

111. The fusion protein of claim 66, wherein the VH domain of both antibody heavy chain polypeptides comprises the amino acid sequence of SEQ ID NO:249, and the VL domain of both antibody light chain polypeptides comprises the amino acid sequence of SEQ ID NO: 250.

112. The fusion protein of claim 62, wherein the VH domain comprises the amino acid sequence of SEQ ID NO:249, and the VL domain comprises the amino acid sequence of SEQ ID NO: 250.

113. The fusion protein of claim 63, wherein the VH domain comprises the amino acid sequence of SEQ ID NO:249, and the VL domain comprises the amino acid sequence of SEQ ID NO: 250.

114. A fusion protein comprising a first moiety that binds to a human CD8b and a second moiety comprising an IL2 polypeptide, wherein the fusion protein comprises four polypeptide chains, wherein:
the first polypeptide chain comprises the amino acid sequence of SEQ ID NO:306, the second polypeptide chain comprises the amino acid sequence of SEQ ID NO:307, the third polypeptide chain comprises the amino acid sequence of SEQ ID NO:308, and the fourth polypeptide chain comprises the amino acid sequence of SEQ ID NO:306; or
the first polypeptide chain comprises the amino acid sequence of SEQ ID NO:306, the second polypeptide chain comprises the amino acid sequence of SEQ ID NO:307, the third polypeptide chain comprises the amino acid sequence of SEQ ID NO:309, and the fourth polypeptide chain comprises the amino acid sequence of SEQ ID NO:306.

115. A pharmaceutical composition comprising the fusion protein according to claim 60 and a pharmaceutically acceptable carrier.

116. A pharmaceutical composition comprising the fusion protein according to claim 111 and a pharmaceutically acceptable carrier.

117. A pharmaceutical composition comprising the fusion protein according to claim 92 and a pharmaceutically acceptable carrier.

118. A pharmaceutical composition comprising the fusion protein according to claim 114 and a pharmaceutically acceptable carrier.

119. The fusion protein of claim 92, wherein the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:336, and the fourth polypeptide chain comprises the amino acid sequence of SEQ ID NO: 334.

120. The fusion protein of claim 92, wherein the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:337, and the fourth polypeptide chain comprises the amino acid sequence of SEQ ID NO: 334.

121. The fusion protein of claim 114, wherein the first polypeptide chain comprises the amino acid sequence of SEQ ID NO:306, the second polypeptide chain comprises the amino acid sequence of SEQ ID NO:307, the third polypeptide chain comprises the amino acid sequence of SEQ ID NO:308, and the fourth polypeptide chain comprises the amino acid sequence of SEQ ID NO: 306.

122. The fusion protein of claim 114, wherein the first polypeptide chain comprises the amino acid sequence of SEQ ID NO:306, the second polypeptide chain comprises the amino acid sequence of SEQ ID NO:307, the third polypeptide chain comprises the amino acid sequence of SEQ ID NO:309, and the fourth polypeptide chain comprises the amino acid sequence of SEQ ID NO: 306.

123. A fusion protein, comprising:
(a) a first moiety comprising a human antibody or antigen-binding fragment thereof that specifically binds CD8b, wherein the antibody or fragment comprises a heavy chain variable (VH) domain and a light chain variable (VL) domain, wherein the VH domain comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; and
(b) a second moiety comprising an IL-2 polypeptide;
wherein the first moiety is fused to the second moiety directly or via a linker.

124. The fusion protein of claim 87, wherein one or both of the antibody Fc domains do not have a C-terminal lysine.

125. The fusion protein of claim 88, wherein one or both of the antibody Fc domains do not have a C-terminal lysine.

126. One or more polynucleotides encoding the fusion protein according to claim 60.

127. One or more vectors comprising the one or more polynucleotides of claim 126.

128. An isolated host cell comprising the one or more polynucleotides of claim 126.

129. A method of producing a fusion protein, comprising culturing the host cell of claim 128 under conditions suitable for production of the fusion protein.

130. A method of treating infection comprising administering to an individual in need thereof an effective amount of the composition of claim 115.

131. The method of claim 130, wherein the infection is a viral infection.

132. One or more polynucleotides encoding the fusion protein according to claim 92.

133. One or more vectors comprising the one or more polynucleotides of claim 132.

134. An isolated host cell comprising the one or more polynucleotides of claim 132.

135. A method of producing a fusion protein, comprising culturing the host cell of claim 134 under conditions suitable for production of the fusion protein.

136. A method of treating infection comprising administering to an individual in need thereof an effective amount of the composition of claim 117.

137. The method of claim 136, wherein the infection is a viral infection.

138. One or more polynucleotides encoding the fusion protein according to claim 111.

139. One or more vectors comprising the one or more polynucleotides of claim 138.

140. An isolated host cell comprising the one or more polynucleotides of claim 138.

141. A method of producing a fusion protein, comprising culturing the host cell of claim 140 under conditions suitable for production of the fusion protein.

142. A method of treating infection comprising administering to an individual in need thereof an effective amount of the composition of claim 116.

143. The method of claim 142, wherein the infection is a viral infection.

144. One or more polynucleotides encoding the fusion protein according to claim 114.

145. One or more vectors comprising the one or more polynucleotides of claim 144.

146. An isolated host cell comprising the one or more polynucleotides of claim 144.

147. A method of producing a fusion protein, comprising culturing the host cell of claim 146 under conditions suitable for production of the fusion protein.

148. A method of treating infection comprising administering to an individual in need thereof an effective amount of the composition of claim 118.

149. The method of claim 148, wherein the infection is a viral infection.


SEQUENCE LISTING (389 sequences)

SEQ ID NO: 1 - protein, 5 aa, synthetic construct
  REGION 1..5: note = Synthetic Construct
KYTMH

SEQ ID NO: 2 - protein, 17 aa, synthetic construct
  REGION 1..17: note = Synthetic Construct
HFNPNNDETKYNQKFTG

SEQ ID NO: 3 - protein, 10 aa, synthetic construct
  REGION 1..10: note = Synthetic Construct
DGLGLRLFAD

SEQ ID NO: 4 - protein, 11 aa, synthetic construct
  REGION 1..11: note = Synthetic Construct
GASENIYGALN

SEQ ID NO: 5 - protein, 7 aa, synthetic construct
  REGION 1..7: note = Synthetic Construct
GATNLAD

SEQ ID NO: 6 - protein, 9 aa, synthetic construct
  REGION 1..9: note = Synthetic Construct
QNILDTPWT

SEQ ID NO: 7 - protein, 5 aa, synthetic construct
  REGION 1..5: note = Synthetic Construct
KYAIS

SEQ ID NO: 8 - protein, 17 aa, synthetic construct
  REGION 1..17: note = Synthetic Construct
HFNPNNDETKYNQKFQG

SEQ ID NO: 9 - protein, 10 aa, synthetic construct
  REGION 1..10: note = Synthetic Construct
DGLGLRLFAD

SEQ ID NO: 10 - protein, 11 aa, synthetic construct
  REGION 1..11: note = Synthetic Construct
RASENIYGALN

SEQ ID NO: 11 - protein, 7 aa, synthetic construct
  REGION 1..7: note = Synthetic Construct
GATNLAD

SEQ ID NO: 12 - protein, 9 aa, synthetic construct
  REGION 1..9: note = Synthetic Construct
QNILDTPWT

SEQ ID NO: 13 - protein, 5 aa, synthetic construct
  REGION 1..5: note = Synthetic Construct
NFAIS

SEQ ID NO: 14 - protein, 17 aa, synthetic construct
  REGION 1..17: note = Synthetic Construct
GIIPGHAKANYAQKFQG

SEQ ID NO: 15 - protein, 10 aa, synthetic construct
  REGION 1..10: note = Synthetic Construct
DGLGIRLFAD

SEQ ID NO: 16 - protein, 11 aa, synthetic construct
  REGION 1..11: note = Synthetic Construct
RASQEIYGALN

SEQ ID NO: 17 - protein, 7 aa, synthetic construct
  REGION 1..7: note = Synthetic Construct
GATNLQS

SEQ ID NO: 18 - protein, 9 aa, synthetic construct
  REGION 1..9: note = Synthetic Construct
QDIYDAPWT

SEQ ID NO: 19 - protein, 5 aa, synthetic construct
  REGION 1..5: note = Synthetic Construct
KFAIS

SEQ ID NO: 20 - protein, 17 aa, synthetic construct
  REGION 1..17: note = Synthetic Construct
GIIPGHAKANYAQKFQG

SEQ ID NO: 21 - protein, 10 aa, synthetic construct
  REGION 1..10: note = Synthetic Construct
DGLGIRLFAD

SEQ ID NO: 22 - protein, 11 aa, synthetic construct
  REGION 1..11: note = Synthetic Construct
RASQEIYGALN

SEQ ID NO: 23 - protein, 7 aa, synthetic construct
  REGION 1..7: note = Synthetic Construct
GATNLQS

SEQ ID NO: 24 - protein, 9 aa, synthetic construct
  REGION 1..9: note = Synthetic Construct
QDIYDAPWT

SEQ ID NO: 25 - protein, 5 aa, synthetic construct
  REGION 1..5: note = Synthetic Construct
KYAIS

SEQ ID NO: 26 - protein, 17 aa, synthetic construct
  REGION 1..17: note = Synthetic Construct
GIIPGHAKANYAQKFQG

SEQ ID NO: 27 - protein, 10 aa, synthetic construct
  REGION 1..10: note = Synthetic Construct
DGLGIRLFAD

SEQ ID NO: 28 - protein, 11 aa, synthetic construct
  REGION 1..11: note = Synthetic Construct
RASQKIYGALN

SEQ ID NO: 29 - protein, 7 aa, synthetic construct
  REGION 1..7: note = Synthetic Construct
GATNLQS

SEQ ID NO: 30 - protein, 9 aa, synthetic construct
  REGION 1..9: note = Synthetic Construct
QNTYDTPWT

SEQ ID NO: 31 - protein, 5 aa, synthetic construct
  REGION 1..5: note = Synthetic Construct
GHAIS

SEQ ID NO: 32 - protein, 17 aa, synthetic construct
  REGION 1..17: note = Synthetic Construct
GIIPGHAKANYAQKFQG

SEQ ID NO: 33 - protein, 10 aa, synthetic construct
  REGION 1..10: note = Synthetic Construct
DGLGIRLFAD

SEQ ID NO: 34 - protein, 11 aa, synthetic construct
  REGION 1..11: note = Synthetic Construct
RASQKIYGALN

SEQ ID NO: 35 - protein, 7 aa, synthetic construct
  REGION 1..7: note = Synthetic Construct
GATNLQS

SEQ ID NO: 36 - protein, 9 aa, synthetic construct
  REGION 1..9: note = Synthetic Construct
QNTYDTPWT

SEQ ID NO: 37 - protein, 5 aa, synthetic construct
  REGION 1..5: note = Synthetic Construct
DYGMS

SEQ ID NO: 38 - protein, 17 aa, synthetic construct
  REGION 1..17: note = Synthetic Construct
DINWSGEITAYADSVKG

SEQ ID NO: 39 - protein, 12 aa, synthetic construct
  REGION 1..12: note = Synthetic Construct
SNSYRWDDALDI

SEQ ID NO: 40 - protein, 11 aa, synthetic construct
  REGION 1..11: note = Synthetic Construct
RASQSVSSNLA

SEQ ID NO: 41 - protein, 7 aa, synthetic construct
  REGION 1..7: note = Synthetic Construct
GASSRAT

SEQ ID NO: 42 - protein, 10 aa, synthetic construct
  REGION 1..10: note = Synthetic Construct
QQYGSSPPVT

SEQ ID NO: 43 - protein, 5 aa, synthetic construct
  REGION 1..5: note = Synthetic Construct
DYAMH

SEQ ID NO: 44 - protein, 17 aa, synthetic construct
  REGION 1..17: note = Synthetic Construct
VISYDGSNKYYADSVKG

SEQ ID NO: 45 - protein, 13 aa, synthetic construct
  REGION 1..13: note = Synthetic Construct
DRIGWYDYDAFDI

SEQ ID NO: 46 - protein, 11 aa, synthetic construct
  REGION 1..11: note = Synthetic Construct
RASHSVGSNLA

SEQ ID NO: 47 - protein, 7 aa, synthetic construct
  REGION 1..7: note = Synthetic Construct
DASNRAT

SEQ ID NO: 48 - protein, 9 aa, synthetic construct
  REGION 1..9: note = Synthetic Construct
QQRSNWPPT

SEQ ID NO: 49 - protein, 7 aa, synthetic construct
  REGION 1..7: note = Synthetic Construct
GYTFTKY

SEQ ID NO: 50 - protein, 6 aa, synthetic construct
  REGION 1..6: note = Synthetic Construct
NPNNDE

SEQ ID NO: 51 - protein, 7 aa, synthetic construct
  REGION 1..7: note = Synthetic Construct
GYRFHNF

SEQ ID NO: 52 - protein, 6 aa, synthetic construct
  REGION 1..6: note = Synthetic Construct
IPGHAK

SEQ ID NO: 53 - protein, 7 aa, synthetic construct
  REGION 1..7: note = Synthetic Construct
GSRFYKF

SEQ ID NO: 54 - protein, 7 aa, synthetic construct
  REGION 1..7: note = Synthetic Construct
GSGFRGH

SEQ ID NO: 55 - protein, 7 aa, synthetic construct
  REGION 1..7: note = Synthetic Construct
GFTFDDY

SEQ ID NO: 56 - protein, 6 aa, synthetic construct
  REGION 1..6: note = Synthetic Construct
NWSGEI

SEQ ID NO: 57 - protein, 6 aa, synthetic construct
  REGION 1..6: note = Synthetic Construct
SYDGSN

SEQ ID NO: 58 - protein, 119 aa, synthetic construct
  REGION 1..119: note = Synthetic Construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLITVSA

SEQ ID NO: 59 - protein, 107 aa, synthetic construct
  REGION 1..107: note = Synthetic Construct
DIQMTQSPASLSASVGETVTITCGASENIYGALNWYQRKQGKSPQLLIFGATNLADGVSSRFSGSGSDRQYSLKISSLHPDDVATYYCQNILDTPWTFGGGTKLEIK

SEQ ID NO: 60 - protein, 119 aa, synthetic construct
  REGION 1..119: note = Synthetic Construct
QVQLVQSGAEVKKPGSSVKVSCKASGYTFTKYAISWVRQAPGQGLEWMGHFNPNNDETKYNQKFQGRVTITADESTSTAYMELSSLRSEDTAVYYCARDGLGLRLFADWGQGTLVTVSS

SEQ ID NO: 61 - protein, 107 aa, synthetic construct
  REGION 1..107: note = Synthetic Construct
DIQMTQSPSSLSASVGDRVTITCRASENIYGALNWYQQKPGKAPKLLIYGATNLADGVPSRFSGSGSGTDFTLTISSLQPEDFATYYCQNILDTPWTFGGGTKLEIK

SEQ ID NO: 62 - protein, 119 aa, synthetic construct
  REGION 1..119: note = Synthetic Construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 63 - protein, 107 aa, synthetic construct
  REGION 1..107: note = Synthetic Construct
DIQMTQSPSSLSASVGDRVTITCRASQEIYGALNWYQQKPGKAPKLLIYGATNLQSGVPSRFSGSGSGTDFTLTISSLQPEDFATYYCQDIYDAPWTFGGGTKVEIK

SEQ ID NO: 64 - protein, 119 aa, synthetic construct
  REGION 1..119: note = Synthetic Construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 65 - protein, 107 aa, synthetic construct; identical to SEQ ID NO: 63
  REGION 1..107: note = Synthetic Construct

SEQ ID NO: 66 - protein, 119 aa, synthetic construct
  REGION 1..119: note = Synthetic Construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 67 - protein, 107 aa, synthetic construct
  REGION 1..107: note = Synthetic Construct
DIQMTQSPSSLSASVGDRVTITCRASQKIYGALNWYQQKPGKAPKLLIYGATNLQSGVPSRFSGSGSGTDFTLTISSLQPEDFATYYCQNTYDTPWTFGGGTKVEIK

SEQ ID NO: 68 - protein, 119 aa, synthetic construct
  REGION 1..119: note = Synthetic Construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 69 - protein, 107 aa, synthetic construct; identical to SEQ ID NO: 67
  REGION 1..107: note = Synthetic Construct

SEQ ID NO: 70 - protein, 121 aa, synthetic construct
  REGION 1..121: note = Synthetic Construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSS

SEQ ID NO: 71 - protein, 108 aa, synthetic construct
  REGION 1..108: note = Synthetic Construct
EIVLTQSPATLSVSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 72 - protein, 122 aa, synthetic construct
  REGION 1..122: note = Synthetic Construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSS

SEQ ID NO: 73 - protein, 107 aa, synthetic construct
  REGION 1..107: note = Synthetic Construct
EIVLTQSPATLSVTPGEGATLSCRASHSVGSNLAWYQQKPGQAPRLLIYDASNRATGIPARFSGSGSGTDFTLTISSLEPEDLAVYYCQQRSNWPPTFGQGTRLEIK

SEQ ID NO: 74 - protein, 5 aa, synthetic construct
  REGION 1..5: note = Synthetic Construct
  VARIANT 1..4: note = Can be present in repeats of 1, 2, 3, 4, 5, 6, 7, 8, 9, 10, 11, or 12
  VARIANT 5: note = Can be present in repeats of 0, 1, 2, or 3
GGGSG

SEQ ID NO: 75 - protein, 6 aa, synthetic construct
  REGION 1..6: note = Synthetic Construct
  VARIANT 1..5: note = Can be present in repeats of 1, 2, 3, 4, 5, 6, 7, 8, 9, 10, 11, or 12
  VARIANT 6: note = Can be present in repeats of 0, 1, 2, or 3
GGGGSG

SEQ ID NO: 76 - protein, 7 aa, synthetic construct
  REGION 1..7: note = Synthetic Construct
  VARIANT 1..6: note = Can be present in repeats of 1, 2, 3, 4, 5, 6, 7, 8, 9, 10, 11, or 12
  VARIANT 7: note = Can be present in repeats of 0, 1, 2, or 3
GGGGGSG

SEQ ID NO: 77 - protein, 6 aa, synthetic construct
  REGION 1..6: note = Synthetic Construct
  VARIANT 1..5: note = Can be present in repeats of 2, 3, or 4
  VARIANT 6: note = Can be present in repeats of 0
GGGGSG

SEQ ID NO: 78 - protein, 6 aa, synthetic construct
  REGION 1..6: note = Synthetic Construct
  VARIANT 1..5: note = Can be present in repeats of 3
  VARIANT 6: note = Can be present in repeats of 0
GGGGSG

SEQ ID NO: 79 - protein, 15 aa, synthetic construct
  REGION 1..15: note = Synthetic Construct
GGGGSGGGGSGGGGS

SEQ ID NO: 80 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 81 - protein, 133 aa, synthetic construct [sequence omitted]

SEQ ID NO: 82 - protein, 272 aa, synthetic construct
  REGION 1..272: note = Synthetic Construct
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 83 - protein, 551 aa, synthetic construct
  REGION 1..551: note = Synthetic Construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 84 - protein, 369 aa, synthetic construct
  REGION 1..369: note = Synthetic Construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 85 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 87 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 88 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 90 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 91 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 92 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 93 - protein, 133 aa, synthetic construct
  REGION 1..133: note = Synthetic Construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 133 aa, synthetic construct
  REGION 1..133: note = Synthetic Construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 96 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 97 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 98 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 99 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 100 - protein, 133 aa, synthetic construct
  REGION 1..133: note = Synthetic Construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 101 - protein, 133 aa, synthetic construct
  REGION 1..133: note = Synthetic Construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 102 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 103 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 104 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 105 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 106 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 107 - protein, 133 aa, synthetic construct
  REGION 1..133: note = Synthetic Construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 108 - protein, 133 aa, synthetic construct
  REGION 1..133: note = Synthetic Construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 109 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 110 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFCQSIISTLT

SEQ ID NO: 111 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFCQSIISTLT

SEQ ID NO: 112 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFCQSIISTLT

SEQ ID NO: 113 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFCQSIISTLT

SEQ ID NO: 114 - protein, 133 aa, synthetic construct
  REGION 1..133: note = Synthetic Construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 115 - protein, 133 aa, synthetic construct
  REGION 1..133: note = Synthetic Construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 116 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCSSIISTLT

SEQ ID NO: 117 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 118 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 119 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 120 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 121 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 122 - protein, 133 aa, synthetic construct; identical to SEQ ID NO: 80
  REGION 1..133: note = Synthetic Construct

SEQ ID NO: 123 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 124 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 125 - protein, 133 aa, synthetic construct
  REGION 1..133: note = Synthetic Construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 126 - protein, 133 aa, synthetic construct
  REGION 1..133: note = Synthetic Construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 127 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 128 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 129 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 130 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 131 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 132 - protein, 133 aa, synthetic construct
  REGION 1..133: note = Synthetic Construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 133 - protein, 133 aa, synthetic construct
  REGION 1..133: note = Synthetic Construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 134 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 135 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 136 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 137 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 138 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 139 - protein, 133 aa, synthetic construct
  REGION 1..133: note = Synthetic Construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 140 - protein, 133 aa, synthetic construct
  REGION 1..133: note = Synthetic Construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 141 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFASSIISTLT

SEQ ID NO: 142 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFAQSIISTLT

SEQ ID NO: 143 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFAQSIISTLT

SEQ ID NO: 144 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFAQSIISTLT

SEQ ID NO: 145 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFAQSIISTLT

SEQ ID NO: 146 - protein, 133 aa, synthetic construct
  REGION 1..133: note = Synthetic Construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 147 - protein, 133 aa, synthetic construct
  REGION 1..133: note = Synthetic Construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 148 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFASSIISTLT

SEQ ID NO: 149 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 150 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 151 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 152 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 153 - protein, 133 aa, synthetic construct
  REGION 1..133: note = Synthetic Construct
APTSSSTKKTQLQLEDLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 154 - protein, 133 aa, synthetic construct
  REGION 1..133: note = Synthetic Construct
APTSSSTKKTQLQLEELLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 155 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 156 - protein, 214 aa, synthetic construct
  REGION 1..214: note = Synthetic Construct
DIQMTQSPASLSASVGETVTITCGASENIYGALNWYQRKQGKSPQLLIFGATNLADGVSSRFSGSGSDRQYSLKISSLHPDDVATYYCQNILDTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 597 aa, synthetic construct
  REGION 1..597: note = Synthetic Construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 158 - protein, 448 aa, synthetic construct
  REGION 1..448: note = Synthetic Construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 214 aa, synthetic construct
  REGION 1..214: note = Synthetic Construct
DIQMTQSPSSLSASVGDRVTITCRASQEIYGALNWYQQKPGKAPKLLIYGATNLQSGVPSRFSGSGSGTDFTLTISSLQPEDFATYYCQDIYDAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 597 aa, synthetic construct
  REGION 1..597: note = Synthetic Construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 161 - protein, 448 aa, synthetic construct
  REGION 1..448: note = Synthetic Construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 214 aa, synthetic construct; identical to SEQ ID NO: 159
  REGION 1..214: note = Synthetic Construct

SEQ ID NO: 163 - protein, 597 aa, synthetic construct
  REGION 1..597: note = Synthetic Construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 164 - protein, 448 aa, synthetic construct
  REGION 1..448: note = Synthetic Construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 214 aa, synthetic construct
  REGION 1..214: note = Synthetic Construct
DIQMTQSPSSLSASVGDRVTITCRASQKIYGALNWYQQKPGKAPKLLIYGATNLQSGVPSRFSGSGSGTDFTLTISSLQPEDFATYYCQNTYDTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 597 aa, synthetic construct
  REGION 1..597: note = Synthetic Construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 167 - protein, 448 aa, synthetic construct
  REGION 1..448: note = Synthetic Construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 214 aa, synthetic construct; identical to SEQ ID NO: 165
  REGION 1..214: note = Synthetic Construct

SEQ ID NO: 169 - protein, 597 aa, synthetic construct
  REGION 1..597: note = Synthetic Construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 170 - protein, 448 aa, synthetic construct
  REGION 1..448: note = Synthetic Construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 215 aa, synthetic construct
  REGION 1..215: note = Synthetic Construct
EIVLTQSPATLSVSPGERATLSCRASQSVSSNLAWYQQKPGQAPRLLIYGASSRATGIPDRFSGSGSGTDFTLTISRLEPEDFAVYYCQQYGSSPPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 599 aa, synthetic construct
  REGION 1..599: note = Synthetic Construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 173 - protein, 450 aa, synthetic construct
  REGION 1..450: note = Synthetic Construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 214 aa, synthetic construct
  REGION 1..214: note = Synthetic Construct
EIVLTQSPATLSVTPGEGATLSCRASHSVGSNLAWYQQKPGQAPRLLIYDASNRATGIPARFSGSGSGTDFTLTISSLEPEDLAVYYCQQRSNWPPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 600 aa, synthetic construct
  REGION 1..600: note = Synthetic Construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 176 - protein, 451 aa, synthetic construct
  REGION 1..451: note = Synthetic Construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 5 aa, synthetic construct
  REGION 1..5: note = Synthetic Construct
SYWMN

SEQ ID NO: 178 - protein, 17 aa, synthetic construct
  REGION 1..17: note = Synthetic Construct
QIYPGDGDTNYNGKFKG

SEQ ID NO: 179 - protein, 13 aa, synthetic construct
  REGION 1..13: note = Synthetic Construct
SGAAFSSYYAMDY

SEQ ID NO: 180 - protein, 11 aa, synthetic construct
  REGION 1..11: note = Synthetic Construct
RASENIYSNLA

SEQ ID NO: 181 - protein, 7 aa, synthetic construct
  REGION 1..7: note = Synthetic Construct
AATNLAD

SEQ ID NO: 182 - protein, 9 aa, synthetic construct
  REGION 1..9: note = Synthetic Construct
QHFWGTPWT

SEQ ID NO: 183 - protein, 7 aa, synthetic construct
  REGION 1..7: note = Synthetic Construct
GYAFSSY

SEQ ID NO: 184 - protein, 6 aa, synthetic construct
  REGION 1..6: note = Synthetic Construct
YPGDGD

SEQ ID NO: 185 - protein, 122 aa, synthetic construct
  REGION 1..122: note = Synthetic Construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSS

SEQ ID NO: 186 - protein, 107 aa, synthetic construct
  REGION 1..107: note = Synthetic Construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIK

SEQ ID NO: 187 - protein, 214 aa, synthetic construct
  REGION 1..214: note = Synthetic Construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 600 aa, synthetic construct
  REGION 1..600: note = Synthetic Construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 189 - protein, 451 aa, synthetic construct
  REGION 1..451: note = Synthetic Construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 191 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 192 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 193 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 194 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 195 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 196 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 197 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 198 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 199 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISGINVIVLELKGSETTFMCEYADETATIVEFLNRWITFCQSIISTLT

SEQ ID NO: 200 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRKLISNINVIVLELKGSETTFMCEYADETATIVEFLNRWITFCQSIISTLT

SEQ ID NO: 201 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRRLISNINVIVLELKGSETTFMCEYADETATIVEFLNRWITFCQSIISTLT

SEQ ID NO: 202 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 203 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 204 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 205 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 206 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 207 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 208 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 209 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 210 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 211 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISGINVIVLELKGSETTFMCEYADETATIVEFLNRWITFAQSIISTLT

SEQ ID NO: 212 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRKLISNINVIVLELKGSETTFMCEYADETATIVEFLNRWITFAQSIISTLT

SEQ ID NO: 213 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRRLISNINVIVLELKGSETTFMCEYADETATIVEFLNRWITFAQSIISTLT

SEQ ID NO: 214 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 215 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 216 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 217 - protein, 449 aa, synthetic construct
  REGION 1..449: note = Synthetic Construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 449 aa, synthetic construct
  REGION 1..449: note = Synthetic Construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 449 aa, synthetic construct
  REGION 1..449: note = Synthetic Construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 449 aa, synthetic construct
  REGION 1..449: note = Synthetic Construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 449 aa, synthetic construct
  REGION 1..449: note = Synthetic Construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 451 aa, synthetic construct
  REGION 1..451: note = Synthetic Construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 452 aa, synthetic construct
  REGION 1..452: note = Synthetic Construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 452 aa, synthetic construct
  REGION 1..452: note = Synthetic Construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 5 aa, synthetic construct
  REGION 1..5: note = Synthetic Construct
SYAIS

SEQ ID NO: 226 - protein, 17 aa, synthetic construct
  REGION 1..17: note = Synthetic Construct
GIIPGAATANYAQKFQG

SEQ ID NO: 227 - protein, 10 aa, synthetic construct
  REGION 1..10: note = Synthetic Construct
DAAGIRLFAD

SEQ ID NO: 228 - protein, 9 aa, synthetic construct
  REGION 1..9: note = Synthetic Construct
QSTYDAPWT

SEQ ID NO: 229 - protein, 5 aa, synthetic construct
  REGION 1..5: note = Synthetic Construct
SYAMS

SEQ ID NO: 230 - protein, 17 aa, synthetic construct
  REGION 1..17: note = Synthetic Construct
DITYAGGSTAYADSVKG

SEQ ID NO: 231 - protein, 12 aa, synthetic construct
  REGION 1..12: note = Synthetic Construct
SNAYAWDDALDI

SEQ ID NO: 232 - protein, 17 aa, synthetic construct
  REGION 1..17: note = Synthetic Construct
GIIPGYATANYAQKFQG

SEQ ID NO: 233 - protein, 10 aa, synthetic construct
  REGION 1..10: note = Synthetic Construct
DAAGIRLFAD

SEQ ID NO: 234 - protein, 11 aa, synthetic construct
  REGION 1..11: note = Synthetic Construct
RASQSIYGALN

SEQ ID NO: 235 - protein, 7 aa, synthetic construct
  REGION 1..7: note = Synthetic Construct
GASNLQS

SEQ ID NO: 236 - protein, 9 aa, synthetic construct
  REGION 1..9: note = Synthetic Construct
QSTYTAPWT

SEQ ID NO: 237 - protein, 17 aa, synthetic construct
  REGION 1..17: note = Synthetic Construct
DISYAGGSTAYADSVKG

SEQ ID NO: 238 - protein, 7 aa, synthetic construct
  REGION 1..7: note = Synthetic Construct
GGTFSSY

SEQ ID NO: 239 - protein, 6 aa, synthetic construct
  REGION 1..6: note = Synthetic Construct
IPGAAT

SEQ ID NO: 240 - protein, 7 aa, synthetic construct
  REGION 1..7: note = Synthetic Construct
GFTFSSY

SEQ ID NO: 241 - protein, 6 aa, synthetic construct
  REGION 1..6: note = Synthetic Construct
TYAGGS

SEQ ID NO: 242 - protein, 12 aa, synthetic construct
  REGION 1..12: note = Synthetic Construct
SNAYAWDDALDI

SEQ ID NO: 243 - protein, 6 aa, synthetic construct
  REGION 1..6: note = Synthetic Construct
IPGYAT

SEQ ID NO: 244 - protein, 6 aa, synthetic construct
  REGION 1..6: note = Synthetic Construct
SYAGGS

SEQ ID NO: 245 - protein, 119 aa, synthetic construct
  REGION 1..119: note = Synthetic Construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSS

SEQ ID NO: 246 - protein, 107 aa, synthetic construct
  REGION 1..107: note = Synthetic Construct
DIQMTQSPSSLSASVGDRVTITCRASQEIYGALNWYQQKPGKAPKLLIYGATNLQSGVPSRFSGSGSGTDFTLTISSLQPEDFATYYCQSTYDAPWTFGGGTKVEIK

SEQ ID NO: 247 - protein, 121 aa, synthetic construct
  REGION 1..121: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSS

SEQ ID NO: 248 - protein, 108 aa, synthetic construct
  REGION 1..108: note = Synthetic Construct
EIVLTQSPGTLSLSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 249 - protein, 121 aa, synthetic construct
  REGION 1..121: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSS

SEQ ID NO: 250 - protein, 108 aa, synthetic construct; identical to SEQ ID NO: 248
  REGION 1..108: note = Synthetic Construct

SEQ ID NO: 251 - protein, 119 aa, synthetic construct
  REGION 1..119: note = Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSS

SEQ ID NO: 252 - protein, 107 aa, synthetic construct
  REGION 1..107: note = Synthetic Construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIK

SEQ ID NO: 253 - protein, 119 aa, synthetic construct; identical to SEQ ID NO: 251
  REGION 1..119: note = Synthetic Construct

SEQ ID NO: 254 - protein, 107 aa, synthetic construct; identical to SEQ ID NO: 246
  REGION 1..107: note = Synthetic Construct

SEQ ID NO: 255 - protein, 121 aa, synthetic construct
  REGION 1..121: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSS

SEQ ID NO: 256 - protein, 108 aa, synthetic construct; identical to SEQ ID NO: 248
  REGION 1..108: note = Synthetic Construct

SEQ ID NO: 257 - protein, 121 aa, synthetic construct
  REGION 1..121: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSS

SEQ ID NO: 258 - protein, 108 aa, synthetic construct; identical to SEQ ID NO: 248
  REGION 1..108: note = Synthetic Construct

SEQ ID NO: 259 - skipped

SEQ ID NO: 260 - protein, 17 aa, synthetic construct
  REGION 1..17: note = Synthetic Construct
  VARIANT 1: note = Xaa = G or H
  VARIANT 2: note = Xaa = I or F
  VARIANT 3: note = Xaa = I, N, or M
  VARIANT 5: note = Xaa = G, N, H, S, R, I, or A
  VARIANT 6: note = Xaa = A, N, H, S, T, F, or Y
  VARIANT 7: note = Xaa = A, D, or G
  VARIANT 8: note = Xaa = T, E, K, V, Q, or A
  VARIANT 9: note = Xaa = A or T
  VARIANT 10: note = Xaa = N or K
  VARIANT 12: note = Xaa = A or N
  VARIANT 16: note = Xaa = Q or T
XXXPXXXXXXYXQKFXG

SEQ ID NO: 261 - skipped

SEQ ID NO: 262 - protein, 11 aa, synthetic construct
  REGION 1..11: note = Synthetic Construct
  VARIANT 1: note = Xaa = R or G
  VARIANT 2: note = Xaa = A or T
  VARIANT 4: note = Xaa = Q or E
  VARIANT 5: note = Xaa = E, N, T, S, A, K, D, G, R, or Q
  VARIANT 7: note = Xaa = Y or S
  VARIANT 9: note = Xaa = A or V
XXSXXIXGXLN

SEQ ID NO: 263 - skipped

SEQ ID NO: 264 - protein, 9 aa, synthetic construct
  REGION 1..9: note = Synthetic Construct
  VARIANT 2: note = Xaa = S, N, D, Q, A, or E
  VARIANT 3: note = Xaa = T, I, or S
  VARIANT 4: note = Xaa = Y, L, or F
  VARIANT 5: note = Xaa = D, G, T, E, Q, A, or Y
  VARIANT 6: note = Xaa = A, T, R, S, K, or Y
QXXXXXPWT

SEQ ID NO: 265-268 - skipped (4 entries)

SEQ ID NO: 269 - protein, 17 aa, synthetic construct
  REGION 1..17: note = Synthetic Construct
  VARIANT 3: note = Xaa = T, N, S, Q, E, H, R, or A
  VARIANT 4: note = Xaa = Y, W, F, or H
  VARIANT 5: note = Xaa = A, S, Q, E, or T
  VARIANT 7: note = Xaa = G or E
  VARIANT 8: note = Xaa = S or I
  VARIANT 10: note = Xaa = A or G
DIXXXGXXTXYADSVKG

SEQ ID NO: 270 - protein, 12 aa, synthetic construct
  REGION 1..12: note = Synthetic Construct
  VARIANT 1: note = Xaa = S or A
  VARIANT 2: note = Xaa = N, H, A, D, L, Q, Y, or R
  VARIANT 3: note = Xaa = A, N, S, or G
  VARIANT 5: note = Xaa = A, V, R, E, or S
  VARIANT 7: note = Xaa = D or S
  VARIANT 8: note = Xaa = D, N, Q, E, S, T, or L
  VARIANT 10: note = Xaa = L, F, or M
  VARIANT 12: note = Xaa = I, Y, or V
XXXYXWXXAXDX

SEQ ID NO: 271 - protein, 7 aa, synthetic construct
  REGION 1..7: note = Synthetic Construct
  VARIANT 5: note = Xaa = S, D, E, Q, S, or A
  VARIANT 6: note = Xaa = S, D, E, A, or Q
GFTFXXY

SEQ ID NO: 272 - skipped

SEQ ID NO: 273 - protein, 12 aa, synthetic construct
  REGION 1..12: note = Synthetic Construct
  VARIANT 1: note = Xaa = S or A
  VARIANT 2: note = Xaa = N, H, A, D, L, Q, Y, or R
  VARIANT 3: note = Xaa = A, N, S, or G
  VARIANT 5: note = Xaa = A, V, R, E, or S
  VARIANT 7: note = Xaa = D or S
  VARIANT 8: note = Xaa = D, N, Q, E, S, T, or L
  VARIANT 10: note = Xaa = L, F, or M
  VARIANT 12: note = Xaa = I, Y, or V
XXXYXWXXAXDX

SEQ ID NO: 274 - protein, 30 aa, synthetic construct
  REGION 1..30: note = Synthetic Construct
QVQLVQSGAEVKKPGSSVKVSCKASGGTFS

SEQ ID NO: 275 - protein, 14 aa, synthetic construct
  REGION 1..14: note = Synthetic Construct
WVRQAPGQGLEWMG

SEQ ID NO: 276 - protein, 32 aa, synthetic construct
  REGION 1..32: note = Synthetic Construct
RVTITADESTSTAYMELSSLRSEDTAVYYCAR

SEQ ID NO: 277 - protein, 11 aa, synthetic construct
  REGION 1..11: note = Synthetic Construct
WGQGTLVTVSS

SEQ ID NO: 278 - protein, 25 aa, synthetic construct [sequence omitted]

SEQ ID NO: 279 - protein, 19 aa, synthetic construct
  REGION 1..19: note = Synthetic Construct
AISWVRQAPGQGLEWMGGI

SEQ ID NO: 280 - protein, 41 aa, synthetic construct
  REGION 1..41: note = Synthetic Construct
ANYAQKFQGRVTITADESTSTAYMELSSLRSEDTAVYYCAR

SEQ ID NO: 281 - protein, 30 aa, synthetic construct
  REGION 1..30: note = Synthetic Construct
EVQLVESGGGLVQPGGSLRLSCAASGFTFS

SEQ ID NO: 282 - protein, 14 aa, synthetic construct
  REGION 1..14: note = Synthetic Construct
WVRQAPGKGLEWVS

SEQ ID NO: 283 - protein, 32 aa, synthetic construct
  REGION 1..32: note = Synthetic Construct
RFTISRDNAKNSLYLQMNSLRAEDTAVYYCAR

SEQ ID NO: 284 - protein, 11 aa, synthetic construct
  REGION 1..11: note = Synthetic Construct
WGQGTMVTVSS

SEQ ID NO: 285 - protein, 11 aa, synthetic construct
  REGION 1..11: note = Synthetic Construct
WGQGTLVTVSS

SEQ ID NO: 286 - protein, 25 aa, synthetic construct [sequence omitted]

SEQ ID NO: 287 - protein, 19 aa, synthetic construct
  REGION 1..19: note = Synthetic Construct
AMSWVRQAPGKGLEWVSDI

SEQ ID NO: 288 - protein, 41 aa, synthetic construct
  REGION 1..41: note = Synthetic Construct
TAYADSVKGRFTISRDNAKNSLYLQMNSLRAEDTAVYYCAR

SEQ ID NO: 289 - protein, 23 aa, synthetic construct
  REGION 1..23: note = Synthetic Construct
DIQMTQSPSSLSASVGDRVTITC

SEQ ID NO: 290 - protein, 15 aa, synthetic construct
  REGION 1..15: note = Synthetic Construct
WYQQKPGKAPKLLIY

SEQ ID NO: 291 - protein, 32 aa, synthetic construct
  REGION 1..32: note = Synthetic Construct
GVPSRFSGSGSGTDFTLTISSLQPEDFATYYC

SEQ ID NO: 292 - protein, 10 aa, synthetic construct
  REGION 1..10: note = Synthetic Construct
FGGGTKVEIK

SEQ ID NO: 293 - protein, 23 aa, synthetic construct
  REGION 1..23: note = Synthetic Construct
EIVLTQSPGTLSLSPGERATLSC

SEQ ID NO: 294 - protein, 15 aa, synthetic construct
  REGION 1..15: note = Synthetic Construct
WYQQKPGQAPRLLIY

SEQ ID NO: 295 - protein, 32 aa, synthetic construct
  REGION 1..32: note = Synthetic Construct
GIPDRFSGSGSGTDFTLTISRLEPEDFAVYYC

SEQ ID NO: 296 - protein, 10 aa, synthetic construct
  REGION 1..10: note = Synthetic Construct
FGQGTKVEIK

SEQ ID NO: 297 - protein, 133 aa, synthetic construct; identical to SEQ ID NO: 126
  REGION 1..133: note = Synthetic Construct

SEQ ID NO: 298 - protein, 214 aa, synthetic construct
  REGION 1..214: note = Synthetic Construct
DIQMTQSPSSLSASVGDRVTITCRASQEIYGALNWYQQKPGKAPKLLIYGATNLQSGVPSRFSGSGSGTDFTLTISSLQPEDFATYYCQSTYDAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 597 aa, synthetic construct
  REGION 1..597: note = Synthetic Construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 300 - protein, 448 aa, synthetic construct
  REGION 1..448: note = Synthetic Construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 301 - protein, 449 aa, synthetic construct
  REGION 1..449: note = Synthetic Construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 215 aa, synthetic construct
  REGION 1..215: note = Synthetic Construct
EIVLTQSPGTLSLSPGERATLSCRASQSVSSNLAWYQQKPGQAPRLLIYGASSRATGIPDRFSGSGSGTDFTLTISRLEPEDFAVYYCQQYGSSPPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 303 - protein, 599 aa, synthetic construct
  REGION 1..599: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 304 - protein, 450 aa, synthetic construct
  REGION 1..450: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 305 - protein, 451 aa, synthetic construct
  REGION 1..451: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 215 aa, synthetic construct; identical to SEQ ID NO: 302
  REGION 1..215: note = Synthetic Construct

SEQ ID NO: 307 - protein, 599 aa, synthetic construct
  REGION 1..599: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 308 - protein, 450 aa, synthetic construct
  REGION 1..450: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 309 - protein, 451 aa, synthetic construct
  REGION 1..451: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 214 aa, synthetic construct
  REGION 1..214: note = Synthetic Construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 597 aa, synthetic construct
  REGION 1..597: note = Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 312 - protein, 448 aa, synthetic construct
  REGION 1..448: note = Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 313 - protein, 449 aa, synthetic construct
  REGION 1..449: note = Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 214 aa, synthetic construct; identical to SEQ ID NO: 298
  REGION 1..214: note = Synthetic Construct

SEQ ID NO: 315 - protein, 597 aa, synthetic construct; identical to SEQ ID NO: 311
  REGION 1..597: note = Synthetic Construct

SEQ ID NO: 316 - protein, 448 aa, synthetic construct; identical to SEQ ID NO: 312
  REGION 1..448: note = Synthetic Construct

SEQ ID NO: 317 - protein, 449 aa, synthetic construct; identical to SEQ ID NO: 313
  REGION 1..449: note = Synthetic Construct

SEQ ID NO: 318 - protein, 215 aa, synthetic construct; identical to SEQ ID NO: 302
  REGION 1..215: note = Synthetic Construct

SEQ ID NO: 319 - protein, 599 aa, synthetic construct
  REGION 1..599: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 320 - protein, 450 aa, synthetic construct
  REGION 1..450: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 451 aa, synthetic construct
  REGION 1..451: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 215 aa, synthetic construct; identical to SEQ ID NO: 302
  REGION 1..215: note = Synthetic Construct

SEQ ID NO: 323 - protein, 599 aa, synthetic construct
  REGION 1..599: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 324 - protein, 450 aa, synthetic construct
  REGION 1..450: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - protein, 451 aa, synthetic construct
  REGION 1..451: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 214 aa, synthetic construct; identical to SEQ ID NO: 298
  REGION 1..214: note = Synthetic Construct

SEQ ID NO: 327 - protein, 597 aa, synthetic construct
  REGION 1..597: note = Synthetic Construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 328 - protein, 448 aa, synthetic construct; identical to SEQ ID NO: 300
  REGION 1..448: note = Synthetic Construct

SEQ ID NO: 329 - protein, 449 aa, synthetic construct; identical to SEQ ID NO: 301
  REGION 1..449: note = Synthetic Construct

SEQ ID NO: 330 - protein, 215 aa, synthetic construct; identical to SEQ ID NO: 302
  REGION 1..215: note = Synthetic Construct

SEQ ID NO: 331 - protein, 599 aa, synthetic construct
  REGION 1..599: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 332 - protein, 450 aa, synthetic construct; identical to SEQ ID NO: 304
  REGION 1..450: note = Synthetic Construct

SEQ ID NO: 333 - protein, 451 aa, synthetic construct; identical to SEQ ID NO: 305
  REGION 1..451: note = Synthetic Construct

SEQ ID NO: 334 - protein, 215 aa, synthetic construct; identical to SEQ ID NO: 302
  REGION 1..215: note = Synthetic Construct

SEQ ID NO: 335 - protein, 599 aa, synthetic construct
  REGION 1..599: note = Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 336 - protein, 450 aa, synthetic construct; identical to SEQ ID NO: 308
  REGION 1..450: note = Synthetic Construct

SEQ ID NO: 337 - protein, 451 aa, synthetic construct; identical to SEQ ID NO: 309
  REGION 1..451: note = Synthetic Construct

SEQ ID NO: 338 - protein, 214 aa, synthetic construct; identical to SEQ ID NO: 310
  REGION 1..214: note = Synthetic Construct

SEQ ID NO: 339 - protein, 597 aa, synthetic construct
  REGION 1..597: note = Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 340 - protein, 448 aa, synthetic construct; identical to SEQ ID NO: 312
  REGION 1..448: note = Synthetic Construct

SEQ ID NO: 341 - protein, 449 aa, synthetic construct; identical to SEQ ID NO: 313
  REGION 1..449: note = Synthetic Construct

SEQ ID NO: 342 - protein, 214 aa, synthetic construct; identical to SEQ ID NO: 298
  REGION 1..214: note = Synthetic Construct

SEQ ID NO: 343 - protein, 597 aa, synthetic construct; identical to SEQ ID NO: 339
  REGION 1..597: note = Synthetic Construct

SEQ ID NO: 344 - protein, 448 aa, synthetic construct; identical to SEQ ID NO: 312
  REGION 1..448: note = Synthetic Construct

SEQ ID NO: 345 - protein, 449 aa, synthetic construct; identical to SEQ ID NO: 313
  REGION 1..449: note = Synthetic Construct

SEQ ID NO: 346 - protein, 215 aa, synthetic construct; identical to SEQ ID NO: 302
  REGION 1..215: note = Synthetic Construct

SEQ ID NO: 347 - protein, 599 aa, synthetic construct
  REGION 1..599: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 348 - protein, 450 aa, synthetic construct; identical to SEQ ID NO: 320
  REGION 1..450: note = Synthetic Construct

SEQ ID NO: 349 - protein, 451 aa, synthetic construct; identical to SEQ ID NO: 321
  REGION 1..451: note = Synthetic Construct

SEQ ID NO: 350 - protein, 215 aa, synthetic construct; identical to SEQ ID NO: 302
  REGION 1..215: note = Synthetic Construct

SEQ ID NO: 351 - protein, 599 aa, synthetic construct
  REGION 1..599: note = Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 352 - protein, 450 aa, synthetic construct; identical to SEQ ID NO: 324
  REGION 1..450: note = Synthetic Construct

SEQ ID NO: 353 - protein, 451 aa, synthetic construct; identical to SEQ ID NO: 325
  REGION 1..451: note = Synthetic Construct

SEQ ID NO: 354 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 355 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 356 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 357 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 358 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 359 - protein, 133 aa, synthetic construct
  REGION 1..133: note = Synthetic Construct
APTSSSTKKTQLQLEHLLLDLQMILNGINNYKNPKLTEMLTAKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 360 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 361 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 362 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 363 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 364 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 365 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 366 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISRINVIVLELKGSETTFMCEYADETATIVEFLNRWITFCQSIISTLT

SEQ ID NO: 367 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISTINVIVLELKGSETTFMCEYADETATIVEFLNRWITFCQSIISTLT

SEQ ID NO: 368 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISDINVIVLELKGSETTFMCEYADETATIVEFLNRWITFCQSIISTLT

SEQ ID NO: 369 - protein, 133 aa, synthetic construct; identical to SEQ ID NO: 356
  REGION 1..133: note = Synthetic Construct

SEQ ID NO: 370 - protein, 133 aa, synthetic construct; identical to SEQ ID NO: 355
  REGION 1..133: note = Synthetic Construct

SEQ ID NO: 371 - protein, 133 aa, synthetic construct; identical to SEQ ID NO: 354
  REGION 1..133: note = Synthetic Construct

SEQ ID NO: 372 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 373 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 374 - protein, 133 aa, synthetic construct
  REGION 1..133: note = Synthetic Construct
APTSSSTKKTQLQLEHLLLDLQMILNGINNYKNPKLTDMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 375 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 376 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 377 - protein, 133 aa, synthetic construct
  REGION 1..133: note = Synthetic Construct
APTSSSTKKTQLQLEHLLLDLQMILNGINNYKNPKLTAMLTKKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 378 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISRINVIVLELKGSETTFMCEYADETATIVEFLNRWITFAQSIISTLT

SEQ ID NO: 379 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISTINVIVLELKGSETTFMCEYADETATIVEFLNRWITFAQSIISTLT

SEQ ID NO: 380 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISDINVIVLELKGSETTFMCEYADETATIVEFLNRWITFAQSIISTLT

SEQ ID NO: 381 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 382 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 383 - protein, 133 aa, synthetic construct
  REGION 1..133: note = Synthetic Construct
APTSSSTKKTQLQLEHLLLDLQMILNGINNYKNPKLTRMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 384 - protein, 98 aa, synthetic construct
  REGION 1..98: note = Synthetic Construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 385 - protein, 95 aa, synthetic construct
  REGION 1..95: note = Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 386 - protein, 6 aa, synthetic construct
  REGION 1..6: note = Synthetic Construct
  VARIANT 2..5: note = Can be present in repeats of 1, 2, 3, 4, 5, 6, 7, 8, 9, 10, 11, or 12
  VARIANT 6: note = Can be present in repeats of 0, 1, 2, or 3
SGGGSG

SEQ ID NO: 387 - protein, 7 aa, synthetic construct
  REGION 1..7: note = Synthetic Construct
  VARIANT 2..6: note = Can be present in repeats of 1, 2, 3, 4, 5, 6, 7, 8, 9, 10, 11, or 12
  VARIANT 7: note = Can be present in repeats of 0, 1, 2, or 3
SGGGGSG

SEQ ID NO: 388 - protein, 8 aa, synthetic construct
  REGION 1..8: note = Synthetic Construct
  VARIANT 2..7: note = Can be present in repeats of 1, 2, 3, 4, 5, 6, 7, 8, 9, 10, 11, or 12
  VARIANT 8: note = Can be present in repeats of 0, 1, 2, or 3
SGGGGGSG

SEQ ID NO: 389 - protein, 16 aa, synthetic construct
  REGION 1..16: note = Synthetic Construct
SGGGGSGGGGSGGGGS